(19) United States
(12) Patent Application Publication
(10) Pub. No.: US 2006/0069110 A1
(43) Pub. Date: Mar. 30, 2006

(54) Substituted heterocyclic compounds and methods of use

(76) Inventors: Denise Lyn Andersen, Simi Valley, CA (US); Catherine H. Chang, Ann Arbor, MI (US); James R. Falsey, Moorpark, CA (US); Michael J. Frohn, Thousand Oaks, CA (US); Fang-Tsao Hong, Thousand Oaks, CA (US); Hongyu Liao, Thousand Oaks, CA (US); Longbin Liu, Thousand Oaks, CA (US); Patricia Lopez, West Hills, CA (US); Daniel Martin Retz, Thousand Oaks, CA (US); Gilbert M. Rishton, Malibu, CA (US); Robert M. Rzasa, Ventura, CA (US); Aaron Siegmund, Oxnard, CA (US); Seifu Tadesse, Simi Valley, CA (US); Nuria Tamayo, Newbury Park, CA (US); Christopher M. Tegley, Thousand Oaks, CA (US)

Correspondence Address: AMGEN INC., MAIL STOP 28-2-C, ONE AMGEN CENTER DRIVE, THOUSAND OAKS, CA, 91320-1799, US

(21) Appl. No.: 11/237,513

(22) Filed: Sep. 27, 2005

Related U.S. Application Data
(60) Provisional application No. 60/613,762, filed on Sep. 27, 2004.

(51) Int. Cl. A61K 31/506; C07D 403/14; C07D 403/04

(52) U.S. Cl. 514/275; 544/296; 544/328

(57) ABSTRACT

The present invention relates to pyridines, pyrimidines and derivatives thereof, and pharmaceutically acceptable salts thereof. Also included is a method of treatment of inflammation, rheumatoid arthritis, Pagets disease, osteoporosis, multiple myeloma, uveititis, acute or chronic myelogenous leukemia, pancreatic β cell destruction, osteoarthritis, rheumatoid spondylitis, gouty arthritis, inflammatory bowel disease, adult respiratory distress syndrome (ARDS), psoriasis, Crohn's disease, allergic rhinitis, ulcerative colitis, anaphylaxis, contact dermatitis, asthma, muscle degeneration, cachexia, Reiter's syndrome, type I diabetes, type II diabetes, bone resorption diseases, graft vs. host reaction, Alzheimer's disease, stroke, myocardial infarction, ischemia reperfusion injury, atherosclerosis, brain trauma, multiple sclerosis, cerebral malaria, sepsis, septic shock, toxic shock syndrome, fever, myalgias due to HIV-1, HIV-2, HIV-3, cytomegalovirus (CMV), influenza, adenovirus, the herpes viruses or herpes zoster infection in a mammal comprising administering an effective amount a compound as described above.

This application claims the benefit of U.S. Provisional Application No. 60/613,762 filed Sep. 27, 2004, which is hereby incorporated by reference.

### BACKGROUND OF THE INVENTION

The present invention comprises a new class of compounds useful in treating diseases, such as TNF-α, IL-1β, IL-6 and/or IL-8 mediated diseases and other maladies, such as pain and diabetes. In particular, the compounds of the invention are useful for the prophylaxis and treatment of diseases or conditions involving inflammation. This invention also relates to intermediates and processes useful in the preparation of such compounds.

Interleukin-1 (IL-1) and Tumor Necrosis Factor α (TNF-α) are pro-inflammatory cytokines secreted by a variety of cells, including monocytes and macrophages, in response to many inflammatory stimuli (e.g., lipopolysaccharide—LPS) or external cellular stress (e.g., osmotic shock and peroxide).

Elevated levels of TNF-α and/or IL-1 over basal levels have been implicated in mediating or exacerbating a number of disease states including rheumatoid arthritis; Pagets disease; osteoporosis; multiple myeloma; uveititis; acute and chronic myelogenous leukemia; pancreatic β cell destruction; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; asthma; muscle degeneration; cachexia; Reiter's syndrome; type I and type II diabetes; bone resorption diseases; graft vs. host reaction; ischemia reperfusion injury; atherosclerosis; brain trauma; multiple sclerosis; cerebral malaria; sepsis; septic shock; toxic shock syndrome; fever, and myalgias due to infection. HIV-1, HIV-2, HIV-3, cytomegalovirus (CMV), influenza, adenovirus, the herpes viruses (including HSV-1, HSV-2), and herpes zoster are also exacerbated by TNF-α.

It has been reported that TNF-α plays a role in head trauma, stroke, and ischemia. For instance, in animal models of head trauma (rat), TNF-α levels increased in the contused hemisphere (Shohami et al., J. Cereb. Blood Flow Metab. 14, 615 (1994)). In a rat model of ischemia wherein the middle cerebral artery was occluded, the levels of TNF-α mRNA of TNF-α increased (Feurstein et al., Neurosci. Lett. 164, 125 (1993)). Administration of TNF-α into the rat cortex has been reported to result in significant neutrophil accumulation in capillaries and adherence in small blood vessels. TNF-α promotes the infiltration of other cytokines (IL-1β, IL-6) and also chemokines, which promote neutrophil infiltration into the infarct area (Feurstein, Stroke 25, 1481 (1994)). TNF-α has also been implicated to play a role in type II diabetes (Endocrinol. 130, 43-52, 1994; and Endocrinol. 136, 1474-1481, 1995).

TNF-α appears to play a role in promoting certain viral life cycles and disease states associated with them. For instance, TNF-α secreted by monocytes induced elevated levels of HIV expression in a chronically infected T cell clone (Clouse et al., J. Immunol. 142, 431 (1989)). Lahdevirta et al., (Am. J. Med. 85, 289 (1988)) discussed the role of TNF-α in the HIV associated states of cachexia and muscle degradation.

TNF-α is upstream in the cytokine cascade of inflammation. As a result, elevated levels of TNF-α may lead to elevated levels of other inflammatory and proinflammatory cytokines, such as IL-1, IL-6, and IL-8.

Elevated levels of IL-1 over basal levels have been implicated in mediating or exacerbating a number of disease states including rheumatoid arthritis; osteoarthritis; rheumatoid spondylitis; gouty arthritis; inflammatory bowel disease; adult respiratory distress syndrome (ARDS); psoriasis; Crohn's disease; ulcerative colitis; anaphylaxis; muscle degeneration; cachexia; Reiter's syndrome; type I and type II diabetes; bone resorption diseases; ischemia reperfusion injury; atherosclerosis; brain trauma; multiple sclerosis; sepsis; septic shock; and toxic shock syndrome. Viruses sensitive to TNF-α inhibition, e.g., HIV-1, HIV-2, HIV-3, are also affected by IL-1.

TNF-α and IL-1 appear to play a role in pancreatic β cell destruction and diabetes. Pancreatic β cells produce insulin which helps mediate blood glucose homeostasis. Deterioration of pancreatic β cells often accompanies type I diabetes. Pancreatic β cell functional abnormalities may occur in patients with type II diabetes. Type II diabetes is characterized by a functional resistance to insulin. Further, type II diabetes is also often accompanied by elevated levels of plasma glucagon and increased rates of hepatic glucose production. Glucagon is a regulatory hormone that attenuates liver gluconeogenesis inhibition by insulin. Glucagon receptors have been found in the liver, kidney and adipose tissue. Thus glucagon antagonists are useful for attenuating plasma glucose levels (WO 97/16442, incorporated herein by reference in its entirety). By antagonizing the glucagon receptors, it is thought that insulin responsiveness in the liver will improve, thereby decreasing gluconeogenesis and lowering the rate of hepatic glucose production.

In rheumatoid arthritis models in animals, multiple intra-articular injections of IL-1 have led to an acute and destructive form of arthritis (Chandrasekhar et al., Clinical Immunol Immunopathol. 55, 382 (1990)). In studies using cultured rheumatoid synovial cells, IL-1 is a more potent inducer of stromelysin than is TNF-α (Firestein, Am. J. Pathol. 140, 1309 (1992)). At sites of local injection, neutrophil, lymphocyte, and monocyte emigration has been observed. The emigration is attributed to the induction of chemokines (e.g., IL-8), and the up-regulation of adhesion molecules (Dinarello, Eur. Cytokine Netw. 5, 517-531 (1994)).

IL-1 also appears to play a role in promoting certain viral life cycles. For example, cytokine-induced increase of HIV expression in a chronically infected macrophage line has been associated with a concomitant and selective increase in IL-1 production (Folks et al., J. Immunol. 136, 40 (1986)). Beutler et al. (J. Immunol. 135, 3969 (1985)) discussed the role of IL-1 in cachexia. Baracos et al. (New Eng. J. Med. 308, 553 (1983)) discussed the role of IL-1 in muscle degeneration.

In rheumatoid arthritis, both IL-1 and TNF-α induce synoviocytes and chondrocytes to produce collagenase and neutral proteases, which leads to tissue destruction within the arthritic joints. In a model of arthritis (collagen-induced arthritis (CIA) in rats and mice), intra-articular administration of TNF-α either prior to or after the induction of CIA led to an accelerated onset of arthritis and a more severe course of the disease (Brahn et al., Lymphokine Cytokine Res. 11, 253 (1992); and Cooper, Clin. Exp. Immunol. 898, 244 (1992)).

IL-8 has been implicated in exacerbating and/or causing many disease states in which massive neutrophil infiltration into sites of inflammation or injury (e.g., ischemia) is mediated by the chemotactic nature of IL-8, including, but not limited to, the following: asthma, inflammatory bowel disease, psoriasis, adult respiratory distress syndrome, cardiac and renal reperfusion injury, thrombosis and glomerulonephritis. In addition to the chemotaxis effect on neutrophils, IL-8 also has the ability to activate neutrophils. Thus, reduction in IL-8 levels may lead to diminished neutrophil infiltration.

Several approaches have been taken to block the effect of TNF-α. One approach involves using soluble receptors for TNF-α (e.g., TNFR-55 or TNFR-75), which have demonstrated efficacy in animal models of TNF-α-mediated disease states. A second approach to neutralizing TNF-α using a monoclonal antibody specific to TNF-α, cA2, has demonstrated improvement in swollen joint count in a Phase II human trial of rheumatoid arthritis (Feldmann et al., Immunological Reviews, pp. 195-223 (1995)). These approaches block the effects of TNF-α and IL-1 by either protein sequestration or receptor antagonism.

U.S. Pat. No. 5,100,897, incorporated herein by reference in its entirety, describes pyrimidinone compounds useful as angiotensin II antagonists wherein one of the pyrimidinone ring nitrogen atoms is substituted with a substituted phenylmethyl or phenethyl radical.

U.S. Pat. No. 5,162,325, incorporated herein by reference in its entirety, describes pyrimidinone compounds useful as angiotensin II antagonists wherein one of the pyrimidinone ring nitrogen atoms is substituted with a substituted phenylmethyl radical.

EP 481448, incorporated herein by reference in its entirety, describes pyrimidinone compounds useful as angiotensin II antagonists wherein one of the pyrimidinone ring nitrogen atoms is substituted with a substituted phenyl, phenylmethyl or phenethyl radical.

CA 2,020,370, incorporated herein by reference in its entirety, describes pyrimidinone compounds useful as angiotensin II antagonists wherein one of the pyrimidinone ring nitrogen atoms is substituted with a substituted biphenylaliphatic hydrocarbon radical.

### BRIEF DESCRIPTION OF THE INVENTION

The present invention comprises a new class of compounds useful in the prophylaxis and treatment of diseases, such as TNF-α, IL-1β, IL-6 and/or IL-8 mediated diseases and other maladies, such as pain and diabetes. In particular, the compounds of the invention are useful for the prophylaxis and treatment of diseases or conditions involving inflammation. Accordingly, the invention also comprises pharmaceutical compositions comprising the compounds; methods for the prophylaxis and treatment of TNF-α, IL-1β, IL-6 and/or IL-8 mediated diseases, such as inflammatory, pain and diabetes diseases, using the compounds and compositions of the invention, and intermediates and processes useful for the preparation of the compounds of the invention.

The compounds of the invention are represented by the following general structure: 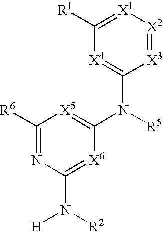
wherein R¹, R², R⁵, R⁶, X¹, X², X³, X⁴, X⁵ and X⁶ are defined herein.

The foregoing merely summarizes certain aspects of the invention and is not intended, nor should it be construed, as limiting the invention in any way. All patents and other publications recited herein are hereby incorporated by reference in their entirety.

### DETAILED DESCRIPTION OF THE INVENTION

In accordance with the present invention, there is provided compounds of the formula: 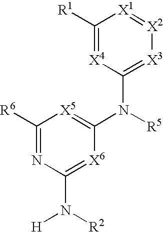
or a pharmaceutically acceptable salt or hydrate thereof, wherein

X¹ is N or CR³;

X² is N or CR⁴; or —X¹═X²— is —C(═O)—N(R^{a})— or —N(R^{a})—C(═O)—;

X³ is N or CR⁴;

X⁴ is N or CR⁴;

X⁵ is N or CR⁶;

X⁶ is N or CR⁶; wherein only 1, 2 or 3 of X¹, X², X³ and X⁴ are N;

R¹ is a saturated, partially saturated or unsaturated 5-, 6- or 7-membered, ring containing 0, 1, 2 or 3 atoms selected from N, O and S, wherein the ring is substituted by 0, 1, 2 or 3 substituents selected from C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a};

R² is C₁₋₈alkyl substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; or

R² is a saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I;

R³ is independently, in each instance, selected from H, R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —NR^{a}R^{e}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a};

R⁴ is independently in each instance H, R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —NR^{a}R^{e}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a};

R⁵ is H, R^{e}, C₁₋₄haloalkyl, —C(═O)R^{b}, —C(═O)OR^{b}, —C(═O)NR^{a}R^{a} or —C(═NR^{a})NR^{a}R^{a};

R⁶ is independently in each instance H, C₁₋₈alkyl, C₁₋₄haloalkyl, —NR^{a}R^{a}, —OR^{a}, or halo;

R^{a} is independently, at each instance, H or R^{b};

R^{b} is independently, at each instance, phenyl, benzyl or C₁₋₆alkyl, the phenyl, benzyl and C₁₋₆alkyl being substituted by 0, 1, 2 or 3 substituents selected from halo, C₁₋₄alkyl, C₁₋₃haloalkyl, —OC₁₋₄alkyl, —NH₂, —NHC₁₋₄alkyl, —N(C₁₋₄alkyl)C₁₋₄alkyl;

R^{d} is independently at each instance C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NRₐC₂₋₆alkylNR^{a}R^{a} or —NR^{a}C₂₋₆alkylOR^{a};

R^{e} is independently at each instance C₁₋₆alkyl substituted by 0, 1, 2 or 3 substituents independently selected from R^{d} and additionally substituted by 0 or 1 substituents selected from R^{g}; and

R^{g} is independently at each instance a saturated, partially saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, wherein the carbon atoms of the ring are substituted by 0, 1 or 2 oxo groups and the ring is substituted by 0, 1, 2 or 3 substituents selected from R^{b}, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from Br, Cl, F and I.

In another embodiment, in conjunction with the above and below embodiments, R¹ is a saturated or unsaturated 5- or 6-membered, ring containing 0, 1, 2 or 3 atoms selected from N, O and S, wherein the ring is substituted by 1, 2 or 3 substituents selected from C₁₋₄alkyl, C₁₋₄haloalkyl, halo, cyano, nitro, —C(═O)R^{b}, —C(═O)OR^{e}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with the above and below embodiments, R¹ is a saturated or unsaturated 5- or 6-membered, ring containing 0, 1, 2 or 3 atoms selected from N, O and S, wherein the ring is substituted by 1, 2 or 3 substituents selected from C₁₋₄alkyl, C₁₋₄haloalkyl, halo, cyano, nitro, —OR^{a}, —OC(═O)R^{b}, —SR^{a}, —S(═O)R^{b}, —S(═O)₂R^{b}, —NR^{a}R^{a} and —N(R^{a})C(═O)R^{b}.

In another embodiment, in conjunction with the above and below embodiments, R¹ is a saturated or unsaturated 5- or 6-membered, ring containing 0, 1, 2 or 3 atoms selected from N, O and S, wherein the ring is substituted by 0, 1, 2 or 3 substituents selected from C₁₋₄alkyl, C₁₋₄haloalkyl and halo.

In another embodiment, in conjunction with the above and below embodiments, R¹ is a saturated or unsaturated 6-membered, ring containing 0, 1, 2 or 3 atoms selected from N, O and S, wherein the ring is substituted by 0, 1, 2 or 3 substituents selected from C₁₋₄alkyl, C₁₋₄haloalkyl and halo.

In another embodiment, in conjunction with the above and below embodiments, R¹ is phenyl substituted by 0, 1, 2 or 3 substituents selected from C₁₋₄alkyl, C₁₋₄haloalkyl and halo.

In another embodiment, in conjunction with the above and below embodiments, R¹ is phenyl.

In another embodiment, in conjunction with the above and below embodiments, R¹ is phenyl substituted by 1, 2 or 3 substituents selected from C₁₋₄alkyl, C₁₋₄haloalkyl and halo.

In another embodiment, in conjunction with the above and below embodiments, R¹ is pyridinyl substituted by 0, 1, 2 or 3 substituents selected from C₁₋₄alkyl, C₁₋₄haloalkyl and halo.

In another embodiment, in conjunction with the above and below embodiments, R¹ is pyrimidinyl substituted by 0, 1, 2 or 3 substituents selected from C₁₋₄alkyl, C₁₋₄haloalkyl and halo.

In another embodiment, in conjunction with the above and below embodiments, R¹ is a saturated or unsaturated 5-membered, ring containing 1 or 2 atoms selected from N, O and S, wherein the ring is substituted by 0, 1, 2 or 3 substituents selected from C₁₋₄alkyl, C₁₋₄haloalkyl and halo.

In another embodiment, in conjunction with the above and below embodiments, R² is C₁₋₈alkyl substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with the above and below embodiments, R² is C₁₋₈alkyl.

In another embodiment, in conjunction with the above and below embodiments, R² is C₁₋₈alkyl substituted by 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkyNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkyNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with the above and below embodiments, R² is C₁₋₈alkyl substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with the above and below embodiments, R² is C₂₋₈alkyl substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with the above and below embodiments, R² is a saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I.

In another embodiment, in conjunction with the above and below embodiments, R² is a saturated, partially saturated or unsaturated 5-, 6- or 7-membered monocyclic ring containing 1, 2 or 3 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I.

In another embodiment, in conjunction with the above and below embodiments, R² is a saturated or partially saturated 5-, 6- or 7-membered monocyclic ring containing 1, 2 or 3 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆akylNR^{a}R^{e}, —OC₂₋₆OR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I.

In another embodiment, in conjunction with the above and below embodiments, R² is a saturated or partially saturated 5-, 6- or 7-membered monocyclic ring containing 1, 2 or 3 atoms selected from N, O and S, wherein the carbon atoms of the rings are substituted by 0, 1 or 2 oxo groups and the rings is substituted by 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I.

In another embodiment, in conjunction with the above and below embodiments, R² is a saturated or partially saturated 5-, 6- or 7-membered monocyclic ring containing 1, 2 or 3 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 1 or 2 C₁₋₈alkyl groups, each being substituted by 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a} C₂₋₆alkylOR^{a}; wherein any part of R² is additionally substituted by 0, 1, 2, 3, 4, 5 or 6 atoms selected from Br, Cl, F and I.

In another embodiment, in conjunction with the above and below embodiments, R² is a saturated or partially saturated 5-, 6- or 7-membered monocyclic ring containing 1 or 2 N atoms, wherein the carbon atoms of the rings are substituted by 0, 1 or 2 oxo groups and the rings is substituted by 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I.

In another embodiment, in conjunction with the above and below embodiments, R³ is independently, in each instance, selected from H, R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —NR^{a}R^{e}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with the above and below embodiments, R³ is H.

In another embodiment, in conjunction with the above and below embodiments, R³ is independently, in each instance, selected from H, C₁₋₆alkyl, C₁₋₄haloalkyl and halo.

In another embodiment, in conjunction with the above and below embodiments, R³ is independently, in each instance, selected from R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —NR^{a}R^{e}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with any of the above and below embodiments, R⁴ is independently in each instance R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —NR^{a}R^{e}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a}.

In another embodiment, in conjunction with any of the above and below embodiments, R⁴ is H.

In another embodiment, in conjunction with any of the above and below embodiments, R⁵ is H.

In another embodiment, in conjunction with any of the above and below embodiments, R⁵ is R^{e}, C₁₋₄haloalkyl, —C(═O)R^{b}, —C(═O)OR^{b}, —C(═O)NR^{a}R^{a} or —C(═NR^{a})NR^{a}R^{a}.

In another embodiment, in conjunction with any of the above and below embodiments, R⁶ is H.

In another embodiment, in conjunction with any of the above and below embodiments, R⁶ is independently in each instance C₁₋₈alkyl, C₁₋₄haloalkyl, —NR^{a}R^{a}, —OR^{a}, or halo.

In another embodiment, in conjunction with any of the above and below embodiments, —X¹═X²— is —C(═O)—N(R^{a})— or —N(R^{a})—C(═O)—.

In another embodiment, in conjunction with any of the above and below embodiments, X¹ is N or CR³ and X² is N or CR⁴.

In another embodiment, in conjunction with any of the above and below embodiments, X¹ is CR³ and X² is N.

In another embodiment, in conjunction with any of the above and below embodiments, X¹ is N and X² is CR⁴.

In another embodiment, in conjunction with any of the above and below embodiments, X¹ is CR³ and X² is CR⁴.

In another embodiment, in conjunction with any of the above and below embodiments, X³ is N and X⁴ is CR⁴.

In another embodiment, in conjunction with any of the above and below embodiments, X³ is CR⁴ and X⁴ is N.

In another embodiment, in conjunction with any of the above and below embodiments, X³ is N and X⁴ is N.

In another embodiment, in conjunction with any of the above and below embodiments, X⁵ is N and X⁶ is CR⁶.

In another embodiment, in conjunction with any of the above and below embodiments, X⁵ is CR⁶ and X⁶ is N.

In another embodiment, in conjunction with any of the above and below embodiments, X⁵ is CR⁶ and X⁶ is CR⁶.

Another aspect of the invention relates to a pharmaceutical composition comprising a compound according to any one of the above embodiments and a pharmaceutically acceptable carrier.

Another aspect of the invention relates to a method of prophylaxis or treatment of inflammation comprising administering an effective amount of a compound according to any one of the above embodiments.

Another aspect of the invention relates to a method of prophylaxis or treatment of rheumatoid arthritis, Pagets disease, osteoporosis, multiple myeloma, uveititis, acute or chronic myelogenous leukemia, pancreatic β cell destruction, osteoarthritis, rheumatoid spondylitis, gouty arthritis, inflammatory bowel disease, adult respiratory distress syndrome (ARDS), psoriasis, Crohn's disease, allergic rhinitis, ulcerative colitis, anaphylaxis, contact dermatitis, asthma, muscle degeneration, cachexia, Reiter's syndrome, type I diabetes, type II diabetes, bone resorption diseases, graft vs. host reaction, Alzheimer's disease, stroke, myocardial infarction, ischemia reperfusion injury, atherosclerosis, brain trauma, multiple sclerosis, cerebral malaria, sepsis, septic shock, toxic shock syndrome, fever, myalgias due to HIV-1, HIV-2, HIV-3, cytomegalovirus (CMV), influenza, adenovirus, the herpes viruses or herpes zoster infection in a mammal comprising administering an effective amount of a compound according to any one of the above embodiments.

Another aspect of the invention relates to a method of lowering plasma concentrations of either or both TNF-a and IL-1 comprising administering an effective amount of a compound according to any one of the above embodiments.

Another aspect of the invention relates to a method of lowering plasma concentrations of either or both IL-6 and IL-8 comprising administering an effective amount of a compound according to any one of the above embodiments.

Another aspect of the invention relates to a method of prophylaxis or treatment of diabetes disease in a mammal comprising administering an effective amount of a compound according to any one of the above embodiments to produce a glucagon antagonist effect.

Another aspect of the invention relates to a method of prophylaxis or treatment of a pain disorder in a mammal comprising administering an effective amount of a compound according to any one of the above embodiments.

Another aspect of the invention relates to a method of decreasing prostaglandins production in a mammal comprising administering an effective amount of a compound according to any one of the above embodiments.

Another aspect of the invention relates to a method of decreasing cyclooxygenase enzyme activity in a mammal comprising administering an effective amount of a compound according to any one of the above embodiments. In another embodiment, the cyclooxygenase enzyme is COX-2.

Another aspect of the invention relates to a method of decreasing cyclooxygenase enzyme activity in a mammal comprising administering an effective amount of the above pharmaceutical composition. In another embodiment the cyclooxygenase enzyme is COX-2.

Another aspect of the invention relates to the manufacture of a medicament comprising a compound according to any one of the above embodiments.

Another aspect of the invention relates to the manufacture of a medicament for the treatment of inflammation comprising administering an effective amount of a compound according to any one of the above embodiments.

Another aspect of the invention relates to the manufacture of a medicament for the treatment of rheumatoid arthritis, Pagets disease, osteoporosis, multiple myeloma, uveititis, acute or chronic myelogenous leukemia, pancreatic β cell destruction, osteoarthritis, rheumatoid spondylitis, gouty arthritis, inflammatory bowel disease, adult respiratory distress syndrome (ARDS), psoriasis, Crohn's disease, allergic rhinitis, ulcerative colitis, anaphylaxis, contact dermatitis, asthma, muscle degeneration, cachexia, Reiter's syndrome, type I diabetes, type II diabetes, bone resorption diseases, graft vs. host reaction, Alzheimer's disease, stroke, myocardial infarction, ischemia reperfusion injury, atherosclerosis, brain trauma, multiple sclerosis, cerebral malaria, sepsis, septic shock, toxic shock syndrome, fever, myalgias due to HIV-1, HIV-2, HIV-3, cytomegalovirus (CMV), influenza, adenovirus, the herpes viruses or herpes zoster infection in a mammal comprising administering an effective amount of a compound according to any one of the above embodiments.

The compounds of this invention may have in general several asymmetric centers and are typically depicted in the form of racemic mixtures. This invention is intended to encompass racemic mixtures, partially racemic mixtures and separate enantiomers and diasteromers.

The specification and claims contain listing of species using the language “selected from . . . and . . . ” and “is . . . or . . . ” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups as needed.

Unless otherwise specified, the following definitions apply to terms found in the specification and claims:
“Aryl” means a phenyl or naphthyl radical, wherein the phenyl may be fused with a C₃₋₄cycloalkyl bridge.
“Benzo group”, alone or in combination, means the divalent radical C₄H₄═, one representation of which is —CH═CH—CH═CH—, that when vicinally attached to another ring forms a benzene-like ring—for example tetrahydronaphthylene, indole and the like.
“C_{α-β}alkyl” means an alkyl group comprising from α to β carbon atoms in a branched, cyclical or linear relationship or any combination of the three. The alkyl groups described in this section may also contain double or triple bonds. Examples of C₁₋₈alkyl include, but are not limited to the following: 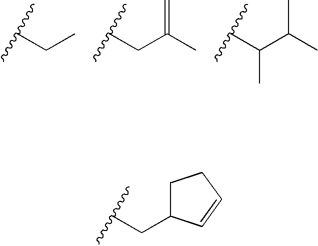
“Halogen” and “halo” mean a halogen atoms selected from F, Cl, Br and I. “C_{α-β}haloalkyl” means an alkyl group, as described above, wherein any number—at least one—of the hydrogen atoms attached to the alkyl chain are replaced by F, Cl, Br or I.
“Heterocycle” means a ring comprising at least one carbon atom and at least one other atom selected from N, O and S. Examples of heterocycles that may be found in the claims include, but are not limited to, the following: 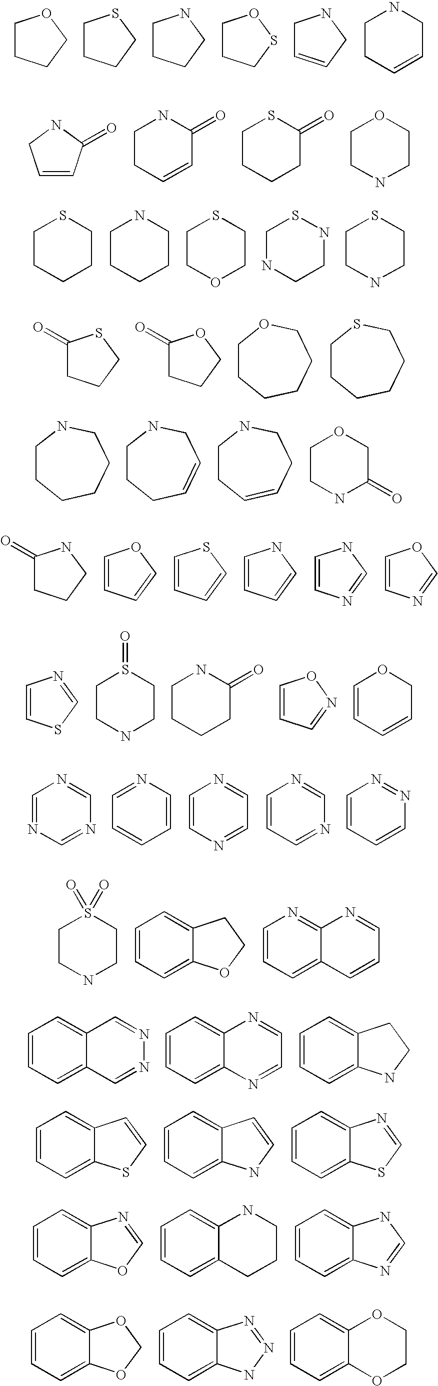 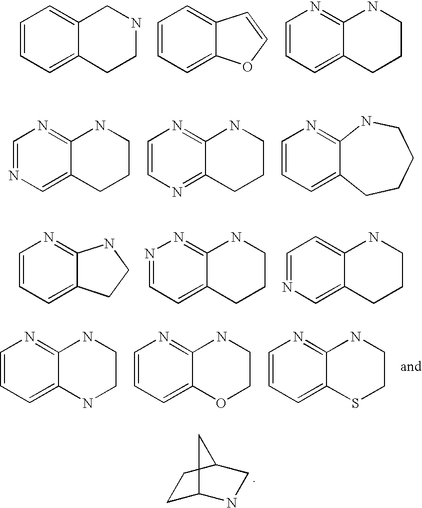
“Pharmaceutically-acceptable salt” means a salt prepared by conventional means, and are well known by those skilled in the art. The “pharmacologically acceptable salts” include basic salts of inorganic and organic acids, including but not limited to hydrochloric acid, hydrobromic acid, sulfuric acid, phosphoric acid, methanesulphonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compounds of the invention include an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et al., J. Pharm. Sci. 66:1 (1977).
“Leaving group” generally refers to groups readily displaceable by a nucleophile, such as an amine, a thiol or an alcohol nucleophile. Such leaving groups are well known in the art. Examples of such leaving groups include, but are not limited to, N-hydroxysuccinimide, N-hydroxybenzotriazole, halides, triflates, tosylates and the like. Preferred leaving groups are indicated herein where appropriate.
“Protecting group” generally refers to groups well known in the art which are used to prevent selected reactive groups, such as carboxy, amino, hydroxy, mercapto and the like, from undergoing undesired reactions, such as nucleophilic, electrophilic, oxidation, reduction and the like. Preferred protecting groups are indicated herein where appropriate. Examples of amino protecting groups include, but are not limited to, aralkyl, substituted aralkyl, cycloalkenylalkyl and substituted cycloalkenyl alkyl, allyl, substituted allyl, acyl, alkoxycarbonyl, aralkoxycarbonyl, silyl and the like. Examples of aralkyl include, but are not limited to, benzyl, ortho-methylbenzyl, trityl and benzhydryl, which can be optionally substituted with halogen, alkyl, alkoxy, hydroxy, nitro, acylamino, acyl and the like, and salts, such as phosphonium and ammonium salts. Examples of aryl groups include phenyl, naphthyl, indanyl, anthracenyl, 9-(9-phenylfluorenyl), phenanthrenyl, durenyl and the like. Examples of cycloalkenylalkyl or substituted cycloalkylenylalkyl radicals, preferably have 6-10 carbon atoms, include, but are not limited to, cyclohexenyl methyl and the like. Suitable acyl, alkoxycarbonyl and aralkoxycarbonyl groups include benzyloxycarbonyl, t-butoxycarbonyl, iso-butoxycarbonyl, benzoyl, substituted benzoyl, butyryl, acetyl, tri-fluoroacetyl, tri-chloro acetyl, phthaloyl and the like. A mixture of protecting groups can be used to protect the same amino group, such as a primary amino group can be protected by both an aralkyl group and an aralkoxycarbonyl group. Amino protecting groups can also form a heterocyclic ring with the nitrogen to which they are attached, for example, 1,2-bis(methylene)benzene, phthalimidyl, succinimidyl, maleimidyl and the like and where these heterocyclic groups can further include adjoining aryl and cycloalkyl rings. In addition, the heterocyclic groups can be mono-, di- or tri-substituted, such as nitrophthalimidyl. Amino groups may also be protected against undesired reactions, such as oxidation, through the formation of an addition salt, such as hydrochloride, toluenesulfonic acid, trifluoroacetic acid and the like. Many of the amino protecting groups are also suitable for protecting carboxy, hydroxy and mercapto groups. For example, aralkyl groups. Alkyl groups are also suitable groups for protecting hydroxy and mercapto groups, such as tert-butyl.

Silyl protecting groups are silicon atoms optionally substituted by one or more alkyl, aryl and aralkyl groups. Suitable silyl protecting groups include, but are not limited to, trimethylsilyl, triethylsilyl, tri-isopropylsilyl, tert-butyldimethylsilyl, dimethylphenylsilyl, 1,2-bis(dimethylsilyl)benzene, 1,2-bis(dimethylsilyl)ethane and diphenylmethylsilyl. Silylation of an amino groups provide mono- or di-silylamino groups. Silylation of aminoalcohol compounds can lead to a N,N,O-tri-silyl derivative. Removal of the silyl function from a silyl ether function is readily accomplished by treatment with, for example, a metal hydroxide or ammonium fluoride reagent, either as a discrete reaction step or in situ during a reaction with the alcohol group. Suitable silylating agents are, for example, trimethylsilyl chloride, tert-butyl-dimethylsilyl chloride, phenyldimethylsilyl chloride, diphenylmethyl silyl chloride or their combination products with imidazole or DMF. Methods for silylation of amines and removal of silyl protecting groups are well known to those skilled in the art. Methods of preparation of these amine derivatives from corresponding amino acids, amino acid amides or amino acid esters are also well known to those skilled in the art of organic chemistry including amino acid/amino acid ester or aminoalcohol chemistry.

Protecting groups are removed under conditions which will not affect the remaining portion of the molecule. These methods are well known in the art and include acid hydrolysis, hydrogenolysis and the like. A preferred method involves removal of a protecting group, such as removal of a benzyloxycarbonyl group by hydrogenolysis utilizing palladium on carbon in a suitable solvent system such as an alcohol, acetic acid, and the like or mixtures thereof. A t-butoxycarbonyl protecting group can be removed utilizing an inorganic or organic acid, such as HCl or trifluoroacetic acid, in a suitable solvent system, such as dioxane or methylene chloride. The resulting amino salt can readily be neutralized to yield the free amine. Carboxy protecting group, such as methyl, ethyl, benzyl, tert-butyl, 4-methoxyphenylmethyl and the like, can be removed under hydroylsis and hydrogenolysis conditions well known to those skilled in the art.

It should be noted that compounds of the invention may contain groups that may exist in tautomeric forms, such as cyclic and acyclic amidine and guanidine groups, heteroatom substituted heteroaryl groups (Y′=O, S, NR), and the like, which are illustrated in the following examples: 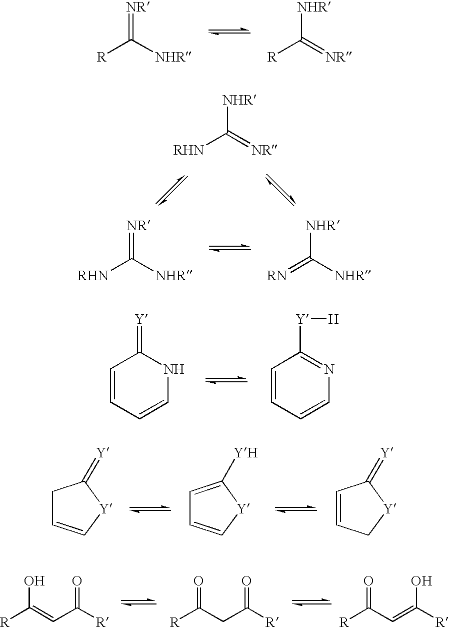
and though one form is named, described, displayed and/or claimed herein, all the tautomeric forms are intended to be inherently included in such name, description, display and/or claim.

Prodrugs of the compounds of this invention are also contemplated by this invention. A prodrug is an active or inactive compound that is modified chemically through in vivo physiological action, such as hydrolysis, metabolism and the like, into a compound of this invention following administration of the prodrug to a patient. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bundgaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, Apr. 11, 1981) discloses Mannich-base hydroxamic acid prodrugs, their preparation and use.
“Cytokine” means a secreted protein that affects the functions of other cells, particularly as it relates to the modulation of interactions between cells of the immune system or cells involved in the inflammatory response. Examples of cytokines include but are not limited to interleukin 1 (IL-1), preferably IL-1β, interleukin 6 (IL-6), interleukin 8 (IL-8) and TNF, preferably TNF-α (tumor necrosis factor-α).
“TNF, IL-1, IL-6, and/or IL-8 mediated disease or disease state” means all disease states wherein TNF, IL-1, IL-6, and/or IL-8 plays a role, either directly as TNF, IL-1, IL-6, and/or IL-8 itself, or by TNF, IL-1, IL-6, and/or IL-8 inducing another cytokine to be released. For example, a disease state in which IL-1 plays a major role, but in which the production of or action of IL-1 is a result of TNF, would be considered mediated by TNF.

Compounds according to the invention can be synthesized according to one or more of the following methods. It should be noted that the general procedures are shown as it relates to preparation of compounds having unspecified stereochemistry. However, such procedures are generally applicable to those compounds of a specific stereochemistry, e.g., where the stereochemistry about a group is (S) or (R). In addition, the compounds having one stereochemistry (e.g., (R)) can often be utilized to produce those having opposite stereochemistry (i.e., (S)) using well-known methods, for example, by inversion. 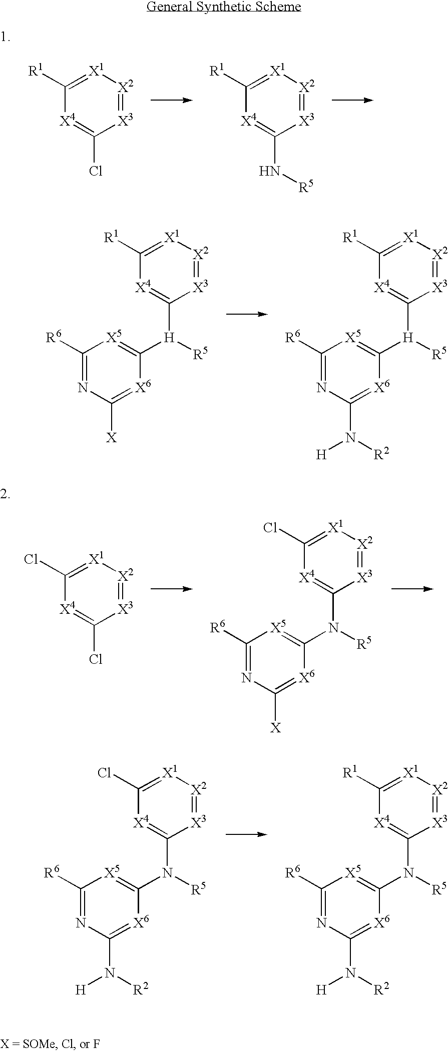
Abbreviations
Ac₂O acetic anhydride
CH₂Cl₂ dichloromethane, methylene chloride
DCM dichloromethane
DCE 1,2-dichloroethane
DME dimethoxyethane, ethylene glycol dimethyl ether
DMF dimethyl formamide
EDC 1-[3-(dimethylamino)propyl]-3-ethylcarbodiimide hydrochloride
Et₂O diethyl ether
EtOH ethanol
EtOAc ethyl acetate
Fmoc 9-fluorenylmethoxycarbonyl
h hour(s)
MeOH methanol
NMP 1-methyl-2-pyrrolidinone
i-PrOH isopropanol
PS-carbodiimide polymer supported carbodiimide resin from Argonaut
RT room temperature
SiO₂ silica
TFA trifluoroacetic acid
THF tetrahydrofuran

### EXAMPLE 1

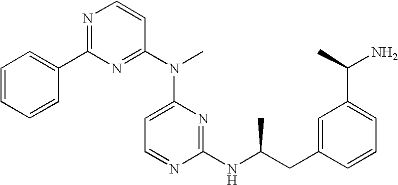 N²-((S)-1-(3-((R)-1-Aminoethyl)phenyl)propan-2-yl)-N⁴-methyl-N⁴-(2-phenylpyrimidin-4-yl)pyrimidine-2,4-diamine
Step A: 2-Phenylpyrimidin-4(3H)-one.

Benzamidine hydrochloride (10 g, 64 mmol), ethyl propioloate (6.26 g, 64 mmol), potassium carbonate (8.85 g, 64 mmol), and ethanol (200 mL) were mixed in a 500 mL roundbottom flask and heated to reflux for 24 h under nitrogen atmosphere. After cooling to RT the mixture was filtered, the filtrate was concentrated under vacuum, and the residue was dissolved in water (75 mL). The solution was taken to pH 3 with conc. HCl and the resulting off-white solid was filtered, washed with water, and air-dried to give 2-phenylpyrimidin-4(3H)-one as an off-white solid. MS m/z 173 (MH)⁺.

Step B: 4-Chloro-2-phenylpyrimidine.

The above pyrimidone (8.83 g, 51.3 mmol) was dissolved in phosphorus oxychloride (40 mL) and heated to 90° C. for 15 h. The mixture was cooled to RT and concentrated under vacuum to about 10 mL total volume. The remainder was poured over ice-water/CH₂Cl₂ mixture (1:1, 200 mL total volume) and the remaining POCl₃ was quenched with saturated sodium bicarbonate solution. The two layers were separated and the aqueous layer was extracted with CH₂Cl₂ two times. The combined extracts were washed with brine, dried (Na₂SO₄), and concentrated under vacuum to give 4-chloro-2-phenylpyrimidine as an orange solid. NMR (CDCl₃) δ: 8.65 (d, J=5.2 Hz, 1H), 8.45 (m, 2H), 7.51 (m, 3H), 7.24 (d, J=5.2 Hz, 1H).

Step C: N-Methyl-2-phenylpyrimidin-4-amine.

Methylamine (42 mmol, 2M in THF) was added to the above chloride (4.0 g, 21 mmol) and 2-propanol (20 mL) in a 300 mL reaction vessel with a Teflon screw cap. The vessel was sealed via the screw cap and the mixture was heated to 80° C. for 15 h. The mixture was cooled to RT, concentrated under vacuum, and the residue was taken up in 3 mL CH₂Cl₂ and 5 mL hexane. The resulting solid was filtered and washed with hexane to give N-methyl-2-phenylpyrimidin-4-amine hydrochloride as an off-white solid. MS m/z 186 (MH)⁺.

Step D: N-(2-Fluoropyrimidin-4-yl)-N-methyl-2-phenylpyrimidin-4-amine.

Lithium hexamethyldisilazide (23 mmol, 23 mL, 1.0M in THF) was added to a solution of aminopyrimidine (3.5 g, 18.9 mmol, freebased) in THF (10 mL) at −78° C. The mixture was stirred at that temperature for 10 min and the difluoropyrimidine (2.64 g, 23 mmol) was added as a solution in THF (10 mL). The orange solution was stirred at −78° C. for 1.5 h, saturated NH₄Cl (20 mL) was added, and the mixture was warmed to RT. The two layers were separated and the aqueous layer was extracted with CH₂Cl₂ two times. The combined extracts were washed with brine, dried (Na₂SO₄), and concentrated under vacuum. Purification by flash column chromatography gave N-2-fluoropyrimidin-4-yl)-N-methyl-2-phenylpyrimidin-4-amine as a white solid. MS m/z 282 (MH)⁺.

Step E: N²-((S)-1-(3-((R)-1-Aminoethyl)phenyl)propan-2-yl)-N⁴-methyl-N⁴(2-phenylpyrimidin-4-yl)pyrimidine-2,4-diamine tert-butyl.

(R)-1-(3-((S)-2-aminopropyl)phenyl)ethylcarbamate (400 mg, 1.44 mmol), N-(2-fluoropyrimidin-4-yl)-N-methyl-2-phenylpyrimidin-4-amine (400 mg, 1.42 mmol) and 1,4-dioxane (3 mL) were mixed in a 25 mL pear-shaped flask equipped with a magnetic stir bar. The mixture was placed under argon atmosphere, heated to 100° C. for 15 h, cooled to RT, and partitioned between saturated sodium bicarbonate (aq.) and CH₂Cl₂. The layers were separated and the organic layer was washed with water three times, brine once, dried (MgSO₄), filtered, concentrated under vacuum, and purified by column chromatography to give tert-butyl (R)-1-(3-((S)-2-(4-(methyl(2-phenylpyrimidin-4-yl)amino)pyrimidin-2-ylamino)propyl)phenyl)ethylcarbamate as a white solid. Trifluoroacetic acid (5 mL), CH₂Cl₂ (5 mL) and the Boc protected amine (374 mg, 0.65 mmol) were mixed in a 100 mL roundbottom flask fitted with a magnetic stir bar. The mixture was stirred at RT for 1 h and the solvent was removed under vacuum. The mixture was partitioned between saturated sodium bicarbonate (aq.) and CH₂Cl₂, the layers were separated, and the aqueous layer was extracted with CH₂Cl₂ three times. The extracts were dried (MgSO₄), filtered, concentrated under vacuum, and purified by column chromatography to give N²-((S)-1-(3-((R)-1-aminoethyl)phenyl)propan-2-yl)-N⁴-methyl-N⁴-(2-phenylpyrimidin-4-yl)pyrimidine-2,4-diamine as a white solid. MS m/z 440 (MH)⁺.

### EXAMPLE 2

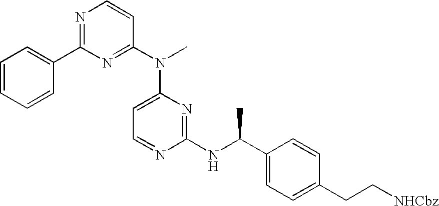 (S)-Benzyl 4-(1-(4-(methyl(2-phenylpyrimidin-4-yl)amino)pyrimidin-2-yl-amino)ethyl)phenethylcarbamate.

(S)-benzyl 4-(1-aminoethyl)phenethylcarbamate (170 mg, 0.56 mmol), N-(2-fluoropyrimidin-4-yl)-N-methyl-2-phenylpyrimidin-4-amine (160 mg, 0.56 mmol) and 1,4-dioxane (1 mL) were mixed in a 25 mL pear-shaped flask equipped with a magnetic stir bar. The mixture was placed under argon atmosphere, heated to 100° C. for 15 h, cooled to RT, and partitioned between saturated sodium bicarbonate (aq.) and CH₂Cl₂. The layers were separated and the organic layer was washed with water three times, brine once, dried (MgSO₄), filtered, concentrated under vacuum, and purified by column chromatography to give (S)-benzyl 4-(1-(4-(methyl(2-phenylpyrimidin-4-yl)amino)pyrimidin-2-ylamino)ethyl)phenethylcarbamate as a white solid. MS m/z 560 (MH)⁺.

### EXAMPLE 3

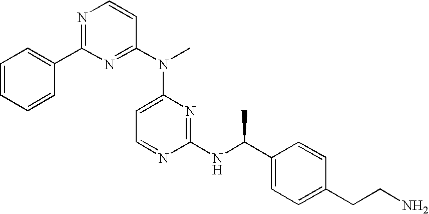 (S)-N²-(1-(4-(2-Aminoethyl)phenyl)ethyl)-N⁴-methyl-N⁴-(2-phenylpyrimidin-4-yl)pyrimidine-2,4-diamine.

(S)-benzyl 4-(1-(4-(methyl(2-phenylpyrimidin-4-yl)amino)pyrimidin-2-ylamino)ethyl)phenethylcarbamate (Example 2) (204 mg, 0.27 mmol) and 10% palladium on carbon (50 mg) in methanol (5 mL) were placed under hydrogen atmosphere and stirred for 15 h. The mixture was carefully filtered through celite, concentrated under vacuum, and purified by flash column chromatography to give (S)-N²-(1-(4-(2-aminoethyl)phenyl)ethyl)-N⁴-methyl-N⁴-(2-phenylpyrimidin-4-yl)pyrimidine-2,4-diamine as a white solid. MS m/z 426 (MH)⁺.

### EXAMPLE 4

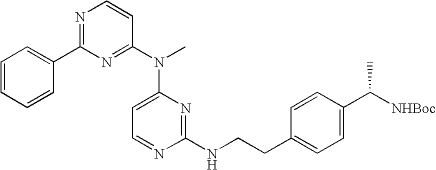 (S)-tert-Butyl 1-(4-(2-(4-(methyl(2-phenylpyrimidin-4-yl)amino)pyrimidin-2-ylamino)ethyl)phenyl)ethyl carbamate.

(S)-tert-butyl 1-(4-(2-aminoethyl)phenyl)-ethylcarbamate (66 mg, 0.25 mmol), N-(2-fluoropyrimidin-4-yl)-N-methyl-2-phenylpyrimidin-4-amine (70 mg, 0.25 mmol) and 1,4-dioxane (2 mL) were mixed in a 25 mL pear-shaped flask equipped with a magnetic stir bar. The mixture was placed under argon atmosphere, heated to 100° C. for 15 h, cooled to RT, and partitioned between saturated sodium bicarbonate (aq.) and CH₂Cl₂. The layers were separated and the organic layer was washed with water three times, brine once, dried (MgSO₄), filtered, concentrated under vacuum, and purified by column chromatography to give (S)-tert-butyl 1-(4-(2-(4-(methyl(2-phenylpyrimidin-4-yl)amino)pyrimidin-2-ylamino)ethyl)phenyl)ethyl carbamate as a white solid. MS m/z 526 (MH)⁺.

### EXAMPLE 5

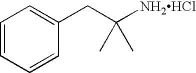 2-Methyl-1-phenylpropan-2-amine hydrochloric acid salt.
Step A: 1-Phenyl-2-propanone.

1-Phenyl-2-propanol (10 mL, 71 mmol) was dissolved in acetone (800 mL), cooled to 10° C. and Jones reagent was added slowly until an orange color persisted. After 5 min, 2-propanol was added to destroy the excess chromium reagent. The reaction mixture was diluted with Et₂O (500 mL) and water (500 mL). The layers were separated and the aqueous layer was extracted once more with Et₂O (200 mL). The ether extracts were combined and washed with brine (2×100 mL), dried over MgSO₄ and evaporated under reduced pressure. The product was obtained as a yellow oil that was used directly in the next step.

Step B: 2-Methyl-3-phenylpropan-2-ol.

Methyllithium (1.45M in THF, 49.4 mL, 72.1 mmol) was added slowly to a −78° C. solution of titanium tetrachloride (7.93 mL, 72.1 mmol) in Et₂O (250 mL). After the addition was complete, the purple-black solution was warmed to −30° C. and a solution of 1-phenyl-2-propanone (8.64 g, 64.4 mmol) in Et₂O (50 mL) was added over 10 min. The solution was warmed to RT and stirring was continued for 4 h before it was quenched with the careful addition of 100 mL of water. The reaction mixture was extracted with Et₂O (3×200 mL) and the combined organic extracts were washed with brine (3×100 mL), dried over MgSO₄ and evaporated in vacuo to a light yellow liquid, which was used directly in the next step.

Step C: 2-Chloro-N-(2-methyl-1-phenylpropan-2-yl)acetamide.

A mixture of 2-methyl-3-phenylpropan-2-ol (8.14 g, 54.2 mmol) and chloroacetonitrile (50 mL) were cooled to 0° C. Acetic acid (26 mL, 0.46 mol) was added followed by the dropwise addition of H₂SO₄ (26 mL, 0.49 mol). After the addition was complete, the reaction mixture slowly warmed to RT and stirring was continued for 40 h. The reaction mixture was then poured into 200 mL of ice-water and extracted with EtOAc (3×75 mL). The pooled organic fractions were washed with water (5×50 mL), dried over MgSO₄ and evaporated under reduced pressure. The title compound was obtained as a light yellow liquid, which is contaminated with acetic acid and chloroacetonitrile. It was used without further purification in the next step.

Step D: 2-Methyl-1-phenylpropan-2-amine hydrochloric acid salt.

A mixture of the chloroacetamide from Step C above (12.2 g, 54.2 mmol), thiourea (2.7 g, 65 mmol) and acetic acid (10.6 mL, 0.325 mol) in absolute ethanol (60 mL) was heated to reflux to 16 h. The reaction mixture was then diluted with water (50 mL) and extracted with EtOAc (2×75 mL). The combined organic fractions were washed with brine (5×50 mL), dried over MgSO₄ and evaporated in vacuo. Distillation under high vacuum provided the free amine (bp 125° C.@6 Torr). This crude material was dissolved in Et₂O and 4 mL of 4N HCl in 1,4-dioxane was added. Filtration of the white precipitate provided the desired compound as its hydrochloric acid salt.

### EXAMPLE 6

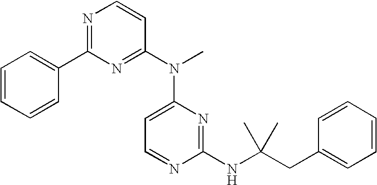 N⁴-Methyl-N²-(2-methyl-1-phenylpropan-2-yl)-N⁴-(2-phenylpyrimidin-4-yl)pyrimidine-2,4-diamine.

(2-Fluoropyrimidin-4-yl)-N-methyl-2-phenylpyrimidin-4-amine (113 mg, 0.40 mmol), 2-methyl-1-phenylpropan-2-amine hydrochloric acid salt (298 mg, 1.6 mmol), diisopropylethylamine (0.28 mL, 1.6 mmol) and N-methylpyrrolidinone (2 mL) were loaded into a sealable vessel. The vessel was sealed and heated to 150° C. for 60 h. The reaction mixture was then cooled to RT, diluted with EtOAc (50 mL), washed with brine (3×50 mL), dried over MgSO₄ and evaporated under reduced pressure. Purification by column chromatography (40 g pre-packed silica gel column, elution with 0-3% MeOH:CH₂Cl₂) provided the title compound as an off-white solid. MS m/z 411 (MH)⁺

### EXAMPLE 7

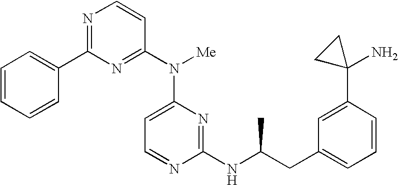 (S)-N²-(1-(3-(1-Aminocyclopropyl)phenyl)propan-2-yl)-N⁴-methyl-N⁴-(2-phenylpyrimidin-4-yl)pyrimidine-2,4-diamine.

Titanium(IV) isopropoxide (0.775 mL, 2.64 mmol) was added to a RT solution of (S)-3-(2-(4-(methyl(2-phenylpyrimidin-4-yl)-amino)pyrimidin-2-ylamino)propyl)benzonitrile (507 mg, 1.20 mmol) in THF (15 mL), followed by the rapid addition of EtMgBr (1.0M in THF, 4.8 mL,, 4.8 mmol). After 30 min, further titanium tetrachloride (0.775 mL, 2.64 mmol) and EtMgBr (1.0M in THF, 4.8 mL, 4.8 mmol) were added. The reaction mixture was stirred for 30 min and BF₃.OEt₂ (1.2 mL, 9.6 mmol) was added and stirring was continued for 10 min. The reaction was then quenched by the addition of 3 mL of 10% NaOH solution and the pH was adjusted to pH 7 with concentrated HCl. The crude product was extracted with Et₂O (2×25 mL) and CHCl₃ (2×25 mL). The organic phases were combined, dried over MgSO₄ and evaporated under reduced pressure. The residue was taken up in CHCl₃, loaded on to a 40 g pre-packed silica gel column and eluted with 0-5% MeOH(contains 10% NH₄OH):CH₂Cl₂. Concentration of the appropriate fractions provided the desired compound as a light-yellow solid. MS m/z 452 (MH)⁺.

### EXAMPLE 8

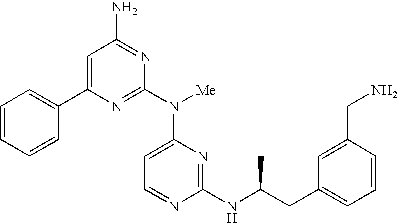 (S)-N⁴-(4-Amino-6-phenylpyrimidin-2-yl)-N²-(1-(3-(aminomethyl)phenyl)propan-2-yl)-N⁴-methylpyrimidine-2,4-diamine
Step A: (S)-3-(2-(4-(Methyl(7-phenyl-[1,2,4]triazolo[1,5-f]pyrimidin-5-yl)amino)-pyrimidin-2-ylamino)propyl)benzonitrile.

(S)-3-(2-Aminopropyl)benzonitrile (1.31 g, 8.2 mmol), N-methyl-N-(2-(methylsulfinyl)pyrimidin-4-yl)-7-phenyl-[1,2,4]triazolo[1,5-f]pyrimidin-5-amine (2.0 g, 5.5 mmol), and 1,4-dioxane (11 mL) were mixed in a 25 mL pear-shaped flask fitted with a magnetic stir bar. The mixture was placed under argon atmosphere, heated to 100° C. for 15 h, cooled to RT, and then partitioned between saturated sodium bicarbonate (aq.) and ethyl acetate. The layers were separated and the organic layer was washed with water three times, brine once, dried (MgSO₄), filtered, concentrated under vacuum, and purified by column chromatography to give (S)-3-(2-(4-(methyl(7-phenyl-[1,2,4]triazolo[1,5-f]pyrimidin-5-yl)amino)pyrimidin-2-ylamino)propyl)benzonitrile as a white solid. MS m/z 462 (MH)⁺.

Step B: (S)-N⁴-(4-Amino-6-phenylpyrimidin-2-yl)-N²-(1-(3-(aminomethyl)phenyl)-propan-2-yl)-N⁴-methylpyrimidine-2,4-diamine.

The above benzonitrile (2.08 g, 4.3 mmol) and raney nickel (10 g) were heated under argon for 2 h, cooled to RT, and carefully filtered through a pad of celite. The celite was washed with methanol several times and the filtrate was concentrated under vacuum. The residue was purified by column chromatography to give (S)-N⁴-(4-amino-6-phenylpyrimidin-2-yl)-N²-(1-(3-(aminomethyl)phenyl)propan-2-yl)-N⁴-methylpyrimidine-2,4-diamine as a white solid. MS m/z 441 (MH)⁺.

### EXAMPLE 9

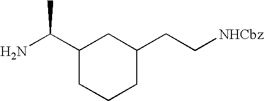 Benzyl 2-(3-((S)-1-aminoethyl)cyclohexyl)ethylcarbamate.
Step A: (S,E)-Methyl 3-(3-(1-(tert-butoxycarbonyl)ethyl)phenyl)acrylate.

(S)-tert-Butyl 1-(3-bromophenyl)ethylcarbamate (7.4 g, 24.5 mmol), methacrylate (4.21 g, 49 mmol), palladium dibenzylidene acetone (1.35 g, 1.47 mmol), tri-tert-butyl phosphine (594 mg, 3.0 mmol), dicyclohexyl methylamine (5.75 g, 29.4 mmol), and 1,4-dioxane (45 mL) were mixed under argon atmosphere in a 250 mL roundbottom flask equipped with a stir bar. The mixture was heated to 80° C. for 3 h, cooled to RT, partitioned between water and ethyl acetate, the layers separated, and the aqueous layer extracted with ethyl acetate twice. The combined extracts were washed with brine, dried (MgSO₄), filtered, concentrated under vacuum, and purified by flash column chromatography to give (S,E)-methyl 3-(3-(1-(tert-butoxycarbonyl)ethyl)phenyl)acrylate as an oil (7.0 g). NMR (CDCl₃) δ: 7.68 (d, J=16.0 Hz, 1H), 7.42 (m, 2H), 7.34 (m, 2H), 6.44 (d, J=16.0 Hz, 1H), 4.81 (br m, 2H), 3.81 (s, 3H), 1.44 (s, 9H), 1.44 (br d, 3H).

Step B: 3-(3-((S)-1-(tert-Butoxycarbonyl)ethyl)cyclohexyl)propanoic acid.

A mixture of (S,E)-methyl 3-(3-(1-(tert-butoxycarbonyl)ethyl)phenyl)acrylate (1.0 g, 3.3 mmol) and rhodium on carbon (300 mg) in methanol (10 mL) was stirred under an atmosphere of hydrogen (1 atm) for 24 h. The mixture was carefully filtered through celite and the filtrate was concentrated under reduced pressure. The mixture of cyclohexanes was taken directly to the next step (963 mg, 93%). The material obtained from the above reaction was dissolved in methanol (10 mL) in a 50 mL roundbottom flask equipped with a stir bar. Sodium hydroxide (15 mmol, 5N in water) was added and the mixture was stirred at 65° C. for 1 h. The reaction was cooled to RT and then partitioned between water and chloroform. The layers were separated and the aqueous layer was washed with chloroform one more time. The aqueous layer was then taken to pH 4 with 10% KHSO₄, and the product was extracted with chloroform several times. Concentration of the extracts gave 3-(3-((S)-(tert-butoxycarbonyl)ethyl)cyclohexyl)propanoic acid as an oil. MS m/z 300 (MH)⁺.

Step C: tert-Butyl (S)-1-(3-(2-Boc-aminoethyl)cyclohexyl)ethylcarbamate.

Ethyl chloroformate (292 mg, 2.7 mmol) was added dropwise to a solution of carboxylic acid (730 mg, 2.44 mmol) and triethylamine (494 mg, 4.9 mmol) in THF (20 mL) at 0° C. The solution was stirred for 1 h at that temperature and sodium azide (176 mg, 2.7 mmol) was added as a solution in water (1 mL). The cooling bath was removed and the mixture was stirred for 2 h before ethyl acetate (20 mL) was added. The mixture was washed with saturated NaHCO₃ (aq.) one time, brine once, dried (MgSO₄), filtered, and concentrated under vacuum. Toluene (8 mL) and benzyl alcohol (395 mg, 3.66 mmol) were added and the reaction was heated to 105° C. for 15 h. The solvent was removed under vacuum and flash column chromatography gave product as a mixture with benzyl alcohol (colorless oil). MS m/z 405(MH)⁺.

Step D: Benzyl 2-(3-((S)-1-aminoethyl)cyclohexyl)ethylcarbamate.

Trifluoroacetic acid (2 mL), CH₂Cl₂ (2 mL) and the Boc protected amine (250 mg, 0.62 mmol) were mixed in a 25 mL roundbottom flask fitted with a magnetic stir bar. The mixture was stirred at RT for 1 h and the solvent was removed under vacuum. The mixture was partitioned between saturated sodium bicarbonate (aq.) and CH₂Cl₂, the layers were separated, and the aqueous layer was extracted with CH₂Cl₂ three times. The extracts were dried (MgSO₄), filtered, concentrated under vacuum, and purified by column chromatography to give two stereoisomers of the title compound. Both show MS m/z 305 (MH)⁺. Stereochemistry of the two compounds was assigned arbitrarily.

### EXAMPLE 10

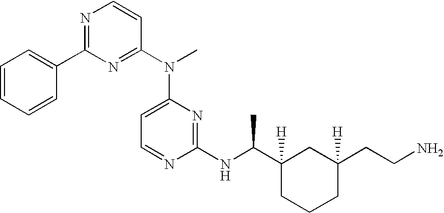 N²-((S)-((1R,3S)-3-(2-Aminoethyl)cyclohexyl)ethyl)-N⁴-methyl-N⁴-(2-phenylpyrimidin-4-yl)pyrimidine-2,4-diamine.

Benzyl 2-((1S,3R)-3-((S)-1-aminoethyl)-cyclohexyl)ethylcarbamate (50 mg, 0.16 mmol), N-(2-fluoropyrimidin-4-yl)-N-methyl-2-phenylpyrimidin-4-amine (46 mg, 0.16 mmol) and 1,4-dioxane (0.5 mL) were mixed in a 25 mL pear-shaped flask equipped with a magnetic stir bar. The mixture was placed under argon atmosphere, heated to 100° C. for 15 h, cooled to RT, and partitioned between saturated sodium bicarbonate (aq.) and CH₂Cl₂. The layers were separated and the organic layer was washed with water three times, brine once, dried (MgSO₄), filtered, concentrated under vacuum, and taken directly to the next step.

The material obtained from the above reaction was dissolved in concentrated HCl (aq.) (1 mL) and heated to 100° C. for 10 min. After cooling to RT, the reaction was quenched with saturated NaHCO₃ (aq., 5 mL) and extracted with chloroform/IPA (4:1, v/v) several times. The combined extracts were concentrated under vacuum and purified by flash column chromatography to give N²-((S)-((1R,3S)-3-(2-aminoethyl)cyclohexyl)ethyl)-N⁴-methyl-N⁴-(2-phenylpyrimidin-4-yl)pyrimidine-2,4-diamine as a single compound. MS m/z 432 (MH)⁺.

### EXAMPLE 11

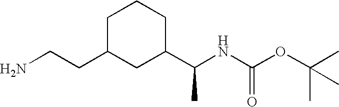 tert-Butyl (S)-1-(3-(2-aminoethyl)cyclohexyl)ethylcarbamate.

The carbamate (240 mg, 0.60 mmol) and 10% palladium on carbon (40 mg) in 1,4-dioxane (10 mL) were placed under hydrogen atmosphere and stirred for 15 h. The mixture was carefully filtered through celite, concentrated under vacuum, and purified by flash column chromatography to give tert-butyl (S)-1-(3-(2-aminoethyl)cyclohexyl)ethyl carbamate as an oil (about 9:1 mixture of two diastereomers). MS m/z 271 (MH)⁺.

### EXAMPLE 12

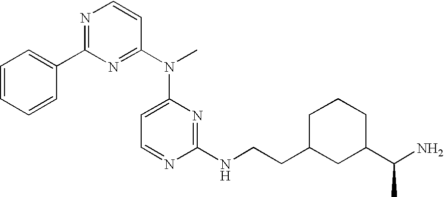 N²-(2-(3-((S)-1-Aminoethyl)cyclohexyl)ethyl)-N⁴-methyl-N⁴-(2-phenylpyrimidin-4-yl)pyrimidine-2,4-diamine.

tert-Butyl (S)-1-(3-(2-aminoethyl)cyclohexyl)-ethylcarbamate (80 mg, 0.3 mmol), N-(2-fluoropyrimidin-4-yl)-N-methyl-2-phenylpyrimidin-4-amine (80 mg, 0.3 mmol) and 1,4-dioxane (1 mL) were mixed in a 25 mL pear-shaped flask equipped with a magnetic stir bar. The mixture was placed under argon atmosphere, heated to 100° C. for 15 h, cooled to RT, and partitioned between saturated sodium bicarbonate (aq.) and CH₂Cl₂. The layers were separated and the organic layer was washed with water three times, brine once, dried (MgSO₄), filtered, concentrated under vacuum, and taken directly to the next step.

Trifluoroacetic acid (2.5 mL), CH₂Cl₂ (2.5 mL) and the Boc protected amine were mixed in a 25 mL round-bottom flask fitted with a magnetic stir bar. The mixture was stirred at RT for 1 h and the solvent was removed under vacuum. The mixture was partitioned between saturated sodium bicarbonate (aq.) and CH₂Cl₂, the layers were separated, and the aqueous layer was extracted with CH₂Cl₂ three times. The extracts were dried (MgSO₄), filtered, concentrated under vacuum, and purified by column chromatography to give N²-(2-(3-((S)-1-aminoethyl)cyclohexyl)ethyl)-N⁴-methyl-N⁴-(2-phenylpyrimidin-4-yl)pyrimidine-2,4-diamine as a white solid (about 9:1 mixture of two stereoisomers). MS m/z 432 (MH)⁺.

### EXAMPLE 13

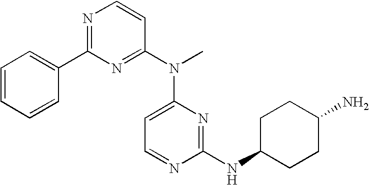 N²-((1r,4r)-4-Aminocyclohexyl)-N⁴-methyl-N⁴-(2-phenylpyrimidin-4-yl)pyrimidine-2,4-diamine.

N-(2-Fluoropyrimidin-4-yl)-N-methyl-2-phenylpyrimidin-4-amine (442 mg, 1.5 mmol), trans-cyclohexane-1,4-diamine (257 mg, 2.3 mmol), and 1,4-dioxane (7 mL) were mixed in a 25 mL roundbottom flask. The mixture was stirred at 100° C. overnight under nitrogen. The resulting white solid was filtered off and the filtrate was concentrated under vacuum. The filtrate was then purified by column chromatography to give N²-(4-aminocyclohexyl)-N⁴-methyl-N⁴-(2-phenyl-pyrimidin-4-yl)pyrimidine-2,4-diamine as a white solid. MS m/z 376 (MH)⁺.

### EXAMPLE 14

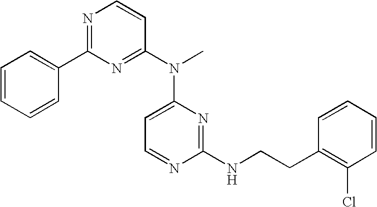 N²-(2-Chlorophenethyl)-N⁴-methyl-N⁴-(2-phenylpyrimidin-4-yl)pyrimidine-2,4-diamine.

N-(2-Fluoropyrimidin-4-yl)-N-methyl-2-phenylpyrimidin-4-amine (442 mg, 1.5 mmol), 2-(2-chlorophenyl)ethanamine (0.32 mL, 2.25 mmol), and dioxane (7 mL) were mixed in a 25 mL roundbottom flask. The mixture was stirred at 100° C. overnight under nitrogen. The reaction was concentrated by vacuum and quenched with saturated NaHCO₃ solution. The white solid was filtered and recrystallized to give N²-(2-chlorophenethyl)-N⁴-methyl-N⁴-(2-phenylpyrimidin-4-yl)pyrimidine-2,4-diamine as a white solid. MS m/z 417 (MH)⁺.

### EXAMPLE 15

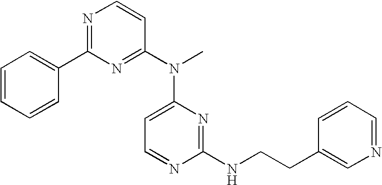 N⁴-Methyl-N⁴-(2-phenylpyrimidin-4-yl)-N²-(2-(pyridin-3-yl)ethyl)pyrimidine-2,4-diamine.

N-(2-Fluoropyrimidin-4-yl)-N-methyl-2-phenylpyrimidin-4-amine (442 mg, 1.5 mmol), 2-(pyridin-3-yl)ethanamine (0.26 mL, 2.25 mmol), and dioxane (7 mL) were mixed in a 25 mL roundbottom flask. The mixture was stirred at 100° C. overnight under nitrogen. The reaction was concentrated under vacuum and re-dissolved in 1:1 CH₂Cl₂/saturated NaHCO₃ solution. The layers were separated and the aqueous layer was extracted with CH₂Cl₂ once. The combined organic layers were washed once with brine, dried (Na₂SO₄), filtered, and concentrated. The residue was purified by column chromatography to give N⁴-methyl-N⁴-(2-phenylpyrimidin-4-yl)-N²-(2-(pyridin-3-yl)ethyl)pyrimidine-2,4-diamine as a yellow oil. MS m/z 383 (MH)⁺.

### EXAMPLE 16

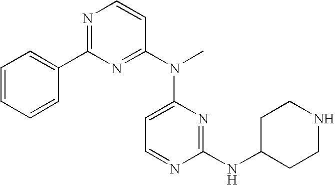 N⁴-Methyl-N⁴-(2-phenylpyrimidin-4-yl)-N²-(piperidin-4-yl)pyrimidine-2,4-diamine.

N-(2-Fluoropyrimidin-4-yl)-N-methyl-2-phenylpyrimidin-4-amine (442 mg, 1.5 mmol), tert-butyl 4-aminopiperidine-1-carboxylate (451 mg, 2.25 mmol), and dioxane (7 mL) were mixed in a 25 mL roundbottom flask. The mixture was stirred at 100° C. overnight under nitrogen. The reaction was concentrated by vacuum and re-dissolved in 1:1 CH₂Cl₂/saturated NaHCO₃ solution. The layers were separated and the aqueous layer was extracted with CH₂Cl₂ once. The combined organic layers were washed once with brine, dried (Na₂SO₄), filtered, and concentrated. The crude product was purified by column chromatography.

The above compound (245 mg, 0.53 mmol) was treated with 1:1 TFA/CH₂Cl₂ (14 mL) for 20 min. The solution was concentrated under vacuum and purified by column chromatography to give N⁴-methyl-N⁴-(2-phenylpyrimidin-4-yl)-N²-(piperidin-4-yl)pyrimidine-2,4-diamine (270 mg) MS m/z 361 (MH)⁺.

### EXAMPLE 17

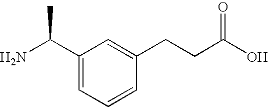 (S)-3-(3-(1-Aminoethyl)phenyl)propanoic acid.
Step A: (S)-3-(3-(1-(tert-Butoxycarbonyl)ethyl)phenyl)propanoic acid.

A mixture of (S,E)-methyl 3-(3-(1-(tert-butoxycarbonyl)ethyl)phenyl)acrylate (5.0 g, 16.4 mmol) and rhodium on carbon (1 g) in methanol (50 mL) was placed under an atmosphere of hydrogen (balloon) and stirred for 12 h. The mixture was then carefully filtered through celite and concentrated to give the saturated ester (4.91 g, 16.0 mmol). The saturated ester was dissolved in a mixture of methanol (50 mL) and aqueous sodium hydroxide (16 mL, 5N) and heated to 65° C. for 1 h. After cooling to RT, about 75% of the methanol was removed under vacuum and the remainder of the reaction medium was partitioned between water and chloroform. The layers were separated and the aqueous layer was washed with chloroform. The aqueous layer was then taken to pH 4 with 10% KHSO₄, and the product was extracted with chloroform several times. Concentration of the extracts gave (S)-methyl 3-(3-(1-(tert-butoxy-carbonyl)ethyl)phenyl)propanoate as an oil. MS m/z 316 (M+Na)⁺.

Step B: (S)-3-(3-(1-Aminoethyl)phenyl)propanoic acid.

Trifluoroacetic acid (2.5 mL), CH₂Cl₂ (2.5 mL) and the Boc protected amine (200 mg, 0.68 mmol) were mixed in a 25 mL roundbottom flask fitted with a magnetic stir bar. The mixture was stirred at RT for 1 h and the solvent was removed under vacuum. The mixture was partitioned between saturated sodium bicarbonate (aq.) and CH₂Cl₂, the layers were separated, and the aqueous layer was extracted with CH₂Cl₂ three times. The extracts were dried (MgSO₄), filtered, concentrated under vacuum, and purified by column chromatography to give the TFA salt of (S)-3-(3-(1-aminoethyl)phenyl)-propanoic acid (200 mg). MS m/z 194 (MH)⁺.

### EXAMPLE 18

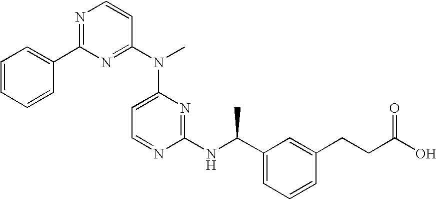 (S)-3-(3-(1-(4-(Methyl(2-phenylpyrimidin-4-yl)amino)pyrimidin-2-ylamino)-ethyl)phenyl)propanoic acid.

N-(2-Fluoropyrimidin-4-yl)-N-methyl-2-phenyl-pyrimidin-4-amine (105 mg, 0.33 mmol) and (S)-3-(3-(1-aminoethyl)phenyl)-propanoic acid (100 mg, 0.33 mmol) were dissolved in 1:1 DMF/dioxane (3 mL) in a 25 mL round-bottom flask. To this Na₂CO₃ (241 mg, 2.28 mmol) was added and the mixture was stirred at 100° C. overnight. The reaction was quenched with saturated solution of NaHCO₃ and extracted twice with CH₂Cl₂. The aqueous layer was then acidified with 2N HCl (aq.) and extracted with 1:4 IPA/CHCl₃ solution four times. The organic layer was dried with Na₂SO₄ and concentrated. Finally, the crude material was purified by column chromatography to give (S)-3-(3-(1-(4-(methyl(2-phenylpyrimidin-4-yl)amino)pyrimidin-2-ylamino)ethyl)phenyl)-propanoic acid. MS m/z 454 (MH)⁺.

### EXAMPLE 19

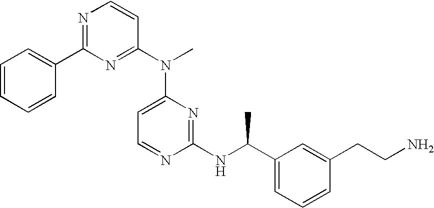 (S)-N²-(1-(3-(2-Aminoethyl)phenyl)ethyl)-N⁴-methyl-N⁴-(2-phenylpyrimidin-4-yl)pyrimidine-2,4-diamine.

N-(2-Fluoropyrimidin-4-yl)-N-methyl-2-phenylpyrimidin-4-amine (108 mg, 0.38 mmol) and (S)-benzyl 3-(1-aminoethyl)-phenethylcarbamate (100 mg, 0.38 mmol) were dissolved in 1:1 DMF/dioxane (3 mL) in a 25 mL round-bottom flask. To this Na₂CO₃ (177 mg, 1.92 mmol) was added and the mixture was stirred at 100° C. overnight. The reaction was cooled to RT and quenched with saturated NaHCO₃ (aq) and extracted twice with CH₂Cl₂. The combined organic layer was washed once with brine, dried (Na₂SO₄), filtered, and concentrated. The crude product (83 mg, 0.15 mmol) was purified by column chromatography.

The above material was re-dissolved in MeOH (0.5 mL) and placed under an atmosphere of H₂ (balloon) in the presence of 10% palladium on carbon (20 mg). The mixture was stirred overnight, filtered through celite and concentrated by vacuum. The crude oil was purified by column chromatography to give (S)-N²-(1-(3-(2-aminoethyl)phenyl)ethyl)-N⁴-methyl-N⁴-(2-phenylpyrimidin-4-yl)pyrimidine-2,4-diamine. MS m/z 425 (MH)⁺.

### EXAMPLE 20

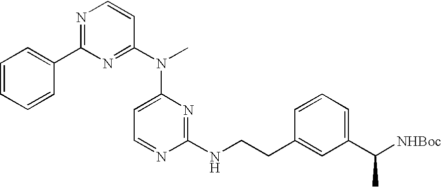 (S)-tert-Butyl 1-(3-(2-(4-(methyl(2-phenylpyrimidin-4-yl)amino)pyrimidin-2-yl-amino)ethyl)phenyl)ethylcarbamate.

N-(2-Fluoropyrimidin-4-yl)-N-methyl-2-phenylpyrimidin-4-amine (558 mg, 1.98 mmol) and (S)-tert-butyl 1-(3-(2-amino-ethyl)phenyl)ethylcarbamate (457 mg, 1.72 mmol) were dissolved in 1:1 DMF/dioxane (3 mL) in a 50 mL round-bottom flask. To this Na₂CO₃ (912 mg, 8.60 mmol) was added and the mixture was stirred at 100° C. overnight. The reaction was cooled to RT and quenched with saturated solution of NaHCO₃ and extracted twice with CH₂Cl₂. The combined organic layer was washed once with brine, dried (Na₂SO₄), filtered, and concentrated. The crude material was purified by column chromatography to give (S)-tert-butyl 1-(3-(2-(4-(methyl(2-phenyl-pyrimidin-4-yl)amino)pyrimidin-2-ylamino)ethyl)phenyl)ethylcarbamate as a white solid. MS m/z 525 (MH)⁺.

### EXAMPLE 21

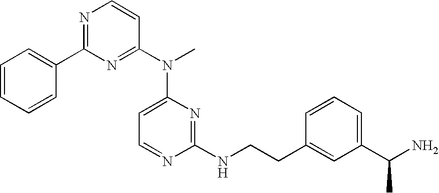 (S)-N²-(3-(1-Aminoethyl)phenethyl)-N⁴-methyl-N⁴-(2-phenylpyrimidin-4-yl)-pyrimidine-2,4-diamine.

Example 20 (264 mg, 0.53 mmol) was treated with 1:1 TFA/CH₂Cl₂ (14 mL) for 20 min. The solution was concentrated and purified by column chromatography to give (S)-N²-(3-(1-aminoethyl)phenethyl)-N⁴-methyl-N⁴-(2-phenylpyrimidin-4-yl)pyrimidine-2,4-diamine. MS m/z 425 (MH)⁺.

### EXAMPLE 22

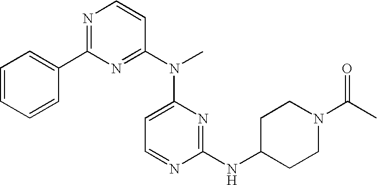 1-(4-(4-(Methyl(2-phenylpyrimidin-4-yl)amino)pyrimidin-2-ylamino)piperidin-1-yl)ethanone.

Triethylamine (0.014 mL, 0.1 mmol) was added to a solution of N⁴-methyl-N⁴-(2-phenylpyrimidin-4-yl)-N²-(piperidin-4-yl)pyrimidine-2,4-diamine (30 mg, 0.083 mmol) and acetic anhydride (0.016 mL, 0.166 mmol) in CH₂Cl₂ (1 mL) at RT, and the mixture was stirred for one hour. The reaction was quenched with 5% citric acid (aq) and extracted with CH₂Cl₂ two times. The organic layer was dried (Na₂SO₄), filtered, and concentrated to give 1-(4-(4-(methyl(2-phenylpyrimidin-4-yl)amino)pyrimidin-2-ylamino)piperidin-1-yl)ethanone. MS m/z 403 (MH)⁺.

### EXAMPLE 23

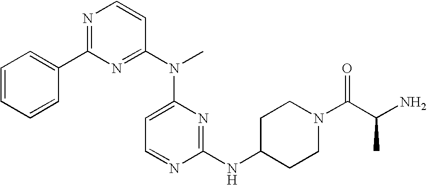 (S)-2-Amino-N-(4-(methyl(2-phenylpyrimidin-4-yl)amino)pyrimidin-2-yl)-propanamide.

To a solution of N⁴-methyl-N⁴-(2-phenylpyrimidin-4-yl)-N²-(piperidin-4-yl)pyrimidine-2,4-diamine (20 mg, 0.055 mmol) in CH₂C₂ (0.6 mL), was added PS-carbodiimide (86 mg, 0.11 mmol) and the mixture was stirred at RT for 15 min. Fmoc-L-Alanine (34.5 mg, 0.11 mmol) was added and the mixture was stirred for 5 h. The resin was filtered off and the filtrate was washed with saturated NH₄Cl (aq) and then brine. The organic layer was dried (Na₂SO₄) and concentrated. The crude material was purified by column chromatography.

The above material (10.8 mg, 0.017 mmol) was treated with piperidine (1 mL) at 70° C. for 30 min. The Piperidine was evaporated under vacuum and the residue was purified by column chromatography to give (S)-2-amino-N-(4-(methyl(2-phenyl-pyrimidin-4-yl)amino)pyrimidin-2-yl)propanamide. MS m/z 432 (MH)⁺.

### EXAMPLE 24

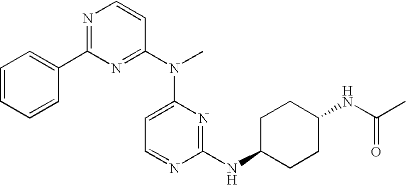 N-(4-(4-(Methyl(2-phenylpyrimidin-4-yl)amino)pyrimidin-2-ylamino)cyclohexyl)-acetamide.

Triethylamine (0.018 mL, 0.128 mmol) was added to a solution of N²-(4-aminocyclohexyl)-N⁴-methyl-N⁴-(2-phenylpyrimidin-4-yl)pyrimidine-2,4-diamine (40 mg, 0.107 mmol) and acetic anhydride (0.02 mL, 0.214 mmol) in CH₂Cl₂ (1 mL), and the mixture was stirred at RT for one hour. The reaction was quenched with 5% citric acid and extracted with CH₂Cl₂ two times. The organic layer was dried (Na₂SO₄), filtered, and concentrated to give N-(4-(4-(methyl(2-phenylpyrimidin-4-yl)amino)pyrimidin-2-ylamino)cyclohexyl)acetamide. MS m/z 417 (MH)⁺.

### EXAMPLE 25

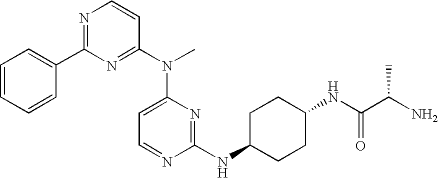 (S)-2-Amino-N-(4-(4-(methyl(2-phenylpyrimidin-4-yl)amino)pyrimidin-2-ylamino)cyclohexyl)propanamide.

To a solution of N²-(4-aminocyclohexyl)-N⁴-methyl-N⁴-(2-phenylpyrimidin-4-yl)pyrimidine-2,4-diamine (40 mg, 0.107 mmol) in CH₂Cl₂ (1.0 mL) was added PS-carbodiimide (167 mg, 0.214 mmol) and the mixture was stirred at RT for 15 min. To this Fmoc-L-Alanine (66 mg, 0.214 mmol) was added and the mixture was stirred for 5 h. The resin was filtered through a fritted funnel and the filtrate was concentrated by vacuum. The crude was purified by column chromatography. The pure intermediate (53 mg, 0.079 mmol) was treated with piperidine (5 mL) at 70° C. for 30 min. Piperidine was evaporated by vacuum and the crude oil was purified by column chromatography to give (S)-2-amino-N-(4-(4-(methyl(2-phenylpyrimidin-4-yl)amino)pyrimidin-2-ylamino)-cyclohexyl)propanamide. MS m/z 446 (MH)⁺.

### EXAMPLE 26

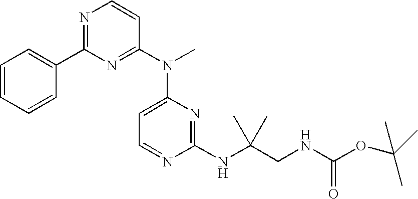 tert-Butyl 2-methyl-2-(4-(methyl(2-phenylpyrimidin-4-yl)amino)pyrimidin-2-yl-amino)propylcarbamate.

N-(2-Fluoropyrimidin-4-yl)-N-methyl-2-phenylpyrimidin-4-amine (660 mg, 2.35 mmol) and tert-butyl 2-amino-2-methylpropylcarbamate (664 mg, 3.53 mmol) were heated to 100° C. in dioxane (15 mL) overnight. The reaction was concentrated under vacuum and re-dissolved in 1:1 CH₂Cl₂/saturated NaHCO₃ solution. The layers were separated and the aqueous layer was extracted with CH₂Cl₂ once. The combined organic layer was washed once with brine, dried in Na₂SO₄, filtered, and concentrated. The crude product was purified by column chromatography to give tert-butyl 2-methyl-2-(4-(methyl(2-phenylpyrimidin-4-yl)-amino)pyrimidin-2-ylamino)propylcarbamate as a light yellow solid. MS 449 m/z (MH)⁺.

### EXAMPLE 27

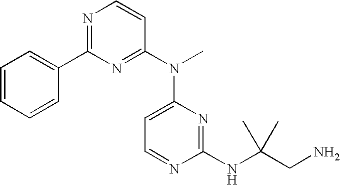 N²-(1-Amino-2-methylpropan-2-yl)-N⁴-methyl-N⁴-(2-phenylpyrimidin-4-yl)-pyrimidine-2,4-diamine.

Trifluoroacetic acid (5 mL) was added to a dichloro-methane solution (5 mL) of tert-butyl 2-methyl-2-(4-(methyl(2-phenylpyrimidin-4-yl)amino)pyrimidin-2-ylamino)propylcarbamate (298.5 mg, 0.66 mmol) in a 25 mL round-bottom flask equipped with a magnetic stir bar. The mixture was stirred at RT for 2 h and the solvent was removed under vacuum. The mixture was partitioned between saturated sodium bicarbonate (aq.) and CH₂Cl₂, the layers were separated, and the aqueous layer was extracted with CH₂Cl₂ three times. The extracts were dried (Na₂SO₄), filtered, concentrated under vacuum, and purified by column chromatography to give N²-(1-amino-2-methylpropan-2-yl)-N⁴-methyl-N⁴-(2-phenylpyrimidin-4-yl)pyrimidine-2,4-diamine. MS m/z 349 (MH)⁺.

### EXAMPLE 28

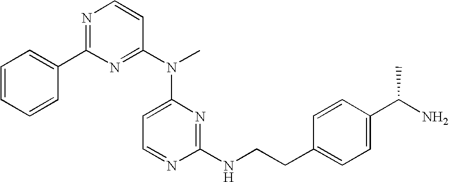 (S)-N²-(4-(1-Aminoethyl)phenethyl)-N⁴-methyl-N⁴-(2-phenylpyrimidin-4-yl)-pyrimidine-2,4-diamine.

(S)-tert-Butyl1-(4-(2-(4-(methyl(2-phenylpyrimidin-4-yl)-amino)pyrimidin-2-ylamino)ethyl)phenyl)ethylcarbamate (525 mg, 0.17 mmol) was treated with a solution of 4M HCl in dioxane (1 mL) and CH₂Cl₂ (1 mL). The mixture was stirred at RT for two hours then quenched with saturated solution of NaHCO₃. The layers were separated and the aqueous layer was extracted with CH₂Cl₂. The combined organic layer was dried (Na₂SO₄) and concentrated under vacuum. The residue was purified by column chromatography to give (S)-N²-(4-(1-aminoethyl)phenethyl)-N⁴-methyl-N⁴-(2-phenylpyrimidin-4-yl)pyrimidine-2,4-diamine. MS m/z 425 (MH)⁺.

### EXAMPLE 29

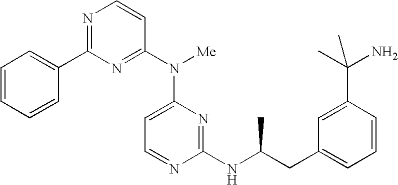 (S)-N²-(1-(3-(2-Aminopropan-2-yl)phenyl)propan-2-yl)-N⁴-methyl-N⁴-(2-phenylpyrimidin-4-yl)pyrimidine-2,4-diamine
Step A: (S)-Benzyl 1-(3-(2-hydroxypropan-2-yl)phenyl)propan-2-ylcarbamate:

To a stirring solution of (S)-benzyl 1-(3-acetylphenyl)propan-2-ylcarbamate (0.5 g, 1.6 mmol) in THF at −78° C. was added 3M methylmagnesium bromide (5.4 mL, 16 mmol) in diethyl ether. After 20 min, the cooling bath was removed, and the solution warmed to 0° C. Reaction quenched with drop-wise addition to saturated ammonium chloride. Organic extracted twice with 50 mL ethyl acetate, dried with magnesium sulfate, and distilled to a residue under reduced pressure. Residue then purified on silica eluting with ethyl acetate/hexanes. Product isolated as colorless oil.

Step B: (S)-Benzyl 1-(3-(2-azidopropan-2-yl)phenyl)propan-2-ylcarbamate.

To stirring 1M hydrazoic acid (15 mL) in toluene at RT was added (S)-benzyl 1-(3-(2-hydroxypropan-2-yl)phenyl)propan-2-ylcarbamate (1.0 g, 3.1 mmol), trifluoroacetic acid (0.5 mL), and magnesium sulfate (400 mg). Mixture stirred for two hours. Solvents distilled under reduced pressure, and residue partitioned between ethyl acetate (50 mL) and 5% aqueous sodium bicarbonate. Organic dried with magnesium sulfate, filtered, and distilled to colorless oil under reduced pressure.

Step C: (S)-1-(3-(2-Aminopropan-2-yl)phenyl)propan-2-amine.

To a stirring solution of (S)-benzyl 1-(3-(2-azidopropan-2-yl)phenyl)propan-2-ylcarbamate (1.0 g, 2.9 mmol) in 30 mL methanol was added 75 mg palladium hydroxide (20% on carbon) and mixture stirred over an atmosphere of hydrogen. After 3 h, the reaction was filtered through a bed of Celite and distilled to colorless oil under reduced pressure.

Step D: (S)-N²-(1-(3-(2-Aminopropan-2-yl)phenyl)propan-2-yl)-N⁴-methyl-N⁴(2-phenylpyrimidin-4-yl)pyrimidine-2,4-diamine.

A solution of (S)-1-(3-(2-amino-propan-2-yl)phenyl)propan-2-amine (250 mg, 1.3 mmol) and N-(2-fluoropyrimidin-4-yl)-N-methyl-2-phenylpyrimidin-4-amine (280 mg, 1.0 mmol) in 1,4-dioxane (10 mL) was heated to 90° C. for 18 h. Solvent distilled under reduced pressure and resulting residue partitioned between dichloromethane (20 mL) and 1N sodium hydroxide (5 mL). Aqueous extracted four times with dichloromethane (5 mL). Combined organics dried over magnesium sulfate, distilled to oil under reduced pressure, then purified on silica. Product isolated as colorless oil. MS m/z 454 (MH)⁺.

### EXAMPLE 30

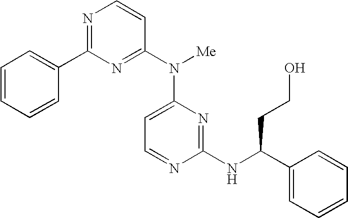 (S)-3-(4-(Methyl(2-phenylpyrimidin-4-yl)amino)pyrimidin-2-ylamino)-3-phenylpropan-1-ol.

The compound was prepared similar to that of (S)-N²-(1-(3-(2-aminopropan-2-yl)phenyl)propan-2-yl)-N⁴-methyl-N⁴-(2-phenylpyrimidin-4-yl)pyrimidine-2,4-diamine (Example 29). MS m/z 413 (MH)⁺.

### EXAMPLE 31

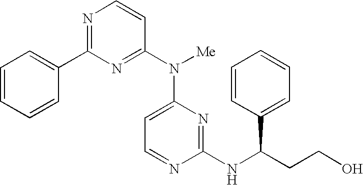 (R)-3-(4-(Methyl(2-phenylpyrimidin-4-yl)amino)pyrimidin-2-ylamino)-3-phenylpropan-1-ol.

The compound was prepared similar to that of (S)-N²-(1-(3-(2-aminopropan-2-yl)phenyl)propan-2-yl)-N⁴-methyl-N⁴-(2-phenylpyrimidin-4-yl)pyrimidine-2,4-diamine (Example 29). MS m/z 413 (MH)⁺.

### EXAMPLE 32

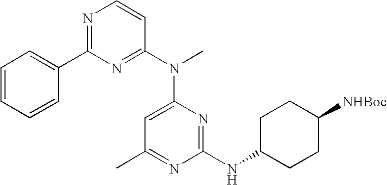 tert-Butyl (1r,4r)-4-(4-methyl-6-(methyl(2-phenylpyrimidin-4-yl)amino)pyrimidin-2-ylamino)cyclohexylcarbamate
Step A: N-(2-Fluoro-6-methylpyrimidin-4-yl)-N-methyl-2-phenylpyrimidin-4-amine.

A solution of N-methyl-2-phenylpyrimidin-4-amine (1.0 g, 4.52 mmol) in N,N-dimethylformamide (DMF) (10 mL) was brought to 0° C. followed by the addition of sodium hydride (NaH 60% in mineral oil) (0.22 g, 5.42 mmol). The resulting redish solution was stirred at 0° C. for 15 min then, 2,4-difluoro-6-methylpyrimidine (0.71 g, 5.42 mmol) was added. The resulting mixture was stirred at 0° C. for 2.5 h more and quenched with water. The resulting orange suspension was extracted with ethyl acetate. The organic extracts were combined, washed with saturated NH₄Cl, brine, dried over magnesium sulfate, concentrated and chromatographed on silica gel using 0-4% MeOH/CH₂Cl₂ to afford a yellow oil MS m/z 296 (MH)⁺.

Step B: tert-Butyl (1r,4r)-4-(4-methyl-6-(methyl(2-phenylpyrimidin-4-yl)amino)-pyrimidin-2-ylamino)cyclohexylcarbamate.

A mixture of N-(2-fluoro-6-methylpyrimidin-4-yl)-N-methyl-2-phenylpyrimidin-4-amine (80 mg, 0.27 mmol), tert-butyl (1r,4r)-4-aminocyclohexylcarbamate (58 mg, 0.27 mmol) and N,N-diisopropylethylamine (47 μL, 0.27 mmol) in dioxane (2 mL) was heated to 95° C. for 15 h. The mixture was brought to RT, diluted in ethyl acetate, washed with saturated NH₄Cl, brine, dried over magnesium sulfate, concentrated and chromatographed on silica gel using 0-4% MeOH/CH₂Cl₂. MS m/z 490 (MH)⁺.

### EXAMPLE 33

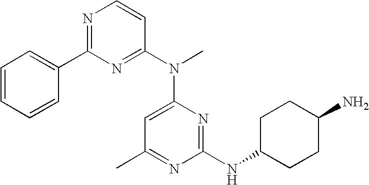 N2-((1r,4r)-4-Aminocyclohexyl)-N4,6-dimethyl-N4-(2-phenylpyrimidin-4-yl)pyrimidine-2,4-diamine.

Procedure same as on Example 27. MS m/z 390 (MH)⁺.

### EXAMPLE 34

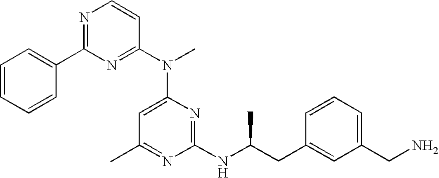 (S)-N²-(1-(3-(Aminomethyl)phenyl)propan-2-yl)-N⁴,6-dimethyl-N⁴-(2-phenylpyrimidin-4-yl)pyrimidine-2,4-diamine
Step A: (S)-3-(2-(4-Methyl-6-(methyl(2-phenylpyrimidin-4-yl)amino)pyrimidin-2-ylamino)propyl)benzonitrile.

Procedure same as on Example 32 step B. MS m/z 436 (MH)⁺.

Step B: (S)-N²-(1-(3-(Aminomethyl)phenyl)propan-2-yl)-⁴,6-dimethyl-N⁴-(2-phenylpyrimidin-4-yl)pyrimidine-2,4-diamine.

A mixture of (S)-3-(2-(4-methyl-6-(methyl(2-phenylpyrimidin-4-yl)amino)pyrimidin-2-ylamino)propyl)benzonitrile (60 mg, 0.14 mmol), Raney-Ni (10 eq) in dioxane (5 mL) was heated to 90° C. for 2.5 h and brought to RT. The mixture was decanted and the remaining Raney-Ni was extracted with aqueous NH₄OH and dichloromethane. The organic extracts were combined, dried over magnesium sulfate, concentrated and chromatographed on silica gel using 0-8% 2N NH₃MeOH/CH₂Cl₂. MS m/z 440 (MH)⁺.

### EXAMPLE 35

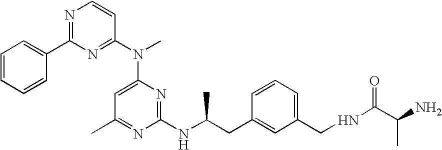 (S)-N-((S)-3-((S)-2-(4-Methyl-6-(methyl(2-phenylpyrimidin-4-yl)amino)pyrimidin-2-ylamino)propyl)benzyl)-2-aminopropanamide.

Procedure same as on Example 23. MS m/z 511 (MH)⁺.

### EXAMPLE 36

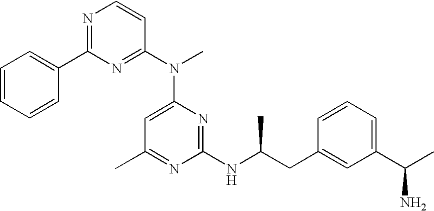 N²-((S)-1-(3-((R)-1-Aminoethyl)phenyl)propan-2-yl)-N⁴,6-dimethyl-N⁴-(2-phenylpyrimidin-4-yl)pyrimidine-2,4-diamine
Step A:

A mixture of (S)-benzyl-1(3-acetylphenyl)propan-2-ylcarbamate (6 g, 19.3 mmol), 2-methyl-2-propanesulfinamide (4.6 g, 38.6 mmol) and titanium (IV) ethoxide (8.1 mL, 38.6 mmol) in THF (60 mL) was heated to 70° C. for 18 h. The mixture was brought to RT and cooled to −48° C. (dry ice/CH₃CN). To this solution was added NaBH₄ (3.5 g, 96.5 mmol) portion wise. The resulting suspension was stirred at −48° C. until complete reduction of the imine (3.5 h). The mixture was brought to RT, quenched with saturated NaHCO₃, brine, dried over magnesium sulfate, and concentrated to be used as is. MS m/z 417 (MH)⁺.

Step B: Benzyl (S)-1-(3-((R)-1-aminoethyl)phenyl)propan-2-ylcarbamate.

A mixture of starting material from Step A (9.56 g, 23 mmol) and 4.0M HCl/dioxane (17.3 mL, 69 mmol) in methanol (20 mL) was stirred at RT for 1.5 h and concentrated. The residue obtained was dissolved in dichloromethane, washed with saturated NaHCO₃, brine, dried over magnesium sulfate, concentrated and chromatographed on silica gel using 0-8% 2N NH₃MeOH/CH₂Cl₂ to afford a very light-yellow oil. MS m/z 313 (MH)⁺.

Step C:

Procedure same as on Example 119 step A. White solid. MS m/z 413 (MH)⁺.

Step D: tert-Butyl (R)-1-(3-((S)-2-aminopropyl)phenyl)ethylcarbamate.

Through a mixture of the starting material (3.9 g, 9.5 mmol), Pd/C (1.3 g) in MeOH (50 mL) was bubbled hydrogen through a balloon for 5 h. The mixture was filtered through celite and concentrated to afford a pale yellow oil. MS m/z 279 (MH)⁺.

Step E: tert-Butyl (R)-1-(3-((S)-2-(4-methyl-6-(methyl(2-phenylpyrimidin-4-yl)-amino)pyrimidin-2-ylamino)propyl)phenyl)ethylcarbamate.

Procedure same as on Example 32, step B. Light yellow oil. MS m/z 554 (MH)⁺.

Step F: N²-((S)-1-(3-((R)-1-aminoethyl)phenyl)propan-2-yl)-N⁴,6-dimethyl-N⁴-(2-phenylpyrimidin-4-yl)pyrimidine-2,4-diamine.

Procedure same as on Example 27. MS m/z 454 (MH)⁺.

### EXAMPLE 37

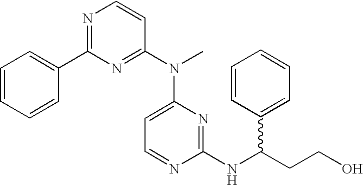 3-(4-(Methyl(2-phenylpyrimidin-4-yl)amino)pyrimidin-2-ylamino)-3-phenylpropan-1-ol.

Procedure same as on Example 32, step B. Very light yellow crystalline solid. MS m/z 413 (MH)⁺.

### EXAMPLE 38

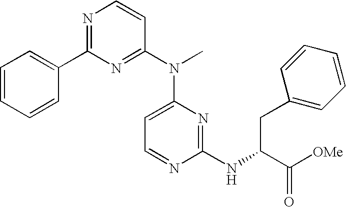 (R)-Methyl-2-(4-(methyl(2-phenylpyrimidin-4-yl)amino)pyrimidin-2-ylamino)3-phenylpropanoate.

Procedure same as on Example 32, step B. Very light yellow crystalline solid. MS m/z 441 (MH)⁺.

### EXAMPLE 39

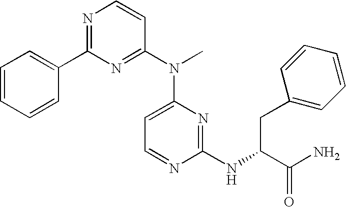 (R)-2-(Methyl(2-phenylpyrimidin-4-yl)amino)pyrimidin-2-ylamino)-3-phenyl-propanamide.

A mixture of (R)-methyl-2-(4-(methyl(2-phenylpyrimidin-4-yl)amino)pyrimidin-2-ylamino)3-phenylpropanoate (0.15 mg, 0.34 mmol), ammonium hydroxide(10 mL) and 2N NH₃MeOH (10 mL) was heated in a sealed tube at 80° C. for 15 h. The mixture was brought to RT, concentrated and chromatographed on silica gel using 0-2% MeOH/CH₂Cl₂ to afford a white solid. MS m/z 426 (MH)⁺.

### EXAMPLE 40

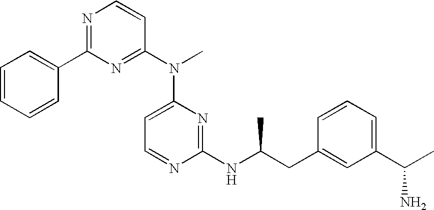 N²-((S)-1-(3-((S)-1-Aminoethyl)phenyl)propan-2-yl)-N⁴-methyl-N⁴-(2-phenyl-pyrimidin-4-yl)pyrimidine-2,4-diamine.

Procedure same as on Example 36. MS m/z 440 (MH)⁺.

### EXAMPLE 41

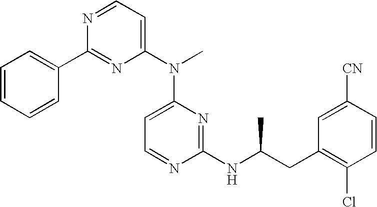 (S)-4-Chloro-3-(2-(4-(methyl(2-phenylpyrimidin-4-yl)amino)pyrimidin-2-ylamino)-propyl)benzonitrile.

A solution of (S)-3-(2-aminopropyl)-4-chlorobenzonitrile (0.060 g, 0.31 mmol), 2-fluoro-N-methyl-N-(2-phenylpyrimidin-4-yl)pyrimidin-4-amine (0.090 g, 0.32 mmol), and Hunig's base (0.30 mL, 1.7 mmol) in dioxane (1.0 mL) was heated to 140° C. in a sand bath for 4 h. The cooled mixture was loaded to a silica column and eluded with hexanes, then hexanes-EtOAc (1:1) to afford the desired product as a yellow solid. MS m/z 456 (MH)⁺.

### EXAMPLE 42

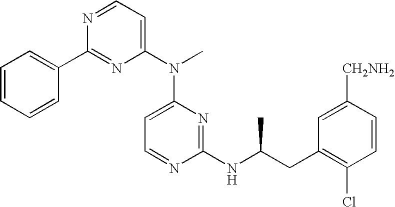 (S)-N²-(1-(5-(Aminomethyl)-2-chlorophenyl)propan-2-yl)-N⁴-methyl-N⁴-(2-phenyl-pyrimidin-4-yl)pyrimidine-2,4-diamine.

A mixture of (S)-4-chloro-3-(2-(4-(methyl-(2-phenylpyrimidin-4-yl)amino)pyrimidin-2-ylamino)propyl)benzonitrile (0.039 g, 0.086 mmol), NiCl₂ (0.015 g, 0.12 mmol), and NaBH₄ (0.010 g, 0.26 mmol) in EtOH (2.0 mL) was purged with nitrogen and stirred at RT. After 25 min, a second batch of the reducing reagents was added and the mixture was stirred for 1.5 h. The reaction mixture was filtered through a pad of Celite, washed with MeOH and concentrated. The resulting residue was partitioned between H₂O and DCM, and the organic phase was concentrated and purified on silica (2-10% 2 M NH₃-MeOH in DCM). Further purification on RP HPLC (10-80% CH₃CN in H₂O) yielded the pure product (2.0 mg). MS m/z 460 (MH)⁺.

### EXAMPLE 43

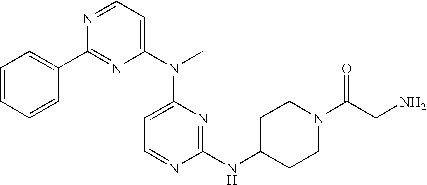 2-Amino-1-(4-(4-(methyl(2-phenylpyrimidin-4-yl)amino)pyrimidin-2-ylamino)-piperidin-1-yl)ethanone.

A mixture of N⁴-methyl-N⁴-(2-phenylpyrimidin-4-yl)-N²-(piperidin-4-yl)pyrimidine-2,4-diamine (350 mg, 0.97 mmol), 2-(tert-butoxy-carbonyl)acetic acid (1 eq), EDC (1 eq), HOBt (1 eq), DIEA (1 eq) in CHCl₃ (10 mL) was stirred at RT for 4 h, the resulting mixture was diluted with CHCl₃ and aq NaHCO₃. The separated organic layer was washed with brine, dried, and concentrated to yield the crude product, which was purified with with flash column chromatography (pure DCM, 5% MeOH in DCM) to afford tert-butyl 2-(4-(4-(methyl(2-phenylpyrimidin-4-yl)amino)pyrimidin-2-ylamino)piperidin-1-yl)-2-oxoethylcarbamate. It was then deprotected with TFA-DCM (RT, 1 h) to provide the title compound as a white solid. MS m/z 419.2 (M+H)⁺.

### EXAMPLE 44

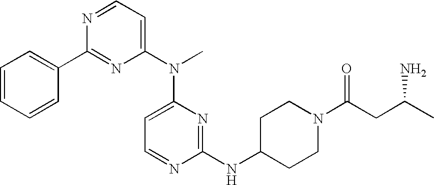 (R)-3-Amino-1-(4-(4-(methyl(2-phenylpyrimidin-4-yl)amino)pyrimidin-2-ylamino)-piperidin-1-yl)butan-1-one.

The title compound was isolated as a white solid according to the similar sequences as described previously from N⁴-methyl-N⁴-(2-phenylpyrimidin-4-yl)-N²-(piperidin-4-yl)pyrimidine-2,4-diamine (Example 43) (0.2 g, 0.55 mmol) and Boc-L-beta-homoalanine (0.11 g, 0.55 mmol). MS m/z 447.3 (MH)⁺.

### EXAMPLE 45

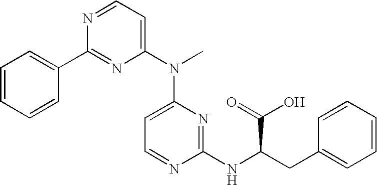 (R)-2-(4-(Methyl(2-phenylpyrimidin-4-yl)amino)pyrimidin-2-ylamino)-3-phenyl-propanoic acid.

(R)-methyl 2-(4-(methyl(2-phenylpyrimidin-4-yl)amino)pyrimidin-2-ylamino)-3-phenylpropanoate (1.2 g, 2.73 mmol) was heated with thiophenol (0.3 g) and K₂CO₃ (5%) in NMP (20 mL) at 190° C. for 30 min. After cooled, the mixture was diluted with NaHCO₃ (aq), and extracted with ether (×3). The aqueous layer was acidified with 6N HCl at 0° C. and then extracted with DCM (×3). Evaporation of the volatile material provided the title compound as a white solid. MS m/z 427.2 (M+H)⁺.

### EXAMPLE 46

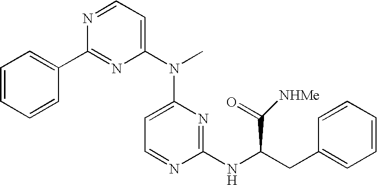 (R)-N-Methyl-2-(4-(methyl(2-phenylpyrimidin-4-yl)amino)pyrimidin-2-ylamino)-3-phenylpropanamide.

The title compound (white solid) was prepared from (R)-2-(4-(methyl(2-phenylpyrimidin-4-yl)amino)pyrimidin-2-ylamino)-3-phenylpropanoic acid (0.2 g, 0.47 mmol) in the similar manner as described previously in Example 51 using PS-carbodiimide as a coupling agent. MS m/z 440.2 (M+H)⁺.

### EXAMPLE 47

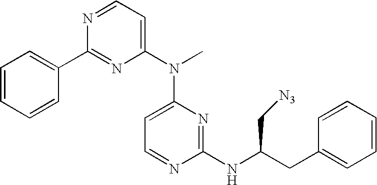 (R)-N²-(3-Azido-1-phenylpropan-2-yl)-N⁴-methyl-N⁴-(2-phenylpyrimidin-4-yl)-pyrimidine-2,4-diamine.

N-(2-Fluoropyrimidin-4-yl)-N-methyl-2-phenylpyrimidin-4-amine (0.32 g, 1.14 mmol) and (R)-1-azido-3-phenylpropan-2-amine (0.32 g, 1.5 eq) was mixed in NMP (5 mL) and heated at 100° C. overnight. The overall solution was concentrated with SiO₂ and purified under a flash column chromatography conditions (pure DCM, 1% MeOH in DCM) to provide the title compound as a pale yellow solid. MS m/z 438.2 (M+H)⁺.

### EXAMPLE 48

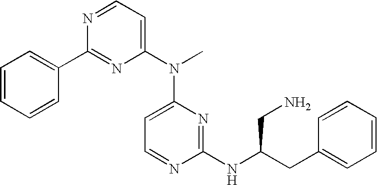 (R)-N²-(1-Amino-3-phenylpropan-2-yl)-N⁴-methyl-N⁴-(2-phenylpyrimidin-4-yl)pyrimidine-2,4-diamine.

A solution of (R)-N²-(3-Azido-1-phenylpropan-2-yl)-N⁴-methyl-N⁴-(2-phenylpyrimidin-4-yl)pyrimidine-2,4-diamine (0.31 g, 0.7 mmol) in THF (10 mL) was added Pd/C (10%, 0.3 g) and the resulting suspension was stirred under a 1 atm H₂ for 16 h. The catalyst was removed over Celite and the filtrated cake was washed with EtOAc, MeOH, and DCM subsequently. The combined organic solvent was concentrated and the residue was purified with flash column chromatography (pure DCM, 5% MeOH in DCM) to afford the title compound as a sticky off-white solid. MS m/z 412.2 (M+H)⁺.

### EXAMPLE 49

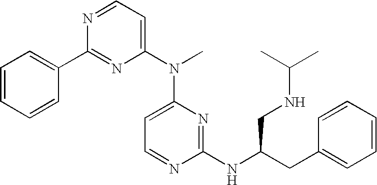 (R)-N²-(3-(Isopropylamino)-1-phenylpropan-2-yl)-N⁴-methyl-N⁴-(2-phenyl-pyrimidin-4-yl)pyrimidine-2,4-diamine.

To a stirred solution of (R)-N²-(1-amino-3-phenylpropan-2-yl)-N⁴-methyl-N⁴-(2-phenylpyrimidin-4-yl)pyrimidine-2,4-diamine (0.25 g, 0.61 mmol) in DCE (5 mL) was added acetone (0.1 mL, 1.22 mmol), acetic acid (3 drops) and sodium triacetoxyborohydride (0.26 g, 1.22 mmol) and the overall mixture was stirred at 50° C. for 2 h prior to being cooled to RT. The resulting solution was washed with saturated NaHCO₃ (aq) and the separated aqueous layer was extracted with DCM. The entire organic solution was dried over sodium sulfate, concentrated and the crude product was purified with flash column chromatography (pure DCM, 5% MeOH in DCM) to afford the title compound as a white sticky foam. MS m/z 454.3 (M+H)⁺.

### EXAMPLE 50

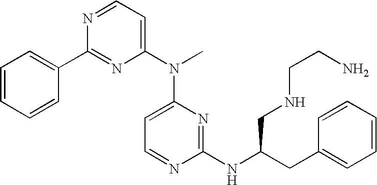 (R)-N²-(3-(2-Aminoethylamino)-1-phenylpropan-2-yl)-N⁴-methyl-N⁴-(2-phenyl-pyrimidin-4-yl)pyrimidine-2,4-diamine.

To a stirred mixture of (R)-N²-(1-amino-3-phenylpropan-2-yl)-N⁴-methyl-N⁴-(2-phenylpyrimidin-4-yl)pyrimidine-2,4-diamine (0.3 g, 0.73 mmol) in acetonitrile (5 mL) was added N-(2-bromoethyl)phthalimide (0.22 g, 0.88 mL) and K₂CO₃(0.2 g, 1.46 mmol) and heated at 85° C. for 16 h. After cooled, the resulting mixture was filtrated and concentrated to give the crude (R)-2-(2-(2-(4-(methyl(2-phenylpyrimidin-4-yl)amino)pyrimidin-2-ylamino)-3-phenylpropylamino)ethyl)isoindoline-1,3-dione, which was treated with excess of hydrazine (1 mL) in EtOH (4 mL) at 70° C. for 1 h. After concentrated, the crude residue was purified with a flash column chromatography (5-10% MeOH in DCM) to afford the title compound as a white solid. MS m/z 455.3 (M+H)⁺.

### EXAMPLE 51

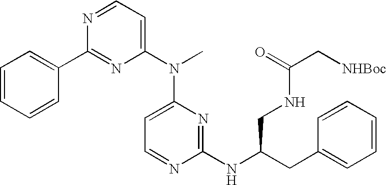 (R)-tert-Butyl 2-(2-(4-(methyl(2-phenylpyrimidin-4-yl)amino)pyrimidin-2-ylamino)-3-phenylpropylamino)-2-oxoethylcarbamate.

A mixture of (R)-N²-(1-amino-3-phenylpropan-2-yl)-N⁴-methyl-N⁴-(2-phenylpyrimidin-4-yl)pyrimidine-2,4-diamine (0.2 g, 0.49 mmol), N-(tert-butoxycarbonyl)-glycine (0.1 g, 0.58 mmol) and polymer-bonded carbodiimide (0.74 g, 2 eq) in DCM (10 mL) was stirred for 16 h at RT and the resulting mixture was filtrated, washed with DCM, and concentrated. The title compound was isolated as an off-white solid after a flash column chromatography (2%-5% MeOH in DCM). MS m/z 569.3 (M+H)⁺.

### EXAMPLE 52

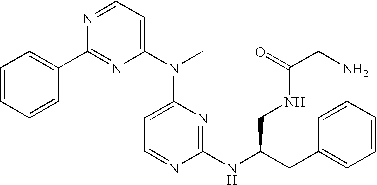 (R)-2-Amino-N-(2-(4-(methyl(2-phenylpyrimidin-4-yl)amino)pyrimidin-2-ylamino)-3-phenylpropyl)acetamide.

(R)-tert-Butyl 2-(2-(4-(methyl(2-phenylpyrimidin-4-yl)amino)pyrimidin-2-ylamino)-3-phenylpropylamino)-2-oxoethylcarbamate (0.1 g, 0.2 mmol) partially dissolved in THF (2 mL) was treated with HCl (4.0M in dioxane, 4 mL) and the overall heterogeneous mixture was stirred at RT for 2 h, concentrated, and azotropically dried with benzene to obtain the title compound as an off-white HCl salt. MS m/z 469.3 (M+H)⁺.

### EXAMPLE 53

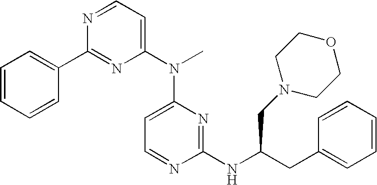 Step A: (R)-2-(tert-butoxycarbonyl)-3-phenylpropyl 4-methylbenzenesulfonate.

A solution of (R)-tert-butyl 1-hydroxy-3-phenylpropan-2-ylcarbamate (5.03 g, 0.021 mol) in DCM (70 mL) was added triethylamine (4.2 mL, 1.5 eq) and TsCl (4.2 g, 1.1 eq) subsequently at 0° C. and the resulting mixture was stirred overnight while allowed to warm up to RT gradually. After quenched with saturated aqueous ammonium chloride, the separated aqueous layer was extracted with DCM and the overall organic phases were dried (Na₂SO₄) and evaporated under a reduced pressure to afford the crude title product as an off-white solid.

Step B: (R)-1-morpholino-3-phenylpropan-2-amine.

Crude tosylate (0.48 g, 1.19 mmol) in acetonitrile (10 mL) was added morpholine (0.21 mL, 2 eq). The overall mixture was heated at 70° C. for 2 h then concentrated to yield a sticky yellow, which was deprotected (4N HCl in dioxane, 4 mL, 2 h) and free of base (PS-carbonate, DCM, 10 min) to yield a crude title compound as a pale yellow oil.

Step C: (R)-N⁴-methyl-N²-(3-morpholino-1-phenylpropan-2-yl)-N⁴-(2-phenylpyrimidin-4-yl)pyrimidine-2,4-diamine.

(2-Fluoropyrimidin-4-yl)-N-methyl-2-phenylpyrimidin-4-amine (0.35 g, 1.26 mmol) and (R)-1-morpholino-3-phenyl-propan-2-amine (0.31 g, 1.39 mmol) was mixed in NMP (5 mL) and heated at 100° C. overnight. The overall solution was concentrated with SiO₂ and purified under a flash column chromatography conditions (pure DCM—5% MeOH in DCM) to provide the title compound as a pale yellow solid. MS m/z 482.3 (M+H)⁺.

### EXAMPLE 54

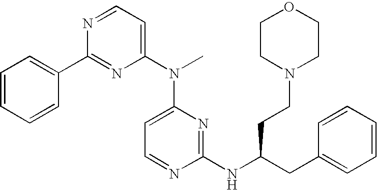 Step A: (R)-4-Morpholino-1-phenylbutan-2-amine.

A suspension of (R)-3-amino-4-phenylbutan-1-ol (2.815 g, 0.017 mol) in dioxane (50 mL) was added 1N NaOH (26 mL, 0.0255 mol) and Boc₂O (4.1 g, 0.0188 mol) subsequently and stirred overnight. The resulting white suspension was diluted with EtOAc and quenched with sat'd NH₄Cl(aq)and the separated aqueous layer was extracted with EtOAc. The overall organic layers were washed with brine and concentrated to provide a crude Boc-carbamate as a semi-solid. A solution of crude carbamate (0.36 g, 1.36 mmol) in DCM (5 mL) was added, at 0° C., Et₃N (0.28 mL, 1.5 eq) and TsCl (0.31 g, 1.2 eq) subsequently and the overall slightly yellow solution was stirred overnight while temperature was warmed to RT naturally. After diluted with sat'd NH₄Cl(aq), the aqueous layer was extracted with DCM and the combined organic layers were washed with brine, dried and evaporated to give the crude tosylate, which was treated with morpholine (0.42 mL, 2 eq) in acetonitrile (3 mL) at RT. The entire solution was heated at 70° C. for 2 h prior to cooled and concentrated. The crude product was slightly purified through a short pad of SiO₂ (2%-5% MeOH in DCM) and the isolated off-white solid was deprotected (4N HCl in dioxane, 4 mL, RT, 1 h), after concentrated, to provide as a white solid.

Step B: (R)-N⁴-Methyl-N²-(4-morpholino-1-phenylbutan-2-yl)-N⁴-(2-phenylpyrimidin-4-yl)pyrimidine-2,4-diamine.

A mixture of crude HCl salt (0.41 g, 1.35 mmol), N-(2-fluoropyrimidin-4-yl)-N-methyl-2-phenylpyrimidin-4-amine (0.30 g, 1.08 mmol) and DIEA (0.71 mL, 3 eq) in NMP was heated at 100° C. overnight. The overall solution was concentrated with SiO₂ and purified under a flash column chromatography conditions (5% MeOH in DCM) to afford the title compound as a light brown foam. MS m/z 496.3 (M+H)⁺.

### EXAMPLE 55

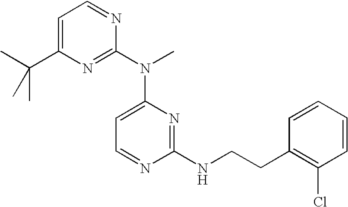 N²-(2-Chlorophenethyl)-N⁴-(4-tert-butylpyrimidin-2-yl)-N⁴-methylpyrimidine-2,4-diamine.

A mixture of N-(4-tert-butylpyrimidin-2-yl)-N-methyl-2-(methylsulfinyl)-pyrimidin-4-amine (0.15 g, 0.5 mmol), 2-(2-chlorophenyl)ethylamine (0.16 g, 1 mmol) pyridine (0.04 g, 0.5 mmol) in DMSO (0.5 mL) was placed in a microwave tube and run in the Personal Chemistry microwave on normal absorption at 150° C. for 15 min. The mixture was diluted with EtOAc, washed with water, sat. sodium chloride, dried over sodium sulfate, concentrated and chromatographed on silica gel with 30% EtOAc/Hexane. MS m/z 397 (MH)⁺.

### EXAMPLE 56

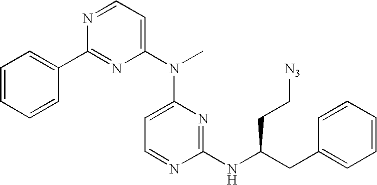 (R)-N²-(4-Azido-1-phenylbutan-2-yl)-N⁴-methyl-N⁴-(2-phenylpyrimidin-4-yl)-pyrimidine-2,4-diamine
Step A: (R)-N²-(4-Azido-1-phenylbutan-2-yl)-N⁴-methyl-N⁴-(2-phenylpyrimidin-4-yl)pyrimidine-2,4-diamine.

The crude tosylate, prepared as described previously from 2 g of crude alcohol (7.55 mmol), was treated with NaN₃ (0.98 g, 15 mmol) in DMF (5 mL) and the overall heterogeneous mixture was stirred at 70° C. for 3 h. After cooled, water was added and extracted with DCM, and the overall extracts were dried and concentrated. The crude pale yellow solid was purified under a flash column chromatographic conditions (1:4 EA/hexanes) to afford (R)-tert-butyl 4-azido-1-phenylbutan-2-ylcarbamate as a white solid.

Step B: (R)-4-Azido-1-phenylbutan-2-amine hydrochloride.

(R)-tert-Butyl 4-azido-1-phenylbutan-2-ylcarbamate (1.8 g, 6.2 mmol) was deprotected (4N HCl in dioxane, 4 mL, RT, 1 h) in dioxane (2 mL) to yield the HCl salt of (R)-4-azido-1-phenylbutan-2-amine as a white solid, which was used directly without purification.

Step C: (R)-N²-(4-Azido-1-phenylbutan-2-yl)-N⁴-methyl-N⁴-(2-phenylpyrimidin-4-yl)pyrimidine-2,4-diamine.

HCl salt of (R)-4-azido-1-phenylbutan-2-amine (1.03 g, 5.43 mmol) was reacted with N-(2-fluoropyrimidin-4-yl)-N-methyl-2-phenyl-pyrimidin-4-amine (1.27 g, 4.53 mmol) in a similar manner as previously described in Example 47, to give, after purification, (R)-N²-(4-azido-1-phenylbutan-2-yl)-N⁴-methyl-N⁴-(2-phenylpyrimidin-4-yl)pyrimidine-2,4-diamine as a white solid. MS m/z 452.2 (M+H)⁺.

### EXAMPLE 57

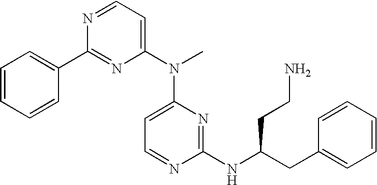 (R)-N²-(4-Amino-1-phenylbutan-2-yl)-N⁴-methyl-N⁴-(2-phenylpyrimidin-4-yl)pyrimidine-2,4-diamine.

Reduction of (R)-N²-(4-azido-1-phenylbutan-2-yl)-N⁴-methyl-N⁴-(2-phenylpyrimidin-4-yl)pyrimidine-2,4-diamine (1.3 g, 2.8 mmol) was conducted in a similar fashion as previously described in Example 48 to provide the title compound as a white foam. MS m/z 426.2 (M+H)⁺.

### EXAMPLE 58

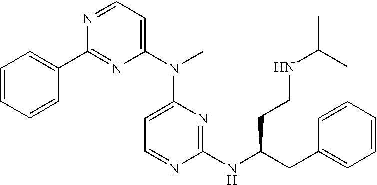 (R)-N²-(4-(Isopropylamino)-1-phenylbutan-2-yl)-N⁴-methyl-N⁴-(2-phenylpyrimidin-4-yl)pyrimidine-2,4-diamine.

The title compound was obtained as a sticky pale yellow film by following the similar method as previously described in Example 49. MS m/z 468 (M+H)⁺.

### EXAMPLE 59

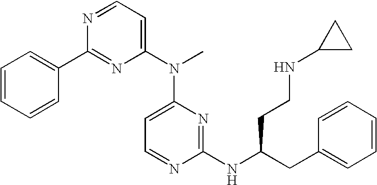 (R)-N²-(4-(Cyclopropylamino)-1-phenylbutan-2-yl)-N⁴-methyl-N⁴-(2-phenyl-pyrimidin-4-yl)pyrimidine-2,4-diamine.

To a stirred solution of (R)-N²-(4-amino-1-phenylbutan-2-yl)-N⁴-methyl-N⁴-(2-phenylpyrimidin-4-yl)pyrimidine-2,4-diamine (0.2 g, 0.47 mmol) in MeOH (5 mL) was added (1-ethoxycyclopropoxy)trimethyl-silane (0.14 mL, 1.5 eq), 4 Å molecule sieves (0.2 g), AcOH (3 drops) and sodium cyanoborohydride (47 mg, 1.5 eq) subsequently and the resulting mixture was stirred at RT for 3 h and 50° C. for 16 h. After cooled and concentrated under reduced pressure, the residue was partitioned between NaHCO₃ (aq, sat'd) and DCM. The separated aqueous layer was extracted with DCM and the combined organic phases were dried (Na₂SO₄) and concentrated. Purification (flash column chromatography, 5% MeOH in DCM) of the crude product gave the title compound as a pale yellow solid. MS m/z 466 (M+H)⁺.

### EXAMPLE 60

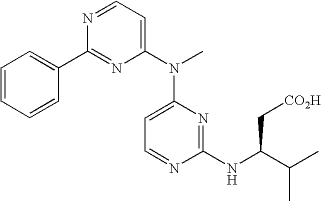 (S)-4-Methyl-3-(4-(methyl(2-phenylpyrimidin-4-yl)amino)pyrimidin-2-ylamino)-pentanoic acid.

L-leucine.HCl (0.32 g, 1.5 eq), DIEA (0.42 mL, 2.4 mmol) and N-(2-fluoropyrimidin-4-yl)-N-methyl-2-phenylpyrimidin-4-amine (0.28 g, 1.14 mmol) was mixed in NMP (2 mL) and heated at 100° C. overnight. The overall solution was concentrated and water was added. The precipitate was collected and washed with water to provide the crude title compound as a pale yellow solid. MS m/z 493 (M+H)⁺.

### EXAMPLE 61

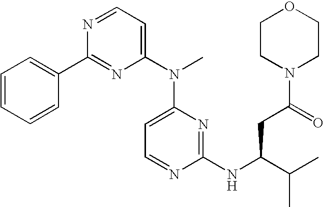 (S)-4-Methyl-3-(4-(methyl(2-phenylpyrimidin-4-yl)amino)pyrimidin-2-ylamine.

A mixture of (S)-4-methyl-3-(4-(methyl(2-phenylpyrimidin-4-yl)amino)pyrimidin-2-ylamino)pentanoic acid. (0.2987 g, 0.76 mmol), morpholine (80 μL, 1.2 mmol) and polymer-bonded carbodiimide (1.5 g, 2.5 eq) in DCM (10 mL) was stirred for 16 h at RT and the resulting mixture was filtrated, washed with DCM, and concentrated. The title compound was isolated as a white foam after a flash column chromatography (2%-5% MeOH in DCM). MS m/z 462 (M+H)⁺.

### EXAMPLE 62

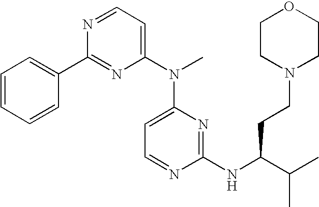 (S)-N⁴-Methyl-N²-(4-methyl-1-morpholinopentan-3-yl)-N⁴-(2-phenylpyrimidin-4-yl)pyrimidine-2,4-diamine.

A solution of (S)-4-methyl-3-(4-(methyl(2-phenyl-pyrimidin-4-yl)amino)pyrimidin-2-ylamino)-1-morpholinopentan-1-one (0.18 g, 0.39 mmol) in THF (5 mL) was added LiAlH₄ (1.0M in THF, 3 eq) at RT and the resulting mixture was stirred at the same temperature for 1 h prior to being cooled to 0° C. and then carefully quenched with aqueous saturated Na₂SO₄ solution (3 drops). The resulting mixture was stirred at RT for an additional 30 min and filtrated. The filtrated cake was washed with EtOAc and DCM subsequently and the combined organic phases were concentrated. Purification of the crude material (flash column chromatography, 5% MeOH in DCM) gave the title compound (55 mg) as a white foam. MS m/z 448 (M+H)⁺.

### EXAMPLE 63

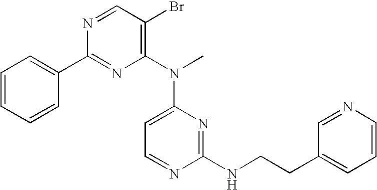 N⁴-(5-Bromo-2-phenylpyrimidin-4-yl)-N⁴-methyl-N²-(2-(pyridin-3-yl)ethyl)-pyrimidine-2,4-diamine
Step A: 5-Bromo-2-phenylpyrimidin-4(3H)one.

Bromine (0.600 mL, 11.6 mmol) was added to a solution of 2-phenylpyrimidin-4(3H)-one (2.00 g, 11.6 mmol) and sodium acetate (3.24 g, 39.4 mmol) in acetic acid (100 mL). The reaction mixture was stirred at RT for 5 h at which time a precipitate had formed. Filtration gave the desired compound. Isolation of a second crop also provided the title compound as a white solid. MS m/z 251 (MH)⁺.

Step B: 5-Bromo-4-chloro-2-phenylpyrimidine.

The pyrimidinone from Step A above (2.30 g, 9.16 mmol) and phosphorus oxychloride (40 mL) were loaded into a vessel equipped with a teflon screw-cap. The vessel was sealed and heated in an oil bath to 120° C. for 24 h. The reaction mixture was cooled to RT and concentrated in vacuo. The residue was then repeatedly combined with toluene and then concentrated (4×50 mL of toluene) to effect azeotropic removal of trace POCl₃. The crude material was dissolved in CH₂Cl₂ (100 mL) and washed with saturated NaHCO₃ solution (1×50 mL). The organic layer was dried over MgSO₄ and concentrated to give the desired compound as an off-white solid. MS m/z 269 (MH)⁺.

Step C: 5-Bromo-N-methyl-2-phenylpyrimidin-4-amine.

The pyrimidine from Step B above (2.47 g, 9.16 mmol), methyl amine (9.16 mL, 18.3 mmol) and IPA (10 mL) were placed in a vessel equipped with a teflon screw-cap. The vessel was sealed and heated in an oil bath to 90° C. for 24 h. The reaction mixture was cooled to RT and acidified to pH 2 with concentrated HCl. The resultant white precipitate was filtered to yield the title compound as the corresponding HCl salt. MS m/z 264 (MH)⁺.

Step D: 5-Bromo-N-(2-fluoropyrimidin-4-yl)-N-methyl-2-phenylpyrimidin-4-amine.

The amine from Step C above (974 mg, 3.24 mmol) was converted to the free amine by partitioning between CHCl₃ (50 mL) and saturated Na₂CO₃ solution (50 mL). The organic layer was dried over MgSO₄ and concentrated. The resultant white solid was then dissolved in THF (30 mL) and cooled to −78° C. Lithium bis-(trimethylsilyl)amide (1.0M in THF, 4.43 mL, 4.43 mmol) was added and after 15 min a solution of 2,4-difluoropyrimidine (505 mg, 4.43 mmol) in THF (5 mL) was added. The brown solution slowly warmed to RT and stirring was continued for 12 h. The reaction mixture was then diluted with Et₂O (50 mL) and 10% NH₄OH solution (50 mL). The organic layer was washed with brine (1×50 mL), dried over MgSO₄ and concentrated. The residue was taken up in CHCl₃, loaded on to a 120 g pre-packed silica gel column and eluted with 2-25% EtOAc:hexanes. Concentration of the appropriate fractions provided the desired compound as a yellow solid. MS m/z 360 (MH)⁺.

Step E: N⁴-(5-Bromo-2-phenylpyrimidin-4-yl)-N⁴-methyl-N²-(2-(pyridin-3-yl)ethyl)pyrimidine-2,4-diamine.

A solution of the pyrimidine from Step D above (0.23 g, 0.63 mmol), 3-(2-aminoethyl)pyridine (92 mg, 0.75 mmol) and N,N-diisopropylethylamine (0.50 mL, 2.5 mmol) in 1,4-dioxane (2 mL) were heated to reflux for 24 h. The reaction mixture was cooled to RT, diluted with CH₂Cl₂ (10 mL) and water (10 mL) and the layers were separated. The aqueous layer was extracted once more with CH₂Cl₂ (10 mL) and the pooled organic phases were washed with brine (1×10 mL), dried over MgSO₄ and concentrated. The residue was taken up in CHCl₃, loaded on to a 40 g pre-packed silica gel column and eluted with 0-5% MeOH(contains 10% NH₄OH):CH₂Cl₂. Concentration of the fractions provided the desired compound as a light yellow solid. MS m/z 462 (MH)⁺.

### EXAMPLE 64

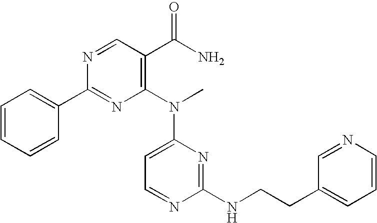 4-(Methyl(2-(2-(pyridin-3-yl)ethylamino)pyrimidin-4-yl)amino)-2-phenyl-pyrimidine-5-carboxamide
Step A: Ethyl 4-hydroxy-2-phenylpyrimidine-5-carboxylate.

A slurry of potassium hydroxide in absolute ethanol (50 mL) was added to a solution of benzamidine hydrochloride (25 g, 0.16 mol) and diethylethoxymethylenemalonate (35 mL, 0.18 mol) in absolute ethanol (150 L). The solution was heated to reflux for 6 h, at which time a white precipitate had formed. The slurry was filtered and the filter cake was washed with cold ethanol. The crude product was dried in a vacuum oven (50° C., 50 Torr) for 24 h, which provided the desired compound as an off-white solid. MS m/z 245 (MH)⁺.

Step B: Ethyl 4-chloro-2-phenylpyrimidine-5-carboxylate.

The pyrimidinone from Step A above (2.71 g, 11.1 mmol) and phosphorus oxychloride (7 mL) were loaded into a vessel equipped with a teflon screw-cap. The vessel was sealed and heated in an oil bath to 100° C. for 24 h. The reaction mixture was cooled to RT and carefully poured over 100 mL of ice. The solution was then neutralized to pH 7 with solid KOH and the resultant brown precipitate was isolated, washed well with water and dried in a vacuum oven (50° C., 50 Torr) for 24 h. The title compound was obtained as a brown solid. MS m/z 263 (MH)⁺.

Step C: Ethyl 4-(methylamino)-2-phenylpyrimidine-5-carboxylate.

The chloro-pyrimidine from Step B above (1.6 g, 6.1 mmol), methyl amine (33% in EtOH, 2.25 mL, 18.3 mmol) and IPA (5 mL) were placed in a vessel equipped with a teflon screw-cap. The vessel was sealed and heated in an oil bath to 90° C. for 7 h, at which time a white precipitate had formed. The reaction mixture was cooled to RT and the solvent and excess reagents were removed in vacuo to afford the title compound as a white solid. MS m/z 258 (MH)⁺.

Step D: 4-(Methylamino)-2-phenylpyrimidine-5-carboxylic acid.

The ester from Step C above (1.57 g, 6.09 mmol) and lithium hydroxide (511 mg, 12.2 mmol) were stirred at 60° C. for 2 days in a mixture of EtOH (50 mL) and water (5 mL). The reaction mixture was cooled to RT and the pH was adjusted to pH 4 with concentrated H₂SO₄. The white precipitate was then filtered and dried in a vacuum oven (50° C., 50 Torr) for 24 h to yield the desired compound. MS m/z 230 (MH)⁺.

Step E: 4-(Methylamino)-2-phenylpyrimidine-5-carboxamide.

The acid from Step D above (1.04 g, 4.54 mmol) was dissolved in CH₂Cl₂ (15 mL) and cooled to 0° C. Oxalyl chloride (0.640 mL, 7.26 mmol) and DMF (34 μL, 0.45 mmol) were then added and the yellow, heterogeneous solution was heated to reflux and stirring was continued for 5 h. The mixture was cooled to RT and the solvent was removed in vacuo. The crude acid chloride was slurried in EtOAc (50 mL) and added to a 0° C. solution of concentrated NH₄OH (10 mL). The off-white heterogeneous mixture was stirred at RT for 18 h and then the EtOAc was removed under reduced pressure. Isolation of the resultant precipitate gave the title compound as a white solid. MS m/z 229 (MH)⁺.

Step F: 4-((2-Fluoropyrimidin-4-yl)(methyl)amino)-2-phenylpyrimidine-5-carboxamide.

Sodium hydride (90 mg of a 60% dispersion in mineral oil, 3.9 mmol) was added to a stirred, 0° C. solution of the methylamino pyrimidine from Step E above (0.44 g, 1.9 mmol) in DMF (15 mL). The reaction mixture was stirred for 5 min then 2,4-difluoropyrimidine (0.34 g, 2.9 mmol) was then added to the yellow slurry and stirring was continued for 90 min. The reaction mixture was quenched with saturated NH₄Cl solution (10 mL) and extracted with chloroform (3×20 mL). The pooled organic layers were washed with brine (5×50 mL), dried over MgSO₄ and concentrated to provide a yellow solid. The residue was purified by preparative thin layer chromatography (5% MeOH:CH₂Cl₂) and center band (R_{f}=0.57) was isolated to give the title compound as a light yellow solid. MS m/z 325 (MH)⁺.

Step G: 4-(Methyl(2-(2-(pyridin-3-yl)ethylamino)pyrimidin-4-yl)amino)-2-phenylpyrimidine-5-carboxamide.

A solution of the pyrimidine from Step F above (0.27 g, 0.84 mmol) and 3-(2-aminoethyl)pyridine (0.80 mL, 6.4 mmol) were stirred at reflux for 25 h in 1,4-dioxane (10 mL). The reaction mixture was cooled to RT, diluted with brine (20 mL) and extracted with chloroform (3×20 mL). The pooled organic layers were dried over MgSO₄ and concentrated. The residue was purified by preparative thin layer chromatography (10% MeOH:CH₂Cl₂) and the appropriate band (R_{f}=0.60) was isolated. The crude product was further purified by recrystallization from CH₂Cl₂:MeOH:hexanes to give the desired product as a white powder. MS m/z 427 (MH)⁺.

### EXAMPLE 65

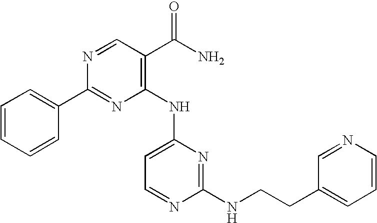 2-Phenyl-4-(2-(2-(pyridin-3-yl)ethylamino)pyrimidin-4-ylamino)pyrimidine-5-carboxamide
Step A: Ethyl 4-amino-2-phenylpyrimidine-5-carboxylate.

The chloropyrimidine (1.97 g, 7.50 mmol) (Example 61) was dissolved in THF (40 mL) and NH₃ was bubbled through for 1 h. The solvent was then removed in vacuo. The title compound was obtained as a white solid which was used directly in the next step.

Step B: 4-Amino-2-phenylpyrimidine-5-carboxylic acid.

The ester from step (a) above (1.8 g, 7.5 mmol) and lithium peroxide (0.70 g, 15.0 mmol) were stirred in a mixture of THF (15 mL) and water (15 mL) at 60° C. for 1 h. The reaction mixture was cooled to RT and the pH was adjusted to pH 4 with 1.6N H₂SO₄. The white precipitate was filtered to provide the desired product. MS m/z 216 (MH)⁺.

Step C: 4-Amino-2-phenylpyrimidine-5-carboxamide.

The acid from Step A above (1.30 g, 6.04 mmol) was dissolved in CH₂Cl₂ (15 mL) and cooled to 0° C. Oxalyl chloride (0.84 mL, 9.7 mmol) and DMF (46 μL, 0.60 mmol) were then added and the yellow, heterogeneous solution was heated to reflux and stirring was continued for 3 h. The mixture was cooled to RT and the solvent was removed in vacuo. The crude acid chloride was slurried in CHCl₃ (10 mL) and added to a 0° C. solution of concentrated NH₄OH (10 mL). The off-white heterogeneous mixture was stirred at 0° C. for 2 h and then the solvent was removed under reduced pressure. Isolation of the resultant precipitate gave the title compound as a white solid (1.2 g). MS m/z 215 (MH)⁺.

Step D: 4-((2-Fluoropyrimidin-4-yl)amino)-2-phenylpyrimidine-5-carboxamide.

Sodium hydride (95%, 0.16 g, 6.4 mmol) was added to a stirred, 0° C. solution of methylamino pyrimidine from Step C above (1.2 g, 5.8 mmol) in DMF (20 mL). The reaction mixture was stirred for 5 min then 2,4-difluoropyrimidine (1.0 g, 8.7 mmol) was then added to the yellow slurry and stirring was continued for 2 h. The reaction mixture was quenched with saturated NH₄Cl solution (10 mL) and extracted with ether (3×30 mL). The pooled organic layers were washed with brine (5×50 mL), dried over MgSO₄ and concentrated to provide a yellow solid. The residue was taken up in CHCl₃ and loaded on to a 40 g pre-packed silica gel column. Elution with 0-5% MeOH (contains 10% NH₄OH):CH₂Cl₂ provided the title compound as a light yellow solid. MS m/z 311 (MH)⁺.

Step E: 2-Phenyl-4-(2-(2-(pyridin-3-yl)ethylamino)pyrimidin-4-ylamino)-pyrimidine-5-carboxamide.

A mixture of the pyrimidine from Step D above (57 mg, 0.18 mmol), 3-(2-aminoethyl)pyridine (0.17 mL, 1.4 mmol) and 1,4-dioxane (3 mL) were loaded into a 5 mL microwave vial. The reaction mixture was subjected to microwave irradiation at 180° C. for 20 min. The solution was cooled and the precipitate was recrystallized from CH₂Cl₂:MeOH:hexanes to give the desired product as a white powder. MS m/z 413 (MH)⁺.

### EXAMPLE 66

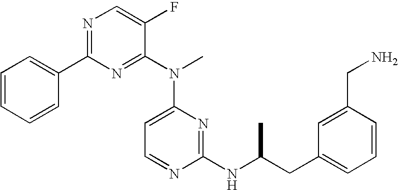 (S)-N²-(1-(3-(Aminomethyl)phenyl)propan-2-yl)-N⁴-(5-fluoro-2-phenylpyrimidin-4-yl)-N⁴-methylpyrimidine-2,4-diamine
Step A: 2-Chloro-5-fluoro-N-methylpyrimidin-4-amine.

To a cooled (−78° C.) solution of 2M MeNH₂ in THF (Aldrich, 125 mL, 0.250 mol) was added 2,4-dichloro-5-fluoro-pyrimidine (Astatech, 15.1 g, 90 mmol) as a solid and the reaction was allowed to warm to RT. After 3 h the solvent was removed in vacuo and the residue was partitioned between CH₂Cl₂ and saturated NaHCO₃. The aqueous layer was extracted with EtOAc (3×) and the combined organics were dried over Na₂SO₄. The solution was filtered and concentrated to dryness to give of a light yellow solid. MS m/z 162 (MH)⁺.

Step B: 5-Fluoro-N-methyl-2-phenylpyrimidin-4-amine.

A mixture of 2-chloro-5-fluoro-N-methylpyrimidin-4-amine (1.54 g, 9.5 mmol), phenyl boronic acid (Aldrich, 1.20 g, 9.8 mmol), Na₂CO₃ (4.48 g, 42.3 mmol) and trans-dichloro-bis(triphenylphosphine) palladium (II), PdCl₂(PPh₃)₂, (Strem, 330 mg, 0.5 mmol) in 28 mL DME/12 mL H₂O/8 mL EtOH was heated to 83° C. After 3 h phenyl boronic acid (600 mg, 4.9 mmol) was added to the reaction. After an additional 4 h the reaction was cooled to RT and diluted with EtOAc (150 mL). The solution was washed with brine (2×50 mL) and dried over Na₂SO₄. The solution was filtered, evaporated onto SiO₂ and purified by flash column chromatography with EtOAc/hexanes (0:1→3:17) as eluant to give the title compound as a white amorphous solid. MS m/z 204 (MH)⁺.

Step C: 5-Fluoro-N-(2-fluoropyrimidin-4-yl)-N-methyl-2-phenylpyrimidin-4-amine.

To a cooled solution of 5-fluoro-N-methyl-2-phenylpyrimidin-4-amine (1.57 g, 7.7 mmol) in 15 mL of THF was added 60% NaH (371 mg, 9.3 mmol) in one portion resulting in gas evolution. The reaction was warmed to RT for 0.5 h and then cooled to −40° C. To the mixture was added 2,4-difluoropyrimidine (1.9 g, 16.4 mmol) and the reaction was warmed to RT for 6 h and then to 50° C. overnight. The reaction was cooled to RT and partitioned between EtOAc and brine. The aqueous layer was extracted with EtOAc (3×) and the combined organics were evaporated onto SiO₂ and purified by flash column chromatography with EtOAc/hexanes (0:1→3:17) as eluant to give the title compound as a white amorphous solid. MS m/z 300 (MH)⁺.

Step D: (S)-tert-Butyl 3-(2-(4-((5-fluoro-2-phenylpyrimidin-4-yl)(methyl)amino)-pyrimidin-2-ylamino)propyl)benzylcarbamate.

A mixture of 5-fluoro-N-(2-fluoro-pyrimidin-4-yl)-N-methyl-2-phenylpyrimidin-4-amine (390 mg, 1.3 mmol) and (S)-tert-butyl 3-(2-aminopropyl)benzylcarbamate (360 mg, 1.4 mmol) in 15 mL of 1,4-dioxane was heated to reflux. After 21 h the reaction was cooled to RT, evaporated onto SiO₂ and purified by flash column chromatography with EtOAc/hexanes (0:1→2:3) as eluant to give the title compound as a white foam. MS m/z 544 (MH)⁺.

Step E: (S)-N²-(1-(3-(Aminomethyl)phenyl)propan-2-yl)-N⁴-(5-fluoro-2-phenyl-pyrimidin-4-yl)-N⁴-methylpyrimidine-2,4-diamine.

To a RT solution of (S)-tert-butyl 3-(2-(4-((5-fluoro-2-phenylpyrimidin-4-yl)(methyl)amino)pyrimidin-2-ylamino)propyl)benzylcarbamate (273 mg, 0.5 mmol) in 2 mL 1,4-dioxane was added 2.5 mL of 1M HCl in Et₂O. 1M HCl in Et₂O was added as needed until the reaction was complete (by TLC). The reaction was diluted with H₂O and washed with Et₂O. The aqueous layer was basified with NaHCO₃ and extracted with EtOAc (3×). The combined organics were dried over Na₂SO₄, evaporated onto SiO₂ and purified by flash column chromatography with 2N NH₃ in MeOH/CH₂Cl₂ (0:1→3:37) as eluant to give the title compound as a colorless glass. MS m/z 444 (MH)⁺.

### EXAMPLE 67

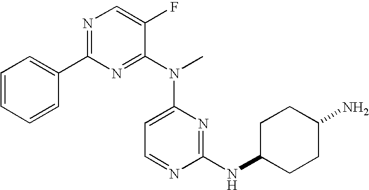 N²-(4-Aminocyclohexyl)-N⁴-(5-fluoro-2-phenylpyrimidin-4-yl)-N⁴-methyl-pyrimidine-2,4-diamine.

This material was prepared according to the method described in Example 66 Step D using 5-fluoro-N-(2-fluoropyrimidin-4-yl)-N-methyl-2-phenylpyrimidin-4-amine (164 mg, 0.55 mmol) and trans-1,4-diaminocyclohexane (Aldrich, 253 mg, 2.2 mmol) in 5 mL 1,4-dioxane. Purification by flash column chromatography with 2N NH₃ in MeOH/CH₂Cl₂ (0:1→3:37) as eluant gave the title compound as a colorless glass. MS m/z 394 (MH)⁺.

### EXAMPLE 68

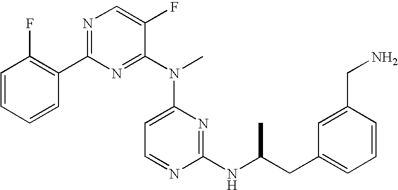 N²-((S)-1-(3-(Aminomethyl)phenyl)propan-2-yl)-N⁴-(5-fluoro-2-(2-fluorophenyl)-pyrimidin-4-yl)-N⁴-methylpyrimidine-2,4-diamine
Step A: 2-Chloro-5-fluoro-N-(2-fluoropyrimidin-4-yl)-N-methylpyrimidin-4-amine.

This material was prepared according to the method described in Example 66, Step C using 2-chloro-5-fluoro-N-methylpyrimidin-4-amine, (944 mg, 6.2 mmol), 60% NaH (283 mg, 7.1 mmol) and 2,4-difluoropyrimidine (1.18 g, 10.2 mmol) 10 mL DMF. Purification by flash column chromatography with EtOAc/hexane (0:1→1:3) as eluant gave the title compound as a white amorphous solid MS m/z 258 (MH)⁺.

Step B: (S)-tert-Butyl 3-(2-(4-((2-chloro-5-fluoropyrimidin-4-yl)(methyl)amino)-pyrimidin-2-ylamino)propyl)benzylcarbamate.

This material was prepared according to the method described in Example 66 Step D using 2-chloro-5-fluoro-N-(2-fluoro-pyrimidin-4-yl)-N-methylpyrimidin-4-amine, (200 mg, 0.8 mmol), (S)-tert-butyl 3-(2-aminopropyl)benzylcarbamate (207 mg, 0.8 mmol) and Et₃N (0.15 mL, 1.08 mmol) in 8 mL of THF. Purification by flash column chromatography with EtOAc/hexane (0:1→1:1) as eluant gave the title compound as a white foam. MS m/z 502 (MH)⁺.

Step C: tert-Butyl (17S)-3-((S)-2-(4-((5-fluoro-2-(2-fluorophenyl)pyrimidin-4-yl)(methyl)amino)pyrimidin-2-ylamino)propyl)benzylcarbamate.

To a RT mixture of (S)-tert-butyl 3-(2-(4-((2-chloro-5-fluoropyrimidin-4-yl)(methyl)amino)-pyrimidin-2-ylamino)propyl)benzylcarbamate (117 mg, 0.2 mmol), Na₂CO₃ (122 mg, 1.2 mmol) and 2-fluorobenzene boronic acid (Lancaster, 43 mg, 0.3 mmol) in 1.4 mL DME/0.6 mL H₂O/0.4 mL EtOH was added PdCl₂(PPh₃)₂ (Strem, 19 mg, 0.03 mmol) and the reaction was heated to 82° C. After 6 h the reaction was cooled to RT and partitioned between EtOAc/brine. The aqueous layer was extracted with EtOAc (2×) and dried over MgSO₄. The solution was filtered, evaporated onto SiO₂ and purified by flash column chromatography with EtOAc/hexanes (0:1→11:9) as eluant to give the title compound as a white foam. MS m/z 562 (MH)⁺.

Step D: N²-((S)-1-(3-(Aminomethyl)phenyl)propan-2-yl)-N⁴-(5-fluoro-2-(2-fluoro-phenyl)pyrimidin-4-yl)-N⁴-methylpyrimidine-2,4-diamine.

This material was prepared according to the method described in Example 66 Step E using tert-butyl (17S)-3-((S)-2-(4-((5-fluoro-2-(2-fluorophenyl)pyrimidin-4-yl)(methyl)amino)-pyrimidin-2-ylamino)propyl)benzylcarbamate (64 mg, 0.1 mmol) and 3.5 mL 1M HCl in Et₂O in 2 mL of 1,4-dioxane. Purification by flash column chromatography with 2N NH₃ in MeOH/CH₂Cl₂ (0:1→3:37) as eluant gave the title compound as a colorless glass. MS m/z 462 (MH)⁺.

### EXAMPLE 69

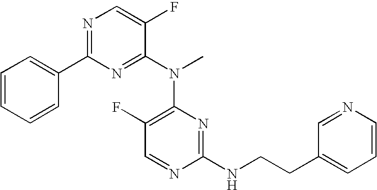 5-Fluoro-N⁴-(5-fluoro-2-phenylpyrimidin-4-yl)-N⁴-methyl-N²-(2-(pyridin-3-yl)-ethyl)pyrimidine-2,4-diamine
Step A: N-(2-Chloro-5-fluoropyrimidin-4-yl)-5-fluoro-N-methyl-2-phenylpyrimidin-4-amine.

This material was prepared according to the method described in Example 66 Step C using 5-fluoro-N-methyl-2-phenylpyrimidin-4-amine, (1.02 g, 5.0 mmol), 60% NaH (338 mg, 8.5 mmol) and 2,4-dichloro-5-fluoro-pyrimidine (Astatech, 1.3 g, 7.6 mmol) in 20 mL of DMF. Purification by flash column chromatography with EtOAc/hexane (0:1→3:17) as eluant gave the title compound as a white amorphous solid. MS m/z 334 (MH)⁺.

Step B: 5-Fluoro-N⁴-(5-fluoro-2-phenylpyrimidin-4-yl)-N⁴-methyl-N²-(2-(pyridin-3-yl)ethyl)pyrimidine-2,4-diamine.

A mixture of N-(2-chloro-5-fluoropyrimidin-4-yl)-5-fluoro-N-methyl-2-phenylpyrimidin-4-amine (166 mg, 0.5 mmol), 3-(2-amino-ethyl)pyridine (TCI, 0.12 mL, 1.0 mmol) and a few crystals of p-toluenesulfonic acid in 2 mL i-PrOH was heated to 140° C. in the microwave for 45 min. The reaction mixture was evaporated onto SiO₂ and purified by flash column chromatography with 2N NH₃ in MeOH/CH₂Cl₂ (0:1→1:19) as eluant to give the title compound as a white amorphous solid. MS m/z 420 (MH)⁺.

### EXAMPLE 70

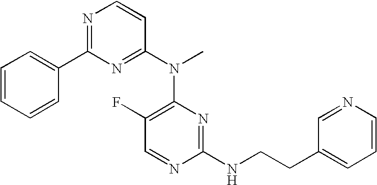 5-Fluoro-N⁴-methyl-N⁴-(2-phenylpyrimidin-4-yl)-N²-(2-(pyridin-3-yl)ethyl)-pyrimidine-2,4-diamine
Step A: N-(2-Chloro-5-fluoropyrimidin-4-yl)-N-methyl-2-phenylpyrimidin-4-amine.

This material was prepared according to the method described in Example 66 Step C using N-methyl-2-phenylpyrimidin-4-amine (235 mg, 1.3 mmol), 60% NaH (77 mg, 1.9 mmol) and 2,4-dichloro-5-fluoro-pyrimidine (Astatech, 338 g, 2.0 mmol) in 10 mL of DMF. Purification by flash column chromatography with EtOAc/hexane (0:1→1:4) as eluant gave the title compound as a white amorphous solid. MS m/z 316 (MH)⁺.

Step B: 5-Fluoro-N⁴-methyl-N⁴-(2-phenylpyrimidin-4-yl)-N²-(2-(pyridin-3-yl)ethyl)-pyrimidine-2,4-diamine.

A mixture of N-(2-chloro-5-fluoropyrimidin-4-yl)-N-methyl-2-phenylpyrimidin-4-amine (153 mg, 0.5 mmol), 3-(2-aminoethyl)pyridine (TCI, 0.13 mL, 1.1 mmol) and a few crystals of p-toluenesulfonic acid in 2 mL i-PROH was heated to 140° C. in the microwave for 75 min. The reaction mixture was evaporated onto SiO₂ and purified by flash column chromatography with 2N NH₃ in MeOH/CH₂Cl₂ (0:1→1:19) as eluant to give the title compound as a colorless oil. MS m/z 402 (MH)⁺.

### EXAMPLE 71

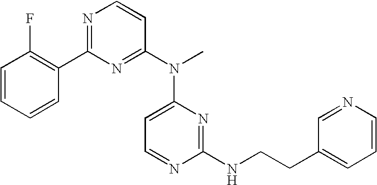 N⁴-(2-(2-Fluorophenyl)pyrimidin-4-yl)-N⁴-methyl-N²-(2-(pyridin-3-yl)ethyl)-pyrimidine-2,4-diamine
Step A: N⁴-(2-Chloropyrimidin-4-yl)-N⁴-methyl-N²-(2-(pyridin-3-yl)ethyl)-pyrimidine-2,4-diamine.

To a solution of 2-chloro-N-(2-fluoropyrimidin-4-yl)-N-methylpyrimidin-4-amine (2.02 g, 8.5 mmol) and 3-(2-aminoethyl)-pyridine (TCI, 1.23 mL, 10.5 mmol) in 20 mL DMF was added Cs₂CO₃ (3.25 g, 10.0 mmol) and the reaction was heated to 80° C. After 2 h the reaction was cooled to RT and poured into H₂O. The solution was extracted with CH₂Cl₂ (3×) and the combined organics were washed with H₂O and dried over MgSO₄. The organic solution was filtered, concentrated and triturated with hexane at 0° C. The yellow-orange solid was filtered, washed with hexane and pentane, and dried in vacuo to give a pale orange amorphous solid. MS m/z 342 (MH)⁺.

Step B: N⁴-(2-(2-Fluorophenyl)pyrimidin-4-yl)-N⁴-methyl-N²-(2-(pyridin-3-yl)ethyl)pyrimidine-2,4-diamine.

A mixture of N-(²-chloropyrimidin-4-yl)-N⁴-methyl-N²-(2-(pyridin-3-yl)ethyl)pyrimidine-2,4-diamine (101 mg, 0.3 mmol), 2-fluorobenzene boronic acid (65 mg, 0.5 mmol), Na₂CO₃ (150 mg, 1.42 mmol) and PdCl₂(PPh₃)₂ (17 mg, 0.02 mmol) in 1.2 mL DME/0.5 mL H₂O/0.3 mL EtOH was heated to 150° C. for 10 min in the microwave. The reaction was evaporated onto SiO₂ and purified by flash column chromatography with 2N NH₃ in MeOH/CH₂Cl₂ (0:1→1:19) as eluant to give the title compound as a colorless glass. MS m/z 402 (MH)⁺.

### EXAMPLE 72

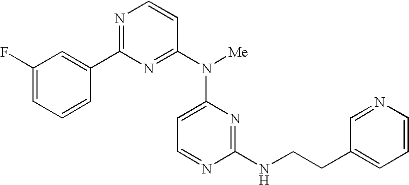 N⁴-(2-(3-Fluorophenyl)pyrimidin-4-yl)-N⁴-methyl-N²-(2-(pyridin-3-yl)ethyl)-pyrimidine-2,4-diamine.

A mixture of N⁴-(2-chloropyrimidin-4-yl)-N⁴-methyl-N²-(2-(pyridin-3-yl)ethyl)pyrimidine-2,4-diamine (150 mg, 0.44 mmol), 3-fluoro-benzeneboronic acid (74 mg, 0.53 mmol, Lancaster), sodium carbonate (139 mg, 1.32 mmol, JT Baker) and Pd(PPh₃)₂Cl₂ (31 mg, 0.044 mmol, Strem) in a mixture of DME, EtOH and H₂O (2.0 mL) was heated to 150° C. for 15 min in the Smith Synthesizer Microwave. The mixture was diluted with MeOH and concentrated over silica gel. Purification by flash chromatography (1.5→3.5% 2N NH₃ in MeOH/CH₂Cl₂) gave the title compound. MS m/z 402 (MH)⁺.

### EXAMPLE 73

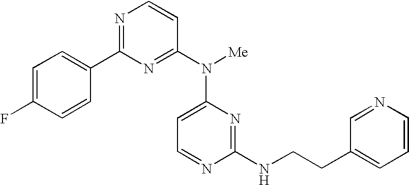 N⁴-(2-(4-Fluorophenyl)pyrimidin-4-yl)-N⁴-methyl-N²-(2-(pyridin-3-yl)ethyl)-pyrimidine-2,4-diamine.

Analogous to the methods used in Example 71 Step B using N⁴-(2-chloropyrimidin-4-yl)-N⁴-methyl-N²-(2-(pyridin-3-yl)ethyl)pyrimidine-2,4-diamine (150 mg, 0.44 mmol), 4-fluorobenzeneboronic acid (74 mg, 0.53 mmol, Aldrich), sodium carbonate (139 mg, 1.32 mmol) and Pd(PPh₃)₂Cl₂ (31 mg, 0.044 mmol) in a mixture of DME, EtOH, and H₂O (7:2:3, 2.0 mL). MS m/z 402 (MH)⁺.

### EXAMPLE 74

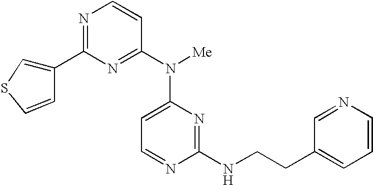 N⁴-Methyl-N²-(2-(pyridin-3-yl)ethyl)-N⁴-(2-(thiophen-3-yl)pyrimidin-4-yl)-pyrimidine-2,4-diamine.

Analogous to the methods used in Example 71, Step B using N⁴-(2-chloropyrimidin-4-yl)-N⁴-methyl-N²-(2-(pyridin-3-yl)ethyl)pyrimidine-2,4-diamine (150 mg, 0.44 mmol), 3-thiopheneboronic acid (67 mg, 0.53 mmol, Aldrich), sodium carbonate (139 mg, 1.32 mmol) and Pd(PPh₃)₂Cl₂ (31 mg, 0.044 mmol) in a mixture of DME, EtOH, and H₂O (7:2:3, 2.0 mL). MS m/z 390 (MH)⁺.

### EXAMPLE 75

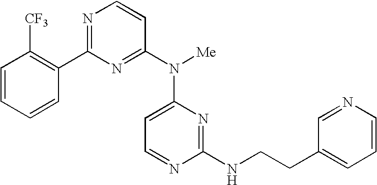 N⁴-Methyl-N²-(2-(pyridin-3-yl)ethyl)-N⁴-(2-(2-(trifluoromethyl)phenyl)pyrimidin-4-yl)pyrimidine-2,4-diamine.

Analogous to the methods used in Example 71, Step B using N⁴-(2-chloropyrimidin-4-yl)-N⁴-methyl-N²-(2-(pyridin-3-yl)ethyl)pyrimidine-2,4-diamine (150 mg, 0.44 mmol), 2-(trifluoromethyl)phenylboronic acid (101 mg, 0.53 mmol, Aldrich), sodium carbonate (139 mg, 1.32 mmol) and Pd(PPh₃)₂Cl₂ (31 mg, 0.044 mmol) in a mixture of DME, EtOH, and H₂O (7:2:3, 2.0 mL). MS m/z 452 (MH)⁺.

### EXAMPLE 76

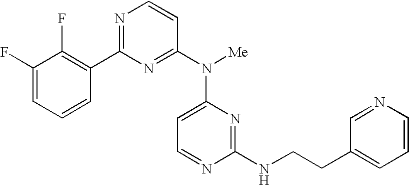 N⁴-(2-(2,3-Difluorophenyl)pyrimidin-4-yl)-N⁴-methyl-N²-(2-(pyridin-3-yl)ethyl)-pyrimidine-2,4-diamine.

Analogous to the methods used in Example 71, Step B using N⁴-(2-chloropyrimidin-4-yl)-N⁴-methyl-N²-(2-(pyridin-3-yl)ethyl)pyrimidine-2,4-diamine (150 mg, 0.44 mmol), 2,3-difluorophenylboronic acid (84 mg, 0.53 mmol, Aldrich), sodium carbonate (139 mg, 1.32 mmol) and Pd(PPh₃)₂Cl₂ (31 mg, 0.044 mmol) in a mixture of DME, EtOH, and H₂O (7:2:3, 2.0 mL). MS m/z 420(MH)⁺.

### EXAMPLE 77

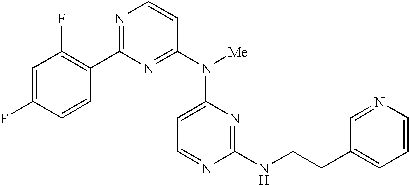 N⁴-(2-(2,4-Difluorophenyl)pyrimidin-4-yl)-N⁴-methyl-N²-(2-(pyridin-3-yl)ethyl)-pyrimidine-2,4-diamine.

Analogous to the methods used in Example 71, Step B using N⁴-(2-chloropyrimidin-4-yl)-N⁴-methyl-N²-(2-(pyridin-3-yl)ethyl)pyrimidine-2,4-diamine (150 mg, 0.44 mmol), 2,4-difluorophenylboronic acid (84 mg, 0.53 mmol, Aldrich), sodium carbonate (139 mg, 1.32 mmol) and Pd(PPh₃)₂Cl₂ (31 mg, 0.044 mmol) in a mixture of DME, EtOH, and H₂O (7:2:3, 2.0 mL). MS m/z 420(MH)⁺.

### EXAMPLE 78

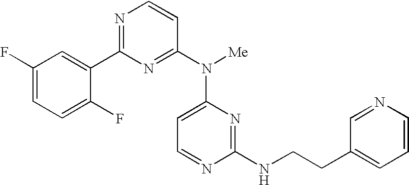 N⁴-(2-(2,5-Difluorophenyl)pyrimidin-4-yl)-N⁴-methyl-N²-(2-(pyridin-3-yl)ethyl)-pyrimidine-2,4-diamine.

Analogous to the methods used in Example 71, Step B using N⁴-(2-chloropyrimidin-4-yl)-N⁴-methyl-N²-(2-(pyridin-3-yl)ethyl)pyrimidine-2,4-diamine (150 mg, 0.44 mmol), 2,5-difluorophenylboronic acid (84 mg, 0.53 mmol, Aldrich), sodium carbonate (139 mg, 1.32 mmol) and Pd(PPh₃)₂Cl₂ (31 mg, 0.044 mmol) in a mixture of DME, EtOH, and H₂O (7:2:3, 2.0 mL). MS m/z 420(MH)⁺.

### EXAMPLE 79

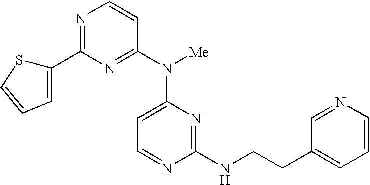 N⁴-Methyl-N²-(2-(pyridin-3-yl)ethyl)-N⁴-(2-(thiophen-2-yl)pyrimidin-4-yl)-pyrimidine-2,4-diamine.

Analogous to the methods used in Example 71, Step B using N⁴-(2-chloropyrimidin-4-yl)-N⁴-methyl-N²-(2-(pyridin-3-yl)ethyl)pyrimidine-2,4-diamine (150 mg, 0.44 mmol), 2-thiopheneboronic acid (68 mg, 0.53 mmol, Aldrich), sodium carbonate (139 mg, 1.32 mmol) and Pd(PPh₃)₂Cl₂ (31 mg, 0.044 mmol) in a mixture of DME, EtOH, and H₂O (7:2:3, 2.0 mL). MS m/z 390 (MH)⁺.

### EXAMPLE 80

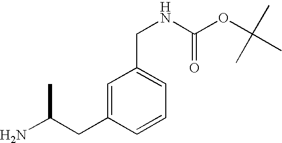 (S)-tert-Butyl 3-(2-aminopropyl)benzylcarbamate
Step A: (S)-Benzyl 1-(3-cyanophenyl)propan-2-ylcarbamate.

A mixture of (S)-benzyl 1-(3-bromophenyl)propan-2-ylcarbamate (16.3 g, 47 mmol, J-Star), zinc cyanide (3.3 g, 28.2 mmol, Aldrich), zinc metal (306 mg, 4.7 mmol, Aldrich), zinc acetate (862 mg, 4.7 mmol, Aldrich), tris(dibenzylideneacetone)dipalladium(0) (862 mg, 0.94 mmol, Aldrich) and 1,1′-bis(diphenylphosphino)ferrocene (1.30 g, 2.35 mmol, Aldrich) in DMF (40 mL) and H₂O (0.4 mL) was heated to 80° C. for 16 h. The mixture was cooled to RT and diluted with EtOAc. The organic layer was washed with 10% aqueous sodium carbonate (3×), dried over Na₂SO₄, filtered and concentrated. Purification by flash chromatography (0→25% EtOAc/Hexanes) gave the title compound.

Step B: (S)-Benzyl 1-(3-(Boc-aminomethyl)phenyl)propan-2-ylcarbamate.

A mixture of (S)-benzyl 1-(3-cyanophenyl)propan-2-ylcarbamate (13.5 g, 46 mmol), di-tert-butyl dicarbonate (20.1 g, 92 mmol, Aldrich) and nickel (II) chloride hexahydrate (1.09 g, 4.6 mmol, Aldrich) was cooled to 0° C. and treated with sodium borohydride (12.16 g, 322 mmol, Aldrich) portionwise. The mixture was stirred 0° C.→RT for 12 h. Diethylenetriamine (4.97 mL, 46 mmol) was added and the mixture was stirred at RT for 1 h. After concentrating the mixture, the residue was dissolved in EtOAc and washed with sat. aq. sodium bicarbonate (2×). The organic layer was dried over Na₂SO₄, filtered and concentrated. MS m/z 299 (M-Boc)⁺.

Step C: (S)-tert-Butyl 3-(2-aminopropyl)benzylcarbamate.

A mixture of (S)-benzyl 1-(3-(Boc-aminomethyl)phenyl)propan-2-ylcarbamate (16.4 g, 41.2 mmol) and 10% Pd/C (1.6 g) in EtOH (250 mL) was stirred under hydrogen atmosphere for 18 h. The mixture was filtered through a pad of Celite, eluting with MeOH, followed by concentration in vacuo. The residue was purified by flash chromatography (0→6% 2N NH₃ in MeOH/CH₂Cl₂) giving a pale-yellow oil.

### EXAMPLE 81

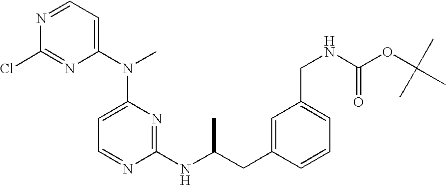 (S)-tert-Butyl 3-(2-(4-((2-chloropyrimidin-4-yl)(methyl)amino)pyrimidin-2-yl-amino)propyl)benzylcarbamate.

A mixture of (S)-tert-butyl 3-(2-aminopropyl)-benzylcarbamate (1.69 g, 6.4 mmol), 2-chloro-N-(2-fluoropyrimidin-4-yl)-N-methylpyrimidin-4-amine (1.5 g, 6.4 mmol) and cesium carbonate (2.5 g, 7.7 mmol, Aldrich) in DMF (30 mL) was heated to 85° C. for 3 h. The mixture was diluted with H₂O and extracted with 25% i-PrOH/CHCl₃ (3×). The combined organics were dried over Na₂SO₄, filtered and concentrated. Purification by flash chromatography (0→50% EtOAc/Hexanes) gave a pale-yellow oil.

### EXAMPLE 82

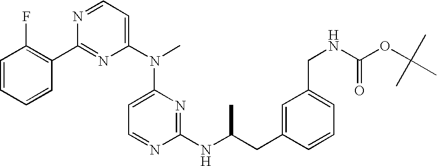 tert-Butyl (7S)-3-((S)-2-(4-((2-(2-fluorophenyl)pyrimidin-4-yl)(methyl)amino)-pyrimidin-2-ylamino)propyl)benzylcarbamate.

Analogous to the methods used in Example 71, Step B using (S)-tert-butyl 3-(2-(4-((2-chloropyrimidin-4-yl)(methyl)-amino)pyrimidin-2-ylamino)propyl)benzylcarbamate (400 mg, 0.83 mmol), 2-fluorobenzeneboronic acid (139 mg, 0.99 mmol, Aldrich), sodium carbonate (263 mg, 2.48 mmol) and Pd(PPh₃)₂Cl₂ (58 mg, 0.083 mmol) in a mixture of DME, EtOH, and H₂O (7:2:3, 3.0 mL). Purification by flash chromatography (0→2.5% MeOH/CH₂Cl₂) gave the title compound. MS m/z 544 (MH)⁺.

### EXAMPLE 83

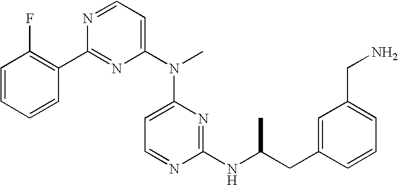 N²-((S)-1-(3-(Aminomethyl)phenyl)propan-2-yl)-N⁴-(2-(2-fluorophenyl)pyrimidin-4-yl)-N⁴-methylpyrimidine-2,4-diamine.

A solution of tert-butyl (7S)-3-((S)-2-(4-((2-(2-fluorophenyl)pyrimidin-4-yl)(methyl)amino)pyrimidin-2-amino)propyl)-benzylcarbamate (330 mg, 0.61) in 50% TFA/CH₂Cl₂ (10 mL) was stirred for 20 h. The volatiles were removed in vacuo and the residue was partitioned between 10% aqueous sodium carbonate and CH₂Cl₂. The organic layer was collected and the aqueous layer was extracted with CH₂Cl₂ (2×). The combined organics were dried over Na₂SO₄, filtered and concentrated. The residue was dissolved in a small amount of CH₂Cl₂ and loaded on a 1 g Agilent AccuBOND II SCX solid phase extraction column. After washing with 10% MeOH/CH₂Cl₂, the product was eluted with 2N NH₃ in MeOH. After the volatiles were removed the residue was purified by flash chromatography (3.0→5.0% 2N NH₃ in MeOH/CH₂Cl₂) to afford the title compound. MS m/z 444 (MH)⁺.

### EXAMPLE 84

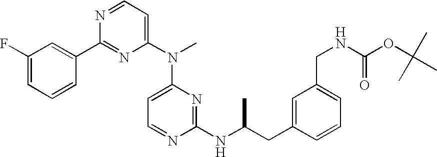 tert-Butyl (7S)-3-((S)-2-(4-((2-(3-fluorophenyl)pyrimidin-4-yl)(methyl)amino)-pyrimidin-2-ylamino)propyl)benzylcarbamate.

Analogous to the methods used Example 71, Step B using (S)-tert-butyl 3-(2-(4-((2-chloropyrimidin-4-yl)(methyl)-amino)pyrimidin-2-ylamino)propyl)benzylcarbamate (400 mg, 0.83 mmol), 3-fluorobenzeneboronic acid (139 mg, 0.99 mmol, Aldrich), sodium carbonate (263 mg, 2.48 mmol) and Pd(PPh₃)₂Cl₂ (58 mg, 0.083 mmol) in a mixture of DME, EtOH, and H₂O (7:2:3, 3.0 mL). Purification by flash chromatography (0→1.5% MeOH/CH₂Cl₂) gave the title compound. MS m/z 544 (MH)⁺.

### EXAMPLE 85

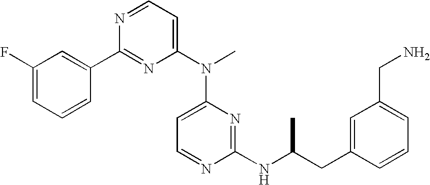 N²-((S)-1-(3-(Aminomethyl)phenyl)propan-2-yl)-N⁴-(2-(3-fluorophenyl)pyrimidin-4-yl)-N⁴-methylpyrimidine-2,4-diamine.

Analogous to the methods used in Example 83 using tert-butyl (7S)-3-((S)-2-(4-((2-(3-fluorophenyl)pyrimidin-4-yl)(methyl)amino)pyrimidin-2-ylamino)propyl)benzylcarbamate (279 mg, 0.51 mmol) in 50% TFA/CH₂Cl₂ (10 mL). Purification by flash chromatography (3.0%→5.0% 2N NH₃ in MeOH/CH₂Cl₂) gave the title compound. MS m/z 444 (MH)⁺.

### EXAMPLE 86

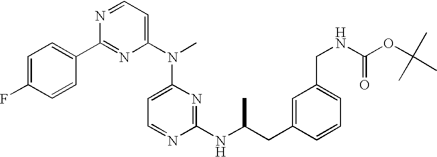 (S)-tert-Butyl 3-(2-(4-((2-(4-fluorophenyl)pyrimidin-4-yl)(methyl)amino)pyrimidin-2-ylamino)propyl)benzylcarbamate.

Analogous to the methods used in Example 71, Step B using (S)-tert-butyl 3-(2-(4-((2-chloropyrimidin-4-yl)(methyl)amino)-pyrimidin-2-ylamino)propyl)benzylcarbamate (400 mg, 0.83 mmol), 4-fluoro-benzeneboronic acid (139 mg, 0.99 mmol, Aldrich), sodium carbonate (263 mg, 2.48 mmol) and Pd(PPh₃)₂Cl₂ (58 mg, 0.083 mmol) in a mixture of DME, EtOH, and H₂O (7:2:3, 3.0 mL). Purification by flash chromatography (0→1.5% MeOH/CH₂Cl₂) gave the title compound. MS m/z 544 (MH)⁺.

### EXAMPLE 87

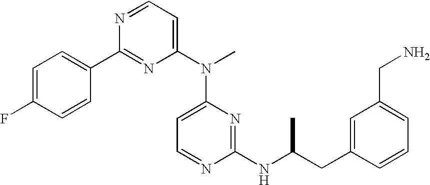 (S)-N²-(1-(3-(Aminomethyl)phenyl)propan-2-yl)-N⁴-(2-(4-fluorophenyl)pyrimidin-4-yl)-N⁴-methylpyrimidine-2,4-diamine.

Analogous to the methods used in Example 83 using (S)-tert-butyl 3-(2-(4-((2-(4-fluorophenyl)pyrimidin-4-yl)(methyl)amino)pyrimidin-2-ylamino)propyl)benzylcarbamate (305 mg, 0.56 mmol) in 50% TFA/CH₂Cl₂ (10 mL). Purification by flash chromatography (3.0%→5.0% 2N NH₃ in MeOH/CH₂Cl₂) gave the title compound. MS m/z 444 (MH)⁺.

### EXAMPLE 88

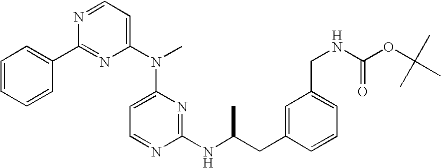 (S)-tert-Butyl 3-(2-(4-(methyl(2-phenylpyrimidin-4-yl)amino)pyrimidin-2-ylamino)propyl)benzylcarbamate.

Analogous to the methods used in Example 71, Step B using (S)-tert-butyl 3-(2-(4-((2-chloropyrimidin-4-yl)(methyl)amino)-pyrimidin-2-ylamino)propyl)benzylcarbamate (400 mg, 0.83 mmol), phenylboronic acid (121 mg, 0.99 mmol, Aldrich), sodium carbonate (263 mg, 2.48 mmol) and Pd(PPh₃)₂Cl₂ (58 mg, 0.083 mmol) in a mixture of DME, EtOH, and H₂O (7:2:3, 3.0 mL). Purification by flash chromatography (0→2.5% MeOH/CH₂Cl₂) gave the title compound. MS m/z 526 (MH)⁺.

### EXAMPLE 89

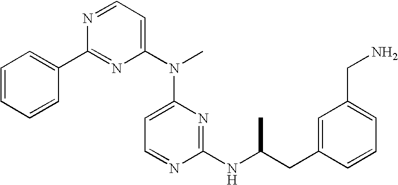 (S)-N²-(1-(3-(Aminomethyl)phenyl)propan-2-yl)-N⁴-methyl-N⁴-(2-phenylpyrimidin-4-yl)pyrimidine-2,4-diamine.

Analogous to the methods used in Example 83 using (S)-tert-butyl 3-(2-(4-(methyl(2-phenylpyrimidin-4-yl)amino)pyrimidin-2-ylamino)-propyl)benzylcarbamate (327 mg, 0.62 mmol) in 50% TFA/CH₂Cl₂ (10 mL). Purification by flash chromatography (3.0%→5.0% 2N NH₃ in MeOH/CH₂Cl₂) gave the title compound. MS m/z 426 (MH)⁺.

### EXAMPLE 90

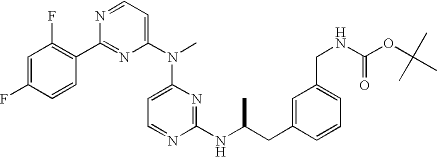 tert-Butyl (7S)-3-((S)-2-(4-((2-(2,4-difluorophenyl)pyrimidin-4-yl)(methyl)amino)-pyrimidin-2-ylamino)propyl)benzylcarbamate.

Analogous to the methods used in Example 71, Step B using (S)-tert-butyl 3-(2-(4-((2-chloropyrimidin-4-yl)(methyl)-amino)pyrimidin-2-ylamino)propyl)benzylcarbamate (400 mg, 0.83 mmol), 2,4-di-fluorobenzeneboronic acid (139 mg, 0.99 mmol, Aldrich), sodium carbonate (263 mg, 2.48 mmol) and Pd(PPh₃)₂Cl₂ (58 mg, 0.083 mmol) in a mixture of DME, EtOH, and H₂O (7:2:3, 3.0 mL). Purification by flash chromatography (0→1.5% MeOH/CH₂Cl₂) gave the title compound.

### EXAMPLE 91

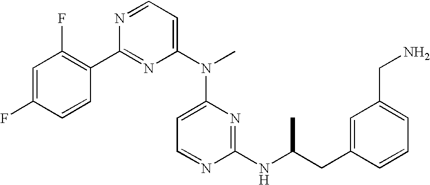 N²-((S)-1-(3-(Aminomethyl)phenyl)propan-2-yl)-N⁴-(2-(2,4-difluorophenyl)-pyrimidin-4-yl)-N⁴-methylpyrimidine-2,4-diamine.

Analogous to the methods used in Example 83 using tert-butyl (7S)-3-((S)-2-(4-((2-(2,4-difluorophenyl)pyrimidin-4-yl)(methyl)amino)pyrimidin-2-ylamino)propyl)benzylcarbamate (341 mg, 0.61 mmol) in 50% TFA/CH₂Cl₂ (10 mL). The residue was dissolved in MeOH and loaded onto a 1 g Agilent AccuBOND II SCX solid phase extraction column. The column was washed with MeOH and eluted with 2N NH₃ in MeOH. The volatiles were removed in vacuo to afford the title compound. MS m/z 462 (MH)⁺.

### EXAMPLE 92

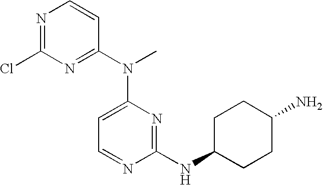 N²-(4-Aminocyclohexyl)-N⁴-(2-chloropyrimidin-4-yl)-N⁴-methylpyrimidine-2,4-diamine.

A mixture of trans-1,4-diaminocyclohexane (239 mg, 2.09 mmol), 2-chloro-N-(2-fluoropyrimidin-4-yl)-N-methylpyrimidin-4-amine (500 mg, 2.09 mmol) and cesium carbonate (815 mg, 2.51 mmol, Aldrich) in DMF (10 mL) was heated to 80° C. for 3 h. After stirring an additional 16 h at RT, the mixture was diluted with H₂O and extracted with 25% i-PrOH/CHCl₃ (4×). The combined organics were dried over Na₂SO₄, filtered and concentrated. Purification by flash chromatography (0→5% MeOH/CH₂Cl₂, 5→10% 2N NH₃ in MeOH/CH₂Cl₂) gave the title compound. MS m/z 334 (MH)⁺.

### EXAMPLE 93

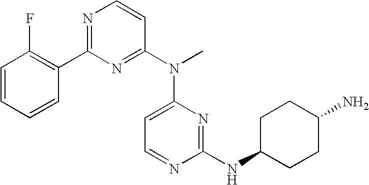 N²-(4-Aminocyclohexyl)-N⁴-(2-(2-fluorophenyl)pyrimidin-4-yl)-N⁴-methyl-pyrimidine-2,4-diamine.

Analogous to the methods used in Example 71, Step B. using N²-(4-aminocyclohexyl)-N⁴-(2-chloropyrimidin-4-yl)-N⁴-methylpyrimidine-2,4-diamine (267 mg, 0.8 mmol), 2-fluorobenzeneboronic acid (134 mg, 0.96 mmol, Lancaster), sodium carbonate (254 mg, 2.40 mmol) and Pd(PPh₃)₂Cl₂ (56 mg, 0.080 mmol) in a mixture of DME, EtOH, and H₂O (7:2:3, 2.0 mL). Purification by flash chromatography (0→8.0% 2N NH₃ in MeOH/CH₂Cl₂) gave an impure mixture. The residue was dissolved in 10% MeOH/CH₂Cl₂ and loaded onto a 1 g Agilent AccuBOND II SCX solid phase extraction column, washing with 10% MeOH/CH₂Cl₂. The title compound was eluted with 2N NH₃ in MeOH. MS m/z 394 (MH)⁺.

### EXAMPLE 94

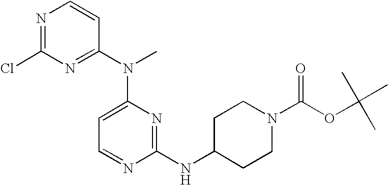 tert-Butyl 4-(4-((2-chloropyrimidin-4-yl)(methyl)amino)pyrimidin-2-ylamino)-piperidine-1-carboxylate.

A mixture of 4-amino-1-N-Boc-piperidine (419 mg, 2.09 mmol), 2-chloro-N-(2-fluoropyrimidin-4-yl)-N-methylpyrimidin-4-amine (500 mg, 2.09 mmol) and cesium carbonate (815 mg, 2.51 mmol, Aldrich) in DMF (10 mL) was heated to 80° C. for 3 h. After stirring an additional 16 h at RT, the mixture was added to H₂O and the solids were collected by filtration. Purification by flash chromatography (0→4% MeOH/CH₂Cl₂) afforded the title compound. MS m/z 420 (MH)⁺.

### EXAMPLE 95

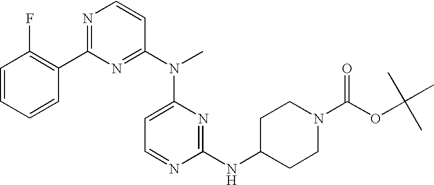 tert-Butyl 4-(4-((2-(2-fluorophenyl)pyrimidin-4-yl)(methyl)amino)pyrimidin-2-yl-amino)piperidine-1-carboxylate.

Analogous to the methods used Example 71, Step B using tert-butyl 4-(4-((2-chloropyrimidin-4-yl)(methyl)amino)pyrimidin-2-ylamino)piperidine-1-carboxylate (640 mg, 1.52 mmol), 2-fluorobenzeneboronic acid (256 mg, 1.82 mmol, Lancaster), sodium carbonate (485 mg, 4.57 mmol) and Pd(PPh₃)₂Cl₂ (107 mg, 0.18 mmol) in a mixture of DME, EtOH, and H₂O (7:2:3, 3.0 mL). Purification by flash chromatography (0→80% EtOAc/Hexanes) gave an impure mixture. The residue was dissolved in 10% MeOH/CH₂Cl₂ and loaded onto a 1 g Agilent AccuBOND II SCX solid phase extraction column, washing with 10% MeOH/CH₂Cl₂. The title compound was eluted with 2N NH₃ in MeOH. MS m/z 480 (MH)⁺.

### EXAMPLE 96

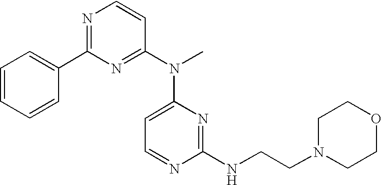 N⁴-Methyl-N²-(2-morpholinoethyl)-N⁴-(2-phenylpyrimidin-4-yl)pyrimidine-2,4-diamine.

A mixture of 2-morpholinoethylamine (167 mg, 1.28 mmol, Aldrich), N-(2-fluoropyrimidin-4-yl)-N-methyl-2-phenylpyrimidin-4-amine (300 mg, 1.07 mmol) and cesium carbonate (416 mg, 1.28 mmol, Aldrich) in DMF (10 mL) was heated to 85° C. for 18 h. The mixture was diluted with H₂O and extracted with CH₂Cl₂ (3×). The combined organics were dried over Na₂SO₄, filtered and concentrated. Purification by flash chromatography (0→5% 2N NH₃ in MeOH/CH₂Cl₂) gave the title compound. MS m/z 334 (MH)⁺.

### EXAMPLE 97

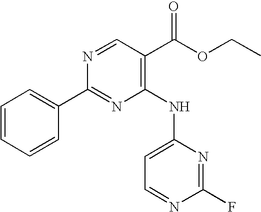 Ethyl 4-(2-fluoropyrimidin-4-ylamino)-2-phenylpyrimidine-5-carboxylate.
Step A: 2-Fluoropyrimidin-4-amine and 4-fluoropyrimidin-2-amine.

Anhydrous ammonia was bubbled through a −78° C. solution of 2,4-difluoropyrimidine (15 g, 129 mmol) for 20 min. The solution was stirred (−78° C.→RT) for 20 h, then diluted with MeOH and concentrated over silica gel. Purification by flash chromatography (0→50%→100% EtOAc/Hexanes) afforded (in order of elution) 4-fluoropyrimidin-2-amine as a white solid [MS m/z 114 (MH)⁺], and 2-fluoro-pyrimidin-4-amine as a white solid [MS m/z 114 (MH)⁺].

Step B: Ethyl 4-chloro-2-phenylpyrimidine-5-carboxylate.

A mixture of ethyl 6-oxo-2-phenyl-1,6-dihydropyrimidine-5-carboxylate (10 g, 41 mmol) and phosphorus oxychloride (20 mL, 215 mmol, Aldrich) was stirred at 105° C. for 3 h. After cooling to RT, the volatiles were removed in vacuo. The residue was partitioned between CH₂Cl₂ and sat aq. NaHCO₃ and stirred for 3 h. The organic layer was collected and the aqueous layer was extracted with CH₂Cl₂ (2×). The combined organics were filtered through a pad of silica gel, eluting with EtOAc. The solution was concentrated to yield the title product. MS m/z 263 (MH)⁺.

Step C: Ethyl 4-(2-fluoropyrimidin-4-ylamino)-2-phenylpyrimidine-5-carboxylate.

A mixture of 2-fluoropyrimidin-4-amine (1.94 g, 17.1 mmol), ethyl 4-chloro-2-phenylpyrimidine-5-carboxylate (3.0 g, 11.4 mmol) and cesium carbonate (5.57 g, 17.1 mmol, Aldrich) was heated to 85° C. for 18 h. The mixture was cooled to RT, diluted with H₂O and extracted with CH₂Cl₂ (2×) and CHCl₃ (2×). The combined organics were dried over Na₂SO₄, Filtered and concentrated. Purifications by flash chromatography (0→15→50% EtOAc/Hexanes) afforded the title product. MS m/z 340 (MH)⁺.

### EXAMPLE 98

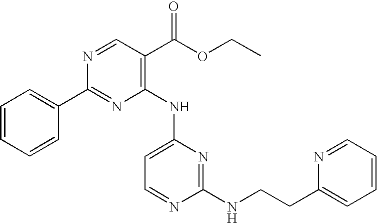 Ethyl 2-phenyl-4-(2-(2-(pyridin-2-yl)ethylamino)pyrimidin-4-ylamino)pyrimidine-5-carboxylate.

A mixture of 2-(2-aminoethyl)pyridine (216 mg, 1.77 mmol), ethyl 4-(2-fluoropyrimidin-4-ylamino)-2-phenylpyrimidine-5-carboxylate (500 mg, 1.47 mmol) and cesium carbonate (815 mg, 2.51 mmol, Aldrich) in DMF (10 mL) was stirred at RT for 24 h. The mixture was diluted with H₂O and extracted with CH₂Cl₂ (3×). The combined organics were dried over Na₂SO₄, filtered and concentrated. Purification by flash chromatography (0→5% 2N NH₃ in MeOH/CH₂Cl₂) gave the title compound. MS m/z 442 (MH)⁺.

### EXAMPLE 99

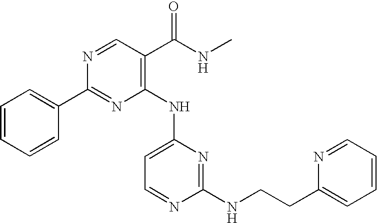 N-Methyl-2-phenyl-4-(2-(2-(pyridin-2-yl)ethylamino)pyrimidin-4-ylamino)-pyrimidine-5-carboxamide.

A mixture of ethyl 2-phenyl-4-(2-(2-(pyridin-2-yl)-ethylamino)pyrimidin-4-ylamino)pyrimidine-5-carboxylate (250 mg, 0.57 mmol), methylamine solution (2.0M in THF, 1.42 mL, 2.83 mmol) and Ti(OEt)₄ (0.06 mL, 0.28 mmol, Aldrich) in THF (1.5 mL) was heated to 150° C. for 1 h in the Smith Synthesizer microwave. The mixture was diluted with MeOH and concentrated over silica gel. Purification by flash chromatography (0→5% 2N NH₃ in MeOH/CH₂Cl₂) afforded the title compound. MS m/z 427 (MH)⁺.

### EXAMPLE 100

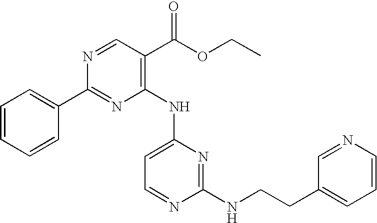 Ethyl 2-phenyl-4-(2-(2-(pyridin-2-yl)ethylamino)pyrimidin-4-ylamino)pyrimidine-5-carboxylate.

Analogous to the methods used in Example 98 using ethyl 4-(2-fluoropyrimidin-4-ylamino)-2-phenylpyrimidine-5-carboxylate (200 mg, 0.59 mmol), 3-(2-aminoethyl)pyridine (72 mg, 0.59 mmol) and cesium carbonate (191 mg, 0.59 mmol) in DMF (5 mL). Purification by flash chromatography (0→2.0% 2N NH₃ in MeOH/CH₂Cl₂) afforded the title compound. MS m/z 442 (MH)⁺.

### EXAMPLE 101

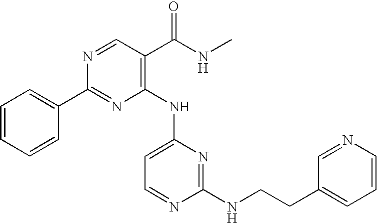 N-Methyl-2-phenyl-4-(2-(2-(pyridin-3-yl)ethylamino)pyrimidin-4-ylamino)-pyrimidine-5-carboxamide.

Analogous to the methods used in Example 99 using ethyl 2-phenyl-4-(2-(2-(pyridin-2-yl)ethylamino)pyrimidin-4-ylamino)pyrimidine-5-carboxylate (210 mg, 0.48 mmol), methylamine solution (2.0M in THF, 0.72 mL, 1.43 mmol) and Ti(OEt)₄ (0.02 mL, 0.096 mmol) in THF (2 mL). Purification by flash chromatography (0→5% 2N NH₃ in MeOH/CH₂Cl₂) afforded the title compound. MS m/z 427 (MH)⁺.

### EXAMPLE 102

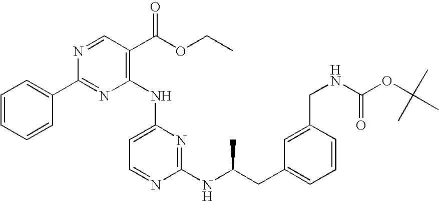 (S)-Ethyl 4-(2-(1-(3-((tert-butoxycarbonyl)methyl)phenyl)propan-2-ylamino)-pyrimidin-4-ylamino)-2-phenylpyrimidine-5-carboxylate.

Analogous to the methods used in Example 98 using ethyl 4-(2-fluoropyrimidin-4-ylamino)-2-phenylpyrimidine-5-carboxylate (500 mg, 1.47 mmol), (S)-tert-butyl 3-(2-amino-propyl)benzylcarbamate (467 mg, 1.77 mmol) and cesium carbonate (575 mg, 1.77 mmol) in DMF (10 mL). Purification by flash chromatography (0→30% EtOAc/Hexanes) afforded the title compound. MS m/z 584 (MH)⁺.

### EXAMPLE 103

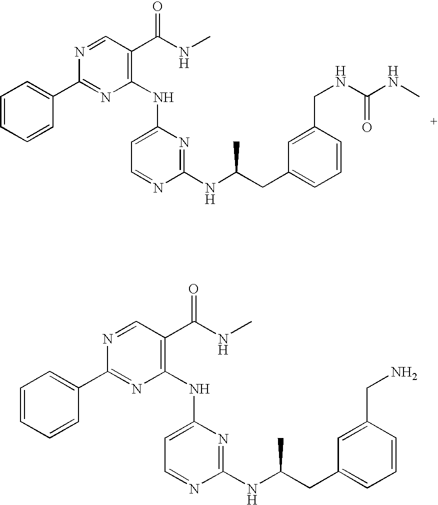 (S)-1-(3-(2-(4-(5-(Methylcarbamoyl)-2-phenylpyrimidin-4-ylamino)pyrimidin-2-ylamino)propyl)benzyl)-3-methylurea and (S)-4-(2-(1-(3-(aminomethyl)phenyl)-propan-2-ylamino)pyrimidin-4-ylamino)-N-methyl-2-phenylpyrimidine-5-carboxamide.

Analogous to the methods used in Example 99 using (S)-ethyl 4-(2-(1-(3-((tert-butoxycarbonyl)methyl)phenyl)propan-2-ylamino)pyrimidin-4-yl-amino)-2-phenylpyrimidine-5-carboxylate (400 mg, 0.68 mmol), methylamine solution (2.0M in THF, 3.4 mL, 6.8 mmol) and Ti(OEt)₄ (0.072 mL, 0.34 mmol). Purification by flash chromatography (0→5% MeOH/CH₂Cl₂) afforded (S)-1-(3-(2-(4-(5-(methylcarbamoyl)-2-phenylpyrimidin-4-ylamino)pyrimidin-2-ylamino)-propyl)benzyl)-3-methylurea. MS m/z 526 (MH)⁺. Further elution (5% 2N NH₃ in MeOH/CH₂Cl₂) afforded (S)-4-(2-(1-(3-(aminomethyl)phenyl)propan-2-ylamino)-pyrimidin-4-ylamino)-N-methyl-2-phenylpyrimidine-5-carboxamide. MS m/z 469 (MH)⁺.

### EXAMPLE 104

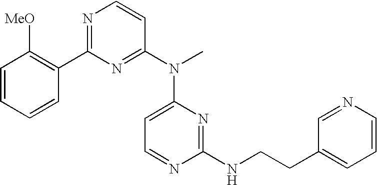 N⁴-(2-(2-Methoxyphenyl)pyrimidin-4-yl)-N⁴-methyl-N²-(2-(pyridin-3-yl)ethyl)-pyrimidine-2,4-diamine.

This material was prepared according to the method described in Example 71, Step B using N⁴-(2-chloropyrimidin-4-yl)-N⁴-methyl-N²-(2-(pyridin-3-yl)ethyl)pyrimidine-2,4-diamine (154 mg, 0.5 mmol), 2-methoxy-benzene boronic acid (111 mg, 0.7 mmol), Na₂CO₃ (223 mg, 2.10 mmol) and PdCl₂(PPh₃)₂ (30 mg, 0.04 mmol) in 1.4 mL DME/0.6 mL H₂O/0.4 mL EtOH. Purification by flash column chromatography with 2N NH₃ in MeOH/CH₂Cl₂ (0:1→1:19) as eluant gave the title compound as a light yellow tar. MS m/z 414 (MH)⁺.

### EXAMPLE 105

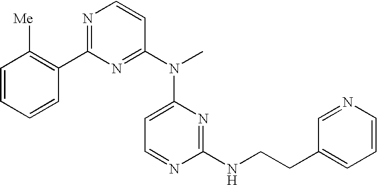 N⁴-Methyl-N²-(2-(pyridin-3-yl)ethyl)-N⁴-(2-o-tolylpyrimidin-4-yl)pyrimidine-2,4-diamine.

This material was prepared according to the method described in Example 71, Step B using N⁴-(2-chloropyrimidin-4-yl)-N⁴-methyl-N²-(2-(pyridin-3-yl)ethyl)-pyrimidine-2,4-diamine (151 mg, 0.4 mmol), o-tolylboronic acid (96 mg, 0.7 mmol), Na₂CO₃ (209 mg, 2.0 mmol) and PdCl₂(PPh₃)₂ (30 mg, 0.04 mmol) in 1.4 mL DME/0.6 mL H₂O/0.4 mL EtOH. Purification by flash column chromatography with 2N NH₃ in MeOH/CH₂Cl₂ (0:1→1:19) as eluant gave the title compound as a colorless glass. MS m/z 398 (MH)⁺.

### EXAMPLE 106

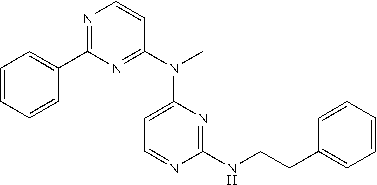 N⁴-Methyl-N²-phenethyl-N⁴-(2-phenylpyrimidin-4-yl)pyrimidine-2,4-diamine.

A mixture N-(2-fluoropyrimidin-4-yl)-N-methyl-2-phenylpyrimidin-4-amine (99 mg, 0.4 mmol) and phenethylamine (Aldrich, 0.12 mL, 1.0 mmol) in 1.5 mL i-PrOH was heated to 140° C. for 15 min in the microwave. The reaction mixture was evaporated onto SiO₂ and purified by flash column chromatography with EtOAc/hexane/2N NH₃ in MeOH/CH₂Cl₂ (0:1:0:0→1:1:0:0→0:0:1:49→0:0:1:19) as eluant to give the title compound as a white amorphous solid. MS m/z 383 (MH)⁺.

### EXAMPLE 107

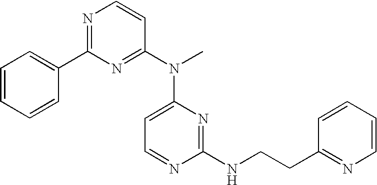 N⁴-Methyl-N⁴-(2-phenylpyrimidin-4-yl)-N²-(2-(pyridin-2-yl)ethyl)pyrimidine-2,4-diamine.

This material was prepared according to the method described in Example 106 using N-(2-fluoropyrimidin-4-yl)-N-methyl-2-phenylpyrimidin-4-amine (103 mg, 0.4 mmol) and 2-(2-aminoethyl)pyridine (Aldrich, 0.12 mL, 1.0 mmol) in 1.5 mL i-PrOH. Purification by flash column chromatography with 2N NH₃ in MeOH/CH₂Cl₂ (0:1→1:19) as eluant gave the title compound as a white amorphous solid. MS m/z 384 (MH)⁺.

### EXAMPLE 108

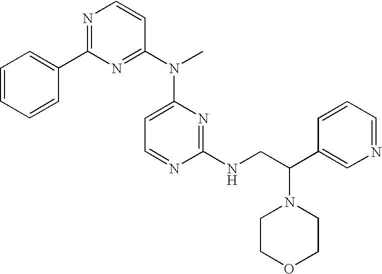 N⁴-Methyl-N²-(2-morpholino-2-(pyridin-3-yl)ethyl)-N⁴-(2-phenylpyrimidin-4-yl)pyrimidine-2,4-diamine.

This material was prepared according to the method described in Example 106. using N-(2-fluoropyrimidin-4-yl)-N-methyl-2-phenylpyrimidin-4-amine (99 mg, 0.4 mmol) and 2-morpholine-4-yl-2-(3-pyridyl)ethylamine (Array-Biopharma, 165 mg, 0.8 mmol) in 1.5 mL i-PrOH. Purification by flash column chromatography with EtOAc/hexane/2N NH₃ in MeOH/CH₂Cl₂ (0:1:0:0→4:6:0:0→0:0:0:1→0:0:1:24) as eluant gave the title compound as a yellow tar. MS m/z 469 (MH)⁺.

### EXAMPLE 109

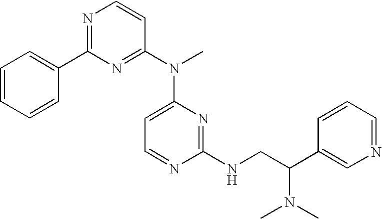 N²-(2-(Dimethylamino)-2-(pyridin-3-yl)ethyl)-N⁴-methyl-N⁴-(2-phenylpyrimidin-4-yl)pyrimidine-2,4-diamine.

This material was prepared according to the method described in Example 106 using N-(2-fluoropyrimidin-4-yl)-N-methyl-2-phenyl-pyrimidin-4-amine (314 mg, 1.1 mmol) and [2-amino-1-(3-pyridyl)ethyl]dimethylamine (Array-Biopharma, 305 mg, 1.9 mmol) in 2.5 mL i-PrOH. Purification by flash column chromatography with 2N NH₃ in MeOH/CH₂Cl₂ (0:1→3:97) as eluant gave the title compound. MS m/z 427 (MH)⁺.

### EXAMPLE 110

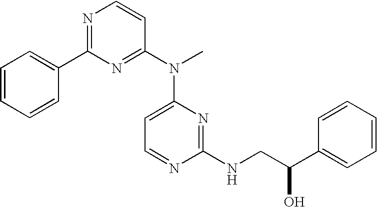 (R)-2-(4-(Methyl(2-phenylpyrimidin-4-yl)amino)pyrimidin-2-ylamino)-1-phenyl-ethanol.

This material was prepared according to the method described in Example 106 using N-(2-fluoropyrimidin-4-yl)-N-methyl-2-phenylpyrimidin-4-amine (102 mg, 0.4 mmol) and (R)-(−)-2-amino-1-phenylethanol (Aldrich, 79 mg, 0.6 mmol) in 1.5 mL i-PrOH. Purification by flash column chromatography with 2N NH₃ in MeOH/CH₂Cl₂ (0:1→1:49) as eluant gave the title compound as a white amorphous solid. MS m/z 399 (MH)⁺.

### EXAMPLE 111

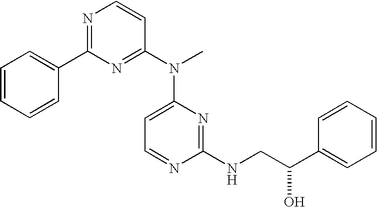 (S)-2-(4-(Methyl(2-phenylpyrimidin-4-yl)amino)pyrimidin-2-ylamino)-1-phenyl-ethanol.

This material was prepared according to the method described in Example 106 using N-(2-fluoropyrimidin-4-yl)-N-methyl-2-phenylpyrimidin-4-amine (102 mg, 0.4 mmol) and (S)-2-amino-1-phenylethanol (Fluka, 69 mg, 0.5 mmol) in 1.5 mL i-PrOH. Purification by flash column chromatography with 2N NH₃ in MeOH/CH₂Cl₂ (0:1→1:39) as eluant gave the title compound as a white amorphous solid. MS m/z 399 (MH)⁺.

### EXAMPLE 112

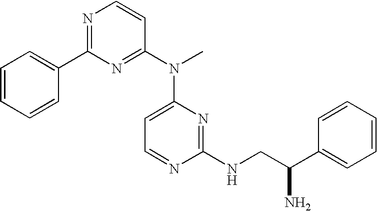 (R)-N²-(2-Amino-2-phenylethyl)-N⁴- -4-yl)pyrimidine-2,4-diamine.

To a cooled (0° C.) solution of (S)-2-(4-(methyl(2-phenylpyrimidin-4-yl)amino)pyrimidin-2-ylamino)-1-phenylethanol (644 mg, 1.6 mmol) and Ph₃P (Aldrich, 851 mg, 3.2 mmol) in 100 mL THF was added diethylazodicarboxylate (Aldrich, 0.5 mL, 3.2 mmol) and diphenylphosphoryl azide (Aldrich, 1.0 mL, 4.6 mmol) sequentially. After 1.5 h the reaction mixture was transferred to a 100 mL round bottom flask and concentrated to an oil. The residue was dissolved in 20 mL EtOAc and 10%-wet palladium/carbon (171 mg) was added. The mixture was evacuated, purged with H₂ and stirred at RT. After 3.5 h the reaction mixture was filtered through Celite, evaporated onto SiO₂ and purified by flash column chromatography with 2N NH₃ in MeOH/CH₂Cl₂ (0:1→7:193) as eluant to give the title compound as a yellow tar. MS m/z 398 (MH)⁺.

### EXAMPLE 113

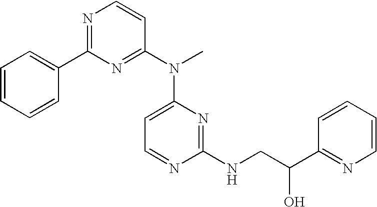 2-(4-(Methyl(2- -(pyridin-2-yl)ethanol.

This material was prepared according to the method described in Example 106 using N-(2-fluoropyrimidin-4-yl)-N-methyl-2-phenylpyrimidin-4-amine (131 mg, 0.5 mmol) and 2-hydroxy-2-pyridylethylamine (Array-Biopharma, 101 mg, 0.7 mmol) in 1.5 mL i-PrOH. Purification by flash column chromatography 2N NH₃ in MeOH/CH₂Cl₂ (0:1→3:97) as eluant gave the title compound as a white amorphous solid. MS m/z 400 (MH)⁺.

### EXAMPLE 114

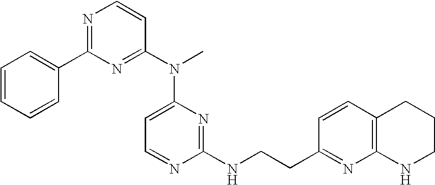 N⁴-Methyl-N⁴-(2-phenylpyrimidin-4-yl)-N²-(2-(5,6,7,8-tetrahydro-1,8-naphthyridin-2-yl)ethyl)pyrimidine-2,4-diamine.

This material was prepared according to the method described in Example 106 using N-(2-fluoropyrimidin-4-yl)-N-methyl-2-phenylpyrimidin-4-amine (164 mg, 0.6 mmol) and 5,6,7,8-tetrahydro-1,8-naphthyridin-2-yl-ethylamine (Astatech, 119 mg, 0.7 mmol) in 7 mL i-PrOH. Purification by flash column chromatography 2N NH₃ in MeOH/CH₂Cl₂ (0:1→1:19) as eluant gave the title compound as a light yellow amorphous solid. MS m/z 439 (MH)⁺.

### EXAMPLE 115

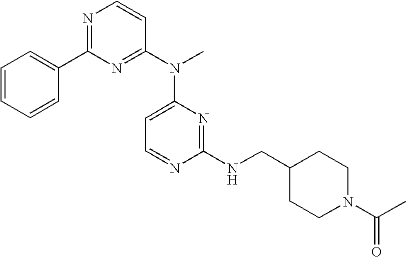 1-(4-((4-(Methyl(2-phenylpyrimidin-4-yl)amino)pyrimidin-2-ylamino)methyl)-piperidin-1-yl)ethanone.

A mixture N-(2-fluoropyrimidin-4-yl)-N-methyl-2-phenylpyrimidin-4-amine (490 mg, 1.7 mmol) and 4-(aminomethyl)-1-BOC-piperidine (Astatech, 520 mg, 2.4 mmol) in 6 mL i-PrOH was heated to 135° C. for 20 min in the microwave. The reaction mixture was evaporated onto SiO₂ and purified by flash column chromatography with 2N NH₃ in MeOH/CH₂Cl₂ (0:1→1:39). The fractions containing the desired coupled product were combined, concentrated and dissolved in 20 mL CH₂Cl₂. To the solution was added 20 mL of 1N HCl in Et₂O. After 6 h the resulting solid was filtered, washed with CH₂Cl₂ and dried in vacuo. The solid was heated in 3 mL CH₂Cl₂ to 55° C. in the presence of Ac₂O (0.1 mL). After 2 h the reaction mixture was cooled to RT, evaporated onto SiO₂ and purified by flash column chromatography with 2N NH₃ in MeOH/CH₂Cl₂ (0:1→7:193) to give a colorless tar. MS m/z 418 (MH)⁺.

### EXAMPLE 116

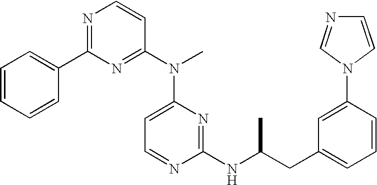 N²-((S)-1-(3-(1 H-Imidazol-1-yl)phenyl)propan-2-yl)-N⁴-methyl-N⁴-(2-phenyl-pyrimidin-4-yl)pyrimidine-2,4-diamine
Step A: (2S)-1-(3-(1H-Imidazol-1-yl)phenyl)propan-2-amine.

A mixture of (S)-benzyl 1-(3-bromophenyl)propan-2-ylcarbamate (1.73 g, 5.0 mmol), CuI, (150 mg, 0.8 mmol), K₂CO₃ (1.52 g, 11.0 mmol) and imidazole (690 mg, 10.1 mmol) in 3 mL NMP was heated to 195° C. for 2 h in the microwave. The reaction was filtered and the solvent was removed in vacuo. The residue was dissolved in MeOH, evaporated onto SiO₂ and purified by flash column chromatography with 2N NH₃ in MeOH/CH₂Cl₂ (0:1→15:185) to give 581 mg of a brown oil. MS m/z 202 (MH)⁺.

Step B: N²-((S)-1-(3-(1H-Imidazol-1-yl)phenyl)propan-2-yl)-N⁴-methyl-N⁴-(2-phenylpyrimidin-4-yl)pyrimidine-2,4-diamine.

A mixture of N-(2-fluoropyrimidin-4-yl)-N-methyl-2-phenylpyrimidin-4-amine (155 mg, 0.6 mmol), (2S)-1-(3-(1H-imidazol-1-yl)phenyl)propan-2-amine (118 mg, 0.6 mmol) and Cs₂CO₃ (161 mg, 0.5 mmol) in 2 mL DMF was heated to 85° C. After 4 h the reaction was cooled to RT and H₂O was added. After extraction with EtOAc (3×), the combined organics were evaporated onto SiO₂ and purified by flash column chromatography with 2N NH₃ in MeOH/CH₂Cl₂ (0:1→1:19) as eluant to give the title compound as a white amorphous solid. MS m/z 463 (MH)⁺.

### EXAMPLE 117

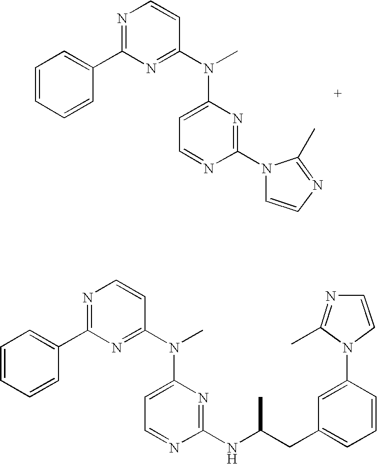 Step A: (2S)-1-(3-(2-Methyl-1H-imidazol-1-yl)phenyl)propan-2-amine.

This material was prepared according to the method described in Example 116, Step A using (S)-benzyl 1-(3-bromophenyl)propan-2-ylcarbamate (1.73 g, 5.0 mmol), CuI, (153 mg, 0.8 mmol), K₂CO₃ (1.53 g, 11.1 mmol) and 2-methylimidazole (851 mg, 10.4 mmol) in 4 mL NMP. Purification by flash column chromatography 2N NH₃ in MeOH/CH₂Cl₂ (0:1→2:23) as eluant gave the title compound as a brown oil. MS m/z 216 (MH)⁺.

Step B: N⁴-Methyl-N²-((S)-1-(3-(2-methyl-1H-imidazol-1-yl)phenyl)propan-2-yl)-N⁴-(2-phenylpyrimidin-4-yl)pyrimidine-2,4-diamine.

This material was prepared according to the method described in Example 19, Step B, using N-(2-fluoro-pyrimidin-4-yl)-N-methyl-2-phenylpyrimidin-4-amine (250 mg, 0.9 mmol), (2S)-1-(3-(2-methyl-1H-imidazol-1-yl)phenyl)propan-2-amine (190 mg, 0.9 mmol) and Cs₂CO₃ (395 mg, 1.2 mmol) in 3.5 mL DMF. Purification by flash column chromatography with 2N NH₃ in MeOH/CH₂Cl₂ (0:1→1:19) as eluant. The early fractions contained N-methyl-N-(2-(2-methyl-1H-imidazol-1-yl)pyrimidin-4-yl)-2-phenylpyrimidin-4-amine as a white amorphous solid. MS m/z 344 (MH)⁺. Later fractions yielded N⁴-methyl-N²-((S)-1-(3-(2-methyl-1H-imidazol-1-yl)phenyl)-propan-2-yl)-N⁴-(2-phenylpyrimidin-4-yl)pyrimidine-2,4-diamine as a white amorphous solid. MS m/z 477 (MH)⁺.

### EXAMPLE 118

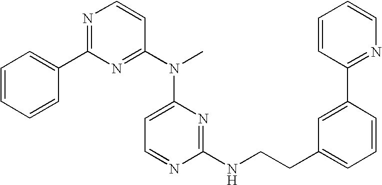 N²-(3-(Pyridin-2-yl)phenethyl)-N⁴-methyl-N⁴-(2-phenylpyrimidin-4-yl)pyrimidine-2,4-diamine
Step A: 3-(Pyridin-2-yl)benzaldehyde.

A mixture of 3-formyl boronic acid (Lancaster, 5.0 g, 33 mmol), 2-bromopyridine (Aldrich, 4.80 mL, 501 mmol), Cs₂CO₃ (37.6 g, 115 mmol) and PdCl₂(PPh₃)₂ (1.02 g, 1.5 mmol) in 90 mL THF was heated to 65° C. After 3 h the reaction was cooled to RT and partitioned between EtOAc/H₂O. The aqueous layer was extracted with EtOAc (3×) and the combined organics were washed with brine and dried over Na₂SO₄. The solution was filtered, evaporated onto SiO₂ and purified by flash column chromatography with EtOAc/hexane (0:1→1:4) as eluant to give the title compound as a light yellow oil. MS m/z 184 (MH)⁺.

Step B: (E)-2-(3-(2-Nitrovinyl)phenyl)pyridine.

To a solution of 3-(pyridin-2-yl)benzaldehyde (813 mg, 4.4 mmol) in 8 mL AcOH was added nitromethane (1.5 mL, 27.9 mmol) followed by NH₄OAc (1.51 g, 19.6 mmol). The mixture was heated to 100° C. for 1 h and then cooled to RT. After removing one-half of the solvent volume in vacuo, the concentrated solution was diluted with 100 mL EtOAc, washed with saturated NaHCO₃ and dried over Na₂SO₄. The solution was filtered, concentrated and purified through a short plug of SiO₂ with 25% EtOAc/hexane as eluant to give a yellow crystalline solid. MS m/z 227 (MH)⁺.

Step C: N²-(3-(Pyridin-2-yl)phenethyl)-N⁴-methyl-N⁴-(2-phenylpyrimidin-4-yl)pyrimidine-2,4-diamine.

To a RT slurry of lithium aluminum hydride (Aldrich, 550 mg, 14.5 mmol) in 5 mL THF was added a solution of (E)-2-(3-(2-nitrovinyl)phenyl)pyridine (550 mg, 2.4 mmol) in 4 mL THF. The addition is exothermic and gas evolution occurs. The reaction was heated to 65° C. for 5 h and then cooled to 0° C. To the mixture was carefully added a 30% NaOH solution until gas evolution ceased. The mixture was diluted three times its volume with EtOAc and stirred vigorously for 1 h. The layers were separated and the organic layer was dried over Na₂SO₄. MS (ESI, pos. ion) m/z: 199 (M+1). A portion of 2-(3-(pyridin-2-yl)phenyl)ethanamine (150 mg, 0.8 mmol) was allowed to react with N-(2-fluoropyrimidin-4-yl)-N-methyl-2-phenylpyrimidin-4-amine (212 mg, 0.8 mmol) in 2.5 mL i-PrOH according to the method described in Example 106. Purification by flash column chromatography 2N NH₃ in MeOH/CH₂Cl₂ (0:1→3:97) as eluant followed by purification by reverse-phase HPLC gave the title compound as a yellow tar. MS m/z 460 (MH)⁺.

### EXAMPLE 119

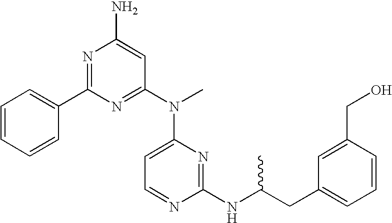 (3-(2-(4-((6-Amino-2-phenylpyrimidin-4-yl)(methyl)amino)pyrimidin-2-ylamino) -propyl)phenyl)methanol
Step A: tert-Butyl-6-chloro-2-phenylpyrimidin-4-ylcarbamate.

A mixture of 6-chloro-2-phenylpyrimidin-4-amine (0.15 mg, 0.73 mmol), di-tert-butyldicarbonate 1.0M in THF (0.95 mL, 0.95 mmol), N,N-diisopropylethylamine (0.15 mL, 0.88 mmol) and a catalytic amount of 4-dimethylaminopyridine in DMF (3 mL) was stirred at RT for 17 h then heated at 45° C. for 5 h and brought to RT. Mixture was poured into water, and extracted with ethyl acetate (EtOAc). The organic extracts were combined, washed with saturated NH₄Cl, brine, dried over magnesium sulfate and concentrated to afford a yellow-brown solid. MS m/z 306 (MH)⁺.

Step B: tert-Butyl-6-(methylamino)-2-phenylpyrimidin-4-ylcarbamate:

A mixture of tert-butyl-6-chloro-2-phenylpyrimidin-4-ylcarbamate (60 mg, 0.20 mmol), methylamine (0.24 g, 3.68 mmol), triethylamine (0.84 mL, 6.16 mmol) in ethanol/DMF (3 mL/2 mL) was heated at 80° C. in a sealed tube for 15 h. The mixture was brought to RT, poured into water and extracted with EtOAc. The organic extracts were combined, washed with saturated NH₄Cl, brine, dried over magnesium sulfate, concentrated and chromatographed on silica gel using hexanes. MS m/z 301 (MH)⁺.

Step C: tert-Butyl-6-(methyl(2-(methylthio)pyrimidin-4-yl)amino)-2-phenyl-pyrimidin-4-ylcarbamate.

A mixture of tert-butyl-6-(methylamino)-2-phenyl-pyrimidin-4-ylcarbamate (60 mg, 0.20 mmol), 4-chloro-2-methylthiopyrimidine (30 μL, 0.26 mmol), tris(dibenzylideneacetone)dipalladium(0) (9.5 mg, 0.01 mmol), rac-2-2′-bis)diphenylphosphino)-1,1′-bynaphthyl (12 mg, 0.02 mmol), sodium tert-butoxide (25 mg, 0.26 mmol) in toluene was heated to 90° C. for 17 h. The mixture was brought to RT, diluted in EtOAc, washed with saturated NH₄Cl, brine, dried over magnesium sulfate, concentrated and chromatographed on silica gel using 0-2% MeOH/CH₂Cl₂. MS m/z 425 (MH)⁺.

Step D: tert-Butyl-6-(methyl(2-(methylsulfinyl)pyrimidin-4-yl)amino)-2-phenyl-pyrimidin-4-ylcarbamate:

A mixture of tert-Butyl-6-(methyl(2-(methylthio)-pyrimidin-4-yl)amino)-2-phenylpyrimidin-4-ylcarbamate (30 mg, 0.071 mmol) and m-chloroperoxybenzoic acid (12 mg, 0.07 mmol) in CH₂Cl₂ (2 mL) was stirred at RT for 2 h. The mixture was washed with saturated NaHCO₃, brine, dried over magnesium sulfate, and concentrated to be used as is. MS m/z 441 (MH)⁺.

Step E: tert-Butyl-6-((2-(1-(3-(hydroxymethyl)phenyl)propan-2-ylamino)pyrimidin-4-yl)(methyl)amino)-2-phenylpyrimidin-4-ylcarbamate.

A mixture of tert-butyl-6-(methyl(2-(methylsulfinyl)pyrimidin-4-yl)amino)-2-phenylpyrimidin-4-ylcarbamate (13 mg, 0.03 mmol), (3-(2-aminopropyl)phenyl)methanol (50 mg, 0.30 mmol) in N-methylpyrrolidone (NMP) (1 mL) was heated to 100° C. for 15 h. The mixture was brought to RT, poured into water, and extracted with EtOAc. The organic extracts were combined, washed with saturated NaHCO₃, brine, dried over magnesium sulfate, concentrated and chromatographed on silica gel using 0-4-8% MeOH/CH₂Cl₂. MS m/z 542 (MH)⁺.

Step F: (3-(2-(4-((6-Amino-2-phenylpyrimidin-4-yl)(methyl)amino)pyrimidin-2-ylamino)propyl)phenyl)methanol.

A mixture of tert-butyl-6-((2-(1-(3-(hydroxy-methyl)phenyl)propan-2-ylamino)pyrimidin-4-yl)(methyl)amino)-2-phenyl-pyrimidin-4-ylcarbamate (6.9 mg, 0.013 mmol) and trifluoroacetic acid (5 mL) in dichloromethane (5 mL) was stirred at RT for 40 min and quenched with saturated NaHCO₃. The organic phase was separated, washed again with saturated NaHCO₃ (4×), brine, dried over magnesium sulfate, concentrated and chromatographed on silica gel using 0-8% MeOH/CH₂Cl₂. MS m/z 442 (MH)⁺.

### EXAMPLE 120

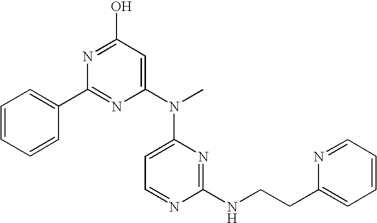 6-(Methyl(2-(2-(pyridin-2-yl)ethylamino)pyrimidin-4-yl)amino)-2-phenylpyrimidin-4-ol
Step A: 6-Amino-2-phenylpyrimidin-4-ol.

To a mixture of benzamidine-HCl (10 g, 65 mmol) and cyanoacetic ester (6.9 mL, 65 mmol) in MeOH (20 mL), cooled in an ice-bath, was added 25 wt % NaOMe (56 mL, 258 mmol) in MeOH. The solution was heated at reflux for 2 h then concentrated in vacuo and dissolved in warm water (80 mL). A solid began to form and the mixture was allowed to stand at RT for 15 h. A white crystalline solid was filtered off and dried on high-vacuum to give 4 g of the desired product. MS m/z 188 (MH)⁺.

Step B: 6-(Methylamino)-2-phenylpyrimidin-4-ol.

The amine (2.72 g, 14.5 mmol) and methylamine hydrochloride (10.80 g, 160 mmol) were melted in a flask until the internal temperature reached 190° C. for 3 h. Cooled to RT then purified by silica flash chromatography (0-10% MeOH/DCM) to yield the desired product. MS m/z 202 (MH)⁺.

Step C: 6-((2-Fluoropyrimidin-4-yl)(methyl)amino)-2-phenylpyrimidin-4-ol.

The amine (1.5 g, 7.425 mmol) was stirred at RT with 2,4-difluoropyrimidine (0.948 g, 8.168 mmol) and potassium carbonate (3.08 g, 22.3 mmol) in NMP (100 mL) overnight. The solution was taken up in ethyl acetate (200 mL) and washed five times with water (50 mL) and twice with brine (50 mL), dried with MgSO₄ and concentrated in vacuo. Purification by silica flash chromatography (20-80% EtOAc/Hexanes) yielded the title compound. MS m/z 298 (MH)⁺.

Step D: 6-(Methyl(2-(2-(pyridin-2-yl)ethylamino)pyrimidin-4-yl)amino)-2-phenyl-pyrimidin-4-ol.

The 2-fluoropyrimidine from previous step (0.125 g, 0.420 mmol) and 2-(2-aminoethyl)pyridine (0.10 mL, 0.840 mmol) were heated to 135° C. in a microwave for 15 min in 5 mL of isopropyl alcohol. The mixture was then concentrated in vacuum and purified by HPLC to give a white crystalline TFA salt. MS m/z 400 (MH)⁺.

### EXAMPLE 121

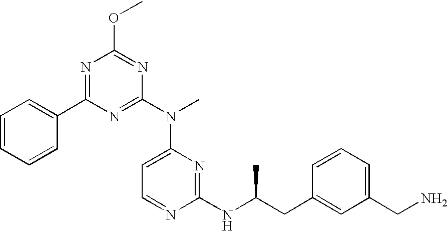 (S)-N²-(1-(3-(Aminomethyl)phenyl)propan-2-yl)-N⁴-(4-methoxy-6-phenyl-1,3,5-triazin-2-yl)-N⁴-methylpyrimidine-2,4-diamine
Step A: 4-Chloro-6-methoxy-N-methyl-1,3,5-triazin-2-amine.

A mixture of 2,4-dichloro-6-methoxypyrimidine (4.4 g, 24.4 mmol) in isopropanol (100 mL) was brought to 0° C. followed by the addition of methylamine (16 mL, 31.7 mmol). The resulting white suspension was stirred for 5 h at 0° C. and gradually brought to RT and stirred for 15 h. The mixture was concentrated and the residue obtained was diluted in dichloromethane, washed with saturated NaHCO₃, brine, dried over magnesium sulfate, concentrated and chromatographed on silica gel using dichloromethane to afford a white solid. MS m/z 175 (MH)⁺.

Step B: 4-Methoxy-N-methyl-6-phenyl-1,3,5-triazin-2-amine.

A mixture of 4-chloro-6-methoxy-N-methyl-1,3,5-triazin-2-amine (0.28 g, 1.61 mmol), phenyl boronic acid (0.39 g, 3.22 mmol), [1,1′-bis(diphenylphosphino)ferrocene]dichloro-palladium(II) (PdCl₂(dppf)₂) (0.13 g, 0.161 mmol), sodium carbonate (Na₂CO₃.H₂O) (0.6 g, 4.83 mmol/in 2.5 mL H₂O), in ethylene glycol dimethyl ether (DME) (10 mL) was heated to reflux for 5 h and brought to RT. The mixture was filtered through celite, concentrated and chromatographed on silica gel using 0-4% MeOH/CH₂Cl₂ to afford a white solid. MS m/z 217 (MH)⁺.

Step C: 4-Methoxy-N-methyl-N-(2-(methylthio)pyrimidin-4-yl)-6-phenyl-1,3,5-triazin-2-amine.

Procedure same as described as on Example 119, Step C. Light-yellow solid. MS m/z 310 (MH)⁺.

Step D: N-Methyl-2-(methylsulfinyl)-N-(6-phenylpyrazin-2-yl)pyrimidin-4-amine.

Procedure same as described as on Example 119, Step D. Light-yellow solid. MS m/z 326 (MH)⁺.

Step E: (S)-3-(2-(4-((4-Methoxy-6-phenyl-1,3,5-triazine-2-yl)(methyl)amino)-pyrimidin-2-ylamino)propyl)benzonitrile.

Procedure same as on Example 119, Step E. MS m/z 422 (MH)⁺.

Step F: (S)-N²-(1-(3-(Aminomethyl)phenyl)propan-2-yl)-N⁴-(4-methoxy-6-phenyl-1,3,5-triazin-2-yl)-N⁴-methylpyrimidine-2,4-diamine.

To a mixture of (S)-3-(2-(4-((4-methoxy-6-phenyl-1,3,5-triazine-2-yl)(methyl)amino)pyrimidin-2-ylamino)-propyl)benzonitrile (60 mg, 0.14 mmol) and 2N NH₃ (2 mL) in methanol (30 mL) was added Raney-Ni (10 eq). The mixture was purged with N₂ and H₂ was bubbled through a balloon for 15 h. The mixture was filtered through celite and the remaining Raney-Ni was extracted with aqueous NH₄₀H and dichloromethane. The organic extracts were combined, dried over magnesium sulfate, concentrated and chromatographed on silica gel using 0-8% 2N NH₃MeOH/CH₂Cl₂ to afford a light yellow solid. MS m/z 457 (MH)⁺.

### EXAMPLE 122

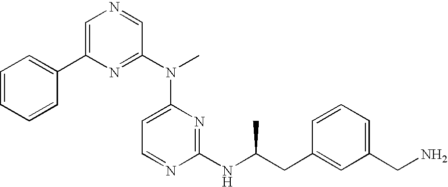 (S)-N²-(1-(3-(Aminomethyl)phenyl)propan-2-yl)-N⁴-methyl-N⁴-(6-phenylpyrazin-2-yl)pyrimidine-2,4-diamine
Step A: 6-Chloro-N-methylpyrazin-2-amine.

Procedure same as on Example 121 Step A. White solid. MS m/z 144 (MH)⁺.

Step B: N-Methyl-6-phenylpyrazin-2-amine.

Procedure same as on Example 121 Step B. Yellow oil. MS m/z 186 (MH)⁺.

Step C: N-Methyl-2-(methylthio)-N-(6-phenylpyrazin-2-yl)pyrimidin-4-amine.

Procedure same as on Example 121 Step C. MS m/z 310 (MH)⁺.

Step D: N-Methyl-2-(methylsulfinyl)-N-(6-phenylpyrazin-2-yl)pyrimidin-4-amine.

Procedure same as on Example 121 Step D. MS m/z 326 (MH)⁺.
Step E: (S)-3-(2-(4-(Methyl(6-phenylpyrazin-2-yl)amino)pyrimidin-2-ylamino)propyl)benzonitrile.

Procedure same Example 121 Step E. MS m/z 422 (MH)⁺.
Step F: (S)-N²-(1-(3-(Aminomethyl)phenyl)propan-2-yl)-N⁴-methyl-N⁴-(6-phenylpyrazin-2-yl)pyrimidine-2,4-diamine.

Procedure same as on Example 121 Step F. MS m/z 426 (MH)⁺.

### EXAMPLE 123

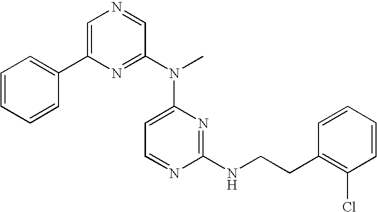 N²-(2-Chlorophenethyl)-N⁴-methyl-N⁴-(6-phenylpyrazin-2-yl)pyrimidine-2,4-diamine
Step A: 6-Chloro-N-methylpyrazin-2-amine.

Procedure same as on Example 121 Step A, reaction 1. White solid. MS m/z 144 (MH)⁺.

Step B: N-Methyl-6-phenylpyrazin-2-amine.

Procedure same as on Example 121 Step B Yellow oil. MS m/z 186 (MH)⁺.

Step C: N-Methyl-2-(methylthio)-N-(6-phenylpyrazin-2-yl)pyrimidin-4-amine.

Procedure same as on Example 121 Step C. MS m/z 310 (MH)⁺.

Step D: N-Methyl-2-(methylsulfinyl)-N-(6-phenylpyrazin-2-yl)pyrimidin-4-amine.

Procedure same as on Example 121 Step D, reaction 4. MS m/z 326 (MH)⁺.

Step E: N²-(2-Chlorophenethyl)-N⁴-methyl-N⁴-(6-phenylpyrazin-2-yl)pyrimidine-2,4-diamine.

Procedure same as on Example 121, Step E. MS m/z 417 (MH)⁺.

### EXAMPLE 124

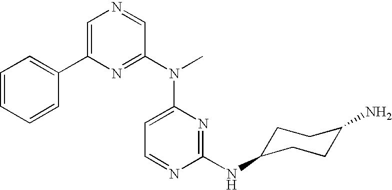 N²-((1r, 4r)-4-Aminocyclohexyl)-N⁴-methyl-N⁴-(6-phenylpyrazine-2-yl)pyrimidine-2,4-diamine.

Procedure same as on Example 121, Step E. MS m/z 376 (MH)⁺.

### EXAMPLE 125

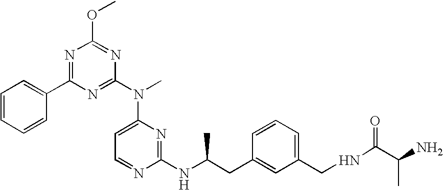 (S)-N-((S)-3-((S)-2-(4-((4-Methoxy-6-phenyl-1,3,5-triazin-2-yl)(methyl)amino)-pyrimidin-2-ylamino)propyl)benzyl)-2-aminopropanamide
Step A: (9H-Fluoren-9-yl)methyl (S)-1-((S)-3-((S)-2-(4-((4-methoxy-6-phenyl-1,3,5-triazin-2-yl)(methyl)amino)pyrimidin-2-ylamino)propyl)benzylamino)-1-oxopropan-2-ylcarbamate.

To a suspension of the Fmoc-alanine (0.12 g, 0.40 mmol) in dichloromethane (2 mL) was added 1-ethyl-3-[3-(dimethylamino)propyl]-carbodiimide hydrochloride (EDCI) (77 mg, 0.40 mmol) followed by the addition of (S)-N²-(1-(3-(aminomethyl)phenyl)propan-2-yl)-N⁴-(4-methoxy-6-phenyl-1,3,5-triazin-2-yl)-N⁴-methylpyrimidine-2,4-diamine (90 mg, 0.20 mmol). The resulting solution was stirred at RT for 15 h. The mixture was diluted in dichloromethane, washed with saturated NH₄Cl, brine, dried over magnesium sulfate, concentrated and chromatographed on silica gel using 0-4% MeOH/CH₂Cl₂ to afford a white solid. MS m/z 750 (MH)⁺.

Step B: (S)-N-((S)-3-((S)-2-(4-((4-Methoxy-6-phenyl-1,3,5-triazin-2-yl)(methyl)-amino)pyrimidin-2-ylamino)propyl)benzyl)-2-aminopropanamide.

A mixture of (9H-fluoren-9-yl)methyl (S)-1-((S)-3-((S)-2-(4-((4-methoxy-6-phenyl-1,3,5-triazin-2-yl)(methyl)amino)pyrimidin-2-ylamino)propyl)benzylamino)-1-oxopropan-2-ylcarbamate (0.16 g, 0.21 mmol) in piperidine (10 mL) was heated to 70° C. for 1 h. The mixture was brought to RT and concentrated. The residue obtained was chromatographed on silica gel using 0-8% 2M NH₃MeOH/CH₂Cl₂ to afford a crystalline white solid. MS m/z 528 (MH)⁺.

### EXAMPLE 126

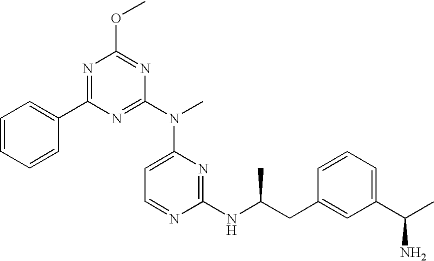 N²-((S)-1-(3-((R)-1-Aminoethyl)phenyl)propan-2-yl)-N⁴-(4-methoxy-6-phenyl-1,3,5-triazin-2-yl)-N⁴-methylpyrimidine-2,4-diamine
Step A: tert-Butyl (R)-1-(3-((S)-2-(4-((4-methoxy-6-phenyl-1,3,5-triazin-2-yl)(methyl)amino)pyrimidin-2-ylamino)propyl)phenyl)ethylcarbamate.

Procedure same as on Example 121, Step E. MS m/z 571 (MH)⁺.

Step B: N²-((S)-1-(3-((R)-1-aminoethyl)phenyl)propan-2-yl)-N⁴-(4-methoxy-6-phenyl-1,3,5-triazin-2-yl)-N⁴-methylpyrimidine-2,4-diamine.

Procedure same as on Example 119, Step F. MS m/z 471 (MH)⁺.

### EXAMPLE 127

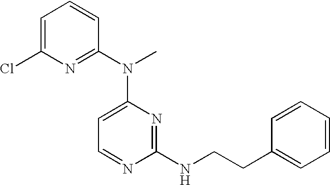 N⁴-(6-Chloropyridin-2-yl)-N⁴-methyl-N²-phenethylpyrimidine-2,4-diamine.
Step A: N-Methyl-2-(methylthio)pyrimidin-4-amine.

4-Chloro-2-methyl sulfanyl pyridine (10 g, 62.5 mmol) and methylamine (2M in methanol, 80 mL) were charged into a sealed tube, the solution was heated to 80° C. for 16 h. The mixture was concentrated under reduced pressure to provide a yellow oil. The oil was poured into 100 mL H₂O, and the heterogeneous solution was filtered out, the title compound was collected as a white solid. MS m/z 156 (MH)⁺.

Step B: N-(6-Chloropyridin-2-yl)-N-methyl-2-(methylthio) pyrimidin-4-amine.

N-methyl-2-(methylthio)pyrimidin-4-amine (4.5 g, 29 mmol), 2,6-dichloropyridine (6.4 g, 43 mmol) and toluene (50 mL) were charged into an oven dried 150 mL round-bottom flask, the solution was degassed by N₂ for 30 min, Pd (OAc)₂ (0.32 g, 1.5 mmol), ras-2,2-bis(diphenylphosphino)-1,1-binaphthyl (0.9 g, 1.5 mmol) and sodium tert-butoxide (5.3 g, 58 mmol) were quickly added, the heterogeneous solution was heated at 100° C. for 16 h. The mixture was concentrated in vacuum; the resulting oil was poured into saturated ammonium chloride and extracted (EtOAc, 2×). The combined organic layers were washed with saturated sodium bicarbonate, followed by brine. The resulting organic layers were collected, dried over Na₂SO₄ and concentrated in vacuum. The crude product was purified by flash chromatography (1:4 EtOAc:Hexane) to give the title compound as an off-white solid. MS m/z 267 (MH)⁺.

Step C: N-(6-Chlropyridin-2-yl)-N-methyl-2-(methylsulfinyl)pyrimidin-4-amine.

3-Chloroperoxybenzoic acid (3.5 g, 15.7 mmol) was added to a cold (0° C. ) solution of N-(6-chloropyridin-2-yl)-N-methyl-2-(methylthio)pyrimidin-4-amine (2.8 g, 10.5 mmol) and DCM (30 mL). The resulting mixture was stirred at same temperature for 1 h. To the crude mixture, DCM and saturated sodium bicarbonate (100 mL) were added. The aqueous layer was extracted with DCM and the combined organic layers were washed by brine. After drying over Na₂SO₄, the crude was concentrated in vacuum to afford the title compound as a yellow solid. MS m/z 282 (MH)⁺.

Step D: N⁴-(6-Chloropyridin-2-yl)-N⁴-methyl-N²-phenethylpyrimidine-2,4-diamine.

Phenylethylamine (2.7 mL, 21 mmol) was added to a stirring solution of N-(6-chloropyridin-2-yl)-N-methyl-2-(methylsulfinyl)pyrimidin-4-amine (3.0 g, 10.5 mmol) in dioxane (50 mL). The mixture was heated to 80° C. for 16 h and then was concentrated in vacuum; the residue oil was poured into 100 mL H₂O. The title compound was collected by filtration as an off-white solid. MS m/z 340 (MH)⁺.

### EXAMPLE 128

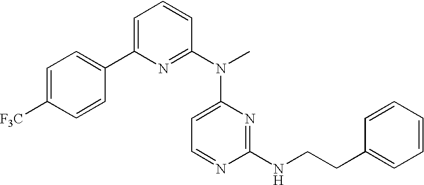 N⁴-Methyl-N²-phenethyl-N⁴-(6-(4-(trifluoromethyl)phenyl)pyridin-2-yl)-pyrimidine-2,4-diamine.

N⁴-(6-Chloropyridin-2-yl)-N⁴-methyl-N²-phenethyl-pyrimidine-2,4-diamine (200 mg, 0.06 mmol), 4-(trifluoromethyl) benzene boronic acid (224 mg, 1.2 mmol), 1,1-bis(diphenylphosphinoferrocene) dichloropalladium (30 mg, 0.04 mmol) and 1:1 DME-2MNa₂CO₃ (10 mL) were charged into a sealed tube. The suspension was heated to 100° C. for 16 h. After the reaction was cooled to RT, DCM and saturated sodium carbonate (20 mL) were added, the aqueous layer was extracted with DCM (×2), and the combined organic layers were washed with brine, dried over Na₂SO₄, and concentrated in vacuum. The crude product was purification by TLC (5% methanol-DCM) to provide the title compound as a pale yellow solid. MS m/z 450 (MH)⁺.

The following compounds were prepared according to the procedure set for Example 128 by using the appropriate boronic acids.

### EXAMPLE 129

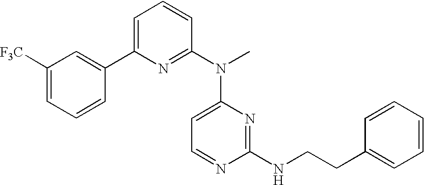

N⁴-Methyl-N²-phenethyl-N⁴-(6-(3-(trifluoromethyl) phenyl)pyridin-2-yl)-pyrimidine-2,4-diamine. MS m/z 450 (MH)⁺.

### EXAMPLE 130

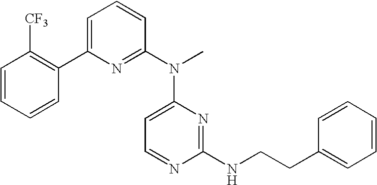

N⁴-Methyl-N²-phenethyl-N⁴-(6-(2-(trifluoromethyl)phenyl)pyridin-2-yl)-pyrimidine-2,4-diamine. MS m/z 450 (MH)⁺.

### EXAMPLE 131

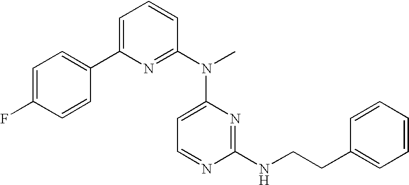

N⁴-(6-(4-Fluorophenyl)pyridin-2-yl)-N⁴-methyl-N²-phenethylpyrimidine-2,4-diamine. MS m/z 400 (MH)⁺.

### EXAMPLE 132

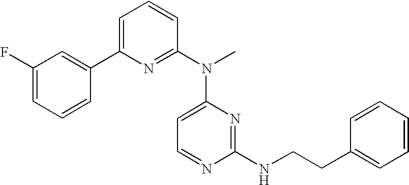

N⁴-(6-(3-Fluorophenyl)pyridin-2-yl)-N⁴-methyl-N²-phenethylpyrimidine-2,4-diamine. MS m/z 400 (MH)⁺.

### EXAMPLE 133

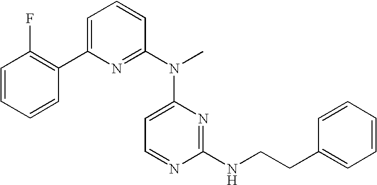

N⁴-(6-(2-Fluorophenyl)pyridin-2-yl)-N⁴-methyl-N²-phenethylpyrimidine-2,4-diamine. MS m/z 400 (MH)⁺.

### EXAMPLE 134

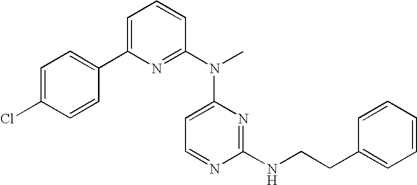

N⁴-(6-(4-Chlorophenyl)pyridin-2-yl)-N⁴-methyl-N²-phenethylpyrimidine-2,4-diamine. MS m/z 416 (MH)⁺.

### EXAMPLE 135

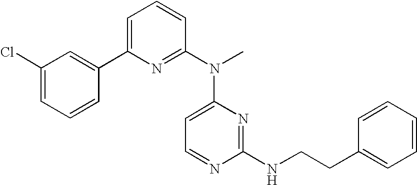

N⁴-(6-(3-chlorophenyl)pyridin-2-yl)-N⁴-methyl-N²-phenethylpyrimidine-2,4-diamine. MS m/z 416 (MH)⁺.

### EXAMPLE 136

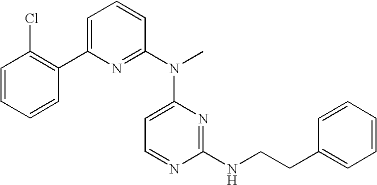

N⁴-(6-(2-Chlorophenyl)pyridin-2-yl)-N⁴-methyl-N²-phenethylpyrimidine-2,4-diamine. MS m/z 416 (MH)⁺.

### EXAMPLE 137

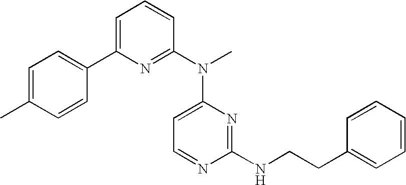

N⁴-Methyl-N²-phenethyl-N⁴-(6-p-tolylpyridin-2-yl)-pyrimidine-2,4-diamine. MS m/z 396 (MH)⁺.

### EXAMPLE 138

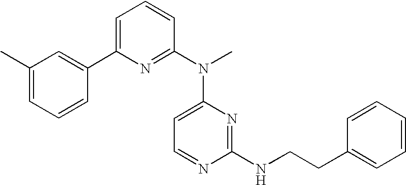

N⁴-Methyl-N²-phenethyl-N⁴-(6-m-tolylpyridin-2-yl)-pyrimidine-2,4-diamine. MS m/z 396 (MH)⁺.

### EXAMPLE 139

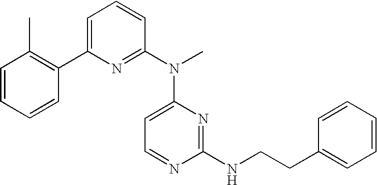

N⁴-Methyl-N²-phenethyl-N⁴-(6-o-tolylpyridin-2-yl)-pyrimidine-2,4-diamine. MS m/z 396 (MH)⁺.

### EXAMPLE 140

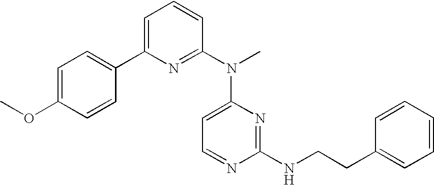

N⁴-(6-(4-Methoxyphenyl)pyridin-2-yl)-N⁴-methyl-N²-phenethylpyrimidine-2,4-diamine. MS m/z 412 (MH)⁺.

### EXAMPLE 141

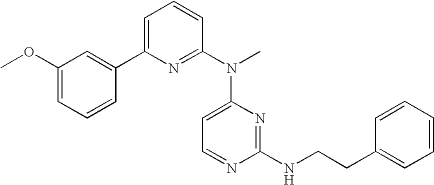

N⁴-(6-(3-Methoxyphenyl)pyridin-2-yl)-N⁴-methyl-N²-phenethylpyrimidine-2,4-diamine. MS m/z 412 (MH)⁺.

### EXAMPLE 142

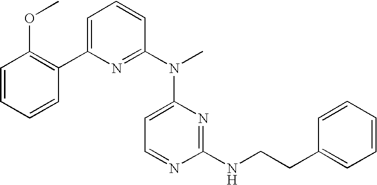

N⁴-(6-(2-Methoxyphenyl)pyridin-2-yl)-N⁴-methyl-N²-phenethylpyrimidine-2,4-diamine. MS m/z 412 (MH)⁺.

### EXAMPLE 143

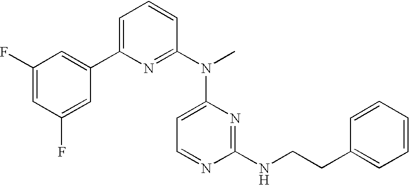

N⁴-(6-(3,5-Difluorophenyl)pyridin-2-yl)-N⁴-methyl-N²-phenethylpyrimidine-2,4-diamine. MS m/z 418 (MH)⁺.

### EXAMPLE 144

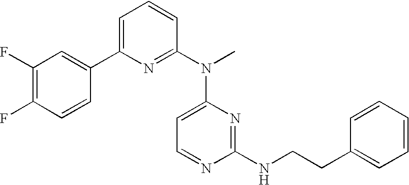

N⁴-(6-(3,4-Difluorophenyl)pyridin-2-yl)-N⁴-methyl-N²-phenethylpyrimidine-2,4-diamine. MS m/z 418 (MH)⁺.

### EXAMPLE 145

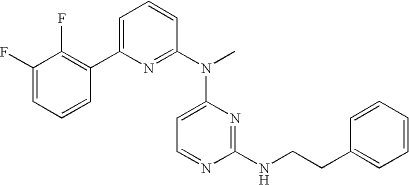

N⁴-(6-(2,3-Difluorophenyl)pyridin-2-yl)-N⁴-methyl-N²-phenethylpyrimidine-2,4-diamine. MS m/z 418 (MH)⁺.

### EXAMPLE 146

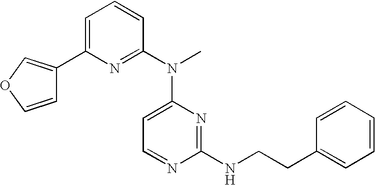

N⁴-(6-(Furan-3-yl)pyridin-2-yl)-N⁴-methyl-N²-phenethylpyrimidine-2,4-diamine. MS m/z 372 (MH)⁺.

### EXAMPLE 147

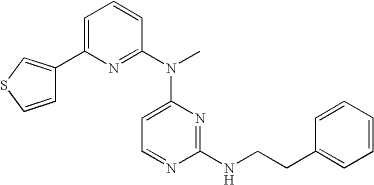

N⁴-<ethyl-N²-phenethyl-N⁴-(6-(thiophen-3-yl)pyridin-2-yl)pyrimidine-2,4-diamine. MS m/z 388 (MH)⁺.

### EXAMPLE 148

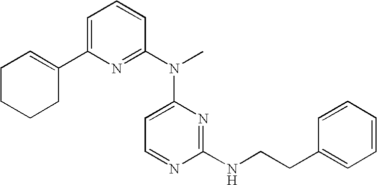

N⁴-(6-cyclohexenylpyridin-2-yl)-N⁴-methyl-N²-phenethylpyrimidine-2,4-diamine. MS m/z 386 (MH)⁺.

### EXAMPLE 149

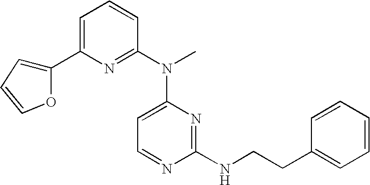 N⁴-(6-(Furan-2-yl)pyridin-2-yl)-N⁴-methyl-N²-phenethylpyrimidine-2,4-diamine.

N⁴-(6-Chloropyridin-2-yl)-N⁴-methyl-N²-phenethylpyrimidine-2,4-diamine (70 mg, 0.2 mmol) and 2 mL toluene was charged in a 5 mL microwave vessel. The mixture was degassed by N₂ for 15 min. 2-(Tribytylstannyl)furan (0.1 mL, 0.3 mmol) and a catalytic amount of tetrakis(triphenylphosphine)palladium(0) were mixed, the reaction was conducted at 130° C. in the microwave for 15 min. To the black suspension, KF (200 mg) was added and stirred for 1 h. The mixture was filtered through a celite pad, washed the celite by DCM, the filtrate was concentrated under vacuum to provide a black oil. The compound was purified by prep TLC (5% methanol) to provide the title compound as yellow oil. MS m/z 372 (MH)⁺.

### EXAMPLE 150

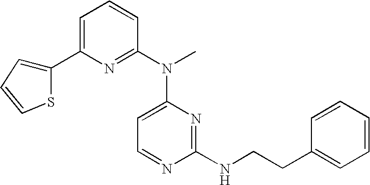 N⁴-Methyl-N²-phenethyl-N⁴-(6-(thiophen-2-yl)pyridin-2-yl)pyrimidine-2,4-diamine

Following the same procedure for preparing N⁴-(6-(furan-2-yl)pyridin-2-yl)-N⁴-methyl-N²-phenethylpyrimidine-2,4-diamine, by in charged with 2-(tributylstannyl)-thiophene (0.1 mL, 0.3 mmol), N⁴-(6-chloropyridin-2-yl)-N⁴-methyl-N²-phenethyl-pyrimidine-2,4-diamine (70 mg, 0.2 mmol), a catalytic amount of tetrakis(triphenyl-phosphine)palladium(0) and 2 mL toluene, the title compound was prepared as yellow oil. MS m/z 388 (MH)⁺.

### EXAMPLE 151

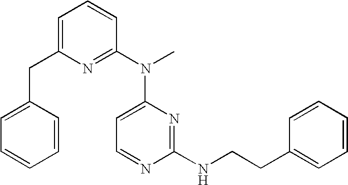 N⁴-(6-Benzylpyridin-2-yl)-N⁴-methyl-N²-phenethylpyrimidine-2,4-diamine.

N-(6-Chloropyridin-2-yl)-N⁴-methyl-N²-phenethylpyrimidine-2,4-diamine (70 mg, 0.2 mmol), benzylzic bromide (0.5M, 1 mL, 0.05 mmol), catalytic amount of Pd(PPh₃)₄ were mixed in dry THF (5 mL), heated in MW at 150° C. for 15 min. The mixture was poured into 20 mL NH₄Cl(sat), extracted by EtOAc (2×). The combined organic layer was washed by NaHCO₃(sat) and brine, dried over MgSO₄ and concentrated under vacuum. The crude product was purified with flash column chromatography (1-3% methanol in DCM) to give the title compound as yellow oil. MS m/z 396 (MH)⁺.

### EXAMPLE 152

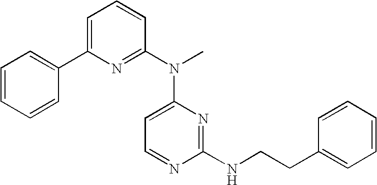 N⁴-Methyl-N²-phenethyl-N⁴-(6-phenylpyridin-2-yl)pyrimidine-2,4-diamine
Step A: 6-Chloro-N-methylpyridin-2-amine.

To a stirring solution of 2,6-dichloro-pyridine (15 g, 0.10 mol) and methylamine (40 wt %, H₂O, 20 mL) in a sealed tube, NaOH (8 g, 0.20 mol) was added, the heterogeneous solution was heated at 120° C. for 16 h, the mixture was cooled down to RT before poured into 200 mL ice-H₂O. After filtration, the title product was collected as an off-white solid. MS m/z 143 (MH)⁺.

Step B: N-Methyl-6-phenylpyridin-2-amine.

6-Chloro-N-methylpyridin-2-mine (11.5 g, 0.081 mol) and phenylboronic acid (16 g, 0.131 mol) were mixed in 160 mL DME, after degassed by N₂ for 10 min, 1,1-bis(diphenylphosphino)ferrocenedichloropalladium(II) (5 g, 6.12 mmol) was mixed, the heterogeneous solution was heated to reflux for 3 h. The mixture was concentrated under vacuum and the resulting oil was poured into saturated ammonium chloride and extracted (EtOAc, 2×). The combined organic layers were washed with saturated sodium bicarbonate, followed by brine. The resulting organic layers collected, dried over Na₂SO₄ and concentrated in vacuum. The crude product was purified with flash column chromatography (4:1 hexane/EtOAc) to give the title compound as an off-white solid. MS m/z 185 (MH)⁺.

Step C: N-Methyl-2-(methylthio)N-(6-phenylpyridin-2-yl)pyrimidin-4-amine.

Following the procedure described in the synthesis of N-(6-chloropyridin-2-yl)-N-methyl-2-(methylthio)pyrimidin-4-amine, by mixing N-methyl-6-phenylpyridin-2-amine (12 g, 65.2 mmol), sodium tert-butoxide (9.0 g, 98 mmol), BINAP (2.0 g, 3.3 mmol), and Pd(OAc)₂ (0.73 g, 3.3 mmol), 4-chloro-2-methylthiopyrimidine (12 mL, 98 mmol) and toluene (150 mL), the resulting heterogeneous solution was stirred at 90° C. overnight and cooled and concentrated, the residue was quenched with ammonium chloride (sat'd aq) and diluted with water and DCM. After filtration, the separated aqueous layer was exacted with DCM. The combined organic layers were washed with brine, dried over Na₂SO₄, and concentrated. The crude product was purified with flash column chromatography (pure hexane→20% EtOAc in hexane) to give the title compound as yellow oil. MS m/z 309 (MH)⁺.

Step D: N-Methyl-2-(methylsulfinyl)-N-(6-phenylpyridin-2-yl)pyrimidin-4-amine.

m-CPBA (4.5 g, ˜70%, 20.3 mmol) was added to a cold (0° C.) solution of N-methyl-2-(methylthio)-N-(6-phenylpyridin-2-yl)pyrimidin-4-amine (5 g, 1.62 mmol) in DCM and the overall mixture was stirred at the same temperature for 1 h prior to being quenched with saturated aqueous sodium bicarbonate. The aqueous layer was extracted with DCM and the combined organic phases were washed by 1N NaOH (aq), following by brine, and then dried over Na₂SO₄. Filtration followed by evaporation provided the title sulfoxide compound, with trace of sulfone, as a yellow solid. MS m/z 325 (MH)⁺.

Step E: N⁴-Methyl-N²-phenethyl-N⁴-(6-phenylpyridin-2-yl)pyrimidine-2,4-diamine.

N-Methyl-2-(methylsulfinyl)-N-(6-phenylpyridin-2-yl)pyrimidin-4-amine (0.2 g, 0.6 mmol) was mixed with phenyl ethylamine (0.2 mL) in dioxane (5 mL). The entire mixture was heated at 80° C. for 14 h and the volatile material was removed by vacuum. The residue was purified with a flashed column chromatography (2%→5% MeOH in DCM) to yield the title compound as an off-whit solid. MS m/z 382 (MH)⁺.

### EXAMPLE 153

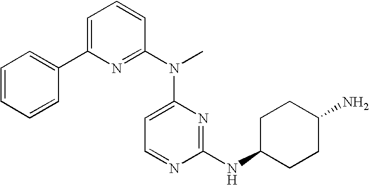 N²-((1s,4s)-4-aminocyclohexyl)-N⁴-methyl-N⁴-(6-phenylpyridin-2-yl)pyrimidine-2,4-diamine.

N-methyl-2-(methylsulfinyl)-N-(6-phenylpyridin-2-yl)pyrimidin-4-amine (0.2 g, 0.6 mmol) was mixed with (1R,4R)-cyclohexane-1,4-diamine (0.17 mL) in dioxane (5 mL). The entire mixture was heated at 110° C. in a sealed tube for 14 h and the volatile material was removed by vacuum. The residue was purified with a flashed column chromatography (2%→5% MeOH in DCM) to yield the title compound as a whit solid. MS m/z 375 (MH)⁺.

### EXAMPLE 154

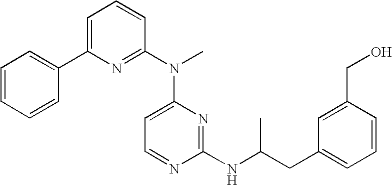 (3-(2-(4-(Methyl (6-phenylpyridin-2-yl)amino)pyrimidin-2-ylamino)propyl)phenyl)-methanol.

N-Methyl-2-(methylsulfinyl)-N-(6-phenylpyridin-2-yl)pyrimidin-4-amine (0.65 g, 2.0 mmol) was mixed with (3-(2-amino-propyl)-phenyl) methanol (0.9 g, 5.4 mmol) in dioxane (10 mL). The entire mixture was heated at 110° C. in a sealed tube for 14 h and the volatile material was removed by vacuum. The residue was purified with a flashed column chromatography (2% MeOH in DCM) to yield the title compound as yellow oil. MS m/z 426 (MH)⁺.

### EXAMPLE 155

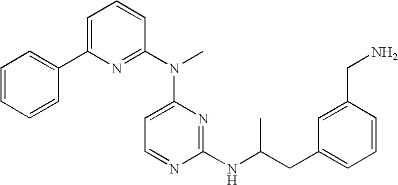 N₂-(1-(3-(aminomethyl)phenyl)propan-2-yl)-N⁴-methyl-N⁴-(6-phenylpyridin-2-yl)pyrimidine-2,4-diamine.

A THF (5 mL) solution of (3-(2-(4-(methyl (6-phenyl-5 pyridin-2-yl)amino)pyrimidine-2-ylamino)propyl)phenyl)methanol (0.5 g, 1.2 mmol) was treated with DBU (0.35 mL, 2.4 mmol) and diphenylphosphoryl azide (0.5 mL, 2.4 mmol) at 0° C. The overall mixture was stirred at RT overnight. After diluted with saturated ammonium chloride aqueous solution, the separated aqueous layer was extracted with ethyl acetate (×2) and the combined organic phases were dried (Na₂SO₄), and concentrated to give a crude azide (MS m/z 451 (MH)⁺) which was immediately treated with 10% Pd/C (cat. amount) in methanol (5 mL) under H₂ (1 atm) at RT overnight. Filtration followed by evaporation provided the crude product, which was subjected to a flash column purification to yield the title compound. MS m/z 425 (MH)⁺.

### EXAMPLE 156

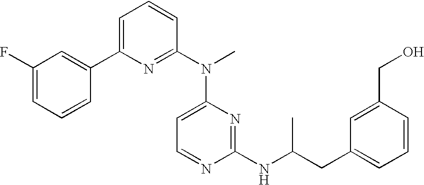 (3-(2-(4-((6-(3-Fluorophenyl)pyridin-2-yl)(methyl)amino)pyrimidin-2-ylamino)propyl)phenyl)methanol
Step A: N-(6-(3-Fluorophenyl)pyridin-2-yl)-N-methyl-2-(methylthio)pyrimidin-4-amine.

Following the procedure for preparing N⁴-methyl-N²-phenethyl-N⁴-(6-(4-(trifluoromethyl)phenyl)pyridin-2-yl)-pyrimidine-2,4-diamine, by using N-(6-chloropyridin-2-yl)-N-methyl-2-(methylthio)pyrimidin-4-amine (0.5 g, 1.9 mmol), 3-fluorophenyl boronic acid (0.5 g, 3.7 mmol), 1,1 bis(diphenylphosphino-ferrocene)dichloropalladium (80 mg, 0.09 mmol) and 1:1 DME-2M Na₂CO₃ (10 mL), the title compound was made as a yellow oil. MS m/z 327 (MH)⁺.

Step B: N-(6-(3-Fluorophenyl)pyridin-2-yl)-N-methyl-2-(methylsulfinyl)pyrimidin-4-amine.

m-CPBA (1.2 g, ˜70%, 5.2 mmol) was added to a cold (0° C.) solution of N-(6-(3-fluorophenyl)pyridin-2-yl)-N-methyl-2-(methylthio)pyrimidin-4-amine (1.1 g, 80%, 3.3 mmol) in DCM and the overall mixture was stirred at the same temperature for 30 min prior to being quenched with saturated aqueous sodium bicarbonate. The aqueous layer was extracted with DCM and the combined organic phases were washed 1N NaOH (aq) and then dried over Na₂SO₄. Filtration followed by evaporation provided the title sulfoxide compound, with trace of sulfone. MS m/z 343 (MH)⁺.

Step C: (3-(2-(4-((6-(3-Fluorophenyl)pyridin-2-yl)(methyl)amino)pyrimidin-2-yl-amino)propyl)phenyl)methanol.

N-(6-(3-Fluorophenyl)pyridin-2-yl)-N-methyl-2-(methylsulfinyl)pyrimidin-4-amine (1.0 g, 2.9 mmol) was mixed with 3-(2-amino-propyl)-phenylmethanol (0.7 g, 3.8 mmol) in NMP (2 mL). The entire mixture was heated at 120° C. in MW for 15 min, and the volatile material was removed by vacuum distillation. The residue was purified with a flashed column chromatography (2%→5% MeOH in DCM) to yield the title compound as yellow oil. MS m/z 444 (MH)⁺.

### EXAMPLE 157

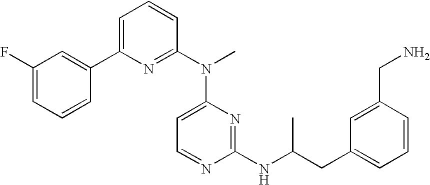 N²-(1-(3-(Aminomethyl)phenyl)propan-2-yl)-N⁴-(6-(3-fluorophenyl)pyridin-2-yl)-N⁴-methylpyrimidine-2,4-diamine.

A THF (5 mL) solution of (3-(2-(4-((6-(3-fluorophenyl)pyridin-2-yl)(methyl)amino)pyrimidin-2-ylamino)propyl)phenyl)-methanol (0.8 g, 1.8 mmol), was treated with DBU (0.6 mL, 4.0 mmol) and diphenylphosphoryl azide (1 mL, 4.6 mmol) at 0° C. The overall mixture was stirred at RT overnight. After diluted with saturated ammonium chloride aqueous solution, the separated aqueous layer was extracted with ethyl acetate (×2) and the combined organic phases were dried (Na₂SO₄), and concentrated to give a crude azide (MS m/z 469 (MH)⁺) which was immediately treated with 10% Pd/C (cat. amount) in methanol (5 mL) under H₂ (1 atm) at RT overnight. Filtration followed by evaporation provided the crude product, which was subjected to a flash column purification to yield the title compound as a white solid. MS m/z 443 (MH)⁺.

### EXAMPLE 158

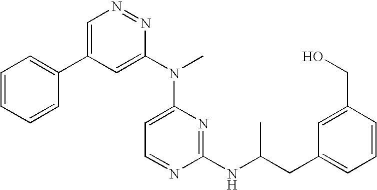 (3-(2-(4-(Methyl(5-phenylpyridazin-3-yl)amino)pyrimidin-2-ylamino)propyl)-phenyl)methanol
Step A: 5-Hydroxy-4-phenylfuran-2(5H)-one.

To a cold (0° C.) solution of glyoxylic acid hydrate (9.2 g, 0.1 mol) and morpholine (8.7 g, 0.1 mol) in dioxane (50 mL), conc. HCl (8.3 mL, 0.1 mol) was added dropwise, followed by slow addition of phenyl acetaldehyde (12.5 mL, 0.1 mol). The heterogeneous solution was heated to reflux for 16 h. After removal the volatile solvent by vacuum, the residue was poured into 500 mL EtOAc and filtrated. The filtrate was washed by NaHCO₃ (sat'd aq), following by brine, dried over MgSO₄. After concentration in vacuum, the title product was collected as a white solid. MS m/z 177 (MH)⁺.

Step B: 5-Phenylpyridazin-3-ol.

To a solution of 5-hydroxy-4-phenylfuran-2(5H)-one (8.6 g, 48.8 mmol) in 60 mL n-BuOH, hydrazine monohydrate (2.8 mL, 58.6 mmol) was mixed, the solution was heated to refluxed for 16 h. After distillation of azeotropic BuOH—H₂O, the residue was concentrated under high vacuum to afford the title compound as a white solid. MS m/z 173 (MH)⁺.

Step C: 3-Chloro-5-phenylpyridazine.

To the suspension of 5-phenylpyridazin-3-ol (7.2 g, 41.86 mmol) in phosphorus oxychloride (72 mL, 0.77 mol), N,N-diisopropylethylamine (7.3 mL) was added slowly, the mixture was heated to reflux for 2 h. After removal of the POCl₃ by distillation, the residue was cooled down prior to being poured into ice, then neutralized by 1N NaOH (aq), the aqueous solution was extracted by EtOAc (3×), the combined organic layers was washed by NaHCO₃ (sat'd, aq) and brine, dried over MgSO₄, concentrated to provide crude product as brown solid, which was then recrystalized from ethanol to afford title compound as a yellow solid. MS m/z 191 (MH)⁺.

Step D: N-Methyl-5-phenylpyridazin-3-amine.

To the solution of 3-chloro-5-phenylpyridazine (8.1 g, 42 mmol) in methylamine (2M in methanol, 60 mL, 120 mmol), N,N-diisopropylethylamine (9.2 mL, 53 mmol) was added in a sealed tube. The resulting mixture was heated to 110° C. for 16 h. After removal the volatile solvent in vacuum, the residue was poured into 200 mL H₂O, after filtration, the title compound was collected as a yellow solid. MS m/z 186 (MH)⁺.

Step E: N-Methyl-N-(2-(methylthio)pyrimidin-4-yl)-5-phenylpyridazin-3-amine.

Following the procedure of preparing N-(6-chloropyridin-2-yl)-N-methyl-2-(methylthio)pyrimidin-4-amine, by using 6.9 g N-methyl-5-phenylpyridazin-3-amine (6.9 g), the title product was prepared as an off-white solid. MS m/z 310 (MH)⁺.

Step F: N-Methyl-N-(2-(methylsulfinyl)pyrimidin-4-yl)-5-phenylpyridazin-3-amine.

m-CPBA (2.3 g, ˜70%, 10.0 mmol) was added to a cold (0° C.) solution of N-methyl-N-(2-(methylthio)pyrimidin-4-yl)-5-phenylpyridazin-3-amine (2.5 g, 8.09 mmol) in DCM and the overall mixture was stirred at the same temperature for 2 h prior to being quenched with saturated aqueous sodium bicarbonate. The aqueous layer was extracted with DCM and the combined organic phases were washed 1N NaOH (aq) and then dried over Na₂SO₄. Filtration followed by evaporation provided the crude sulfoxide, with trace of sulfone. MS m/z 326 (MH)⁺.

Step G: (3-(2-(4-(Methyl(5-phenylpyridazin-3-yl)amino)pyrimidin-2-ylamino)propyl)phenyl)methanol.

N-Methyl-N-(2-(methylsulfinyl)pyrimidin-4-yl)-5-phenylpyridazin-3-amine (0.45 g, 1.4 mmol) mixed with 3-(2-amino-propyl)-phenyl methanol (0.5 g, 3 mmol) in dioxane (5 mL), the solution was heated to reflux for 14 h, After removal of the volatile solvent by vacuum, the residue was purified by flash column chromatography (2% methanol in DCM) to afford the title compound as yellow oil. MS m/z 427 (MH)⁺.

### EXAMPLE 159

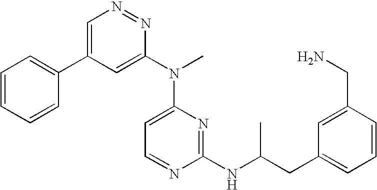 N²-(1-(3-(Aminomethyl)phenyl)propan-2-yl)-N⁴-methyl-N⁴-(5-phenylpyridazin-3-yl)pyrimidine-2,4-diamine.

A THF (5 mL) solution of (3-(2-(4-(methyl(5-phenyl-pyridazin-3-yl)amino)pyrimidin-2-ylamino)propyl)phenyl)methanol, (0.18 g, 0.42 mmol), was treated with DBU (0.1 mL, 0.65 mmol) and diphenylphosphoryl azide (0.2 mL, 0.92 mmol) at 0° C. The overall mixture was stirred at RT overnight. After diluted with saturated ammonium chloride aqueous solution, the separated aqueous layer was extracted with ethyl acetate (×2) and the combined organic phases were dried (Na₂SO₄), and concentrated to give a crude azide (MS m/z 452 (MH)⁺) which was immediately treated with 10% Pd/C (cat. amount) in methanol (5 mL) under H₂ (1 atm) at RT overnight. Filtration followed by evaporation provided the crude product, which was subjected to a flash column purification to yield the title compound. MS m/z 426 (MH)⁺.

### EXAMPLE 160

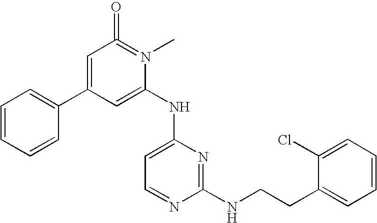 6-(2-(2-Chlorophenethylamino)pyrimidin-4-ylamino)-1-methyl-4-phenylpyridin-2(1H)-one
Step A: 6-Amino-1-methyl-4-phenyl-1H-pyridin-2-one.

Crude 4-cyano-3-phenyl-but-3-enoic acid ethyl ester (12.32 g, 0.057 mol) was added to a stirred solution of Na/MeOH (30% solution, 16 mL) followed by slowed addition of MeNH₂ (18 mL, 2.0M in MeOH). The resulting solution was stirred at RT overnight prior to being poured into ice. The separated aqueous layer was extracted with DCM. The combined organic phases were washed with brine, dried over Na₂SO₄ and concentrated under reduced pressure to afford the crude material, which was wased with ethyl acetate and the precipitate was collected to provide the title compound as a brownish yellow solid. MS m/z 201 (M+H)⁺.

Step B: 1-Methyl-6-(2-methylsulfanyl-pyrimidin-4-ylamino)-4-phenyl-1H-pyridin-2-one.

Following the procedure described in the synthesis of 1-(2-methylsulfanyl-pyrimidin-4-yl)-8-phenyl-1,2,3,4-tetrahydro-pyrido[1,2-a]pyrimidin-6-one, but using 6-amino-1-methyl-4-phenyl-1H-pyridin-2-one (1.36 g, 6.8 mmol), sodium tert-butoxide (1.31 g, 13.16 mmol), BINAP (0.22 g, 0.34 mmoL), and Pd(OAc)₂ (76 mg, 0.34 mmol) was added toluene (30 mL) and 4-chloro-2-methylthio-pyrimidine (1.3 mL, 10.9 mmol). After stirred at 90° C. overnight and cooled, was quenched with ammonium chloride (sat'd aq) and diluted with water and DCM. After filtrated, the separated aqueous layer was exacted with DCM. The combined organic layers were washed with brine, dried over Na₂SO₄, and concentrated. Purification of the crude product with flash column chromatography (pure DCM→5% MeOH in DCM) gave the title compound as a pale yellow solid. MS m/z 325 (M+H)⁺.

Step C: 1-Methyl-6-[methyl-(2-methylsulfanyl-pyrimidin-4-yl)-amino]-4-phenyl-1H-pyridin-2-one.

To a stirred mixture of 1-methyl-6-(2-methylsulfanyl-pyrimidin-4-ylamino)-4-phenyl-1H-pyridin-2-one (0.74 g) and K₂CO₃ (1.89 g, 6.85 mmol) in DMF (10 mL) was added MeI (0.43 mL) at 0° C. After stirred at the same temperature for 10 min and RT for 1.5 h, the reaction mixture was diluted with water and DCM and the separated aqueous layer was extracted with DCM. The combined organic layers were washed with water, dried over Na₂SO₄ and concentrated under reduced pressure to afford the crude product which was purified with flash column chromatography (2% MeOH in DCM) to provide the title compound (0.73 g) as a pale yellow solid. MS m/e 339 (M+H)⁺.

Step D: 6-(2-(2-Chlorophenethylamino)pyrimidin-4-ylamino)-1-methyl-4-phenyl-pyridin-2(1H)-one.

m-CPBA (0.23 g, ˜70%, 1.26 mmol) was added to a cold (0° C.) solution of 1-methyl-6-(2-methylsulfanyl-pyrimidin-4-ylamino)-4-phenyl-1H-pyridin-2-one (0.3 g, 0.86 mmol) in DCM and the overall mixture was stirred at the same temperature for 30 min prior to being quenched with saturated aqueous sodium bicarbonate. The aqueous layer was extracted with DCM and the combined organic phases were washed 1N NaOH(aq) and then dried over Na₂SO₄. Filtration followed by evaporation provided the crude sulfoxide, with trace of sulfone. The crude mixture (0.16 g) was mixed with 2-(2-chlorophenyl)ethanamine (0.15 mL) in NMP (1 mL). The entire mixture was heated at 100° C. for 4 h and the volatile material was removed by vacuum distillation. The residue was purified with a flashed column chromatography (2%→5% MeOH in DCM) to yield the title compound as a pale yellow solid. MS m/z 432 (M+H)⁺.

### EXAMPLE 161

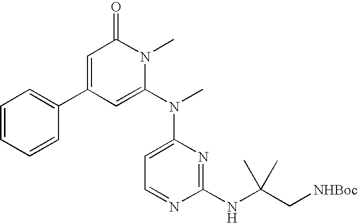 tert-Butyl 2-methyl-2-(4-(methyl(1-methyl-6-oxo-4-phenyl-1,6-dihydropyridin-2-yl)amino)pyrimidin-2-ylamino)propylcarbamate.

Oxidation of the 1-methyl-6-[methyl-(2-methylsulfanyl-pyrimidin-4-yl)-amino]-4-phenyl-1H-pyridin-2-one (0.11 g, 0.3 mmol) and subsequent displacement with tert-butyl 2-amino-2-methylpropylcarbamate (0.16 g, 0.847 mmol) were conducted with the similar fashion as described previously in 160, step D to afford, after chromatographic purification (5% MeOH in DCM) to provide the title compound as a pale yellow solid. MS m/z 479 (M+H)⁺.

### EXAMPLE 162

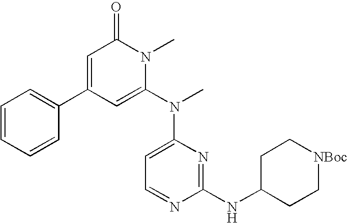 tert-Butyl 4-(4-(methyl(1-2-yl)amino)pyrimidin-2-ylamino)piperidine-1-carboxylate.

Oxidation of the 1-methyl-6-[methyl-(2-methylsulfanyl-pyrimidin-4-yl)-amino]-4-phenyl-1H-pyridin-2-one (0.39 g, 1.2 mmol) and subsequent displacement with tert-butyl 4-aminopiperidine-1-carboxylate (0.385 g, 1.93 mmol) were conducted with the similar fashion as described previously in 160, step D to afford, after chromatographic purification (pure DCM→3% MeOH in DCM) to provide the title compound as a off white solid. MS m/z 491 (M+H)⁺.

### EXAMPLE 163

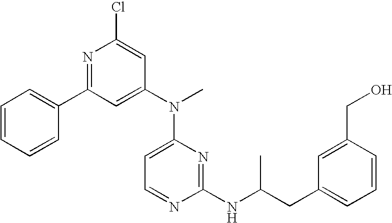 (3-(2-(4-((2-Chloro-6-phenylpyridin-4-yl)(methyl)amino)pyrimidin-2-ylamino)-propyl)phenyl)methanol
Step A: N-(2,6-Dichloropyridin-4-yl)-N-methyl-2-(methylthio)pyrimidin-4-amine

2,6-Dichloropyridin-4-amine (3.26 g, 0.02 mol) was mixed with rac-BINAP (0.62 g, 1.0 mmol)), Pd(OAc)₂ (0.22 g, 1.0 mmol) and sodium tert-butoxide (2.7 g, 0.028 mol) in a reaction vial. After purged with N₂ for 10 min, toluene (30 mL) was added followed by 4-chloro-2-thiomethylpyrimidine (2.8 mL, 0.024 mol). The mixture was sealed and heated at 80° C. for 16 h. After cooled, the reaction was quenched with ammonium chloride (sat'd aq) and diluted with water and DCM. After filtrated, the separated aqueous layer was exacted with DCM. The combined organic layers were washed with brine, dried over Na₂SO₄, and concentrated. Removal of volatile material provided the crude product part of which (0.496 g, 1.73 mmol) was suspended in THF (5 mL) and treated with KOtBu (2.1 mL, 1.0M in THF) at 0° C. The resulting yellow solution was stirred at the same temperature for additional 30 min prior to the introduction of MeI (0.16 mL, 2.6 mmol). After stirred for more 1 h at 0° C., the mixture was quenched with ammonium chloride (sat'd aq) and diluted with water and extracted with EtOAc. The combined organic layers were washed with brine, dried (Na₂SO₄), and concentrated to give a crude product, which was washed with ether to provide the title compound as a white. MS m/z 301 (M+H)⁺.

Step B: N-(2-Chloro-6-phenylpyridin-4-yl)-N-methyl-2-(methylthio)pyrimidin-4-amine.

To a mixture of N-(2,6-dichloropyridin-4-yl)-N-methyl-2-(methylthio)-pyrimidin-4-amine (1.14 g, 3.8 mmol), phenylboronic acid (0.51 g, 4.18 mmol), Cs₂CO₃ (1.86 g, 5.7 mmol) was added Pd(PPh₃)₂Cl₂ (0.13 g, 0.19 mmol) and then THF (7 mL). The suspension was heated at 65° C. for 16 h and then cooled to RT. The reaction was diluted with saturated aqueous ammonium chloride solution and extracted with ethyl acetate. The overall organic phases were washed with water, brine, and then dried (Na₂SO₄). Filtration and concentration provided the crude product which was purified with flash column chromatography (pure hexanes→1:5 EA/hexanes) to afford the title compound as a pale yellow foam along with recovered starting material. MS m/z 343 (M+H)⁺.

Step C: (3-(2-(4-((2-Chloro-6-phenylpyridin-4-yl)(methyl)amino)pyrimidin-2-ylamino)propyl)phenyl)methanol.

Oxidation of the N-(2-chloro-6-phenylpyridin-4-yl)-N-methyl-2-(methylthio)pyrimidin-4-amine (0.2524 g, 7.37 mmol) and subsequent displacement with (3-(2-aminopropyl)phenyl)methanol (0.263 g, 1.47 mmol) were conducted with the similar fashion as described previously in Example 160, Step E to afford, after chromatographic purification (pure DCM→2% MeOH in DCM) to provide the title compound as a pale yellow solid. MS m/z 460 (M+H)⁺.

### EXAMPLE 164

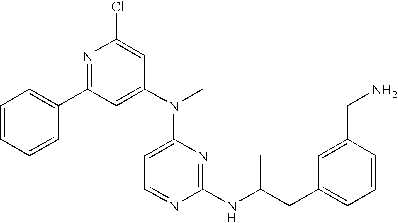 N²-(1-(3-(Aminomethyl)phenyl)propan-2-yl)-N⁴-(2-chloro-6-phenylpyridin-4-yl)-N4-methylpyrimidine-2,4-diamine.

A THF (5 mL) solution of the crude benzylic alcohol (0.28 g, 0.61 mmol) was treated with DBU (0.2 mL, 1.22 mmoL) and diphenylphosphoryl azide (0.2 mL, 0.91 mmol) at 0° C. and the overall mixture was stirred at RT overnight. After diluted with saturated ammonium chloride aqueous solution, the separated aqueous layer was extracted with ethyl acetate (×2) and the combined organic phases were dried (Na₂SO₄), filtrated, and concentrated to give a crude azide which was immediately treated with 10% Pd/C (0.2 g) in dioxane (5 mL) under H₂ (1 atm) at RT overnight. Filtration followed by evaporation provided the crude product, which was subjected to a flash column purification to yield the title compound. MS m/z 459 (MH)⁺.

### EXAMPLE 165

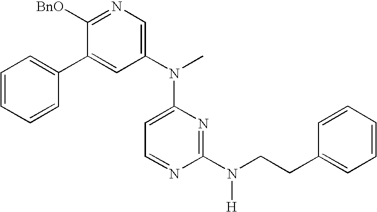 N⁴-(6-(Benzyloxy)-5-phenylpyridin-3-yl)-N⁴-methyl-N²-phenethylpyrimidine-2,4-diamine
Step A: 6-(Benzyloxy)-5-phenylpyridin-3-amine.

To a mixture of 2-(benzyloxy)-3-chloro-5-nitropyridine (0.77 g, 2.91 mmol), phenylboronic acid (0.53 g, 4.37 mmol), Cs₂CO₃ (1.90 g, 5.82 mmol) was added Pd(PPh₃)₂Cl₂ (0.10 g, 0.15 mmol) and then THF (10 mL). The suspension was heated at 80° C. for 4 h and then cooled to RT. The reaction mixture was filtrated through Celite, washed with EtOAc, and concentrated to provide the crude product which was purified with flash column chromatography (pure hexanes→1:20 EA/hexanes) to afford the title compound (0.57 g) as a white crystalline. MS m/z 307.1 (M+H)⁺. This nitropyridine compound (1.06 g, 3.58 mmol) was suspended in a THF-H₂O (10 mL each) mixture and was then treated with AcOH (2 mL) and iron (1 g) at 0° C. The reaction was allowed to stirred at RT for 2 h and then filtrated with Celite and the filtrated cake was washed with EtOAc several times. The overall organic phases were washed with NaHCO₃(aq), brine, and concentrated to yield the title compound as a pale yellow sold. MS m/z 277 (M+H)⁺.

Step B: N-(6-(Benzyloxy)-5-phenylpyridin-3-yl)-N-methyl-2-(methylthio)pyrimidin-4-amine.

To a stirred solution of 6-(benzyloxy)-5-phenylpyridin-3-amine (0.14 g, 0.51 mmol) and 4-chloro-2-thiomethylpyrimidine (89 μL, 1.5 eq) in THF (2 mL) was added LHMDS (1.0M in THF, 1.5 mL, 3 eq) at 0° C. and the resulting mixture was stirred at the same temperature for 10 min. MeI (47 μL, 1.5 eq) was then introduced and the entire solution was stirred for an additional 15 min at 0° C. The reaction mixture was quenched with ammonium chloride (sat'd aq) and diluted with water and EtOAc. The separated aqueous layer was exacted with EtOAc and the combined organic layers were washed with brine, dried over Na₂SO₄, and concentrated. Removal of volatile material provided the crude product, which was purified with flash column chromatography (pure hexanes→1:5 EtOAc/hexanes) to provide the title compound (0.2 g) as a light brown foam. MS m/z 415 (M+H)⁺.

Step C: N⁴-(6-(Benzyloxy)-5-phenylpyridin-3-yl)-N⁴-methyl-N²-phenethyl-pyrimidine-2,4-diamine.

The title compound (pale yellow solid) was obtained, after a flash column chromatographic purification (0.5% MeOH in DCM), with the similar manner as described in Example 160, Step E from N-(6-(benzyloxy)-5-phenylpyridin-3-yl)-N-methyl-2-(methylthio)pyrimidin-4-amine (0.106 g, 0.254 mmol) and phenethanylamine (3 eq). MS m/z 488 (M+H)⁺.

### EXAMPLE 166

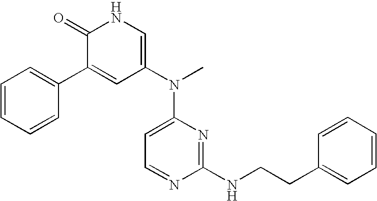 5-(Methyl(2-(phenethylamino)pyrimidin-4-yl)amino)-3-phenylpyridin-2(1H)-one.

A mixture of N⁴-(6-(benzyloxy)-5-phenylpyridin-3-yl)-N⁴-methyl-N²-phenethyl-pyrimidine-2,4-diamine (57 mg, 0.12 mmol) in EtOH-toluene (2 mL each) was added 6N HCl (2 mL) and the overall mixture was heated at 70° C. for 2 h. After cooled and concentrated under reduced pressure, the crude material was diluted with water and ether. The separated aqueous layer was basified with 5N NaOH and extracted with DCM, and the extracts were dried (Na₂SO₄), and evaporated. The precipitate was collected by washing the residue with EtOAc and dried under vacuum. The title compound was obtained as a pale yellow solid. MS m/z 398 (M+H)⁺.

### EXAMPLE 167

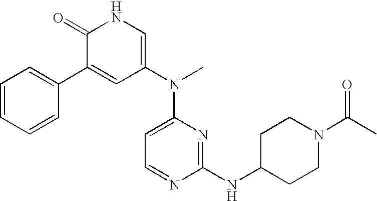 5-((2-(1-Acetylpiperidin-4-ylamino)pyrimidin-4-yl)(methyl)amino)-3-phenyl-pyridin-2(1H)-one.
Step A:

tert-Butyl 4-(4-((6-(benzyloxy)-5-phenylpyridin-3-yl)(methyl)amino)-pyrimidin-2-ylamino)piperidine-1-carboxylate was obtained similarly as described previously described on Example 160, Step E from N-(6-(benzyloxy)-5-phenyl-pyridin-3-yl)-N-methyl-2-(methylthio)pyrimidin-4-amine (1.0 g, 2.32 mmol) and 4-amino-1-N-Boc-piperidine (0.56 g, 2.79 mmol) and DIEA (0.61 mL, 3.49 mmol), after purified by a flash column chromatography (pure DCM→3% MeOH in DCM), as a pale yellow solid. MS m/z 567 (M+H)⁺.

Step B: N⁴-(6-(Benzyloxy)-5-phenylpyridin-3-yl)-N⁴-methyl-N²-(piperidin-4-yl)-pyrimidine-2,4-diamine.

A mixture of tert-Butyl 4-(4-((6-(benzyloxy)-5-phenyl-pyridin-3-yl)(methyl)amino)pyrimidin-2-ylamino)piperidine-1-carboxylate (0.23 g, 0.41 mmol) in dioxane (1 mL) was added 4N HCl (in dioxane, 1 mL) and stirred at RT for 30 min. The resulting mixture was concentrated and redissolved in DCM. After washed with saturated aqueous NaHCO₃, and brine, the solvent was evaporated and purified with 4% MeOH in DCM to give the title compound as a pale yellow solid. MS m/z 467.3 (M+H)⁺.

Step C: 1-(4-(4-((6-(Benzyloxy)-5-phenylpyridin-3-yl)(methyl)amino)pyrimidin-2-ylamino)piperidin-1-yl)ethanone.

A slurry of N⁴-(6-(benzyloxy)-5-phenylpyridin-3-yl)-N⁴-methyl-N²-(piperidin-4-yl)pyrimidine-2,4-diamine (0.23 g, 0.49 mmol), AcOH (39 mM, 0.64 mmol), PS-carbodiimide (0.76 g, 0.98 mmol) in DCM (15 mL) was stirred at RT overnight. The resulting mixture was filtrated and the filtrated cake was washed with DCM, and the overall solution was evaporated to give the crude title compound. MS m/z 509.3 (M+H)⁺.

Step D: 5-((2-(1-Acetylpiperidin-4-ylamino)pyrimidin-4-yl)(methyl)amino)-3-phenylpyridin-2(1H)-one.

The crude product obtained from the previous step was treated with neat TFA (2 mL) at RT for 30 min prior to being concentrated and rediluted with water. The mixture was extracted with EtOAc, the separated aqueous layer was basified with 5N NaOH and extracted with DCM. The extracts were washed with brine and concentrated, and title compound was obtained as a pale yellow solid after a flash column chromatography (5→10% MeOH in DCM). MS m/z 419.2 (M+H)⁺.

### EXAMPLE 168

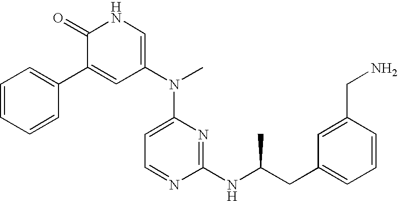 (S)-5-((2-(1-(3-(Aminomethyl)phenyl)propan-2-ylamino)pyrimidin-4-yl)(methyl)-amino)-3-phenylpyridin-2(1H)-one.

The title compound (off-white solid) was obtained, after a flash column chromatographic purification (7% MeOH in DCM), with the similar method as described previously from N-(6-(benzyloxy)-5-phenyl-pyridin-3-yl)-N-methyl-2-(methylthio)pyrimidin-4-amine (0.21 g, 0.5 mmol) and (S)-tert-butyl 3-(2-aminopropyl)benzylcarbamate (1.5 eq). MS m/z 441.2 (M+H)⁺.

### EXAMPLE 169

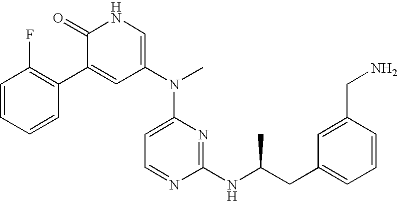 (S)-5-((2-(2(3-(Aminomethyl)phenyl)propan-2-ylamino)pyrimidin-4-yl)(methyl)-amino)-3-(2-fluorophenyl)pyridin-2(1H)-one
Step A: tert-Butyl (16S)-3-((S)-2-(4-((6-(benzyloxy)-5-(2-fluorophenyl)pyridin-3-yl)(methyl)amino)pyrimidin-2-ylamino)propyl)benzylcarbamate.

To a mixture of (S)-tert-butyl 3-(2-(4-((6-(benzyloxy)-5-chloropyridin-3-yl)(methyl)amino)-pyrimidin-2-ylamino)propyl)benzylcarbamate (0.43 g, 73 mmol) in toluene/ethanol (4/1) 2-fluorophenylboronic acid (0.23 g, 1.4 mmol), K₂CO₃ (2.0M in water) Pd(PPh₃)₄ (0.036 g, 0.031 mmol) was added. The vial was sealed and heated under microwave at 140° C. for 10 min. The mixture was partitioned between CH₂Cl₂ and 1N NaOH. The layers were separated and the aqueous layer was extracted with CH₂Cl₂. The combined organic layers were dried (Na₂SO₄) concentrated and purified with ISCO (pure DCM/MeOH 95/5) to afford the title compound as a white crystalline. MS m/z 649 (M+H)⁺.

Step B: (S)-5-((2-(1-(3-(Aminomethyl)phenyl)propan-2-ylamino)pyrimidin-4-yl)(methyl)amino)-3-(2-fluorophenyl)pyridin-2(1H)-one.

Deprotection of Bn- and Boc-groups of (S)-5-((2-(1-(3-(aminomethyl)phenyl)propan-2-ylamino)pyrimidin-4-yl)(methyl)amino)-3-(2-fluorophenyl)pyridin-2(1H)-one (100 mg, 0.17 mmol) was conducted with 4N HCl in dioxane (RT, 1 h) and the crude product was purified with ISCO followed by HPLC (pure DCM/MeOH 90/10) to afford the title compound as a white crystalline. MS m/z 459 (M+H)⁺.

### EXAMPLE 170

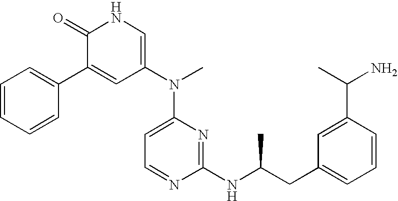 5-((2-((S)-1-(3-((S)-1-Aminoethyl)phenyl)propan-2-ylamino)pyrimidin-4-yl)-(methyl)amino)-3-phenylpyridin-2(1H)-one.

The title compound (off-white solid) was obtained, after a flash column chromatographic purification (7% MeOH in DCM), with the similar method as described previously from N-(6-(benzyloxy)-5-phenylpyridin-3-yl)-N-methyl-2-(methylthio)pyrimidin-4-amine (0.21 g, 0.5 mmol) and tert-butyl (S)-1-(3-((S)-2-aminopropyl)phenyl)ethylcarbamate (0.21 g, 0.75 mmol) followed by deprotection. MS m/z 455 (M+H)⁺.

5-((2-((S)-1-(3-((R)-1-Aminoethyl)phenyl)propan-2-ylamino)pyrimidin-4-yl)-(methyl)amino)-3-phenylpyridin-2(1H)-one. Similarly, the title compound was isolated as a yellow solid from N-(6-(benzyloxy)-5-phenylpyridin-3-yl)-N-methyl-2-(methylthio)pyrimidin-4-amine (0.45 g, 1.1 mmol)and tert-butyl (S)-1-(3-((S)-2-aminopropyl)phenyl)ethylcarbamate (0.41 g, 1.5 mmol) followed by deprotection. MS m/z 455 (M+H)⁺.

### EXAMPLE 171

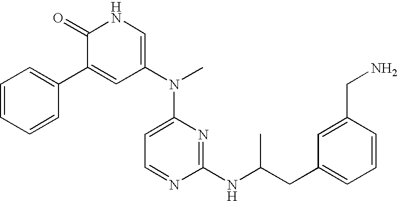 5-((2-(1-(3-(Aminomethyl)phenyl)propan-2-ylamino)pyrimidin-4-yl)(methyl)-amino)-3-phenylpyridin-2(1H)-one
Step A: (3-(2-(4-((6-(Benzyloxy)-5-phenylpyridin-3-yl)(methyl)amino)pyrimidin-2-ylamino)propyl)phenyl)methanol.

The title compound was obtained with the similar manner as described previously in example 160, Step E with (3-(2-aminopropyl)-phenyl)methanol (1.5 eq) and N-(6-(benzyloxy)-5-phenylpyridin-3-yl)-N-methyl-2-(methylthio)pyrimidin-4-amine to give an off-white solid. MS m/z 532 (M+H)⁺.

Step B: 5-((2-(1-(3-(Aminomethyl)phenyl)propan-2-ylamino)pyrimidin-4-yl)(methyl)amino)-3-phenylpyridin-2(1H)-one.

A THF (3 mL) solution of benzylic alcohol (0.14 g, 0.26 mmol) obtained above was treated with DBU (80 μL, 0.53 mmoL) and diphenylphosphoryl azide (85 μL, 0.4 mmol) at 0° C. and the overall mixture was stirred at RT overnight. After diluted with saturated ammonium chloride aqueous solution, the separated aqueous layer was extracted with ethyl acetate (×2) and the combined organic phases were dried (Na₂SO₄), filtrated, and concentrated to give a crude azide which was immediately treated with 10% Pd/C (0.15 g) in EtOAc (5 mL) under H₂ (1 atm) at RT overnight. Filtration followed by evaporation provided the crude crude benzylic amine product, which was subjected to a de-benzyl conditions similar as described in Example 167. After flash column purification (pure DCM→3% MeOH in DCM), the title compound was yielded as an off-white solid. MS m/z 441 (M+H)⁺.

### EXAMPLE 172

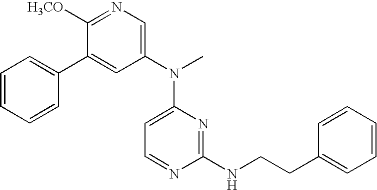 N⁴-(6-Methoxy-5-phenylpyridin-3-yl)-N⁴-methyl-N²-phenethylpyrimidine-2,4-diamine
Step A: 6-Methoxy-5-phenylpyridin-3-amine.

The title compound was obtained as a pale brown solid by following the similar method described in Example 165 step A, but using 3-bromo-2-methoxy-5-nitropyridine (0.88 g, 3.8 mmol) as a starting material. MS m/z 201 (M+H)⁺.

Step B: N-(6-Methoxy-5-phenylpyridin-3-yl)-N-methyl-2-(methylthio)pyrimidin-4-amine.

6-Methoxy-5-phenylpyridin-3-amine (1.7932 g, 8.97 mmol) was mixed with rac-BINAP (0.28 g, 0.45 mmol)), Pd(OAc)₂ (0.1 g, 0.45 mmol) and sodium tert-butoxide (1.04 g, 10.76 mmol) in a reaction vial. After purged with N₂ for 10 min, toluene (10 mL) was added followed by 4-chloro-2-thiomethylpyrimidine (1.1 mL, 8.97 mmol). The mixture was sealed and heated at 90° C. for 24 h. After cooled, the reaction was quenched with ammonium chloride (sat'd aq) and diluted with water and DCM. After filtrated, the separated aqueous layer was exacted with DCM. The combined organic layers were washed with brine, dried over Na₂SO₄, and concentrated. Purification of the crude material with a flash column chromatography (1:3→1:2 EA/hexanes) afforded N-(6-methoxy-5-phenylpyridin-3-yl)-2-(methylthio)pyrimidin-4-amine (1.82 g) as an off-white crystalline, part of which (1.613 g, 4.97 mmol) was suspended in THF (10 mL) and treated with KO^{t}Bu (7.5 mL, 1.0M in THF) at 0 ° C. The resulting yellow solution was stirred at the same temperature for additional 30 min prior to the introduction of MeI (0.62 mL, 9.94 mmol). After stirred for more 1 h at 0 ° C., the mixture was quenched with ammonium chloride (sat'd aq) and diluted with water and extracted with EtOAc. The combined organic layers were washed with brine, dried (Na₂SO₄), and concentrated to give a crude product as a pale brown foam, which was used without further purification. MS m/z 339 (M+H)⁺.

Step C: N⁴-(6-Methoxy-5-phenylpyridin-3-yl)-N⁴-methyl-N²-phenethylpyrimidine-2,4-diamine.

The title compound (pale yellow solid) was obtained, after a flash column chromatographic purification (pure DCM→3% MeOH in DCM), with the similar manner as described previously in Example 160 step E from N-(6-methoxy-5-phenylpyridin-3-yl)-N-methyl-2-(methylthio)pyrimidin-4-amine (0.36 g, 1.07 mmol) and phenethanylamine (3 eq). MS m/z 412 (M+H)⁺.

### EXAMPLE 173

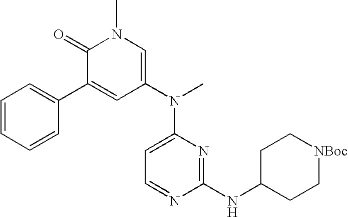 Step A: 3-Iodo-1-methyl-5-nitropyridin-2(1H)-one.

To a suspension of sodium hydride (2 eq, 150.35 mmol) in DMF (150 mL) was added 3-iodo-5-nitropyridone-2-(1H)-one (20.0 g, 75.19 mmol) portion wise. When effervescence subsided iodomethane (1.5 eq, 112.78 mmol) was added. The mixture was stirred at RT for 1 h, quenched with water slowly, added ethyl acetate, wash the ethyl acetate layer with water. The separated organic layer was washed with saturated sodium chloride solution and dry over sodium sulfate. After crystallization dark yellow solid was collected. MS m/z 281 (M+H)⁺.

Step B: 5-Amino-3-iodo-1-methyl-5-nitropyridin-2(1H)-one.

To a mixture of 3-iodo-1-methyl-5-nitropyridin-2(1H)-one (15 g, 53.59 mmol), THF (150 mL), water (150 mL) Acetic acid (30 mL) was added. To the resulting solution iron (5 eq. 267.95 mmol) was added. The suspension was stirred at RT for 3 h, dilute with water, then filtrated with Celite wash with ethyl acetate, concentrate, extract the residue with ethyl acetate, DCM, concentrate to afford the title compound as a dark solid. MS m/z 251.0 (M+H)⁺.

Step C: 3-Iodo-1-methyl-5-(methyl(3-(methylthio)phenyl)amino)pyridin-2(1H)-one.

The title compound was obtained in the similar manner as described previously in Example 165 step B. Thus, 8.7 g of 5-amino-3-Iodo-1-methyl-5-nitropyridin-2(1H)-one (34.8 mmol) was converted to the title compound (precipitate collected from EtOAc) as a brown solid. MS m/z 367 (M+H)⁺.

Step D: 1-Methyl-5-(methyl(2-(methylthio)pyrimidin-4-yl)amino)-3-phenylpyridin-2(1H)-one.

The title compound also has been prepared individually from 3-Iodo-1-methyl-5-(methyl(3-(methylthio)phenyl)amino)pyridin-2(1H)-one. Thus, 3-iodo-1-methyl-5-(methyl(3-(methylthio)phenyl)amino)pyridin-2(1H)-one (4.0 g, 10.3 mmol), phenylboronic acid (1.8 g, 14.4 mmol) and Pd(PPh₃)₄ (0.48 g, 0.4 mmol) were charged in a microwave reaction vessel, purged with N₂ for 10 min, then toluene/EtOH (4:1, 5 mL) and K₂CO₃ (2M aq, 7 mL) was added, and the entire mixture was heated under microwave irradiation at 150° C. for 10 min. After cooled, the solvent was removed, and the residue was partitioned between DCM and sat'd aqueous NaHCO₃. The separated aqueous layer was extracted with DCM and the combined organic layers were dried (Na₂SO₄) and concentrated to give the title compound as a dark brown solid, which was used without further purification. By following the similar sequences as described previously in Example 160 step E (oxidation with mCPBA; displacement with various amines in NMP), 1-methyl-5-(methyl(2-(methylthio)pyrimidin-4-yl)amino)-3-phenylpyridin-2(1H)-one was converted to the corresponding 2-alkylamino-analogues listed below.

Step D: tert-Butyl 4-(4-(Methyl(1-methyl-6-oxo-5-phenyl-1,6-dihydropyridin-3-yl)amino)pyrimidin-2-ylamino)piperidine-1-carboxylate.

Light yellow solid. MS m/z 491.3 (M+H)⁺.

The compound from example 173 was converted to the following two compounds with the methods similar to those of Example 167.

### EXAMPLE 174

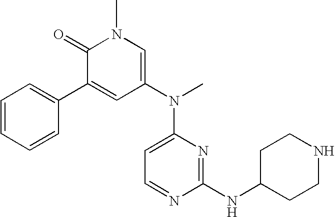 1-Methyl-5-(methyl(2-(piperidin-4-ylamino)pyrimidin-4-yl)amino)-3-phenylpyridin-2(1H)-one.

Off-white solid. MS m/z 391 (M+H)⁺.

### EXAMPLE 175

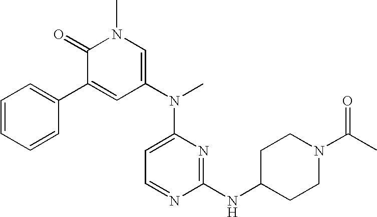 5-((2-(1-Acetylpiperidin-4-ylamino)pyrimidin-4-yl)(methyl)amino)-1-methyl-3-phenylpyridin-2(1H)-one.

Light yellow solid. MS m/z 433 (M+H)⁺.

### EXAMPLE 176

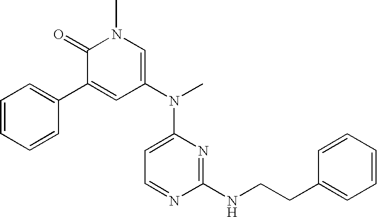 1-Methyl-5-(methyl(2-(phenethylamino)pyrimidin-4-yl)amino)-3-phenylpyridin-2(1H)-one.

Tan solid. MS m/z 412.2 (M+H)⁺.

### EXAMPLE 177

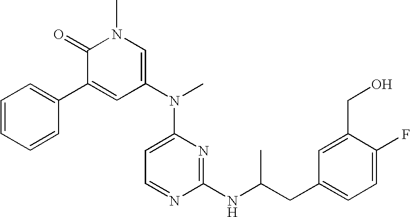 5-((2-(1-(4-Fluoro-3-(hydroxymethyl)phenyl)propan-2-ylamino)pyrimidin-4-yl)(methyl)amino)-1-methyl-3-phenylpyridin-2(1 H)-one
Step A: (E)-Methyl 2-fluoro-5-(2-nitroprop-1-enyl)benzoate.

Methyl 2-fluoro-5-formylbenzoate (3 g, 16.5 mmol) and ammonium acetate (1.27 g, 16.5 mmol) was suspended in nitroethane (65 mL) was heated at 130° C. for 1.5 h. After cooled, the volatile material was removed and the residue was partitioned between EtOAc and saturated aqueous NaHCO₃ (aq). The organic layer was washed with brine, dried (Na₂SO₄), and concentrated to afford a crude oil which was purified with a flash column chromatography (pure hexanes→1:5 EA/hexanes) to yield the title compound as a yellow needle. MS m/z 240 (M+H)⁺.

Step B: (5-(2-Aminopropyl)-2-fluorophenyl)methanol.

A solution of (E)-methyl 2-fluoro-5-(2-nitroprop-1-enyl)benzoate (2.64 g, 0.011 mmol) in THF (40 mL) was treated with LiAlH₄ (40 mL, 1.0M in THF) dropwise at 0° C. After added completely, the entire mixture was warmed to reflux for 16 h. After cooled, the overall mixture was filtrated and the filtrated cake was washed with EtOAc and the combined solvents were concentrated to give the title compound as a yellow solid. MS m/z 184 (M+H)⁺.

Step C: 5-((2-(1-(4-Fluoro-3-(hydroxymethyl)phenyl)propan-2-ylamino)pyrimidin-4-yl)(methyl)amino)-1-methyl-3-phenylpyridin-2(1H)-one.

The coupling of 1-methyl-5-(methyl(2-(methylsulfinyl)pyrimidin-4-yl)amino)-3-phenylpyridin-2(1H)-one (0.2 g, 0.56 mmol) and (5-(2-aminopropyl)-2-fluorophenyl)methanol (1.2 eq) was conducted in the similar manner as described previously to provide the title compound as a pale yellow solid. MS m/z 474 (M+H)⁺.

### EXAMPLE 178

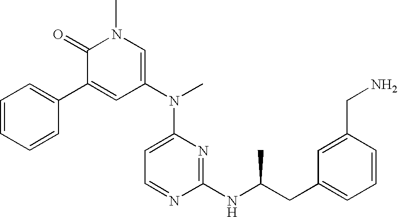 (S)-5-((2-(1-(3-(Aminomethyl)phenyl)propan-2-ylamino)pyrimidin-4-yl)(methyl)-amino)-1-methyl-3-phenylpyridin-2(1H)-one
Step A: (S)-tert-Butyl -3-(2-(4-(methyl(1-methyl-6-oxo-5-phenyl-1,6-dihydro-pyridin-3-yl)amino)pyrimidin-2-ylamino)propyl)benzylcarbamate.

Tan solid. MS m/z 555.3 (M+H)⁺.

Step B (S)-5-((2-(1-(3-(Aminomethyl)phenyl)propan-2-ylamino)pyrimidin-4-yl)-(methyl)amino)-1-methyl-3-phenylpyridin-2(1H)-one.

Deprotection (4N HCl in dioxane, RT) of the compound isolated above afforded (S)-5-((2-(1-(3-(amino-ethyl)phenyl)propan-2-ylamino)pyrimidin-4-yl)(methyl)amino)-1-methyl-3-phenylpyridin-2(1H)-one. Yellow solid. MS m/z 455 (M+H)⁺.

### EXAMPLE 179

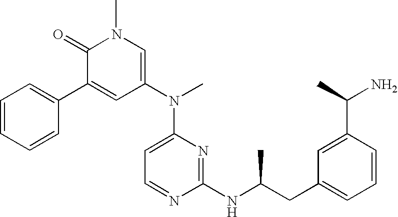 5-((2-((S)-1-(3-((R)-1-Aminoethyl)phenyl)propan-2-ylamino)pyrimidin-4-yl)(methyl)amino)-1-methyl-3-phenylpyridin-2(1H)-one.

Yellow solid. MS m/z 469 (M+H)⁺.

### EXAMPLE 180

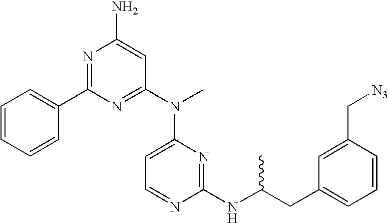 N⁴-(2-(1-(3-(Azidomethyl)phenyl)propan-2-ylamino)pyrimidine-4-yl)-N⁴-methyl-2-phenylpyrimidine-4,6-diamine.

A mixture of (3-(2-(4-((6-amino-2-phenyl-pyrimidin-4-yl)(methyl)amino)pyrimidin-2-ylamino)propyl)phenyl)methanol (100 mg, 0.23 mmol), diphenylphosphoryl azide (140 mg, 0.5 mmol), DBU (70 mg, 0.5 mmol) in THF (5 mL) was heated to 60° C. for 18 h. The THF was evaporated, the residue dissolved in chloroform and washed with 10% sodium carbonate. The organic layer was dried over sodium sulfate, concentrated and chromatographed on silica gel using 1% 2N NH₃ in MeOH/CHCl₃. MS m/z 467 (MH)⁺.

### EXAMPLE 181

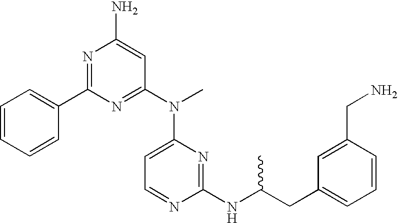 N⁴-(2-(1-(3-(Aminomethyl)phenyl)propan-2-ylamino)pyrimidine-4-yl)-N⁴-methyl-2-phenylpyrimidine-4,6-diamine.

A mixture of N⁴-(2-(1-(3-(azidomethyl)phenyl)-propan-2-ylamino)pyrimidine-4-yl)-N⁴-methyl-2-phenylpyrimidine-4,6-diamine (50 mg, 0.1 mmol), zinc (13 mg, 0.2 mmol), ammonium chloride (20 mg, 0.4 mmol) in ethanol (5 mL) was heated to 80° C. for 1 h. The solvent was evaporated, the residue dissolved in chloroform and washed with 10% sodium carbonate. The organic layer was dried over sodium sulfate, concentrated and chromatographed on silica gel using 3-6% 2N NH₃ in MeOH/CHCl₃. MS m/z 441 (MH)⁺.

### EXAMPLE 182

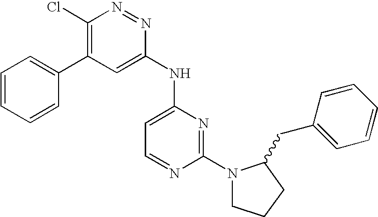 N-(2-(2-Benzylpyrrolidin-1-yl)pyrimidin-4-yl)-6-chloro-5-phenylpyridazin-3-amine.
Step A: 4-Phenyl-1,2-dihydropyridazine-3,6-dione.

A mixture of phenylmaleic acid (5.2 g, 0.03 mol), hydrazine (1.2 g, 0.036 mol) in acetic acid (60 mL) was stirred at RT for 24 h. The solvent was concentrated, the solid washed with sat. sodium bicarbonate, filtered and oven dried. MS m/z 189 (MH)⁺.

Step B: 3,6-Dichloro-4-phenylpyridazine.

A mixture of 4-phenyl-1,2-dihydro-pyridazine-3,6-dione (3.8 g, 0.02 mol), in phosphoryl chloride (61 g, 0.4 mol) was heated to 100° C. for 4 h. The solvent was concentrated and the residue dissolved in chloroform. The organic layer was washed with 10% sodium bicarbonate, dried over sodium sulfate and concentrated. The residue was chomatographed on silica gel with 50% ClCl₃/Hex. MS m/z 226 (MH)⁺.

Step C: 6-Chloro-5-phenylpyridazin-3-amine.

A mixture of 3,6-dichloro-4-phenyl-pyridazine (1.1 g, 5 mmol) in ammonia (1 mL) and isopropanol (5 mL) was heated to 110° C. for 24 h. The mixture was dissolved in chloroform, washed with water, dried over sodium sulfate and concentrated. The residue was chomatographed on silica gel with 2% ClCl₃/MeOH. MS m/z 206 (MH)⁺.

Step D: 6-Chloro-N-(2-(methylthio)pyrimidin-4-yl)-5-phenylpyridazin-3-amine.

A mixture of 6-chloro-5-phenylpyridazin-3-amine (0.82 g, 4 mmol), 4-chloro-2-methylthio-pyrimidine (0.77 g, 4.8 mmol), sodium t-butoxide (0.57 g, 5.6 mmol) in toluene (4 mL) was placed in a microwave tube and run in the Personal Chemistry microwave on normal absorption at 140° C. for 30 min. The mixture was washed with chloroform, filtered and dried. MS m/z 330 (MH)⁺.

Step E: 6-Chloro-N-(2-(methylsulfinyl)pyrimidin-4-yl)-5-phenylpyridazin-3-amine.

A mixture of 6-chloro-N-(2-(methylthio)pyrimidin-4-yl)-5-phenylpyridazin-3-amine (0.33 g, 1 mmol), 70% m-chloroperbenzoic acid (0.26 g, 1.5 mmol) in chloroform (10 mL) was stirred at RT for 3 h. The solvent was concentrated, the solid washed with ethyl acetate, filtered and dried. MS m/z 346 (MH)⁺.

Step F: N-(2-(2-Benzylpyrrolidin-1-yl)pyrimidin-4-yl)-6-chloro-5-phenylpyridazin-3-amine.

A mixture of 6-chloro-N-(2-(methylsulfinyl)pyrimidin-4-yl)-5-phenyl-pyridazin-3-amine (0.07 g, 0.2 mmol), 2-benzylpyrrolydine (0.06 g, 0.4 mmol) in DMF (0.5 mL) was placed in a microwave tube and run in the Personal Chemistry microwave on normal absorption at 170° C. for 30 min. The solvent was concentrated chromatographed on silica gel with 2% MeOH/CHCl₃. MS m/z 443 (MH)⁺.

### EXAMPLE 183

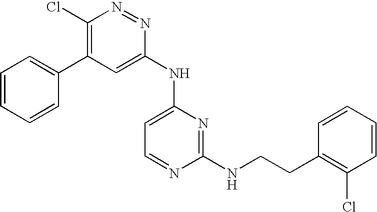 N²-(2-Chlorophenethyl)-N4-(6-chloro-5-phenylpyridazin-3-yl)pyrimidine-2,4-di-amine.

A mixture of 6-chloro-N-(2-(methylsulfinyl)pyrimidin-4-yl)-5-phenylpyridazin-3-amine (0.1 g, 0.3 mmol), 2-(2-chlorophenyl)ethylamine (0.09 g, 0.6 mmol) in DMF (0.5 mL) was placed in a microwave tube and run in the Personal Chemistry microwave on normal absorption at 160° C. for 15 min. The solvent was concentrated chromatographed on silica gel with 2% MeOH/CHCl₃. MS m/z 438 (MH)⁺.

### EXAMPLE 184

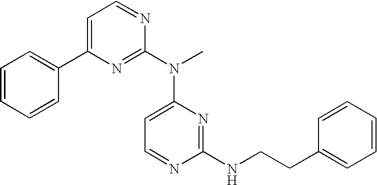 N⁴-Methyl-N²-phenethyl-N⁴-(4-phenylpyrimidin-2-yl)pyrimidine-2,4-diamine.
Step A: 2-Chloro-4-phenylpyrimidine.

A mixture of 2-chloropyrimidine (2.2 g, 0.02 mol) in THF (40 mL) was cooled to −78° C. Phenyl lithium (15 mL, 1.5M, 0.022 mol) was added and stirred 2 h warming to 0° C. DDQ (5 g, 0.22 mol) was added and stirring continued for 1 h. The solvent was concentrated and the residue dissolved in ether. The ether layer was washed with 2.5M sodium hydroxide, sat sodium chloride, dried over magnesium sulfate, concentrated and chromatographed on silica gel with 10% EtOAc/Hexane. MS m/z 191 (MH)⁺.

Step B: N-Methyl-4-phenylpyrimidin-2-amine.

A mixture of 2-chloro-4-phenyl-pyrimidine (1.8 g, 9.5 mmol) and 33% methylamine in ethanol (10 mL) was placed in a sealed tube and heated to 60° C. for 3 h. The mixture was concentrated and the solid dissolved in chloroform. The organic layer was washed with 10% sodium carbonate, dried over sodium sulfate and concentrated. MS m/z 186 (MH)⁺.

Step C: N-Methyl-2-(methylthio)-N-(4-phenylpyrimidin-2-yl)pyrimidin-4-amine.

A mixture of N-methyl-4-phenylpyrimidin-2-amine (0.86 g, 4.5 mmol), 4-chloro-2-methylthio-pyrimidine (0.87 g, 5.4 mmol), sodium t-butoxide (0.6 g, 6.3 mmol), palladium acetate (0.04 g, 0.2 mmol), rac-BINAP (0.25 g, 0.4 mmol) in toluene (10 mL) was heated to 80° C. for 1 h. The mixture was diluted with ethyl acetate, washed with sat. sodium bicarbonate, dried over sodium sulfate, concentrated and chromatographed on silica gel with 15% EtOAC/Hexanes. MS m/z 310 (MH)⁺.

Step D: N-Methyl-2-(methylsulfinyl)-N-(4-phenylpyrimidin-2-yl)pyrimidin-4-amine.

A mixture of N-methyl-2-(methylthio)-N-(4-phenylpyrimidin-2-yl)-pyrimidin-4-amine (0.31 g, 1 mmol), 70% m-chloroperbenzoic acid (0.26 g, 1.5 mmol) in chloroform (10 mL) was stirred at RT for 3 h. The solvent was concentrated, the residue dissolved in ethyl acetate, washed with sat. sodium bicarbonate, dried over sodium sulfate and concentrated. MS m/z 326 (MH)⁺.

Step E: N⁴-Methyl-N²-phenethyl-N⁴-(4-phenylpyrimidin-2-yl)pyrimidine-2,4-diamine.

A mixture of N-methyl-2-(methylsulfinyl)-N-(4-phenylpyrimidin-2-yl)pyrimidin-4-amine (0.16 g, 0.5 mmol), phenethylamine (0.12 g, 1 mmol) pyridine (0.04 g, 0.5 mmol) in DMSO (0.5 mL) was placed in a microwave tube and run in the Personal Chemistry microwave on normal absorption at 180° C. for 15 min. The mixture was diluted with EtOAc, washed with water, sat. sodium chloride, dried over sodium sulfate, concentrated and chromatographed on silica gel with 35% EtOAc/Hexane. MS m/z 383 (MH)⁺.

### EXAMPLE 185

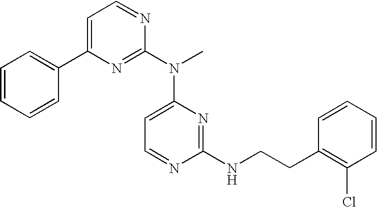 N²-(2-Chlorophenethyl)-N⁴-methyl-N⁴-(4-phenylpyrimidin-2-yl)pyrimidine-2,4-diamine.

A mixture of N-methyl-2-(methylsulfmyl)-N-(4-phenylpyrimidin-2-yl)pyrimidin-4-amine (0.16 g, 0.5 mmol), 2-(2-chlorophenyl)ethylamine (0.16 g, 1 mmol) pyridine (0.04 g, 0.5 mmol) in DMSO (0.5 mL) was placed in a microwave tube and run in the Personal Chemistry microwave on normal absorption at 180° C. for 15 min. The mixture was diluted with EtOAc, washed with water, sat. sodium chloride, dried over sodium sulfate, concentrated and chromatographed on silica gel with 35% EtOAc/Hexane. MS m/z 417 (MH)⁺.

### EXAMPLE 186

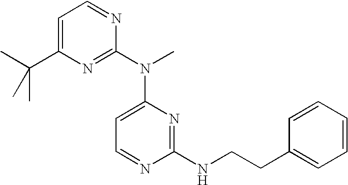 N⁴-(4-tert-Butylpyrimidin-2-yl)-N⁴-methyl-N²-phenethylpyrimidine-2,4-diamine.
Step A: 4-tert-Butyl-2-chloropyrimidine.

A mixture of 2-chloropyrimidine (5.7 g, 0.05 mol) in THF (50 mL) was cooled to −78° C. t-Butyl lithium (32 mL 1.7M, 0.055 mol) was added and stirred 2 h warming to 0° C. Acetic acid (5 mL, 50%) was added followed by DDQ (5 g, 0.22 mol) and stirring continued for 1 h. The solvent was concentrated and the residue dissolved in ether. The ether layer was washed with 10% sodium hydroxide, sat sodium chloride, dried over magnesium sulfate, concentrated and chromatographed on silica gel with 10% EtOAc/Hexane. MS m/z 191 (MH)⁺.

Step B: 4-tert-Butyl-N-methylpyrimidin-2-amine.

A mixture of 4-tert-butyl-2-chloropyrimidine (1.1 g, 6.4 mmol), 33% methylamine in ethanol (1 mL) in ethanol (4 mL) was placed in a sealed tube and heated to 60° C. for 3 h. The mixture was concentrated and the solid dissolved in chloroform. The organic layer was washed with water, dried over sodium sulfate, concentrated and chromatographed on silica gel with 30% EtOAc/Hexane. MS m/z 165 (MH)⁺.

Step C: N-(4-tert-Butylpyrimidin-2-yl)-N-methyl-2-(methylthio)pyrimidin-4-amine.

A mixture of 4-tert-butyl-N-methylpyrimidin-2-amine (0.17 g, 1 mmol), 4-chloro-2-methylthio-pyrimidine (0.22 g, 1.4 mmol), sodium t-butoxide (0.13 g, 1.4 mmol), palladium acetate (11 mg, 5 mol %), rac-BINAP (60 mg, 10 mol %) in toluene (2 mL) was heated to 100° C. for 18 h. The mixture was diluted with dichloromethane, washed with sat. sodium bicarbonate, dried over sodium sulfate, concentrated and chromatographed on silica gel with 15% EtOAC/Hexanes. MS m/z 290 (MH)⁺.

Step D: N-(4-tert-Butylpyrimidin-2-yl)-N-methyl-2-(methylsulfinyl)pyrimidin-4-amine.

A mixture of N-(4-tert-butylpyrimidin-2-yl)-N-methyl-2-(methylthio)-pyrimidin-4-amine (0.29 g, 1 mmol), 70% m-chloroperbenzoic acid (0.4 g, 2.4 mmol) in chloroform (10 mL) was stirred at RT for 4 h. The solvent was concentrated, the residue dissolved in ethyl acetate, washed with sat. sodium bicarbonate, dried over sodium sulfate and concentrated. MS m/z 305 (MH)⁺.

Step E: N⁴-(4-tert-Butylpyrimidin-2-yl)-N⁴-methyl-N²-phenethylpyrimidine-2,4-diamine.

A mixture of N-(4-tert-butylpyrimidin-2-yl)-N-methyl-2-(methylsulfinyl)-pyrimidin-4-amine (0.15 g, 0.5 mmol), phenethylamine (0.12 g, 1 mmol) pyridine (0.04 g, 0.5 mmol) in DMSO (0.5 mL) was placed in a microwave tube and run in the Personal Chemistry microwave on normal absorption at 150° C. for 15 min. The mixture was diluted with EtOAc, washed with water, sat. sodium chloride, dried over sodium sulfate, concentrated and chromatographed on silica gel with 30% EtOAc/Hexane. MS m/z 363 (MH)⁺.

Biological Assays

The following assays were used to characterize the ability of compounds of the invention to inhibit the production of TNF-α and IL-1-β. The second assay can be used to measure the inhibition of TNF-α and/or IL-1-β in mice after oral administration of the test compounds. The third assay, a glucagon binding inhibition in vitro assay, can be used to characterize the ability of compounds of the invention to inhibit glucagon binding. The fourth assay, a cyclooxygenase enzyme (COX-1 and COX-2) inhibition activity in vitro assay, can be used to characterize the ability of compounds of the invention to inhibit COX-1 and/or COX-2. The fifth assay, a Raf-kinase inhibition assay, can be used to characterize the compounds of the invention to inhibit phosphorylation of MEK by activated Raf-kinase.

Lipopolysaccharide-Activated Monocyte TNF Production Assay

Isolation of Monocytes

Test compounds were evaluated in vitro for the ability to inhibit the production of TNF by monocytes activated with bacterial lipopolysaccharide (LPS). Fresh residual source leukocytes (a byproduct of plateletpheresis) were obtained from a local blood bank, and peripheral blood mononuclear cells (PBMCs) were isolated by density gradient centrifugation on Ficol-Paque Plus (Pharmacia). PBMCs were suspended at 2×10⁶/mL in DMEM supplemented to contain 2% FCS, 10 mM, 0.3 mg/ml glutamate, 100 U/mL penicillin G and 100 mg/mL streptomycin sulfate (complete media). Cells were plated into Falcon flat bottom, 96 well culture plates (200 μL/well) and cultured overnight at 37° C. and 6% CO₂. Non-adherent cells were removed by washing with 200 μl/well of fresh medium. Wells containing adherent cells (˜70% monocytes) were replenished with 100 μL of fresh medium.

Preparation of Test Compound Stock Solutions

Test compounds were dissolved in DMZ. Compound stock solutions were prepared to an initial concentration of 10-50 μM. Stocks were diluted initially to 20-200 μM in complete media. Nine two-fold serial dilutions of each compound were then prepared in complete medium.

Treatment of Cells with Test Compounds and Activation of TNF Production with Lipopolysaccharide

One hundred microliters of each test compound dilution were added to microtiter wells containing adherent monocytes and 100 μL complete medium. Monocytes were cultured with test compounds for 60 min at which time 25 μL of complete medium containing 30 ng/mL lipopolysaccharide from E. coli K532 were added to each well. Cells were cultured an additional 4 hrs. Culture supernatants were then removed and TNF presence in the supernatants was quantified using an ELISA.

TNF ELISA

Flat bottom, 96 well Corning High Binding ELISA plates were coated overnight (4° C.) with 150 μL/well of 3 μg/mL murine anti-human TNF-α MAb (R&D Systems #MAB210). Wells were then blocked for 1 h at RT with 200 μL/well of CaCl₂-free ELISA buffer supplemented to contain 20 mg/mL BSA (standard ELISA buffer: 20 mM, 150 mM NaCl, 2 mM CaCl₂, 0.15 mM thimerosal, pH 7.4). Plates were washed and replenished with 100 μL of test supernatants (diluted 1:3) or standards. Standards consisted of eleven 1.5-fold serial dilutions from a stock of 1 ng/mL recombinant human TNF (R&D Systems). Plates were incubated at RT for 1 h on orbital shaker (300 rpm), washed and replenished with 100 μL/well of 0.5 μg/mL goat anti-human TNF-α (R&D systems #AB-210-NA) biotinylated at a 4:1 ratio. Plates were incubated for 40 min, washed and replenished with 100 μL/well of alkaline phosphatase-conjugated streptavidin (Jackson ImmunoResearch #016-050-084) at 0.02 μg/mL. Plates were incubated 30 min, washed and replenished with 200 μL/well of 1 mg/mL of p-nitrophenyl phosphate. After 30 min, plates were read at 405 nm on a Vₘₐₓ plate reader.

Data Analysis

Standard curve data were fit to a second order polynomial and unknown TNF-α concentrations determined from their OD by solving this equation for concentration. TNF concentrations were then plotted vs. test compound concentration using a second order polynomial. This equation was then used to calculate the concentration of test compounds causing a 50% reduction in TNF production.

Compounds of the invention can also be shown to inhibit LPS-induced release of IL-1β, IL-6 and/or IL-8 from monocytes by measuring concentrations of IL-1β, IL-6 and/or IL-8 by methods well known to those skilled in the art. In a similar manner to the above described assay involving the LPS induced release of TNF-α from monocytes, compounds of this invention can also be shown to inhibit LPS induced release of IL-1β, IL-6 and/or IL-8 from monocytes by measuring concentrations of IL-1β, IL-6 and/or IL-8 by methods well known to those skilled in the art. Thus, the compounds of the invention may lower elevated levels of TNF-α, IL-1, IL-6, and IL-8 levels. Reducing elevated levels of these inflammatory cytokines to basal levels or below is favorable in controlling, slowing progression, and alleviating many disease states. All of the compounds are useful in the methods of treating disease states in which TNF-α, IL-1β, IL-6, and IL-8 play a role to the full extent of the definition of TNF-α-mediated diseases described herein.

Lipopolysaccharide-Activated THP1 Cell TNF Production Assay

THP1 cells are resuspended in fresh THP1 media (RPMI 1640, 10% heat-inactivated FBS, 1×PGS, 1×NEAA, plus 30 μM βME) at a concentration of 1E6/mL. One hundred microliters of cells per well are plated in a polystyrene 96-well tissue culture. One microgram per mL of bacterial LPS is prepared in THP1 media and is transferred to the wells. Test compounds are dissolved in 100% DMSO and are serially diluted 3 fold in a polypropylene 96-well microtiter plate (drug plate). HI control and LO control wells contain only DMSO. One microliter of test compound from the drug plate followed by 10 μL of LPS are transferred to the cell plate. The treated cells are induced to synthesize and secrete TNF-α at 37° C. for 3 h. Forty microliters of conditioned media are transferred to a 96-well polypropylene plate containing 110 μL of ECL buffer (50 mM Tris-HCl pH 8.0, 100 mM NaCl, 0.05% Tween 20, 0.05% NaN₃ and 1% FBS) supplemented with 0.44 nM MAB610 monoclonal Ab (R&D Systems), 0.34 nM ruthenylated AF210NA polyclonal Ab (R&D Systems) and 44 μg/mL sheep anti-mouse M280 Dynabeads (Dynal). After a 2 h incubation at RT with shaking, the reaction is read on the ECL M8 Instrument (IGEN Inc.). A low voltage is applied to the ruthenylated TNF-α immune complexes, which in the presence of TPA (the active component in Origlo), results in a cyclical redox reaction generating light at 620 nM. The amount of secreted TNF-α in the presence of compound compared with that in the presence of DMSO vehicle alone (HI control) is calculated using the formula: % control (POC)=(cpd−average LO)/(average HI−average LO)*100. Data (consisting of POC and inhibitor concentration in μM) is fitted to a 4-parameter equation (y=A+((B−A)/(1+((×/C)ôD))), where A is the minimum y (POC) value, B is the maximum y (POC), C is the x (cpd concentration) at the point of inflection and D is the slope factor) using a Levenburg-Marquardt non-linear regression algorithm.

The following compounds exhibit activities in the THP1 cell assay (LPS induced TNF release) with IC₅₀ values of 20 μM or less:
(1R)-2-((4-(methyl(2-phenyl-4-pyrimidinyl)amino)-2-pyrimidinyl)amino)-1-phenylethanol;
(1S)-2-((4-(methyl(2-phenyl-4-pyrimidinyl)amino)-2-pyrimidinyl)amino)-1-phenylethanol;
(1S)-2-((4-(methyl(2-phenyl-4-pyrimidinyl)amino)-2-pyrimidinyl)amino)-1-(2-pyridinyl)ethanol;
(2R)-2-((4-(methyl(2-phenyl-4-pyrimidinyl)amino)-2-pyrimidinyl)amino)-3-phenyl-1-propanol;
(3-((2R)-2-((4-((2-chloro-6-phenyl-4-pyridinyl)(methyl)amino)-2-pyrimidinyl)amino)propyl)phenyl)methanol;
(3-((2R)-2-((4-((6-(3-fluorophenyl)-2-pyridinyl)(methyl)amino)-2-pyrimidinyl)amino)propyl)phenyl)methanol;
(3-((2S)-2-((4-((6-amino-2-phenyl-4-pyrimidinyl)(methyl)amino)-2-pyrimidinyl)amino)propyl)phenyl)methanol;
(3R)-3-((4-(methyl(1-methyl-6-oxo-5-phenyl-1,6-dihydro-3-pyridinyl)amino)-2-pyrimidinyl)amino)-4-phenylbutanoic acid;
(3R)-3-((4-(methyl(2-phenyl-4-pyrimidinyl)amino)-2-pyrimidinyl)amino)-4-phenyl-1-butanol;
(3R)-3-((4-(methyl(2-phenyl-4-pyrimidinyl)amino)-2-pyrimidinyl)amino)-3-phenyl-1-propanol;
(3S)-3-((4-(methyl(2-phenyl-4-pyrimidinyl)amino)-2-pyrimidinyl)amino)-3-phenyl-1-propanol;
(3S)-3-((4-(methyl(2-phenyl-4-pyrimidinyl)amino)-2-pyrimidinyl)amino)-3-phenyl-1-propanol;
1,1-dimethylethyl (1S)-1-(3-(2-((4-(methyl(2-phenyl-4-pyrimidinyl)amino)-2-pyrimidinyl)amino)ethyl)phenyl)ethylcarbamate;
1,1-dimethylethyl (1S)-1-(4-(2-((4-(methyl(2-phenyl-4-pyrimidinyl)amino)-2-pyrimidinyl)amino)ethyl)phenyl)ethylcarbamate;
1,1-dimethylethyl (3-((2S)-2-((4-(methyl(1-methyl-6-oxo-5-phenyl-1,6-dihydro-3-pyridinyl)amino)-2-pyrimidinyl)amino)propyl)phenyl)methylcarbamate;
1,1-dimethylethyl 2-methyl-2-((4-(methyl(1-methyl-6-oxo-4-phenyl-1,6-dihydro-2-pyridinyl)amino)-2-pyrimidinyl)amino)propylcarbamate;
1,1-dimethylethyl 2-methyl-2-((4-(methyl(2-phenyl-4-pyrimidinyl)amino)-2-pyrimidinyl)amino)propylcarbamate;
1,1-dimethylethyl 4-((4-(methyl(1-methyl-6-oxo-4-phenyl-1,6-dihydro-2-pyridinyl)amino)-2-pyrimidinyl)amino)-1-piperidinecarboxylate;
1,1-dimethylethyl 4-((4-(methyl(1-methyl-6-oxo-5-phenyl-1,6-dihydro-3-pyridinyl)amino)-2-pyrimidinyl)amino)-1-piperidinecarboxylate;
1-methyl-5-((2-(methylsulfanyl)-4-pyrimidinyl)amino)-3-phenyl-2(1H)-pyridinone;
1-methyl-5-(methyl(2-((2-phenylethyl)amino)-4-pyrimidinyl)amino)-3-phenyl-2(1H)-pyridinone;
1-methyl-5-(methyl(2-(4-piperidinylamino)-4-pyrimidinyl)amino)-3-phenyl-2(1H)-pyridinone;
2-phenyl-4-((2-((2-(3-pyridinyl)ethyl)amino)-4-pyrimidinyl)amino)-5-pyrimidinecarboxamide;
3-(3-((1S)-1-((4-(methyl(2-phenyl-4-pyrimidinyl)amino)-2-pyrimidinyl)amino)ethyl)phenyl)propanoic acid;
4-((2-(((1S)-2-(3-(aminomethyl)phenyl)-1-methylethyl)amino)-4-pyrimidinyl)amino)-N-methyl-2-phenyl-5-pyrimidinecarboxamide;
4-(methyl(2-((2-(3-pyridinyl)ethyl)amino)-4-pyrimidinyl) amino)-2-phenyl-5-pyrimidinecarboxamide;
4-chloro-3-((2S)-2-((4-(methyl(2-phenyl-4-pyrimidinyl)amino)-2-pyrimidinyl)amino)propyl)benzonitrile;
5-((2-(((1R)-2-(3-(aminomethyl)phenyl)-1-methylethyl)amino)-4-pyrimidinyl)(methyl)amino)-3-phenyl-2(1H)-pyridinone;
5-((2-(((1R)-2-(4-fluoro-3-(hydroxymethyl)phenyl)-1-methylethyl)amino)-4-pyrimidinyl)(methyl)amino)-1-methyl-3-phenyl-2(1 H)-pyridinone;
5-((2-(((1S)-2-(3-((1R)-1-aminoethyl)phenyl)-1-methylethyl)amino)-4-pyrimidinyl)(methyl)amino)-3-phenyl-2(1H)-pyridinone;
5-((2-(((1S)-2-(3-((1R)-1-aminoethyl)phenyl)-1-methylethyl)amino)-4-pyrimidinyl)(methyl)amino)-1-methyl-3-phenyl-2(1H)-pyridinone;
5-((2-(((1S)-2-(3-((1S)-1-aminoethyl)phenyl)-1-methylethyl)amino)-4-pyrimidinyl)(methyl)amino)-3-phenyl-2(1H)-pyridinone;
5-((2-(((1S)-2-(3-(aminomethyl)phenyl)-1-methylethyl)amino)-4-pyrimidinyl)(methyl)amino)-3-phenyl-2(1H)-pyridinone;
5-((2-(((1S)-2-(3-(aminomethyl)phenyl)-1-methylethyl)amino)-4-pyrimidinyl)(methyl)amino)-1-(1-methylethyl)-3-phenyl-2(1H)-pyridinone;
5-((2-(((1S)-2-(3-(aminomethyl)phenyl)-1-methylethyl)amino)-4-pyrimidinyl)(methyl)amino)-1-methyl-3-phenyl-2(1H)-pyridinone;
5-((2-(((1S)-2-(3-(aminomethyl)phenyl)-1-methylethyl)amino)-4-pyrimidinyl)(methyl)amino)-3-(2-fluorophenyl)-2(1H)-pyridinone;
5-((2-((1-acetyl-4-piperidinyl)amino)-4-pyrimidinyl)(methyl)amino)-3-phenyl-2(1H)-pyridinone;
5-((2-((1-acetyl-4-piperidinyl)amino)-4-pyrimidinyl)(methyl)amino)-1-methyl-3-phenyl-2(1H)-pyridinone;
5-(methyl(2-((2-phenylethyl)amino)-4-pyrimidinyl)amino)-3-phenyl-2(1H)-pyridinone;
5-fluoro-N-4-(5-fluoro-2-phenyl-4-pyrimidinyl)-N-4-methyl-N-2-(2-(3-pyridinyl)ethyl)-2,4-pyrimidinedianine;
5-fluoro-N-4-methyl-N-4-(2-phenyl-4-pyrimidinyl)-N-2-(2-(3-pyridinyl)ethyl)-2,4-pyrimidinediamine;
6-((2-((2-(2-chlorophenyl)ethyl)amino)-4-pyrimidinyl)amino)-1-methyl-4-phenyl-2(1H)-pyridinone;
6-(methyl(2-((2-(2-pyridinyl)ethyl)amino)-4-pyrimidinyl)amino)-2-phenyl-4-pyrimidinol;
6-chloro-5-phenyl-N-(2-((2S)-2-(phenylmethyl)-1-pyrrolidinyl)-4-pyrimidinyl)-3-pyridazinamine;
ethyl 2-phenyl-4-((2-((2-(2-pyridinyl)ethyl)amino)-4-pyrimidinyl)amino)-5-pyrimidinecarboxylate;
N-(4-((4-(methyl(2-phenyl-4-pyrimidinyl)amino)-2-pyrimidinyl)amino)cyclohexyl)acetamide;
N-(4-(methyl(2-phenyl-4-pyrimidinyl)amino)-2-pyrimidinyl)-D-phenylalaninamide;
N-(4-(methyl(2-phenyl-4-pyrimidinyl)amino)-2-pyrimidinyl)-D-phenylalanine;
N-1-((2R)-2-((4-(methyl(2-phenyl-4-pyrimidinyl)amino)-2-pyrimidinyl)amino)-3-phenylpropyl)glycinamide;
N-1-((3-((2S)-2-((4-(methyl(4-(methyloxy)-6-phenyl-1,3,5-triazin-2-yl)amino)-2-pyrimidinyl)amino)propyl)phenyl)methyl)-L-alaninamide;
N-1-((3-((2S)-2-((4-methyl-6-(methyl(2-phenyl-4-pyrimidinyl)amino)-2-pyrimidinyl)amino)propyl)phenyl)methyl)-L-alaninamide;
N-1-(4-((4-(methyl(2-phenyl-4-pyrimidinyl)amino)-2-pyrimidinyl)amino)cyclohexyl)-L-alaninamide;
N-2-((1-acetyl-4-piperidinyl)methyl)-N-4-methyl-N-4-(2-phenyl-4-pyrimidinyl)-2,4-pyrimidinediamine;
N-2-((1R)-2-((2-aminoethyl)amino)-1-(phenylmethyl)ethyl)-N-4-methyl-N-4-(2-phenyl-4-pyrimidinyl)-2,4-pyrimidinediamine;
N-2-((1R)-2-(3-(aminomethyl)phenyl)-1-methylethyl)-N-4-(2-chloro-6-phenyl-4-pyridinyl)-N-4-methyl-2,4-pyrimidinediamine;
N-2-((1R)-2-(3-(aminomethyl)phenyl)-1-methylethyl)-N-4-(6-(3-fluorophenyl)-2-pyridinyl)-N-4-methyl-2,4-pyrimidinediamine;
N-2-((1R)-2-amino-1-(phenylmethyl)ethyl)-N-4-methyl-N-4-(2-phenyl-4-pyrimidinyl)-2,4-pyrimidinediamine;
N-2-((1R)-3-(cyclopropylamino)-1-(phenylmethyl)propyl)-N-4-methyl-N-4-(2-phenyl-4-pyrimidinyl)-2,4-pyrimidinediamine;
N-2-((1R)-3-amino-1-(phenylmethyl)propyl)-N-4-methyl-N-4-(2-phenyl-4-pyrimidinyl)-2,4-pyrimidinediamine;
N-2-((1S)-1-((1R,3S)-3-(2-aminoethyl)cyclohexyl)ethyl)-N-4-methyl-N-4-(2-phenyl-4-pyrimidinyl)-2,4-pyrimidinediamine;
N-2-((1S)-1-(3-(2-aminoethyl)phenyl)ethyl)-N-4-methyl-N-4-(2-phenyl-4-pyrimidinyl)-2,4-pyrimidinediamine;
N-2-((l1S)-1-(4-(2-aminoethyl)phenyl)ethyl)-N-4-methyl-N-4-(2-phenyl-4-pyrimidinyl)-2,4-pyrimidinediamine;
N-2-((1S)-2-(3-((1R)-1-aminoethyl)phenyl)-1-methylethyl)-N-4-,6-dimethyl-N-4-(2-phenyl-4-pyrimidinyl)-2,4-pyrimidinediamine;
N-2-((1S)-2-(3-((1R)-1-aminoethyl)phenyl)-1-methylethyl)-N-4-methyl-N-4-(2-phenyl-4-pyrimidinyl)-2,4-pyrimidinediamine;
N-2-((1S)-2-(3-((1R)-1-aminoethyl)phenyl)-1-methylethyl)-N-4-methyl-N-4-(4-(methyloxy)-6-phenyl-1,3,5-triazin-2-yl)-2,4-pyrimidinediamine;
N-2-((1S)-2-(3-((1S)-1-aminoethyl)phenyl)-1-methylethyl)-N-4-methyl-N-4-(2-phenyl-4-pyrimidinyl)-2,4-pyrimidinediamine;
N-2-((1S)-2-(3-(1-amino-1-methylethyl)phenyl)-1-methylethyl)-N-4-methyl-N-4-(2-phenyl-4-pyrimidinyl)-2,4-pyrimidinediamine;
N-2-((1S)-2-(3-(1-aminocyclopropyl)phenyl)-1-methylethyl)-N-4-methyl-N-4-(2-phenyl-4-pyrimidinyl)-2,4-pyrimidinediamine;
N-2-((1S)-2-(3-(1H-imidazol-1-yl)phenyl)-1-methylethyl)-N-4-methyl-N-4-(2-phenyl-4-pyrimidinyl)-2,4-pyrimidinediamine;
N-2-((1S)-2-(3-(aminomethyl)phenyl)-1-methylethyl)-N-4-(2-(2,4-difluorophenyl)-4-pyrimidinyl)-N-4-methyl-2,4-pyrimidinediamine;
N-2-((1S)-2-(3-(aminomethyl)phenyl)-1-methylethyl)-N-4-(2-(2-fluorophenyl)-4-pyrimidinyl)-N-4-methyl-2,4-pyrimidinediamine;
N-2-((1S)-2-(3-(aminomethyl)phenyl)-1-methylethyl)-N-4-(2-(3-fluorophenyl)-4-pyrimidinyl)-N-4-methyl-2,4-pyrimidinediamine,
N-2-((1S)-2-(3-(aminomethyl)phenyl)-1-methylethyl)-N-4-(2-(4-fluorophenyl)-4-pyrimidinyl)-N-4-methyl-2,4-pyrimidinediamine;
N-2-((1S)-2-(3-(aminomethyl)phenyl)-1-methylethyl)-N-4-(5-fluoro-2-phenyl-4-pyrimidinyl)-N-4-methyl-2,4-pyrimidinediamine;
N-2-((1S)-2-(3-(aminomethyl)phenyl)-1-methylethyl)-N-4-(5-fluoro-2-(2-fluorophenyl)-4-pyrimidinyl)-N-4-methyl-2,4-pyrimidinediamine;
N-2-((1S)-2-(3-(aminomethyl)phenyl)-1-methylethyl)-N-4-(6-amino-2-phenyl-4-pyrimidinyl)-N-4-methyl-2,4-pyrimidinediamine;
N-2-((1S)-2-(3-(aminomethyl)phenyl)-1-methylethyl)-N-4-methyl-N-4-(2-phenyl-4-pyrimidinyl)-2,4-pyrimidinediamine;
N-2-((1S)-2-(3-(aminomethyl)phenyl)-1-methylethyl)-N-4-methyl-N-4-(4-(methyloxy)-6-phenyl-1,3,5-triazin-2-yl)-2,4-pyrimidinediamine;
N-2-((1S)-2-(3-(aminomethyl)phenyl)-1-methylethyl)-N-4-methyl-N-4-(6-phenyl-2-pyrazinyl)-2,4-pyrimidinediamine;
N-2-((1S)-2-(5-(aminomethyl)-2-chlorophenyl)-1-methylethyl)-N-4-methyl-N-4-(2-phenyl-4-pyrimidinyl)-2,4-pyrimidinediamine;
N-2-((2R)-2-(dimethylamino)-2-(3-pyridinyl)ethyl)-N-4-methyl-N-4-(2-phenyl-4-pyrimidinyl)-2,4-pyrimidinediamine;
N-2-((2R)-2-amino-2-phenylethyl)-N-4-methyl-N-4-(2-phenyl-4-pyrimidinyl)-2,4-pyrimidinediamine;
N-2-((2S)-2-(dimethylamino)-2-(3-pyridinyl)ethyl)-N-4-methyl-N-4-(2-phenyl-4-pyrimidinyl)-2,4-pyrimidinediamine;
N-2-((2S)-2-(dimethylamino)-2-(3-pyridinyl)ethyl)-N-4-methyl-N-4-(2-phenyl-4-pyrimidinyl)-2,4-pyrimidinediamine;
N-2-(1-((2S)-2-aminopropanoyl)-4-piperidinyl)-N-4-methyl-N-4-(2-phenyl-4-pyrimidinyl)-2,4-pyrimidinediamine;
N-2-(1-((3R)-3-aminobutanoyl)-4-piperidinyl)-N-4-methyl-N-4-(2-phenyl-4-pyrimidinyl)-2,4-pyrimidinediamine;
N-2-(1-(aminoacetyl)-4-piperidinyl)-N-4-methyl-N-4-(2-phenyl-4-pyrimidinyl)-2,4-pyrimidinediamine;
N-2-(1,1-dimethyl-2-phenylethyl)-N-4-methyl-N-4-(2-phenyl-4-pyrimidinyl)-2,4-pyrimidinediamine;
N-2-(1,1-dimethyl-2-phenylethyl)-N-4-methyl-N-4-(4-phenyl-2-pyrimidinyl)-2,4-pyrimidinediamine;
N-2-(1-acetyl-4-piperidinyl)-N-4-methyl-N-4-(2-phenyl-4-pyrimidinyl)-2,4-pyrimidinediamine;
N-2-(2-(((1S)-2-(3-(aminomethyl)phenyl)-1-methylethyl)amino)-4-pyrimidinyl)-N-2-methyl-6-phenyl-2,4-pyrimidinediamine;
N-2-(2-((1R,3S)-3-((1S)-1-aminoethyl)cyclohexyl)ethyl)-N-4-methyl-N-4-(2-phenyl-4-pyrimidinyl)-2,4-pyrimidinediamine;
N-2-(2-(2-chlorophenyl)ethyl)-N-4-(4-(1,1-dimethylethyl)-2-pyrimidinyl)-N-4-methyl-2,4-pyrimidinediamine;
N-2-(2-(2-chlorophenyl)ethyl)-N-4-(6-chloro-5-phenyl-3-pyridazinyl)-2,4-pyrimidinediamine;
N-2-(2-(2-chlorophenyl)ethyl)-N-4-methyl-N-4-(2-phenyl-4-pyrimidinyl)-2,4-pyrimidinediamine;
N-2-(2-(2-chlorophenyl)ethyl)-N-4-methyl-N-4-(4-phenyl-2-pyrimidinyl)-2,4-pyridinediamine;
N-2-(2-(2-chlorophenyl)ethyl)-N-4-methyl-N-4-(6-phenyl-2-pyrazinyl)-2,4-pyrimidinediamine;
N-2-(2-(3-((1S)-1-aminoethyl)phenyl)ethyl)-N-4-methyl-N-4-(2-phenyl-4-pyrimidinyl)-2,4-pyrimidinediamine;
N-2-(2-(4-((1S)-1-aminoethyl)phenyl)ethyl)-N-4-methyl-N-4-(2-phenyl-4-pyrimidinyl)-2,4-pyrimidinediamine;
N-2-(2-amino-1,1-dimethylethyl)-N-4-methyl-N-4-(2-phenyl-4-pyrimidinyl)-2,4-pyrimidinediamine;
N-2-(4-aminocyclohexyl)-N-4-(2-(2-fluorophenyl)-4-pyrimidinyl)-N-4-methyl-2,4-pyrimidinediamine;
N-2-(4-aminocyclohexyl)-N-4-(5-fluoro-2-phenyl-4-pyrimidinyl)-N-4-methyl-2,4-pyrimidinediamine;
N-2-(4-aminocyclohexyl)-N-4-,6-dimethyl-N-4-(2-phenyl-4-pyrimidinyl)-2,4-pyrimidinediamine;
N-2-(4-aminocyclohexyl)-N-4-methyl-N-4-(2-phenyl-4-pyrimidinyl)-2,4-pyrimidinediamine;
N-2-(4-aminocyclohexyl)-N-4-methyl-N-4-(2-phenyl-4-pyrimidinyl)-2,4-pyrimidinediamine;
N-2-(4-aminocyclohexyl)-N-4-methyl-N-4-(6-phenyl-2-pyrazinyl)-2,4-pyrimidinediamine;
N-2-(4-aminocyclohexyl)-N-4-methyl-N-4-(6-phenyl-2-pyridinyl)-2,4-pyrimidinediamine;
N-4-(2-(2,3-difluorophenyl)-4-pyrimidinyl)-N-4-methyl-N-2-(2-(3-pyridinyl)ethyl)-2,4-pyrimidinediamine;
N-4-(2-(2,4-difluorophenyl)-4-pyrimidinyl)-N-4-methyl-N-2-(2-(3-pyridinyl)ethyl)-2,4-pyrimidinediamine;
N-4-(2-(2,5-difluorophenyl)-4-pyrimidinyl)-N-4-methyl-N-2-(2-(3-pyridinyl)ethyl)-2,4-pyrimidinediamine;
N-4-(2-(2-fluorophenyl)-4-pyrimidinyl)-N-4-methyl-N-2-(2-(3-pyridinyl)ethyl)-2,4-pyrimidinediamine;
N-4-(2-(3-fluorophenyl)-4-pyrimidinyl)-N-4-methyl-N-2-(2-(3-pyridinyl)ethyl)-2,4-pyrimidinediamine;
N-4-(2-(4-fluorophenyl)-4-pyrimidinyl)-N-4-methyl-N-2-(2-(3-pyridinyl)ethyl)-2,4-pyrimidinediamine;
N-4-(4-(1,1-dimethylethyl)-2-pyrimidinyl)-N-4-methyl-N-2-(2-phenylethyl)-2,4-pyrimidinediamine;
N-4-(5-bromo-2-phenyl-4-pyrimidinyl)-N-4-methyl-N-2-(2-(3-pyridinyl)ethyl)-2,4-pyrimidinediamine;
N-4-(6-(1-cyclohexen-1-yl)-2-pyridinyl)-N-4-methyl-N-2-(2-phenylethyl)-2,4-pyrimidinediamine;
N-4-(6-(2,3-difluorophenyl)-2-pyridinyl)-N-4-methyl-N-2-(2-phenylethyl)-2,4-pyrimidinediamine;
N-4-(6-(2-chlorophenyl)-2-pyridinyl)-N-4-methyl-N-2-(2-phenylethyl)-2,4-pyrimidinediamine;
N-4-(6-(2-furanyl)-2-pyridinyl)-N-4-methyl-N-2-(2-phenylethyl)-2,4-pyrimidinediamine;
N-4-(6-(3,4-difluorophenyl)-2-pyridinyl)-N-4-methyl-N-2-(2-phenylethyl)-2,4-pyrimidinediamine;
N-4-(6-(3,5-difluorophenyl)-2-pyridinyl)-N-4-methyl-N-2-(2-phenylethyl)-2,4-pyrimidinediamine;
N-4-(6-(3-chlorophenyl)-2-pyridinyl)-N-4-methyl-N-2-(2-phenylethyl)-2,4-pyrimidinediamine;
N-4-(6-(3-furanyl)-2-pyridinyl)-N-4-methyl-N-2-(2-phenylethyl)-2,4-pyrimidinediamine;
N-4-(6-(4-chlorophenyl)-2-pyridinyl)-N-4-methyl-N-2-(2-phenylethyl)-2,4-pyrimidinediamine;
N-4-methyl-N-2-((1R)-2-((1-methylethyl)amino)-1-(phenylmethyl)ethyl)-N-4-(2-phenyl-4-pyrimidinyl)-2,4-pyrimidinediamine;
N-4-methyl-N-2-((1R)-2-(4-morpholinyl)-1-(phenylmethyl)ethyl)-N-4-(2-phenyl-4-pyrimidinyl)-2,4-pyrimidinediamine;
N-4-methyl-N-2-((1R)-3-((1-methylethyl)amino)-1-(phenylmethyl)propyl)-N-4-(2-phenyl-4-pyrimidinyl)-2,4-pyrimidinediamine;
N-4-methyl-N-2-((1R)-3-(4-morpholinyl)-1-(phenylmethyl)propyl)-N-4-(2-phenyl-4-pyrimidinyl)-2,4-pyrimidinediamine;
N-4-methyl-N-2-((1S)-1-(1-methylethyl)-3-(4-morpholinyl)-3-oxopropyl)-N-4-(2-phenyl-4-pyrimidinyl)-2,4-pyrimidinediamine;
N-4-methyl-N-2-((1S)-1-methyl-2-(3-(2-methyl-1H-imidazol-1-yl)phenyl)ethyl)-N-4-(2-phenyl-4-pyrimidinyl)-2,4-pyrimidinediamine;
N-4-methyl-N-2-((1S)-2-methyl-1-(2-(4-morpholinyl)ethyl)propyl)-N-4-(2-phenyl-4-pyrimidinyl)-2,4-pyrimidinediamine;
N-4-methyl-N-2-((2S)-2-(4-morpholinyl)-2-(3-pyridinyl)ethyl)-N-4-(2-phenyl-4-pyrimidinyl)-2,4-pyrimidinediamine;
N-4-methyl-N-2-(2-(3-pyridinyl)ethyl)-N-4-(2-(2-(trifluoromethyl)phenyl)-4-pyrimidinyl)-2,4-pyrimidinediamine;
N-4-methyl-N-2-(2-(3-pyridinyl)ethyl)-N-4-(2-(2-thienyl)-4-pyrimidinyl)-2,4-pyrimidinediamine;
N-4-methyl-N-2-(2-(3-pyridinyl)ethyl)-N-4-(2-(3-thienyl)-4-pyrimidinyl)-2,4-pyrimidinediamine;
N-4-methyl-N-2-(2-(4-morpholinyl)ethyl)-N-4-(2-phenyl-4-pyrimidinyl)-2,4-pyrimidinediamine;
N-4-methyl-N-2-(2-phenylethyl)-N-4-(2-phenyl-4-pyrimidinyl)-2,4-pyrimidinediamine;
N-4-methyl-N-2-(2-phenylethyl)-N-4-(4-phenyl-2-pyrimidinyl)-2,4-pyrimidinediamine;
N-4-methyl-N-2-(2-phenylethyl)-N-4-(6-(2-(trifluoromethyl)phenyl)-2-pyridinyl)-2,4-pyrimidinediamine;
N-4-methyl-N-2-(2-phenylethyl)-N-4-(6-(2-thienyl)-2-pyridinyl)-2,4-pyrimidinediamine;
N-4-methyl-N-2-(2-phenylethyl)-N-4-(6-(3-(trifluoromethyl)phenyl)-2-pyridinyl)-2,4-pyrimidinediamine;
N-4-methyl-N-2-(2-phenylethyl)-N-4-(6-(3-thienyl)-2-pyridinyl)-2,4-pyrimidinediamine;
N-4-methyl-N-2-(2-phenylethyl)-N-4-(6-(4-(trifluoromethyl)phenyl)-2-pyridinyl)-2,4-pyrimidinediamine;
N-4-methyl-N-2-(2-phenylethyl)-N-4-(6-(phenylmethyl)-2-pyridinyl)-2,4-pyrimidinediamine;
N-4-methyl-N-4-(2-(2-(methyloxy)phenyl)-4-pyrimidinyl)-N-2-(2-(3-pyridinyl)ethyl)-2,4-pyrimidinediamine;
N-4-methyl-N-4-(2-(2-methylphenyl)-4-pyrimidinyl)-N-2-(2-(3-pyridinyl)ethyl)-2,4-pyrimidinediamine;
N-4-methyl-N-4-(2-phenyl-4-pyrimidinyl)-N-2-(2-(2-pyridinyl)ethyl)-2,4-pyrimidinediamine;
N-4-methyl-N-4-(2-phenyl-4-pyrimidinyl)-N-2-(2-(3-(2-pyridinyl)phenyl)ethyl)-2,4-pyrimidinediamine;
N-4-methyl-N-4-(2-phenyl-4-pyrimidinyl)-N-2-(2-(3-pyridinyl)ethyl)-2,4-pyrimidinediamine;
N-4-methyl-N-4-(2-phenyl-4-pyrimidinyl)-N-2-(2-(5,6,7,8-tetrahydro-1,8-naphthyridin-2-yl)ethyl)-2,4-pyrimidinediamine;
N-4-methyl-N-4-(2-phenyl-4-pyrimidinyl)-N-2-(4-piperidinyl)-2,4-pyrimidinediamine;
N-4-methyl-N-4-(5-phenyl-6-((phenylmethyl)oxy)-3-pyridinyl)-N-2-(4-piperidinyl)-2,4-pyrimidinediamine;
N-4-methyl-N-4-(6-(2-(methyloxy)phenyl)-2-pyridinyl)-N-2-(2-phenylethyl)-2,4-pyrimidinediamine;
N-4-methyl-N-4-(6-(2-methylphenyl)-2-pyridinyl)-N-2-(2-phenylethyl)-2,4-pyrimidinediamine;
N-4-methyl-N-4-(6-(3-(methyloxy)phenyl)-2-pyridinyl)-N-2-(2-phenylethyl)-2,4-pyrimidinediamine;
N-4-methyl-N-4-(6-(3-methylphenyl)-2-pyridinyl)-N-2-(2-phenylethyl)-2,4-pyrimidinediamine;
N-4-methyl-N-4-(6-(4-(methyloxy)phenyl)-2-pyridinyl)-N-2-(2-phenylethyl)-2,4-pyrimidinediamine;
N-4-methyl-N-4-(6-(4-methylphenyl)-2-pyridinyl)-N-2-(2-phenylethyl)-2,4-pyrimidinediamine;
N-4-methyl-N-4-(6-(methyloxy)-5-phenyl-3-pyridinyl)-N-2-(2-phenylethyl)-2,4-pyrimidinediamine;
N-methyl-2-(2-methyl-1H-imidazol-1-yl)-N-(2-phenyl-4-pyrimidinyl)-4-pyrimidinamine;
N-methyl-2-phenyl-4-((2-((2-(2-pyridinyl)ethyl)amino)-4-pyrimidinyl)amino)-5-pyrimidinecarboxamide;
N-methyl-2-phenyl-4-((2-((2-(3-pyridinyl)ethyl)amino)-4-pyrimidinyl)amino)-5-pyrimidinecarboxamide;
N-methyl-2-phenyl-N-(2-((2R)-2-(phenylmethyl)-1-azetidinyl)-4-pyrimidinyl)-4-pyrimidinamine;
N-methyl-4-((2-(((1S)-1-methyl-2-(3-((((methylamino)carbonyl)amino)methyl)phenyl)ethyl)amino)-4-pyrimidinyl)amino)-2-phenyl-5-pyrimidinecarboxamide; and
N-methyl-N-(4-(methyl(2-phenyl-4-pyrimidinyl)amino)-2-pyrimidinyl)-D-phenylalaninamide.
   Inhibition of LPS-Induced TNF-α Production in Mice

Male DBA/1LACJ mice are dosed with vehicle or test compounds in a vehicle (the vehicle consisting of 0.5% tragacanth in 0.03 N HCl) 30 minutes prior to lipopolysaccharide (2 mg/Kg, I.V.) injection. Ninety minutes after LPS injection, blood is collected and the serum is analyzed by ELISA for TNF-α levels.

Compounds of the invention may be shown to have anti-inflammatory properties in animal models of inflammation, including carageenan paw edema, collagen induced arthritis and adjuvant arthritis, such as the carageenan paw edema model (C. A. Winter et al Proc. Soc. Exp. Biol. Med. (1962) vol 111, p 544; K. F. Swingle, in R. A. Scherrer and M. W. Whitehouse, Eds., Anti-inflammatory Agents, Chemistry and Pharmacology, Vol. 13-II, Academic, New York, 1974, p. 33) and collagen induced arthritis (D. E. Trentham et al J. Exp. Med. (1977) vol. 146, p 857; J. S. Courtenay, Nature (New Biol.) (1980), Vol 283, p 666).

¹²⁵I-Glucagon Binding Screen with CHO/hGLUR Cells

The assay is described in WO 97/16442, which is incorporated herein by reference in its entirety.

Reagents

The reagents can be prepared as follows: (a) prepare fresh 1M o-Phenanthroline (Aldrich) (198.2 mg/mL ethanol); (b) prepare fresh 0.5M DTT (Sigma); (c) Protease Inhibitor Mix (1000×): 5 mg leupeptin, 10 mg benzamidine, 40 mg bacitracin and 5 mg soybean trypsin inhibitor per mL DMSO and store aliquots at −20° C.; (d) 250 μM human glucagon (Peninsula): solubilize 0.5 mg vial in 575 μl 0.1N acetic acid (1 μL yields 1 μM final concentration in assay for non-specific binding) and store in aliquots at −20° C.; (e) Assay Buffer: 20 mM Tris (pH 7.8), 1 mM DTT and 3 mM o-phenanthroline; (f) Assay Buffer with 0.1% BSA (for dilution of label only; 0.01% final in assay): 10 μL 10% BSA (heat-inactivated) and 990 μL Assay Buffer; (g) ¹²⁵I-Glucagon (NEN, receptor-grade, 2200 Ci/mmol): dilute to 50,000 cpm/25 μL in assay buffer with BSA (about 50 pM final concentration in assay).

Harvesting of CHO/hGLUR Cells for Assay

1. Remove media from confluent flask then rinse once each with PBS (Ca, Mg-free) and Enzyme-free Dissociation Fluid (Specialty Media, Inc.).

2. Add 10 mL Enzyme-free Dissoc. Fluid and hold for about 4 min at 37° C.

3. Gently tap cells free, triturate, take aliquot for counting and centrifuge remainder for 5 min at 1000 rpm.

4. Resuspend pellet in Assay Buffer at 75000 cells per 100 μL.

Membrane preparations of CHO/hGLUR cells can be used in place of whole cells at the same assay volume. Final protein concentration of a membrane preparation is determined on a per batch basis.

Assay

The determination of inhibition of glucagon binding can be carried out by measuring the reduction of I¹²⁵-glucagon binding in the presence of compounds of Formula I. The reagents are combined as follows:

| | Compound/ | 250 μM | | CHO/hGLUR |
|---|---|---|---|---|
| | Vehicle | Glucagon | ¹²⁵I-Glucagon | Cells |
| Total | —/5 μl | — | 25 μL | 100 μL |
| Binding | | | | |
| +Compound | 5 μl/— | — | 25 μL | 100 μL |
| Nonspecific | —/5 μl | 1 μl | 25 μL | 100 μL |
| Binding | | | | |

The mixture is incubated for 60 min at 22° C. on a shaker at 275 rpm. The mixture is filtered over pre-soaked (0.5% polyethylimine (PEI)) GF/C filtermat using an Innotech Harvester or Tomtec Harvester with four washes of ice-cold 20 mM Tris buffer (pH 7.8). The radioactivity in the filters is determined by a gamma-scintillation counter.

Thus, compounds of the invention may also be shown to inhibit the binding of glucagon to glucagon receptors.

Cyclooxygenase Enzyme Activity Assay

The human monocytic leukemia cell line, THP-1, differentiated by exposure to phorbol esters expresses only COX-1; the human osteosarcoma cell line 143B expresses predominantly COX-2. THP-1 cells are routinely cultured in RPMI complete media supplemented with 10% FBS and human osteosarcoma cells (HOSC) are cultured in minimal essential media supplemented with 10% fetal bovine serum (MEM-10% FBS); all cell incubations are at 37° C. in a humidified environment containing 5% CO₂.

COX-1 Assay

In preparation for the COX-1 assay, THP-1 cells are grown to confluency, split 1:3 into RPMI containing 2% FBS and 10 mM phorbol 12 -myristate 13-acetate (TPA), and incubated for 48 h on a shaker to prevent attachment. Cells are pelleted and resuspended in Hank's Buffered Saline (HBS) at a concentration of 2.5×10⁶ cells/mL and plated in 96-well culture plates at a density of 5×10⁵ cells/mL. Test compounds are diluted in HBS and added to the desired final concentration and the cells are incubated for an additional 4 hours. Arachidonic acid is added to a final concentration of 30 mM, the cells incubated for 20 minutes at 37° C., and enzyme activity determined as described below.

COX-2 Assay

For the COX-2 assay, subconfluent HOSC are trypsinized and resuspended at 3×10⁶ cells/mL in MEM-FBS containing 1 ng human IL-1b/mL, plated in 96-well tissue culture plates at a density of 3×10⁴ cells per well, incubated on a shaker for 1 hour to evenly distribute cells, followed by an additional 2 hour static incubation to allow attachment. The media is then replaced with MEM containing 2% FBS (MEM-2% FBS) and 1 ng human IL-1b/mL, and the cells incubated for 18-22 hours. Following replacement of media with 190 mL MEM, 10 mL of test compound diluted in HBS is added to achieve the desired concentration and the cells incubated for 4 hours. The supernatants are removed and replaced with MEM containing 30 mM arachidonic acid, the cells incubated for 20 minutes at 37° C., and enzyme activity determined as described below.

COX Activity Determined

After incubation with arachidonic acid, the reactions are stopped by the addition of 1N HCl, followed by neutralization with 1N NaOH and centrifugation to pellet cell debris. Cyclooxygenase enzyme activity in both HOSC and THP-1 cell supernatants is determined by measuring the concentration of PGE₂ using a commercially available ELISA (Neogen #404110). A standard curve of PGE₂ is used for calibration, and commercially available COX-1 and COX-2 inhibitors are included as standard controls.

Raf Kinase Assay

In vitro Raf kinase activity is measured by the extent of phosphorylation of the substrate MEK (Map kinase/ERK kinase) by activated Raf kinase, as described in GB 1,238,959 (incorporated herein by reference in its entirety). Phosphorylated MEK is trapped on a filter and incorporation of radiolabeled phosphate is quantified by scintillation counting.

Materials:

Activated Raf is produced by triple transfection of Sf9 cells with baculoviruses expressing “Glu-Glu”-epitope tagged Raf,val¹²-H-Ras, and Lck. The “Glu-Glu”-epitope, Glu-Try-Met-Pro-Met-Glu, was fused to the carboxy-terminus of full length c-Raf.
Catalytically inactive MEK (K97A mutation) is produced in Sf9 cells transfected with a baculovirus expressing c-terminus “Glu-Glu” epitope-tagged K97A MEK1.
Anti “Glu-Glu” antibody was purified from cells grown as described in: Grussenmeyer, et al., Proceedings of the National Academy of Science, U.S.A. pp 7952-7954, 1985.
Column buffer: 20 mM Tris pH 8, 100 MM NaCl, 1 mM EDTA, 2.5 mM EGTA, 10 mM MgCl₂, 2 mM DTT, 0.4 mM AEBSF, 0.1% n-octylglucopyranoside, 1 nM okadeic acid, and 10 μg/mL each of benzamidine, leupeptin, pepstatin, and aprotinin.
5× Reaction buffer: 125 mM HEPES pH=8, 25 mM MgCl₂, 5 mM EDTA, 5 mM Na₃VO₄, 100 μg/mL BSA.
Enzyme dilution buffer: 25 mM HEPES pH 8, 1 mM EDTA, 1 mM Na₃VO₄, 400 μg/mL BSA.
Stop solution: 100 mM EDTA, 80 mM sodium pyrophosphate.
Filter plates: Milipore multiscreen # SE3MO78E3, Immobilon-P (PVDF).
   Methods:
Protein purification: Sf9 cells were infected with baculovirus and grown as described in Williams, et al., Proceedings of the National Academy of Science, U.S.A. pp 2922-2926, 1992. All subsequent steps were preformed on ice or at 4° C. Cells were pelleted and lysed by sonication in column buffer. Lysates were spun at 17,000×g for 20 min, followed by 0.22 μm filtration. Epitope tagged proteins were purified by chromatography over GammaBind Plus affinity column to which the “Glu-Glu” antibody was coupled. Proteins were loaded on the column followed by sequential washes with two column volumes of column buffer, and eluted with 50 μg/mL Glu-Tyr-Met-Pro-Met-Glu in column buffer.
Raf kinase assay: Test compounds were evaluated using ten 3-fold serial dilutions starting at 10-100μM. 10 μL of the test inhibitor or control, dissolved in 10% DMSO, was added to the assay plate followed by the addition of 30 μL of the a mixture containing 10 μL 5× reaction buffer, 1 mM ³³P-γ-ATP (20 μCi/mL), 0.5 μL MEK (2.5 mg/mL), 1 μL 50 mM β-mercaptoethanol. The reaction was started by the addition of 10 μL of enzyme dilution buffer containing 1 mM DTT and an amount of activated Raf that produces linear kinetics over the reaction time course. The reaction was mixed and incubated at RT for 90 min and stopped by the addition of 50 μL stop solution. 90 μL aliquots of this stopped solution were transferred onto GFP-30 cellulose microtiter filter plates (Polyfiltronics), the filter plates washed in four well volumes of 5% phosphoric acid, allowed to dry, and then replenished with 25 μL scintillation cocktail. The plates were counted for ³³P gamma emission using a TopCount Scintillation Reader.

While the compounds of the invention can be administered as the sole active pharmaceutical agent, they can also be used in combination with one or more compounds of the invention or other agents. When administered as a combination, the therapeutic agents can be formulated as separate compositions that are given at the same time or different times, or the therapeutic agents can be given as a single composition.

The foregoing is merely illustrative of the invention and is not intended to limit the invention to the disclosed compounds. Variations and changes, which are obvious to one skilled in the art, are intended to be within the scope and nature of the invention, which are defined, in the appended claims.

From the foregoing description, one skilled in the art can easily ascertain the essential characteristics of this invention, and without departing from the spirit and scope thereof, can make various changes and modifications of the invention to adapt it to various usages and conditions.

For the treatment of TNF-α, IL-1β, IL-6, and IL-8 mediated diseases, cancer, and/or hyperglycemia, the compounds of the present invention may be administered orally, parentally, by inhalation spray, rectally, or topically in dosage unit formulations containing conventional pharmaceutically acceptable carriers, adjuvants, and vehicles. The term parenteral as used herein includes, subcutaneous, intravenous, intramuscular, intrasternal, infusion techniques or intraperitoneally.

Treatment of diseases and disorders herein is intended to also include the prophylactic administration of a compound of the invention, a pharmaceutical salt thereof, or a pharmaceutical composition of either to a subject (i.e., an animal, preferably a mammal, most preferably a human) believed to be in need of preventative treatment, such as, for example, pain, inflammation and the like.

The dosage regimen for treating a TNF-α, IL-1, IL-6, and IL-8 mediated diseases, cancer, and/or hyperglycemia with the compounds of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods. Dosage levels of the order from about 0.01 mg to 30 mg per kilogram of body weight per day, preferably from about 0.1 mg to 10 mg/kg, more preferably from about 0.25 mg to 1 mg/kg are useful for all methods of use disclosed herein.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a capsule, a tablet, a suspension, or liquid. The pharmaceutical composition is preferably made in the form of a dosage unit containing a given amount of the active ingredient. For example, these may contain an amount of active ingredient from about 1 to 2000 mg, preferably from about 1 to 500 mg, more preferably from about 5 to 150 mg. A suitable daily dose for a human or other mammal may vary widely depending on the condition of the patient and other factors, but, once again, can be determined using routine methods.

The active ingredient may also be administered by injection as a composition with suitable carriers including saline, dextrose, or water. The daily parenteral dosage regimen will be from about 0.1 to about 30 mg/kg of total body weight, preferably from about 0.1 to about 10 mg/kg, and more preferably from about 0.25 mg to 1 mg/kg.

Injectable preparations, such as sterile injectable aqueous or oleaginous suspensions, may be formulated according to the known are using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

A suitable topical dose of active ingredient of a compound of the invention is 0.1 mg to 150 mg administered one to four, preferably one or two times daily. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

Formulations suitable for topical administration include liquid or semi-liquid preparations suitable for penetration through the skin (e.g., liniments, lotions, ointments, creams, or pastes) and drops suitable for administration to the eye, ear, or nose.

For administration, the compounds of this invention are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinyl-pyrrolidine, and/or polyvinyl alcohol, and tableted or encapsulated for conventional administration. Alternatively, the compounds of this invention may be dissolved in saline, water, polyethylene glycol, propylene glycol, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be made up in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting, sweetening, flavoring, and perfuming agents.

What is claimed is:

1. A compound of the formula 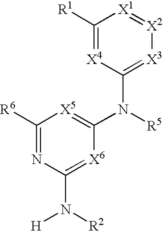
or a pharmaceutically acceptable salt or hydrate thereof, wherein
X¹ is N or CR³;
X² is N or CR⁴; or −X¹═X²— is —C(═O)—N(R^{a})— or —N(R^{a})—C(═O)—;
X³ is N or CR⁴;
X⁴ is N or CR⁴;
X⁵ is N or CR⁶;
X⁶ is N or CR⁶; wherein only 1, 2 or 3 of X¹, X², X³ and X⁴ are N;
R¹is a saturated, partially saturated or unsaturated 5-, 6- or 7-membered, ring containing 0, 1, 2 or 3 atoms selected from N, O and S, wherein the ring is substituted by 0, 1, 2 or 3 substituents selected from C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a};
R² is C₁₋₈alkyl substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NRₐRₐ, —NR^{a}C₂₋₆alkylNR^{a}R^{a} and —NR^{a}C₂₋₆alkylOR; or
R² is a saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1 or 2 C₁₋₈alkyl groups, each being substituted by 0, 1, 2 or 3 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —C(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e} and —N(R^{a})C(═O)NR^{a}R^{e}, and additionally substituted by 0, 1 or 2 saturated, partially saturated or unsaturated 5-, 6- or 7-membered monocyclic or 6-, 7-, 8-, 9-, 10- or 11-membered bicyclic rings containing 0, 1, 2, 3 or 4 atoms selected from N, O and S, wherein the carbon atoms of the rings are substituted by 0, 1 or 2 oxo groups and the rings is substituted by 0, 1, 2 or 3 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —ORₐ, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I;
R³ is independently, in each instance, selected from H, R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —NR^{a}R^{e}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R_{b}, —N(R^{a})S(═O)₂NR^{a}R^{a}, —NR^{a}C₂₋₆alkylNR^{a}R^{a} and —NR^{a}C₂₋₆alkylOR^{a};
R⁴ is independently in each instance H, R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{b}, —OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —NR^{a}R^{e}, —N(R^{a})C(═O)₂R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a};
R⁵ is H, R^{e}, C₁₋₄haloalkyl, —C(═O)R^{b}, —C(═O)OR^{b}, —C(═O)NR^{a}R^{a} or —C(═NR^{a})NR^{a}R^{a};
R⁶ is independently in each instance H, C₁₋₈alkyl, C₁₋₄haloalkyl, —NR^{a}R^{a}, —OR^{a}, or halo;
R^{a} is independently, at each instance, H or R^{b};
R^{b} is independently, at each instance, phenyl, benzyl or C₁₋₆alkyl, the phenyl, benzyl and C₁₋₆alkyl being substituted by 0, 1, 2 or 3 substituents selected from halo, C₁₋₄alkyl, C₁₋₃haloalkyl, —OC₁₋₄alkyl, —NH₂, —NHC₁₋₄alkyl, —N(C₁₋₄alkyl)C₁₋₄alkyl;
R^{d} is independently at each instance C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylOR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a};
R^{e} is independently at each instance C₁₋₆alkyl substituted by 0, 1, 2 or 3 substituents independently selected from R^{d} and additionally substituted by 0 or 1 substituents selected from R^{g}; and
R^{g} is independently at each instance a saturated, partially saturated or unsaturated 5-, 6- or 7-membered monocyclic or 6-, 7-, 8-, 9-, 10- or 11 -membered bicyclic ring containing 0, 1, 2, 3 or 4 atoms selected from N, O and S, wherein the carbon atoms of the ring are substituted by 0, 1 or 2 oxo groups and the ring is substituted by 0, 1, 2 or 3 substituents selected from R^{b}, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from Br, Cl, F and I.

2. The compound according to Claim 1, wherein
R¹ is a ring selected from phenyl, pyridyl, pyrimidinyl, pyridazine, pyrazine, pyrazole, imidazole, triazole, thiophene, furan, thiazole and oxazole, wherein the ring is substituted by 0, 1, 2 or 3 substituents selected from C₁₋₄alkyl, C₁₋₄ haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR;
R² is C₂₋₈ alkyl substituted by 1 or 2 substituents selected from C₁₋₂haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, —C(═O)R^{g}, —C(═O)OR^{g}, —C(═O)NR^{a}R^{g}, —C(═NR^{a})NR^{a}R^{g}, —OR^{g}, —OC(═O)R^{g}, —OC(═O)NR^{a}R^{g}, —OC(═O)N(R^{a})S(═O)₂R^{g}, —OC₂₋₆alkylNR^{a}R^{g}, —OC₂₋₆alkylOR^{g}, —SR^{g}, —S(═O)R^{g}, —S(═O)₂R^{g}, —S(═O)₂NR^{a}R^{g}, —NR^{a}R^{g}, —N(R^{a})C(═O)R^{g}, —N(R^{a})C(═O)OR^{g}, —N(R^{a})C(═O)NR^{a}R^{g}, —C(═O)R^{e}, —C(═O)OR^{e}, —C(═O)NR^{a}R^{e}, —C(═NR^{a})NR^{a}R^{e}, —OR^{e}, —OC(═O)R^{e}, —OC(═O)NR^{a}R^{e}, —OC(═O)N(R^{a})S(═O)₂R^{e}, —OC₂₋₆alkylNR^{a}R^{e}, —OC₂₋₆alkylOR^{e}, —SR^{e}, —S(═O)R^{e}, —S(═O)₂R^{e}, —S(═O)₂NR^{a}R^{e}, —NR^{a}R^{e}, —N(R^{a})C(═O)R^{e}, —N(R^{a})C(═O)OR^{e}, —N(R^{a})C(═O)NR^{a}R^{e} and a ring selected from phenyl, pyridyl, pyrimidinyl, pyridazine, pyrazine, pyrazole, imidazole, triazole, thiophene, furan, thiazole and oxazole, wherein the ring is substituted by 0, 1 or 2 substituents selected from R^{e}, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR_{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein any part of R² is additionally substituted by 0, 1, 2, 3, 4, 5 or 6 atoms selected from Br, Cl, F and I;
R³ is independently, in each instance, selected from H, R^{e}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OR^{e}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —(═O)₂N(R^{a})C(═O)OR^{b}, —S(═O)₂N(R^{a})C(═O)NR^{a}R^{a}, —NR^{a}R^{a}, —NR^{a}R^{e}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a};
R⁴ is H, R^{d}, R^{e} or R^{g};
R⁵ is H, R^{e} or R^{g}; and
R⁶ is H.

3. The compound according to Claim 1 that is selected from:
(1R)-2-((4-(methyl(2-phenyl-4-pyrimidinyl)amino)-2-pyrimidinyl)amino)-1-phenylethanol;
(1S)-2-((4-(methyl(2-phenyl-4-pyrimidinyl)amino)-2-pyrimidinyl)amino)-1-phenylethanol;
(1S)-2-((4-(methyl(2-phenyl-4-pyrimidinyl)amino)-2-pyrimidinyl)amino)-1-(2-pyridinyl)ethanol;
(2R)-2-((4-(methyl(2-phenyl-4-pyrimidinyl)amino)-2-pyrimidinyl)amino)-3-phenyl-1-propanol;
(3-((2R)-2-((4-((2-chloro-6-phenyl-4-pyridinyl)(methyl)amino)-2-pyrimidinyl)amino)propyl)phenyl)methanol;
(3-((2R)-2-((4-((6-(3-fluorophenyl)-2-pyridinyl)(methyl)amino)-2-pyrimidinyl)amino)propyl)phenyl)methanol;
(3-((2S)-2-((4-((6-amino-2-phenyl-4-pyrimidinyl)(methyl)amino)-2-pyrimidinyl)amino)propyl)phenyl)methanol;
(3R)-3-((4-(methyl(1-methyl-6-oxo-5-phenyl-1,6-dihydro-3-pyridinyl)amino)-2-pyrimidinyl)amino)-4-phenylbutanoic acid;
(3R)-3-((4-(methyl(2-phenyl-4-pyrimidinyl)amino)-2-pyrimidinyl)amino)-4-phenyl-1-butanol;
(3R)-3-((4-(methyl(2-phenyl-4-pyrimidinyl)amino)-2-pyrimidinyl)amino)-3-phenyl-1-propanol;
(3S)-3-((4-(methyl(2-phenyl-4-pyrimidinyl)amino)-2-pyrimidinyl)amino)-3-phenyl-1-propanol;
(3S)-3-((4-(methyl(2-phenyl-4-pyrimidinyl)amino)-2-pyrimidinyl)amino)-3-phenyl-1-propanol;
1,1-dimethylethyl (1S)-1-(3-(2-((4-(methyl(2-phenyl-4-pyrimidinyl)amino)-2-pyrimidinyl)amino)ethyl)phenyl)ethylcarbamate;
1,1-dimethylethyl(1S)-1-(4-(2-((4-(methyl(2-phenyl-4-pyrimidinyl)amino)-2-pyrimidinyl)amino)ethyl)phenyl)ethylcarbamate;
1,1-dimethylethyl(3-((2S)-2-((4-(methyl(1-methyl-6-oxo-5-phenyl-1,6-dihydro-3-pyridinyl)amino)-2-pyrimidinyl)amino)propyl)phenyl)methylcarbamate;
1,1-dimethylethyl 2-methyl-2-((4-(methyl(1-methyl-6-oxo-4-phenyl-1,6-dihydro-2-pyridinyl)amino)-2-pyrimidinyl)amino)propylcarbamate;
1,1-dimethylethyl-2-methyl-2-((4-(methyl(2-phenyl-4-pyrimidinyl)amino)-2-pyrimidinyl)amino)propylcarbamate;
1,1-dimethylethyl-4-((4-(methyl(1-methyl-6-oxo-4-phenyl-1,6-dihydro-2-pyridinyl)amino)-2-pyrimidinyl)amino)-1-piperidinecarboxylate;
1,1-dimethylethyl-4-((4-(methyl(1-methyl-6-oxo-5-phenyl-1,6-dihydro-3-pyridinyl)amino)-2-pyrimidinyl)amino)-1-piperidinecarboxylate;
1-methyl-5-((2-(methylsulfanyl)-4-pyrimidinyl)amino)-3-phenyl-2(1H)-pyridinone;
1-methyl-5-(methyl(2-((2-phenylethyl)amino)-4-pyrimidinyl)amino)-3-phenyl-2(1H)-pyridinone;
1-methyl-5-(methyl(2-(4-piperidinylamino)-4-pyrimidinyl)amino)-3-phenyl-2(1H)-pyridinone;
2-phenyl-4-((2-((2-(3-pyridinyl)ethyl)amino)-4-pyrimidinyl)amino)-5-pyrimidinecarboxamide;
3-(3-((1S)-1-((4-(methyl(2-phenyl-4-pyrimidinyl)amino)-2-pyrimidinyl)amino)ethyl)phenyl)propanoic acid;
4-((2-(((1S)-2-(3-(aminomethyl)phenyl)-1-methylethyl)amino)-4-pyrimidinyl)amino)-N-methyl-2-phenyl-5-pyrimidinecarboxamide;
4-(methyl(2-((2-(3-pyridinyl)ethyl)amino)-4-pyrimidinyl)amino)-2-phenyl-5-pyrimidinecarboxamide;
4-chloro-3-((2S)-2-((4-(methyl(2-phenyl-4-pyrimidinyl)amino)-2-pyrimidinyl)amino)propyl)benzonitrile;
5-((2-(((1R)-2-(3-(aminomethyl)phenyl)-1-methylethyl)amino)-4-pyrimidinyl)(methyl)amino)-3-phenyl-2(1H)-pyridinone;
5-((2-(((1R)-2-(4-fluoro-3-(hydroxymethyl)phenyl)-1-methylethyl)amino)-4-pyrimidinyl)(methyl)amino)-1-methyl-3-phenyl-2(1H)-pyridinone;
5-((2-(((1S)-2-(3-((1R)-1-aminoethyl)phenyl)-1-methylethyl)amino)-4-pyrimidinyl)(methyl)amino)-3-phenyl-2(1H)-pyridinone;
5-((2-(((1S)-2-(3-((1R)-1-aminoethyl)phenyl)-1-methylethyl)amino)-4-pyrimidinyl)(methyl)amino)-1-methyl-3-phenyl-2(1H)-pyridinone;
5-((2-(((1S)-2-(3-((1S)-1-aminoethyl)phenyl)-1-methylethyl)amino)-4-pyrimidinyl)(methyl)amino)-3-phenyl-2(1H)-pyridinone;
5-((2-(((1S)-2-(3-(aminomethyl)phenyl)-1-methylethyl)amino)-4-pyrimidinyl)(methyl)amino)-3-phenyl-2(1H)-pyridinone;
5-((2-(((1S)-2-(3-(aminomethyl)phenyl)-1-methylethyl)amino)-4-pyrimidinyl)(methyl)amino)-1-(1-methylethyl)-3-phenyl-2(1H)-pyridinone;
5-((2-(((1S)-2-(3-(aminomethyl)phenyl)-1-methylethyl)amino)-4-pyrimidinyl)(methyl)amino)-1-methyl-3-phenyl-2(1H)-pyridinone;
5-((2-(((1S)-2-(3-(aminomethyl)phenyl)-1-methylethyl)amino)-4-pyrimidinyl)(methyl)amino)-3-(2-fluorophenyl)-2(1H)-pyridinone;
5-((2-((1-acetyl-4-piperidinyl)amino)-4-pyrimidinyl)(methyl)amino)-3-phenyl-2(1H)-pyridinone;
5-((2-((1-acetyl-4-piperidinyl)amino)-4-pyrimidinyl)(methyl)amino)-1-methyl-3-phenyl-2(1H)-pyridinone;
5-(methyl(2-((2-phenylethyl)amino)-4-pyrimidinyl)amino)-3-phenyl-2(1H)-pyridinone;
5-fluoro-N-4-(5-fluoro-2-phenyl-4-pyrimidinyl)-N-4-methyl-N-2-(2-(3-pyridinyl)ethyl)-2,4-pyrimidinediamine;
5-fluoro-N-4-methyl-N-4-(2-phenyl-4-pyrimidinyl)-N-2-(2-(3-pyridinyl)ethyl)-2,4-pyrimidinediamine;
6-((2-((2-(2-chlorophenyl)ethyl)amino)-4-pyrimidinyl)amino)-1-methyl-4-phenyl-2(1H)-pyridinone;
6-(methyl(2-((2-(2-pyridinyl)ethyl)amino)-4-pyrimidinyl)amino)-2-phenyl-4-pyrimidinol;
6-chloro-5-phenyl-N-(2-((2S)-2-(phenylmethyl)-1-pyrrolidinyl)-4-pyrimidinyl)-3-pyridazinamine;
ethyl 2-phenyl-4-((2-((2-(2-pyridinyl)ethyl)amino)-4-pyrimidinyl)amino)-5-pyrimidinecarboxylate;
N-(4-((4-(methyl(2-phenyl-4-pyrimidinyl)amino)-2-pyrimidinyl)amino)cyclohexyl)acetamide;
N-(4-(methyl(2-phenyl-4-pyrimidinyl)amino)-2-pyrimidinyl)-D-phenylalaninamide;
N-(4-(methyl(2-phenyl-4-pyrimidinyl)amino)-2-pyrimidinyl)-D-phenylalanine;
N-1-((2R)-2-((4-(methyl(2-phenyl-4-pyrimidinyl)amino)-2-pyrimidinyl)amino)-3-phenylpropyl)glycinamide;
N-1-((3-((2S)-2-((4-(methyl(4-(methyloxy)-6-phenyl-1,3,5-triazin-2-yl)amino)-2-pyrimidinyl)amino)propyl)phenyl)methyl)-L-alaninamide;
N-1-((3-((2S)-2-((4-methyl-6-(methyl(2-phenyl-4-pyrimidinyl)amino)-2-pyrimidinyl)amino)propyl)phenyl)methyl)-L-alaninamide;
N-1-(4-((4-(methyl(2-phenyl-4-pyrimidinyl)amino)-2-pyrimidinyl)amino)cyclohexyl)-L-alaninamide;
N-2-((1-acetyl-4-piperidinyl)methyl)-N-4-methyl-N-4-(2-phenyl-4-pyrimidinyl)-2,4-pyrimidinediamine;
N-2-((1R)-2-((2-aminoethyl)amino)-1-(phenylmethyl)ethyl)-N-4-methyl-N-4-(2-phenyl-4-pyrimidinyl)-2,4-pyrimidinediamine;
N-2-((1R)-2-(3-(aminomethyl)phenyl)-1-methylethyl)-N-4-(2-chloro-6-phenyl-4-pyridinyl)-N-4-methyl-2,4-pyrimidinediamine;
N-2-((1R)-2-(3-(aminomethyl)phenyl)-1-methylethyl)-N-4-(6-(3-fluorophenyl)-2-pyridinyl)-N-4-methyl-2,4-pyrimidinediamine;
N-2-((1R)-2-amino-1-(phenylmethyl)ethyl)-N-4-methyl-N-4-(2-phenyl-4-pyrimidinyl)-2,4-pyrimidinediamine;
N-2-((1R)-3-(cyclopropylamino)-1-(phenylmethyl)propyl)-N-4-methyl-N-4-(2-phenyl-4-pyrimidinyl)-2,4-pyrimidinediamine;
N-2-((1R)-3-amino-1-(phenylmethyl)propyl)-N-4-methyl-N-4-(2-phenyl-4-pyrimidinyl)-2,4-pyrimidinediamine;
N-2-((1S)-1-((1R,3S)-3-(2-aminoethyl)cyclohexyl)ethyl)-N-4-methyl-N-4-(2-phenyl-4-pyrimidinyl)-2,4-pyrimidinediamine;
N-2-((1S)-1-(3-(2-aminoethyl)phenyl)ethyl)-N-4-methyl-N-4-(2-phenyl-4-pyrimidinyl)-2,4-pyrimidinediamine;
N-2-((1S)-1-(4-(2-aminoethyl)phenyl)ethyl)-N-4-methyl-N-4-(2-phenyl-4-pyrimidinyl)-2,4-pyrimidinediamine;
N-2-((1S)-2-(3-((1R)-1-aminoethyl)phenyl)-1-methylethyl)-N-4-,6-dimethyl-N-4-(2-phenyl-4-pyrimidinyl)-2,4-pyrimidinediamine;
N-2-((1S)-2-(3-((1R)-1-aminoethyl)phenyl)-1-methylethyl)-N-4-methyl-N-4-(2-phenyl-4-pyrimidinyl)-2,4-pyrimidinediamine;
N-2-((1S)-2-(3-((1R)-1-aminoethyl)phenyl)-1-methylethyl)-N-4-methyl-N-4-(4-(methyloxy)-6-phenyl-1,3,5-triazin-2-yl)-2,4-pyrimidinediamine;
N-2-((1S)-2-(3-((1S)-1-aminoethyl)phenyl)-1-methylethyl)-N-4-methyl-N-4-(2-phenyl-4-pyrimidinyl)-2,4-pyrimidinediamine;
N-2-((1S)-2-(3-(1-amino-1-methylethyl)phenyl)-1-methylethyl)-N-4-methyl-N-4-(2-phenyl-4-pyrimidinyl)-2,4-pyrimidinediamine;
N-2-((1S)-2-(3-(1-aminocyclopropyl)phenyl)-1-methylethyl)-N-4-methyl-N-4-(2-phenyl-4-pyrimidinyl)-2,4-pyrimidinediamine;
N-2-((1S)-2-(3-(1H-imidazol-1-yl)phenyl)-1-methylethyl)-N-4-methyl-N-4-(2-phenyl-4-pyrimidinyl)-2,4-pyrimidinediamine;
N-2-((1S)-2-(3-(aminomethyl)phenyl)-1-methylethyl)-N-4-(2-(2,4-difluorophenyl)-4-pyrimidinyl)-N-4-methyl-2,4-pyrimidinediamine;
N-2-((1S)-2-(3-(aminomethyl)phenyl)-1-methylethyl)-N-4-(2-(2-fluorophenyl)-4-pyrimidinyl)-N-4-methyl-2,4-pyrimidinediamine;
N-2-((1S)-2-(3-(aminomethyl)phenyl)-1-methylethyl)-N-4-(2-(3-fluorophenyl)-4-pyrimidinyl)-N-4-methyl-2,4-pyrimidinediamine;
N-2-((1S)-2-(3-(aminomethyl)phenyl)-1-methylethyl)-N-4-(2-(4-fluorophenyl)-4-pyrimidinyl)-N-4-methyl-2,4-pyrimidinediamine;
N-2-((1S)-2-(3-(aminomethyl)phenyl)-1-methylethyl)-N-4-(5-fluoro-2-phenyl-4-pyrimidinyl)-N-4-methyl-2,4-pyrimidinediamine;
N-2-((1S)-2-(3-(aminomethyl)phenyl)-1-methylethyl)-N-4-(5-fluoro-2-(2-fluorophenyl)-4-pyrimidinyl)-N-4-methyl-2,4-pyrimidinediamine;
N-2-((1S)-2-(3-(aminomethyl)phenyl)-1-methylethyl)-N-4-(6-amino-2-phenyl-4-pyrimidinyl)-N-4-methyl-2,4-pyrimidinediamine;
N-2-((1S)-2-(3-(aminomethyl)phenyl)-1-methylethyl)-N-4-methyl-N-4-(2-phenyl-4-pyrimidinyl)-2,4-pyrimidinediamine;
N-2-((1S)-2-(3-(aminomethyl)phenyl)-1-methylethyl)-N-4-methyl-N-4-(4-(methyloxy)-6-phenyl-1,3 ,5-triazin-2-yl)-2,4-pyrimidinediamine;
N-2-((1S)-2-(3-(aminomethyl)phenyl)-1-methylethyl)-N-4-methyl-N-4-(6-phenyl-2-pyrazinyl)-2,4-pyrimidinediamine;
N-2-((1S)-2-(5-(aminomethyl)-2-chlorophenyl)-1-methylethyl)-N-4-methyl-N-4-(2-phenyl-4-pyrimidinyl)-2,4-pyrimidinediamine;
N-2-((2R)-2-(dimethylamino)-2-(3-pyridinyl)ethyl)-N-4-methyl-N-4-(2-phenyl-4-pyrimidinyl)-2,4-pyrimidinediamine;
N-2-((2R)-2-amino-2-phenylethyl)-N-4-methyl-N-4-(2-phenyl-4-pyrimidinyl)-2,4-pyrimidinediamine;
N-2-((2S)-2-(dimethylamino)-2-(3-pyridinyl)ethyl)-N-4-methyl-N-4-(2-phenyl-4-pyrimidinyl)-2,4-pyrimidinediamine;
N-2-((2S)-2-(dimethylamino)-2-(3-pyridinyl)ethyl)-N-4-methyl-N-4-(2-phenyl-4-pyrimidinyl)-2,4-pyrimidinediamine;
N-2-(1-((2S)-2-aminopropanoyl)-4-piperidinyl)-N-4-methyl-N-4-(2-phenyl-4-pyrimidinyl)-2,4-pyrimidinediamine;
N-2-(1-((3R)-3-aminobutanoyl)-4-piperidinyl)-N-4-methyl-N-4-(2-phenyl-4-pyrimidinyl)-2,4-pyrimidinediamine;
N-2-(1-(aminoacetyl)-4-piperidinyl)-N-4-methyl-N-4-(2-phenyl-4-pyrimidinyl)-2,4-pyrimidinediamine;
N-2-(1,1-dimethyl-2-phenylethyl)-N-4-methyl-N-4-(2-phenyl-4-pyrimidinyl)-2,4-pyrimidinediamine;
N-2-(1,1-dimethyl-2-phenylethyl)-N-4-methyl-N-4-(4-phenyl-2-pyrimidinyl)-2,4-pyrimidinediamine;
N-2-(1-acetyl-4-piperidinyl)-N-4-methyl-N-4-(2-phenyl-4-pyrimidinyl)-2,4-pyrimidinediamine;
N-2-(2-(((1S)-2-(3-(aminomethyl)phenyl)-1-methylethyl)amino)-4-pyrimidinyl)-N-2-methyl-6-phenyl-2,4-pyrimidinediamine;
N-2-(2-((1R,3S)-3-((1S)-1-aminoethyl)cyclohexyl)ethyl)-N-4-methyl-N-4-(2-phenyl-4-pyrimidinyl)-2,4-pyrimidinediamine;
N-2-(2-(2-chlorophenyl)ethyl)-N-4-(4-(1,1-dimethylethyl)-2-pyrimidinyl)-N-4-methyl-2,4-pyrimidinediamine;
N-2-(2-(2-chlorophenyl)ethyl)-N-4-(6-chloro-5-phenyl-3-pyridazinyl)-2,4-pyrimidinediamine;
N-2-(2-(2-chlorophenyl)ethyl)-N-4-methyl-N-4-(2-phenyl-4-pyrimidinyl)-2,4-pyrimidinediamine;
N-2-(2-(2-chlorophenyl)ethyl)-N-4-methyl-N-4-(4-phenyl-2-pyrimidinyl)-2,4-pyrimidinediamine;
N-2-(2-(2-chlorophenyl)ethyl)-N-4-methyl-N-4-(6-phenyl-2-pyrazinyl)-2,4-pyrimidinediamine;
N-2-(2-(3-((1S)-1-aminoethyl)phenyl)ethyl)-N-4-methyl-N-4-(2-phenyl-4-pyrimidinyl)-2,4-pyrimidinediamine;
N-2-(2-(4-((1S)-1-aminoethyl)phenyl)ethyl)-N-4-methyl-N-4-(2-phenyl-4-pyrimidinyl)-2,4-pyrimidinediamine;
N-2-(2-amino-1,1-dimethylethyl)-N-4-methyl-N-4-(2-phenyl-4-pyrimidinyl)-2,4-pyrimidinediamine;
N-2-(4-aminocyclohexyl)-N-4-(2-(2-fluorophenyl)-4-pyrimidinyl)-N-4-methyl-2,4-pyrimidinediamine;
N-2-(4-aminocyclohexyl)-N-4-(5-fluoro-2-phenyl-4-pyridinyl)-N-4-methyl-2,4-pyrimidinediamine;
N-2-(4-aminocyclohexyl)-N-4-,6-dimethyl-N-4-(2-phenyl-4-pyrimidinyl)-2,4-pyrimidinediamine;
N-2-(4-aminocyclohexyl)-N-4-methyl-N-4-(2-phenyl-4-pyrimidinyl)-2,4-pyrimidinediamine;
N-2-(4-aminocyclohexyl)-N-4-methyl-N-4-(2-phenyl-4-pyrimidinyl)-2,4-pyrimidinediamine;
N-2-(4-aminocyclohexyl)-N-4-methyl-N-4-(6-phenyl-2-pyrazinyl)-2,4-pyrimidinediamine;
N-2-(4-aminocyclohexyl)-N-4-methyl-N-4-(6-phenyl-2-pyridinyl)-2,4-pyrimidinediamine;
N-4-(2-(2,3-difluorophenyl)-4-pyrimidinyl)-N-4-methyl-N-2-(2-(3-pyridinyl)ethyl)-2,4-pyrimidinediamine;
N-4-(2-(2,4-difluorophenyl)-4-pyrimidinyl)-N-4-methyl-N-2-(2-(3-pyridinyl)ethyl)-2,4-pyrimidinediamine;
N-4-(2-(2,5-difluorophenyl)-4-pyrimidinyl)-N-4-methyl-N-2-(2-(3-pyridinyl)ethyl)-2,4-pyrimidinediamine;
N-4-(2-(2-fluorophenyl)-4-pyrimidinyl)-N-4-methyl-N-2-(2-(3-pyridinyl)ethyl)-2,4-pyrimidinediamine;
N-4-(2-(3-fluorophenyl)-4-pyrimidinyl)-N-4-methyl-N-2-(2-(3-pyridinyl)ethyl)-2,4-pyrimidinediamine;
N-4-(2-(4-fluorophenyl)-4-pyrimidinyl)-N-4-methyl-N-2-(2-(3-pyridinyl)ethyl)-2,4-pyrimidinediamine;
N-4-(4-(1,1-dimethylethyl)-2-pyrimidinyl)-N-4-methyl-N-2-(2-phenylethyl)-2,4-pyrimidinediamine;
N-4-(5-bromo-2-phenyl-4-pyrimidinyl)-N-4-methyl-N-2-(2-(3-pyridinyl)ethyl)-2,4-pyrimidinediamine;
N-4-(6-(1-cyclohexen-1-yl)-2-pyridinyl)-N-4-methyl-N-2-(2-phenylethyl)-2,4-pyrimidinediamine;
N-4-(6-(2,3-difluorophenyl)-2-pyridinyl)-N-4-methyl-N-2-(2-phenylethyl)-2,4-pyrimidinediamine;
N-4-(6-(2-chlorophenyl)-2-pyridinyl)-N-4-methyl-N-2-(2-phenylethyl)-2,4-pyrimidinediamine;
N-4-(6-(2-furanyl)-2-pyridinyl)-N-4-methyl-N-2-(2-phenylethyl)-2,4-pyrimidinediamine;
N-4-(6-(3,4-difluorophenyl)-2-pyridinyl)-N-4-methyl-N-2-(2-phenylethyl)-2,4-pyrimidinediamine;
N-4-(6-(3,5-difluorophenyl)-2-pyridinyl)-N-4-methyl-N-2-(2-phenylethyl)-2,4-pyrimidinediamine;
N-4-(6-(3-chlorophenyl)-2-pyridinyl)-N-4-methyl-N-2-(2-phenylethyl)-2,4-pyrimidinediamine;
N-4-(6-(3-furanyl)-2-pyridinyl)-N-4-methyl-N-2-(2-phenylethyl)-2,4-pyrimidinediamine;
N-4-(6-(4-chlorophenyl)-2-pyridinyl)-N-4-methyl-N-2-(2-phenylethyl)-2,4-pyrimidinediamine;
N-4-methyl-N-2-((1R)-2-((1-methylethyl)amino)-1-(phenylmethyl)ethyl)-N-4-(2-phenyl-4-pyrimidinyl)-2,4-pyrimidinediamine;
N-4-methyl-N-2-((1R)-2-(4-morpholinyl)-1-(phenylmethyl)ethyl)-N-4-(2-phenyl-4-pyrimidinyl)-2,4-pyrimidinediamine;
N-4-methyl-N-2-((1R)-3-((1-methylethyl)amino)-1-(phenylmethyl)propyl)-N-4-(2-phenyl-4-pyrimidinyl)-2,4-pyrimidinediamine;
N-4-methyl-N-2-((1R)-3-(4-morpholinyl)-1-(phenylmethyl)propyl)-N-4-(2-phenyl-4-pyrimidinyl)-2,4-pyrimidinediamine;
N-4-methyl-N-2-((1S)-1-(1-methylethyl)-3-(4-morpholinyl)-3-oxopropyl)-N-4-(2-phenyl-4-pyrimidinyl)-2,4-pyrimidinediamine;
N-4-methyl-N-2-((1S)-1-methyl-2-(3-(2-methyl-1H-imidazol-1-yl)phenyl)ethyl)-N-4-(2-phenyl-4-pyrimidinyl)-2,4-pyrimidinediamine;
N-4-methyl-N-2-((1S)-2-methyl-1-(2-(4-morpholinyl)ethyl)propyl)-N-4-(2-phenyl-4-pyrimidinyl)-2,4-pyrimidinediamine;
N-4-methyl-N-2-((2S)-2-(4-morpholinyl)-2-(3-pyridinyl)ethyl)-N-4-(2-phenyl-4-pyrimidinyl)-2,4-pyrimidinediamine;
N-4-methyl-N-2-(2-(3-pyridinyl)ethyl)-N-4-(2-(2-(trifluoromethyl)phenyl)-4-pyrimidinyl)-2,4-pyrimidinediamine;
N-4-methyl-N-2-(2-(3-pyridinyl)ethyl)-N-4-(2-(2-thienyl)-4-pyrimidinyl)-2,4-pyrimidinediamine;
N-4-methyl-N-2-(2-(3-pyridinyl)ethyl)-N-4-(2-(3-thienyl)-4-pyrimidinyl)-2,4-pyrimidinediamine;
N-4-methyl-N-2-(2-(4-morpholinyl)ethyl)-N-4-(2-phenyl-4-pyrimidinyl)-2,4-pyrimidinediamine;
N-4-methyl-N-2-(2-phenylethyl)-N-4-(2-phenyl-4-pyrimidinyl)-2,4-pyrimidinediamine;
N-4-methyl-N-2-(2-phenylethyl)-N-4-(4-phenyl-2-pyrimidinyl)-2,4-pyrimidinediamine;
N-4-methyl-N-2-(2-phenylethyl)-N-4-(6-(2-(trifluoromethyl)phenyl)-2-pyridinyl)-2,4-pyrimidinediamine;
N-4-methyl-N-2-(2-phenylethyl)-N-4-(6-(2-thienyl)-2-pyridinyl)-2,4-pyrimidinediamine;
N-4-methyl-N-2-(2-phenylethyl)-N-4-(6-(3-(trifluoromethyl)phenyl)-2-pyridinyl)-2,4-pyrimidinediamine;
N-4-methyl-N-2-(2-phenylethyl)-N-4-(6-(3-thienyl)-2-pyridinyl)-2,4-pyrimidinediamine;
N-4-methyl-N-2-(2-phenylethyl)-N-4-(6-(4-(trifluoromethyl)phenyl)-2-pyridinyl)-2,4-pyrimidinediamine;
N-4-methyl-N-2-(2-phenylethyl)-N-4-(6-(phenylmethyl)-2-pyridinyl)-2,4-pyrimidinediamine;
N-4-methyl-N-4-(2-(2-(methyloxy)phenyl)-4-pyrimidinyl)-N-2-(2-(3-pyridinyl)ethyl)-2,4-pyrimidinediamine;
N-4-methyl-N-4-(2-(2-methylphenyl)-4-pyrimidinyl)-N-2-(2-(3-pyridinyl)ethyl)-2,4-pyrimidinediamine;
N-4-methyl-N-4-(2-phenyl-4-pyrimidinyl)-N-2-(2-(2-pyridinyl)ethyl)-2,4-pyrimidinediamine;
N-4-methyl-N-4-(2-phenyl-4-pyrimidinyl)-N-2-(2-(3-(2-pyridinyl)phenyl)ethyl)-2,4-pyrimidinediamine;
N-4-methyl-N-4-(2-phenyl-4-pyrimidinyl)-N-2-(2-(3-pyridinyl)ethyl)-2,4-pyrimidinediamine;
N-4-methyl-N-4-(2-phenyl-4-pyrimidinyl)-N-2-(2-(5,6,7,8-tetrahydro-1,8-naphthyridin-2-yl)ethyl)-2,4-pyrimidinediamine;
N-4-methyl-N-4-(2-phenyl-4-pyrimidinyl)-N-2-(4-piperidinyl)-2,4-pyrimidinediamine;
N-4-methyl-N-4-(5-phenyl-6-((phenylmethyl)oxy)-3-pyridinyl)-N-2-(4-piperidinyl)-2,4-pyrimidinediamine;
N-4-methyl-N-4-(6-(2-(methyloxy)phenyl)-2-pyridinyl)-N-2-(2-phenylethyl)-2,4-pyrimidinediamine;
N-4-methyl-N-4-(6-(2-methylphenyl)-2-pyridinyl)-N-2-(2-phenylethyl)-2,4-pyrimidinediamine;
N-4-methyl-N-4-(6-(3-(methyloxy)phenyl)-2-pyridinyl)-N-2-(2-phenylethyl)-2,4-pyrimidinediamine;
N-4-methyl-N-4-(6-(3-methylphenyl)-2-pyridinyl)-N-2-(2-phenylethyl)-2,4-pyrimidinediamine;
N-4-methyl-N-4-(6-(4-(methyloxy)phenyl)-2-pyridinyl)-N-2-(2-phenylethyl)-2,4-pyrimidinediamine;
N-4-methyl-N-4-(6-(4-methylphenyl)-2-pyridinyl)-N-2-(2-phenylethyl)-2,4-pyrimidinediamine;
N-4-methyl-N-4-(6-(methyloxy)-5-phenyl-3-pyridinyl)-N-2-(2-phenylethyl)-2,4-pyrimidinediamine;
N-methyl-2-(2-methyl-1H-imidazol-1-yl)-N-(2-phenyl-4-pyrimidinyl)-4-pyrimidinediamine;
N-methyl-2-phenyl-4-((2-((2-(2-pyridinyl)ethyl)amino)-4-pyridinyl)amino)-5-pyrimidinecarboxamide;
N-methyl-2-phenyl-4-((2-((2-(3-pyridinyl)ethyl)amino)-4-pyrimidinyl)amino)-5-pyrimidinecarboxamide;
N-methyl-2-phenyl-N-(2-((2R)-2-(phenylmethyl)-1-azetidinyl)-4-pyrimidinyl)-4-pyrimidinamine;
N-methyl-4-((2-(((1S)-1-methyl-2-(3-((((methylamino)carbonyl)amino)methyl)phenyl)ethyl)amino)-4-pyrimidinyl)amino)-2-phenyl-5-pyrimidinecarboxamide; and
N-methyl-N-(4-(methyl(2-phenyl-4-pyrimidinyl)amino)-2-pyrimidinyl)-D-phenylalaninamide; or a pharmaceutically-acceptable salt or hydrate thereof.

4. A pharmaceutical composition comprising a compound according to claim 1 and a pharmaceutically acceptable carrier.

5. A method of treatment of inflammation comprising administering an effective amount of a compound according to claim 1.

6. A method of treatment of rheumatoid arthritis, Pagets disease, osteoporosis, multiple myeloma, uveititis, acute or chronic myelogenous leukemia, pancreatic β cell destruction, osteoarthritis, rheumatoid spondylitis, gouty arthritis, inflammatory bowel disease, adult respiratory distress syndrome (ARDS), psoriasis, Crohn's disease, allergic rhinitis, ulcerative colitis, anaphylaxis, contact dermatitis, asthma, muscle degeneration, cachexia, Reiter's syndrome, type I diabetes, type II diabetes, bone resorption diseases, graft vs. host reaction, Alzheimer's disease, stroke, myocardial infarction, ischemia reperfusion injury, atherosclerosis, brain trauma, multiple sclerosis, cerebral malaria, sepsis, septic shock, toxic shock syndrome, fever, myalgias due to HIV-1, HIV-2, HIV-3, cytomegalovirus (CMV), influenza, adenovirus, the herpes viruses or herpes zoster infection in a mammal comprising administering an effective amount of a compound according to claim 1.

7. A method of lowering plasma concentrations of either or both TNF-α and IL-1 comprising administering an effective amount of a compound according to claim 1.

8. A method of lowering plasma concentrations of either or both IL-6 and IL-8 comprising administering an effective amount of a compound according to claim 1.

9. A method of treatment of diabetes disease in a mammal comprising administering an effective amount of a compound according to claim 1 to produce a glucagon antagonist effect.

10. A method of treatment of a pain disorder in a mammal comprising administering an effective amount of a compound according to claim 1.

11. A method of decreasing prostaglandins production in a mammal comprising administering an effective amount of a compound according to claim 1.

12. A method of decreasing cyclooxygenase enzyme activity in a mammal comprising administering an effective amount of a compound according to claim 1.
